(19) United States
(12) Patent Application Publication
(10) Pub. No.: US 2003/0060611 A1
(43) Pub. Date: Mar. 27, 2003

(54) Method and reagent for the inhibition of NOGO gene

(76) Inventors: Lawrence Blatt, Boulder, CO (US); James McSwiggen, Boulder, CO (US); Bharat M. Chowrira, Broomfield, CO (US); Peter Haeberli, Berthoud, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/780,533

(22) Filed: Feb. 9, 2001

Related U.S. Application Data
(60) Provisional application No. 60/181,797, filed on Feb. 11, 2000.

(51) Int. Cl. C07H 21/02; C07H 21/04; A61K 39/00; A61K 39/38

(52) U.S. Cl. 536/23.1; 424/184.1

(57) ABSTRACT

The present invention relates to nucleic acid molecules, including antisense and enzymatic nucleic acid molecules, such as hammerhead ribozymes, DNAzymes, and antisense, which modulate the expression of NOGO gene.

### BACKGROUND OF THE INVENTION

This invention claims priority from Blatt, U.S. Ser. No. (60/181,797), filed Feb. 11, 2000, entitled “METHOD AND REAGENT FOR THE INHIBITION OF NOGO GENE”. This application is hereby incorporated by reference herein in its entirety including the drawings.

The present invention concerns compounds, compositions, and methods for the study, diagnosis, and treatment of conditions relating to the expression of NOGO gene. In particular, the invention relates to nucleic acid molecules that are used to modulate the expression of NOGO gene products.

The following is a brief description of the current understanding of NOGO. The discussion is not meant to be complete and is provided only to assist understanding the invention that follows. The summary is not an admission that any of the work described below is prior art to the claimed invention.

The ceased growth of neurons following development has severe implications for lesions of the central nervous system (CNS) caused by neurodegenerative disorders and traumatic accidents. Although CNS neurons have the capacity to rearrange their axonal and dendritic foci in the developed brain, the regeneration of severed CNS axons spanning distance does not exist. Axonal growth following CNS injury is limited by the local tissue environment rather than intrinsic factors, as indicated by transplantation experiments (Richardson et al., 1980, Nature, 284, 264-265). Non-neuronal glial cells of the CNS, including oligodendrocytes and astrocytes, have been shown to inhibit the axonal growth of dorsal root ganglion neurons in culture (Schwab and Thoenen, 1985, J. Neurosci., 5, 2415-2423). Cultured dorsal root ganglion cells can extend their axons across glial cells from the peripheral nervous system, (ie; Schwann cells), but are inhibited by oligodendrocytes and myelin of the CNS (Schwab and Caroni, 1988, J. Neurosci., 8, 2381-2393).

The non-conductive properties of CNS tissue in adult vertebrates is thought to result from the existence of inhibitory factors rather than the lack of growth factors. The identification of proteins with neurite outgrowth inhibitory or repulsive properties include NI-35, NI-250 (Caroni and Schwab, 1988, Neuron, 1, 85-96), myelin-associated glycoprotein (Genbank Accession No M29273), tenascin-R (Genbank Accession No X98085), and NG-2 (Genbank Accession No X61945). Monoclonal antibodies (mAb IN-1) raised against NI-35/250 have been shown to partially neutralize the growth inhibitory effect of CNS myelin and oligodendrocytes. IN-1 treatment in vivo has resulted in long distance fiber regeneration in lesioned adult mammalian CNS tissue (Weibel et al., 1994, Brain Res., 642, 259-266). Additionally, IN-1 treatment in vivo has resulted in the recovery of specific reflex and locomotor functions after spinal cord injury in adult rats (Bregman et al., 1995, Nature, 378, 498-501).

Recently, the cloning of NOGO-A (Genbank Accession No AJ242961), the rat complementary DNA encoding NI-220/250 has been reported (Chen et al., 2000, Nature, 403, 434-439). The NOGO gene encodes at least three major protein products (NOGO-A, B, and C) resulting from both alternative promoter usage and alternative splicing. Recombinant NOGO-A inhibits neurite outgrowth from dorsal root ganglia and the spreading of 3T3 firboblasts. Monoclonal antibody IN-1 recognizes NOGO-A and neutralizes NOGO-A inhibition of neuronal growth in vitro. Evidence supports the proposal that NOGO-A is the previously described rat NI-250 since NOGO-A contains all six peptide sequences obtained from purified bNI-220, the bovine equivalent of rat NI-250 (Chen et al supra).

Prinjha et al., 2000, Nature, 403, 383-384, report the cloning of the human NOGO gene which encodes three different NOGO isoforms that are potent inhibitors of neurite outgrowth. Using oligonucleotide primers to amplify and clone overlapping regions of the open reading frame of NOGO cDNA, Phrinjha et al., supra identified three forms of cDNA clone corresponding to the three protein isoforms. The longest ORF of 1,192 amino acids corresponds to NOGO-A (Accession No. AJ251383). An intermediate-length splice variant that lacks residues 186-1,004 corresponds to NOGO-B (Accession No. AJ251384). The shortest splice variant, NOGO-C (Accession No. AJ251385), appears to be the previously described rat vp20 (Accession No. AF051335) and foocen-s (Accession No. AF132048), and also lacks residues 186-1,004. According to Prinjha et al., supra, the NOGO amino-terminal region shows no significant homology to any known protein, while the carboxy-terminal tail shares homology with neuroendicrine-specific proteins and other members of the reticulon gene family. In addition, the carboxy-terminal tail contains a consensus sequence that may serve as an endoplasmic-reticulum retention region. Based on the NOGO protein sequence, Prinjha et al., supra, postulate NOGO to be a membrane associated protein comprising a putative large extracellular domain of 1,024 residues with seven predicted N-linked glycosylation sites, two or three transmembrane domains, and a short carboxy-terminal region of 43 residues.

Grandpre et al., 2000, Nature, also report the identification of NOGO as a potent inhibitor of axon regeneration. The 4.1 kilobase NOGO human cDNA clone identified by Grandpre et al., supra, KIAA0886, is homologous to a cDNA derived from a previous effort to sequence random high molecular-weight brain derived cDNAs (Nagase et al., 1998, DNA Res., 31, 355-364). This cDNA clone encodes a protein that matches all six of the peptide sequences derived from bovine NOGO. Grandpre et al., supra demonstrate that NOGO expression is predominantly associated with the CNS and not the peripheral nervous system (PNS). Cellular localization of NOGO protein appears to be predominantly reticluar in origin, however, NOGO is found on the surface of some oligodentrocytes. An active domain of NOGO has been identified, defined as residues 31-55 of a hydrophilic 66-residue lumenal/extracellular domain. A synthetic fragment corresponding to this sequence exhibits growth-cone collapsing and outgrowth inhibiting activities (Grandpre et al., supra).

Hauswirth and Flannery, International PCT Publication No. WO 98/48027, describe materials and methods for the specific expression of proteins in retinal photoreceptor cells consisting of an adeno-associated viral vector contacting a rod or cone-opsin promoter. In addition, ribozymes which degrade mutant mRNA are described for use in the treatment of retinitis pigmentosa.

Fechteler et al., Interanational PCT Publication No. WO 00/03004 describe ribozymes targeting presenilin-2 RNA for the use in treating neurodegenerative diseases such as Alzheimer's disease.

Eldadah et al., 2000, J. Neurosci., 20, 179-186, describe the protection of cerebellar granule cells from apoptosis induced by serum-potassium deprivation from ribozyme mediated inhibition of caspase-3.

Seidman et al., 1999, Antisense Nucleic Acid Drug Dev., 9, 333-340, describe in general terms, the use of antisense and ribozyme constructs for treatment of neurodegenerative diseases.

Denman et al, 1994, Nucleic Acids Research, 22, 2375-82, describe the ribozyme mediated degradation of beta-amyloid peptide precursor mRNA in COS-7 cells.

### SUMMARY OF THE INVENTION

The invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups] and methods for their use to modulate the expression of genes, for example, those encoding certain myelin proteins that inhibit or are involved in the inhibition of neurite growth, including axonal regeneration in the CNS. In particular, the instant invention features nucleic-acid based techniques to inhibit the expression of NOGO-A (Accession No. AJ251383), B (Accession No. AJ251384), and/or C (Accession No. AJ251385), NOGO-66 receptor (Accession No AF283463, Fournier et al., 2001, Nature, 409, 341-346), NI-35, 220, and/or 250, myelin-associated glycoprotein (Genbank Accession No M29273), tenascin-R (Genbank Accession No X98085), and NG-2 (Genbank Accession No X61945).

In a preferred embodiment, the invention features the use of one or more of the nucleic acid-based techniques independently or in combination to inhibit the expression of the gene(s) encoding NOGO-A, B, C, NI-35, 220, 250, myelin-associated glycoprotein, tenascin-R, NG-2 and/or their corresponding receptors. Specifically, the invention features the use of nucleic acid-based techniques to specifically inhibit the expression of NOGO gene (Genbank Accession No. AB020693) and NOGO-66 receptor (Genbank Accession No. AF283463).

In another preferred embodiment, the invention features the use of an enzymatic nucleic acid molecule, preferably in the hammerhead, NCH, G-cleaver, amberzyme, zinzyme and/or DNAzyme motif, to inhibit the expression of NOGO and/or NOGO receptor genes.

By “inhibit” it is meant that the activity of NOGO or level of RNAs or equivalent RNAs encoding one or more protein subunits of NOGO-A, B, C and/or NOGO receptors is reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of NOGO genes with the nucleic acid molecule of the instant invention is greater than in the presence of the nucleic acid molecule than in its absence.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention may have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel el al., EP0360257; Berzal-Herrance et al., 1993, EMBO J. 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 2 represents an Inosine nucleotide, preferably a ribo-Inosine or xylo-Inosine nucleoside.

By “G-cleaver” motif is meant, an enzymatic nucleic acid molecule comprising a motif as is generally described as G-cleaver Rz in FIG. 2. G-cleavers possess endonuclease activity to cleave RNA substrates having a cleavage triplet NYN/, where N is a nucleotide, Y is uridine or cytidine and/represents the cleavage site. G-cleavers may be chemically modified as is generally shown in FIG. 2.

By “amberzyme” motif is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and/represents the cleavage site. Zinzymes may be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition.

For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to NOGO is meant to include those naturally occurring RNA molecules having homology (partial or complete) to NOGO-A, B, C and/or NOGO receptor proteins or encoding for proteins with similar function as NOGO or NOGO receptor proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et a., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7,17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant a RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art.

Several varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in NOGO and NOGO receptor-specific RNAs represent a novel therapeutic approach to treat a variety of pathologic indications, including but not limited to CNS injury and cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO expression.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Ser. No. 09/301,511) and Zinzyme (FIG. 4) (Beigelman et al., U.S. Ser. No. 09/301,511), all included by reference herein including drawings, can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule of the instant invention can be between 13 and 100 nucleotides in length. Exemplary enzymatic nucleic acid molecules of the invention are shown in Table III-XIII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that down regulates the replication of NOGO comprises between 12 and 100 bases complementary to a RNA molecule of NOGO. Even more preferably, a nucleic acid molecule that down regulates the replication of NOGO comprises between 14 and 24 bases complementary to a RNA molecule of NOGO.

In a preferred embodiment the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding NOGO-A, B, C and/or receptor proteins (specifically NOGO and NOGO receptor genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g., bacterial cell) or eukaryotic (e.g., mammalian or plant cell).

By “NOGO proteins” is meant, a protein, protein receptor or a mutant protein derivative thereof, comprising neuronal inhibitor activity, preferably CNS neuronal growth inhibitor activity.

By “highly conserved sequence region” is meant, a nucleotide sequence of one or more regions in a target gene does not vary significantly from one generation to the other or from one biological system to the other.

The nucleic acid-based inhibitors of NOGO and NOGO receptor expression are useful for the prevention and/or treatment of diseases and conditions such CNS injury, cerebrovascular accident (CVA, stroke), Alzheimer's disease, dementia, multiple sclerosis (MS), chemotherapy-induced neuropathy, muscular dystrophy and any other diseases or conditions that are related to or will respond to the levels of NOGO in a cell or tissue, alone or in combination with other therapies. In addition, NOGO inhibition may be used as a therapeutic target for abrogating CNS neuronal growth inhibition; a situation that can selectively regenerate damaged or lesioned CNS tissue to restore specific reflex and/or locomotor functions.

By “related” is meant that the reduction of NOGO expression (specifically NOGO and/or NOGO receptor gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these tables.

In yet another embodiment, the invention features antisense nucleic acid molecules and 2-5A chimera including sequences complementary to the substrate sequences shown in Tables III to VIII. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 6666), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of≧2 nucleotides in length, preferably 3, 4, 5, 6, 7, 8, 9, 10, 15, 20, 26, 30, where the nucleotides can preferably be internally base-paired to form a stem of preferably≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, ribozymes or antisense molecules that interact with target RNA molecules and inhibit NOGO (specifically NOGO gene) activity are expressed from transcription units inserted into DNA or RNA vectors. The recombinant vectors are preferably DNA plasmids or viral vectors. Ribozyme or antisense expressing viral vectors can be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors can be mused that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of NOGO, the patient can be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat CNS injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO expression.

In another preferred embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., NOGO) capable of progression and/or maintenance of CNS injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage. —indicate the target sequence. Lines interspersed with dots are meant to indicate tertiary interactions. —is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al, 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: : I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

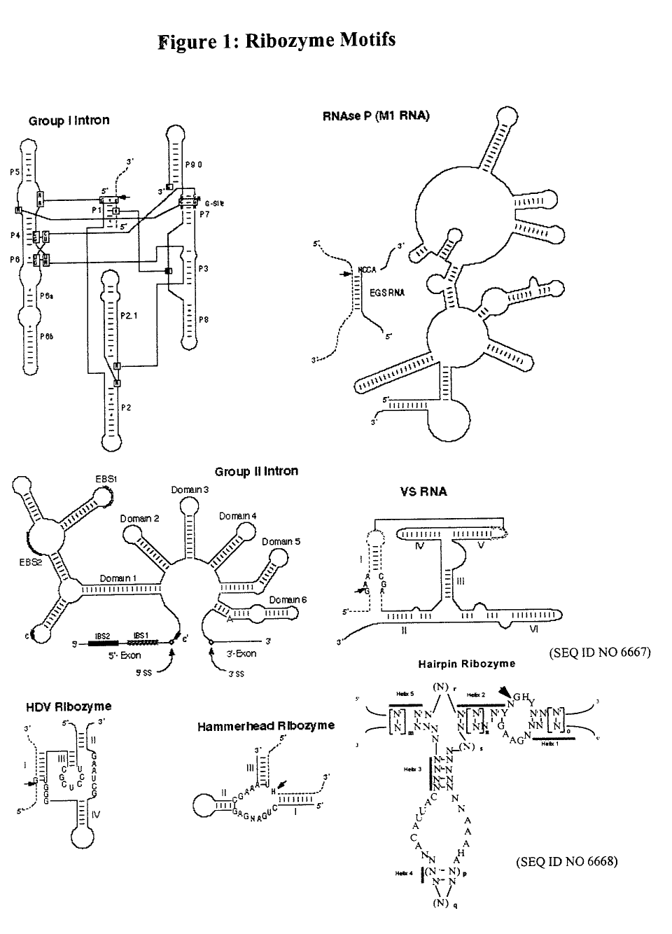

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

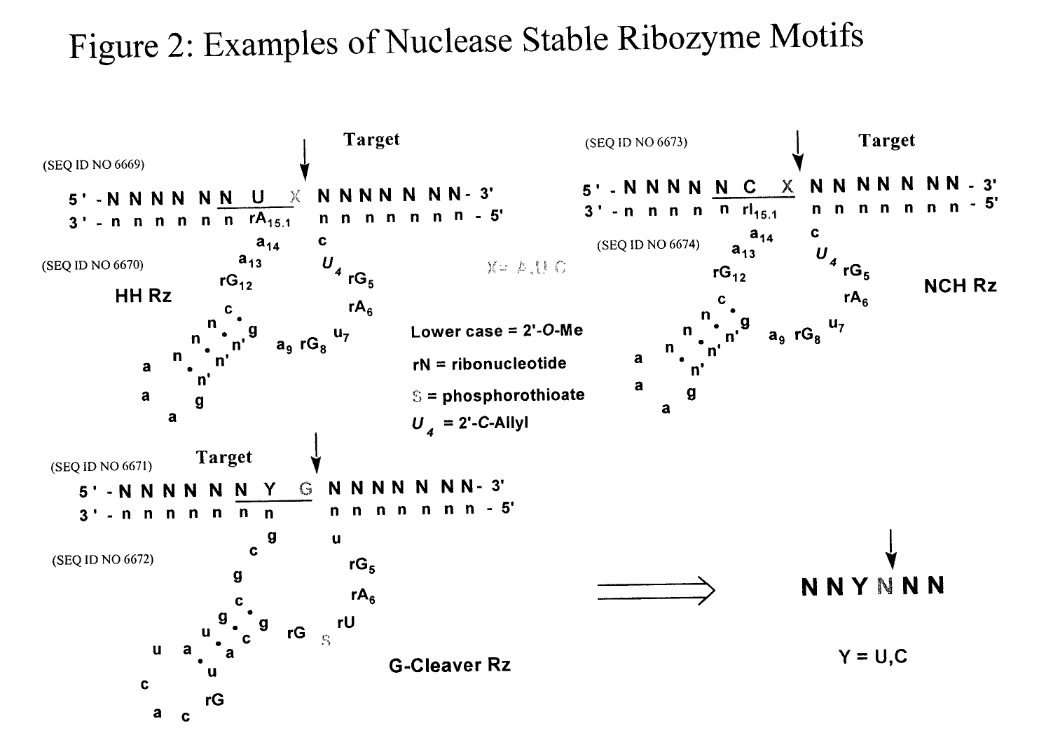

FIG. 3 shows an example of the Amberzyme ribozyme motif that is chemically stabilized (see for example Beigelman et al., International PCT publication No. WO 99/55857).

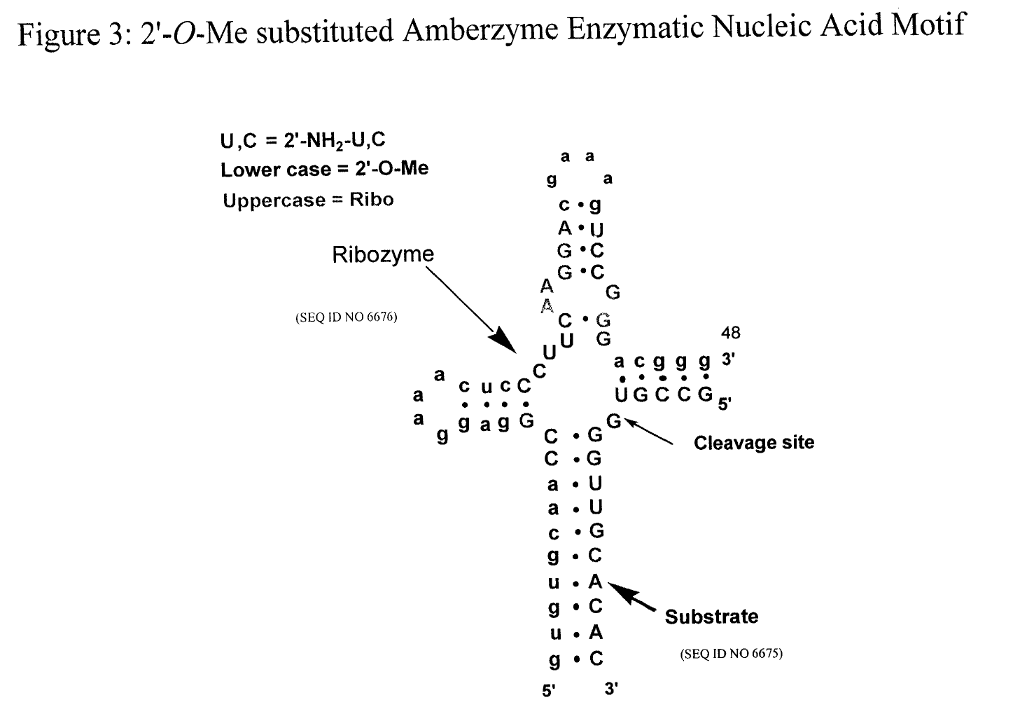

FIG. 4 shows an example of the Zinzyme A ribozyme motif that is chemically stabilized (see for example Beigelman et al., Beigelman et al., International PCT publication No. WO 99/55857).

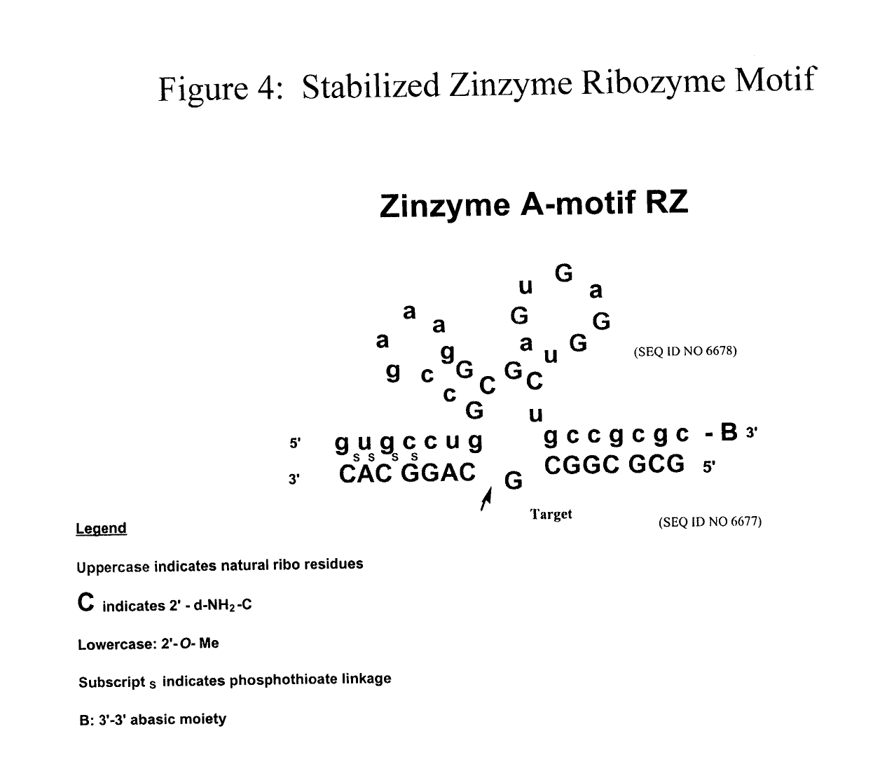

FIG. 5 shows an example of a DNAzyme motif described by Santoro et al., 1997, PNAS, 94, 4262.

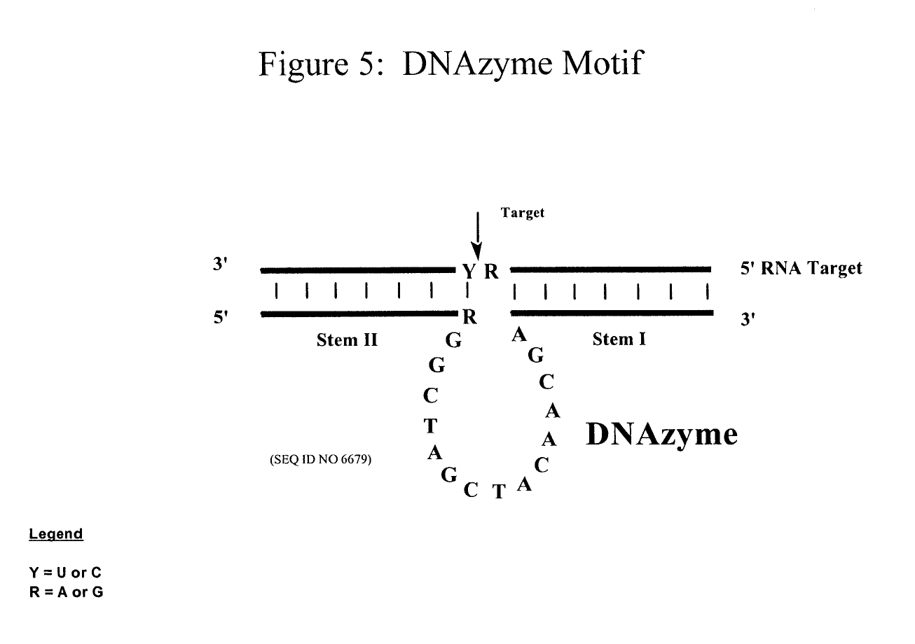

### MECHANISM OF ACTION OF NUCLEIC ACID MOLECULES OF THE INVENTION

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra).

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al, 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent NOGO-A, B, and/or C protein expression and can be used to treat disease or diagnose disease associated with the levels of NOGO-A, B, and/or C.

The enzymatic nature of a ribozyme has significant advantages, one advantage being that the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes can be used as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250.

The nucleic acid molecules of the instant invention are also referred to as GeneBloc reagents, which are essentially nucleic acid molecules (eg; ribozymes, antisense) capable of down-regulating gene expression.

Target sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Ribozymes and antisense to such targets are designed as described in those applications and synthesized to be tested in vitro and in vivo, as also described. The sequences of human NOGO RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to VIII (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules are individually analyzed by computer folding (Jaeger et al, 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (eg; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al, 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I², 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the ribozymes that are chemically synthesized, are shown in Tables III to VIII. The sequences of the antisense constructs that are chemically synthesized, are complementary to the Substrate sequences shown in Tables III to VIII. Those in the art will recognize that these sequences are representative only of many more such sequences where the enzymatic portion of the ribozyme (all but the binding arms) is altered to affect activity. The ribozyme and antisense construct sequences listed in Tables III to VIII can be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another preferred embodiment, nucleic acid catalysts having chemical modifications which maintain or enhance enzymatic activity is provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both terminus. In non-limiting examples: the 5′-cap is selected from the group comprising inverted abasic residue (moiety), 4′,5′-methylene nucleotide; 1-(beta-D-erythrofuiranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO2 or N(CH3)2, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO2 or N(CH3)2, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyunridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O— NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules can lead to better treatment of the disease progression by affording the possibility of combination therapies (e.g., multiple ribozymes targeted to different genes, ribozymnes coupled with known small molecule inhibitors, or intermittent treatment with combinations of ribozymes (including different ribozyme motifs) and/or other chemical or biological molecules). The treatment of patients with nucleic acid molecules can also include combinations of different types of nucleic acid molecules. Therapies can be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Many examples in the art describe CNS delivery methods of oligonucleotides by osmotic pump, (see Chun et al., 1998, Neuroscience Letters, 257, 135-138, D'Aldin et al., 1998, Mol. Brain Research, 55, 151-164, Dryden et al., 1998, J. Endocrinol., 157, 169-175, Ghirnikar et al., 1998, Neuroscience Letters, 247, 21-24) or direct infusion (Broaddus et al., 1997, Neurosurg. Focus, 3, article 4). Other routes of delivery include, but are not limited to oral (tablet or pill form) and/or intrathecal delivery (Gold, 1997, Neuroscience, 76, 1153-1158). For a comprehensive review on drug delivery strategies including broad coverage of CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al, International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al, 1992, Antisense Res. Dev., 2, 3-15; Dropulic et al., 1992, J. Virol., 66, 1432-41; Weerasinghe et al., 1991, J. virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-medicated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within NOGO RNA.

Nucleic Acid Inhibition of NOGO Target RNA

The lack of axon regeneration capacity in the adult CNS manifests as a limiting factor in the treatment of CNS injury, cerebrovascular accident (CVA, stroke), chemotherapy-induced neuropathy, and possibly in neurodegenerative diseases such as Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, and/or muscular dystrophy. Neuron growth inhibition results from physical barriers imposed by glial scars, a lack of neurotrophic factors, and growth-inhibitory molecules associated with myelin. The abrogation of neurite growth inhibition creates the potential to treat conditions for which there is currently no definitive medical intervention. In these studies, the inhibition of NOGO (Genbank Accession No AB020693) is investigated.

### Example 1

Identification of Potential Target Sites in Human NOGO RNA

The sequence of human NOGO is screened for accessible sites using a computer-folding algorithm. Regions of the RNA that do not form secondary folding structures and contained potential ribozyme and/or antisense binding/cleavage sites are identified. The sequences of these binding/cleavage sites are shown in Tables III-VIII.

### Example 2

Selection of Enzymatic Nucleic Acid Cleavage Sites in Human NOGO RNA

Ribozyme target sites are chosen by analyzing sequences of Human NOGO (Genbank accession No: AB020693) and prioritizing the sites on the basis of folding. Ribozymes are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

Chemical Synthesis and Purification of Ribozymes and Antisense for Efficient Cleavage and/or Blocking of NOGO RNA

Ribozymes and antisense constructs are designed to anneal to various sites in the RNA message. The binding arms of the ribozymes are complementary to the target site sequences described above, while the antisense constructs are fully complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs are also synthesized from DNA templates using bacteriophage T7 RNA polymerase (Milligan and Uhlenbeck, 1989, Methods Enzymol. 180, 51). Ribozymes and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized ribozymes used in this study are shown below in Table III-VIII. The sequences of the chemically synthesized antisense constructs used in this study are complimentary sequences to the Substrate sequences shown below as in Table III-VIII.

### Example 4

Ribozyme Cleavage of NOGO RNA Target in vitro

Ribozymes targeted to the human NOGO RNA are designed and synthesized as described above. These ribozymes can be tested for cleavage activity in vitro, for example using the following procedure. The target sequences and the nucleotide location within the NOGO RNA are given in Tables III-VIII.

Cleavage Reactions: Full-length or partially full-length, internally-labeled target RNA for ribozyme cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

Nucleic Acid Inhibition of NOGO Target RNA in vivo

Nucleic acid molecules targeted to the human NOGO RNA are designed and synthesized as described above. These nucleic acid molecules can be tested for cleavage activity in vivo, for example using the procedures described below. The target sequences and the nucleotide location within the NOGO RNA are given in Tables III-VIII.

Cell Culture

Spillmann et al., 1998, J. Biol. Chem., 273, 19283-19293, describe the purification and biochemical characterization of a high molecular mass protein of bovine spinal cord myelin (bNI-220) which exerts potent inhibition of neurite outgrowth of NGF-primed PC12 cells and chick DRG cells. This protein can be used to inhibit spreading of 3T3 fibroblasts and to induce collapse of chick DRG growth cones. The monoclonal antibody, mAb IN-1, can be used to fully neutralize the inhibitory activity of bNI-220, which is a presumed NOGO gene product. As such, nucleic acid molecules of the instant invention directed at the inhibition of NOGO expression can be used in place of mAb IN-1 in studying the inhibition of bNI-220 in cell culture experiments described in detail by Spillmann et al., supra. Criteria used in these experiments include the evaluation of spreading behavior of 3T3 fibroblasts, the nuerite outgrowth response of PC12 cells, and the growth cone motility of chick DRG growth cones

Animal Models

Bregman et al., 1995, Nature, 378, 498-501, describe a rat based system for evaluating the role of myelin-associated neurite growth inhibitory proteins in vivo. Young adult Lewis rats receive a mid-thoracic microsurgical spinal cord lesion. These animals are then treated with mAb IN-1 secreting hybridoma cell explants. A control population receive hybridoma explants which secrete horsreradish peroxidase (HRP) antibodies. Cyclosporin is used during the treatment period to allow hybridoma survival. Additional control rats receive either the spinal cord lesion without any further treatment or no lesion. After a 4-6 week recovery period, behavioral training is followed by the quantitative analysis of reflex and locomotor function. IN-1 treated animals demonstrate growth of corticlspinal axons around the lesion site and into the spinal cord which persist past the longest time point of analysis (12 weeks). Furthermore, both reflex and locomotor function is restored in IN-1 treated animals. As such, a robust animal model as described by Bregman et al stipra, can be used to evaluate nucleic acid molecules of the instant invention when used in place of or in conjunction with mAb IN-1 toward use as modulators of neurite growth inhibitor function (eg. NOGO) in vivo.

Indications

Particular degenerative and disease states that can be associated with NOGO expression modulation include but are not limited to CNS injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob diseases muscular dystrophy, and/or other neurodegenerative disease states which respond to the modulation of NOGO expression.

The present body of knowledge in NOGO research indicates the need for methods to assay NOGO activity and for compounds that can regulate NOGO expression for research, diagnostic, and therapeutic use.

The use of monoclonal antibody (eg; mAb IN-1) treatment is a non-limiting example of a method that can be combined with or used in conjunction with the nucleic acid molecules (e.g. ribozymes and antisense molecules) of the instant invention. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., ribozymes) can be used as diagnostic tools to examine genetic drift and mutations within diseased cells or to detect the presence of NOGO RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one can map nucleotide changes which are important to RNA structure and function in vitro, as well as in cells and tissues. Cleavage of target RNAs with ribozymes can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with NOGO-related condition. Such RNA is detected by determining the presence of a cleavage product after treatment with a ribozyme using standard methodology.

In a specific example, ribozymes which cleave only wild-type or mutant forms of the target RNA are used for the assay. The first ribozyme is used to identify wild-type RNA present in the sample and the second ribozyme is used to identify mutant RNA in the sample. As reaction controls, synthetic substrates of both wild-type and mutant RNA are cleaved by both ribozymes to demonstrate the relative ribozyme efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two ribozymes, two substrates and one unknown sample which is be combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., NOGO) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively.

Additional Uses

Potential uses of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintainance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic com-
parisons, mutagenesis, and biochemical studies [^{i, ii}].
Complete kinetic framework established for one ribozyme [^{iii, iv, v, vi}].
Studies of ribozyme folding and substrate docking underway [^{vii, viii, ix}].
Chemical modification investigation of important residues well
established [^{x, xi}].
The small (4-6 nt) binding site may make this ribozyme too non-specific
for targeted RNA cleavage, however, the Tetrahymena group I intron has
been used to repair a “defective” β-galactosidase message by the ligation
of new β-galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺—OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv, xv}]
Important phosphate and 2′ OH contacts recently identified [^{xvi, xvii}]
Group II Introns
Size: >1000 nucleotides.
Trans cleavage of target RNAs recently demonstrated [^{xviii, xix}].
Sequence requirements not fully determined.
Reaction mechanism: 2′-OH of an internal adenosine generates cleavage
products with 3′-OH and a “lariat” RNA containing a 3′-5′ and a 2′-5′
branch point.
Only natural ribozyme with demonstrated participation in DNA cleavage
[^{xx, xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi, xxvii}]
Minimal ligation activity demonstrated (for engineering through in vitro
selection) [^{xxviii}]
Complete kinetic framework established for two or more ribozymes [^{xxix}].
Chemical modification investigation of important residues well established
[^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi, xxxii, xxxiii, xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues begun
[^{xxxvii, xxxviii}].
Hepatitis Delta Virus (HDV) Ribozyme
Size: ˜6O nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}].
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{xli}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H.. The time dependence of chemical modification reveals slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R.. Ribozyme-mediated repair of defective mRNA by targeted trans-splicing. Nature (London) (1994), 371(6498), 619-22.
^{xiii}Robertson, H. D.; Altman, S.; Smith, J. D. J. Biol. Chem., 247, 5243-5251 (1972).
^{xiv}Forster, Anthony C.; Altman, Sidney. External guide sequences for an RNA enzyme. Science (Washington, D.C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R.. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. T.; Collins, Richard A.. Efficient trans-cleavage of a stem-loop RNA substrate by a ribozyme derived from Neurospora VS RNA. EMBO J. (1995), 14(2), 368-76.
^{xxvi}Scott, W. G., Finch, J. T., Aaron, K. The crystal structure of an all RNA hammerhead ribozyme: A proposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx}Beigelman, L., et al., Chemical modifications of hammerhead ribozymes. J. Biol. Chem., (1995) 270, 25702-25708.
^{xxxi}Hampel, Arnold; Tritz, Richard; Hicks, Margaret; Cruz, Philip. ‘Hairpin’ catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl}Perrotta, Anne T.; Been, Michael D.. A pseudoknot-like structure required for efficient sell-cleavage of hepatitis delta virus RNA. Nature (London) (1991), 350(6317), 434-6.
^{xli}Puttaraju, M.; Perrotta, Anne T.; Been, Michael D.. A circular trans-acting hepatitis delta virus ribozyme. Nucleic Acids Res. (1993), 21(18), 4253-8.

TABLE II

| | | | | | |
|---|---|---|---|---|---|
| Reagent | Equivalents | Amount | Wait Time* DNA | Wait Time* 2′-O-methyl | Wait Time*RNA |

A. 2.5 μmol Synthesis C cle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| Phosphoramidites | 15 | 31 μL | 45 sec | 233 sec | 465 sec |
|---|---|---|---|---|---|
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |
| | Equivalents: | | | | |
| | DNA/ | Amount: DNA/2′-O- | | Wait Time* 2′-O- | |
| Reagent | 2′-O-methyl/Ribo | methyl/Ribo | Wait Time* DNA | methyl | Wait Time* Ribo |

C. 0.2 pmol Synthesis Cycle 96 well Instrument

| | | | | | |
|---|---|---|---|---|---|
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec 200 sec | |
| Acetonitrile | NA | 1150/1150/1150 μL | NA | NA | NA |

*Wait time does not include contact time during delivery.

TABLE III

Human NOGO Hammerhead Ribozyme and Substrate Sequence

| | | | | |
|---|---|---|---|---|
| | | | | Rz |
| | | Seq | | Seq |
| Pos | Substrate | ID | Ribozyme | ID |

| | | | | |
|---|---|---|---|---|
| 10 | ACCACAGU A GGUCCCUC | 1 | GAGGGACC CUGAUGAG GCCGUUAGGC CGAA ACUGUGGU | 2702 |
| 14 | CAGUAGGU C CCUCGGCU | 2 | AGCCGAGG CUGAUGAG GCCGUUAGGC CGAA ACCUACUG | 2703 |
| 18 | AGGUCCCU C GGCUCAGU | 3 | ACUGAGCC CUGAUGAG GCCGUUAGGC CGAA AGGGACCU | 2704 |
| 23 | CCUCGGCU C AGUCGGCC | 4 | GGCCGACU CUGAUGAG GCCGUUAGGC CGAA AGCCGAGG | 2705 |
| 27 | GGCUCAGU C GGCCCAGC | 5 | GCUGGGCC CUGAUGAG GCCGUUAGGC CGAA ACUGAGGC | 2706 |
| 40 | CAGCCCCU C UCAGUCCU | 6 | AGGACUGA CUGAUGAG GCCGUUAGGC CGAA AGGGGCUG | 2707 |
| 42 | GCCCCUCU C AGUCCUCC | 7 | GGAGGACU CUGAUGAG GCCGUUAGGC CGAA AGAGGGGC | 2708 |
| 46 | CUCUCAGU C CUCCCCAA | 8 | UUGGGGAG CUGAUGAG GCCGUUAGGC CGAA ACUGAGAG | 2709 |
| 49 | UCAGUCCU C CCCAACCC | 9 | GGGUUGGG CUGAUGAG GCCGUUAGGC CGAA AGGACUGA | 2710 |
| 76 | CCGCGGCU C UGAGACGC | 10 | GCGUCUCA CUGAUGAG GCCGUUAGGC CGAA AGCCGCGG | 2711 |
| 117 | UGCAGCAU C AUCUCCAC | 11 | GUGGAGAU CUGAUGAG GCCGUUAGGC CGAA AUGCUGCA | 2712 |
| 120 | AGCAUCAU C UCCACCCU | 12 | AGGGUGGA CUGAUGAG GCCGUUAGGC CGAA AUGAUGCU | 2713 |
| 122 | CAUCAUCU C CACCCUCC | 13 | GGAGGGUG CUGAUGAG GCCGUUAGGC CGAA AGAUGAUG | 2714 |
| 129 | UCCACCCU C CAGCCAUG | 14 | CAUGGCUG CUGAUGAG GCCGUUAGGC CGAA AGGGUGGA | 2715 |
| 154 | GGACCAGU C UCCUCUGG | 15 | CCAGAGGA CUGAUGAG GCCGUUAGGC CGAA ACUGGUCC | 2716 |
| 156 | ACCAGUCU C CUCUGGUC | 16 | GACCAGAG CUGAUGAG GCCGUUAGGC CGAA AGACUGGU | 2717 |
| 159 | AGUCUCCU C UGGUCUCG | 17 | CGAGACCA CUGAUGAG GCCGUUAGGC CGAA AGGAGACU | 2718 |
| 164 | CCUCUGGU C UCGUCCUC | 18 | GAGGACGA CUGAUGAG GCCGUUAGGC CGAA ACCAGAGG | 2719 |
| 166 | UCUGGUCU C GUCCUCGG | 19 | CCGAGGAC CUGAUGAG GCCGUUAGGC CGAA AGACCAGA | 2720 |
| 169 | GGUCUCGU C CUCGGACA | 20 | UGUCCGAG CUGAUGAG GCCGUUAGGC CGAA ACGAGACC | 2721 |
| 172 | CUCGUCCU C GGACAGCC | 21 | GGCUGUCC CUGAUGAG GCCGUUAGGC CGAA AGGACGAG | 2722 |
| 202 | GCCCGCGU U CAAGUACC | 22 | GGUACUUG CUGAUGAG GCCGUUAGGC CGAA ACGCGGGC | 2723 |
| 203 | CCCGCGUU C AAGUACCA | 23 | UGGUACUU CUGAUGAG GCCGUUAGGC CGAA AACGCGGG | 2724 |
| 208 | GUUCAAGU A CCAGUUCG | 24 | CGAACUGG CUGAUGAG GCCGUUAGGC CGAA ACUUGAAC | 2725 |
| 214 | GUACCAGU U CGUGAGGG | 25 | CCCUCACG CUGAUGAG GCCGUUAGGC CGAA ACUGGUAC | 2726 |
| 215 | UACCAGUU C GUGAGGGA | 26 | UCCCUCAC CUGAUGAG GCCGUUAGGC CGAA AACUGGUA | 2727 |
| 325 | CGGGCUGU C CGCGGCCC | 27 | GGGCCGCG CUGAUGAG GCCGUUAGGC CGAA ACAGCCCG | 2728 |
| 376 | GAUGGACU U CGGAAAUG | 28 | CAUUUCCG CUGAUGAG GCCGUUAGGC CGAA AGUCCAUC | 2729 |
| 377 | AUGGACUU C GGAAAUGA | 29 | UCAUUUCC CUGAUGAG GCCGUUAGGC CGAA AAGUCCAU | 2730 |
| 388 | AAAUGACU U CGUGCCGC | 30 | GCGGCACG CUGAUGAG GCCGUUAGGC CGAA AGUCAUUU | 2731 |
| 389 | AAUGACUU C GUGCCGCC | 31 | GGCGGCAC CUGAUGAG GCCGUUAGGC CGAA AAGUCAUU | 2732 |
| 426 | CGGCCGCU C CCCCCGUC | 32 | GACGGGGG CUGAUGAG GCCGUUAGGC CGAA AGCGGCCG | 2733 |
| 434 | CCCCCCGU C GCCCCGGA | 33 | UCCGGGGC CUGAUGAG GCCGUUAGGC CGAA ACGGGGGG | 2734 |
| 454 | GCAGCCGU C UUGGGACC | 34 | GGUCCCAA CUGAUGAG GCCGUUAGGC CGAA ACGGCUGC | 2735 |
| 456 | AGCCGUCU U GGGACCCG | 35 | CGGGUCCC CUGAUGAG GCCGUUAGGC CGAA AGACGGCU | 2736 |
| 475 | CCCGGUGU C GUCGACCG | 36 | CGGUCGAC CUGAUGAG GCCGUUAGGC CGAA ACACCGGG | 2737 |
| 478 | GGUGUCGU C GACCGUGC | 37 | GCACGGUC CUGAUGAG GCCGUUAGGC CGAA ACGACACC | 2738 |
| 496 | CGCGCCAU C CCCGCUGU | 38 | ACAGCGGG CUGAUGAG GCCGUUAGGC CGAA AUGGCGCG | 2739 |
| 505 | CCCGCUGU C UGCUGCCG | 39 | CGGCAGCA CUGAUGAG GCCGUUAGGC CGAA ACAGCGGG | 2740 |
| 518 | GCCGCAGU C UCGCCCUC | 40 | GAGGGCGA CUGAUGAG GCCGUUAGGC CGAA ACUGCGGC | 2741 |
| 520 | CGCAGUCU C GCCCUCCA | 41 | UGGAGGGC CUGAUGAG GCCGUUAGGC CGAA AGACUGCG | 2742 |
| 526 | CUCGCCCU C CAAGCUCC | 42 | GGAGCUUG CUGAUGAG GCCGUUAGGC CGAA AGGGCGAG | 2743 |
| 533 | UCCAAGCU C CCUGAGGA | 43 | UCCUCAGG CUGAUGAG GCCGUUAGGC CGAA AGCUUGGA | 2744 |
| 552 | ACGAGCCU C CGGCCCGG | 44 | CCGGGCCG CUGAUGAG GCCGUUAGGC CGAA AGGCUCGU | 2745 |
| 564 | CCCGGCCU C CCCCUCCU | 45 | AGGAGGGG CUGAUGAG GCCGUUAGGC CGAA AGGCCGGG | 2746 |
| 570 | CUCCCCCU C CUCCCCCG | 46 | CGGGGGAG CUGAUGAG GCCGUUAGGC CGAA AGGGGGAG | 2747 |
| 573 | CCCCUCCU C CCCCGGCC | 47 | GGCCGGGG CUGAUGAG GCCGUUAGGC CGAA AGGAGGGG | 2748 |
| 630 | CCCCGGCU C CCGCCGCG | 48 | CGCGGCGG CUGAUGAG GCCGUUAGGC CGAA AGCCGGGG | 2749 |
| 646 | GCCCCCCU C CACCCCGG | 49 | CCGGGGUG CUGAUGAG GCCGUUAGGC CGAA AGGGGGGC | 2750 |
| 676 | CAGGGGCU C CUCGGGCU | 50 | AGCCCGAG CUGAUGAG GCCGUUAGGC CGAA AGCCCCUG | 2751 |
| 679 | GGGCUCCU C GGGCUCAG | 51 | CUGAGCCC CUGAUGAG GCCGUUAGGC CGAA AGGAGCCC | 2752 |
| 665 | CUCGGGCU C AGUGGAUG | 52 | CAUCCACU CUGAUGAG GCCGUUAGGC CGAA AGCCCGAG | 2753 |
| 701 | GAGACCCU U UUUGCUCU | 53 | AGAGCAAA CUGAUGAG GCCGUUAGGC CGAA AGGGUCUC | 2754 |
| 702 | AGACCCUU U UUGCUCUU | 54 | AAGAGCAA CUGAUGAG GCCGUUAGGC CGAA AAGGGUCU | 2755 |
| 703 | GACCCUUU U UGCUCUUC | 55 | GAAGAGCA CUGAUGAG GCCGUUAGGC CGAA AAAGGGUC | 2756 |
| 704 | ACCCUUUU U GCUCUUCC | 56 | GGAAGAGC CUGAUGAG GCCGUUAGGC CGAA AAAAGGGU | 2757 |
| 708 | UUUUUGCU C UUCCUGCU | 57 | AGCAGGAA CUGAUGAG GCCGUUAGGC CGAA AGCAAAAA | 2758 |
| 710 | UUUGCUCU U CCUGCUGC | 58 | GCAGCAGG CUGAUGAG GCCGUUAGGC CGAA AGAGCAAA | 2759 |
| 711 | UUGCUCUU C CUGCUGCA | 59 | UGCAGCAG CUGAUGAG GCCGUUAGGC CGAA AAGAGCAA | 2760 |
| 721 | UGCUGCAU C UGAGCCUG | 60 | CAGGCUCA CUGAUGAG GCCGUUAGGC CGAA AUGCAGCA | 2761 |
| 734 | CCUGUGAU A CGCUCCUC | 61 | GAGGAGCG CUGAUGAG GCCGUUAGGC CGAA AUCACAGG | 2762 |
| 739 | GAUACGCU C CUCUGCAG | 62 | CUGCAGAG CUGAUGAG GCCGUUAGGC CGAA AGCGUAUC | 2763 |
| 742 | ACGCUCCU C UGCAGAAA | 63 | UUUCUGCA CUGAUGAG GCCGUUAGGC CGAA AGGAGCGU | 2764 |
| 753 | CAGAAAAU A UGGACUUG | 64 | CAAGUCCA CUGAUGAG GCCGUUAGGC CGAA AUUUUCUG | 2765 |
| 760 | UAUGGACU U GAAGGAGC | 65 | GCUCCUUC CUGAUGAG GCCGUUAGGC CGAA AGUCCAUA | 2766 |
| 777 | AGCCAGGU A ACACUAUU | 66 | AAUAGUGU CUGAUGAG GCCGUUAGGC CGAA ACCUGGCU | 2767 |
| 783 | GUAACACU A UUUCGGCU | 67 | AGCCGAAA CUGAUGAG GCCGUUAGGC CGAA AGUGUUAC | 2768 |
| 785 | AACACUAU U UCGGCUGG | 68 | CCAGCCGA CUGAUGAG GCCGUUAGGC CGAA AUAGUGUU | 2769 |
| 786 | ACACCAUC U CGGCUGGU | 69 | ACCAGCCG CUGAUGAG GCCGUUAGGC CGAA AAUAGUGU | 2770 |
| 787 | CACUAUUU C GGCUGGUC | 70 | GACCAGCC CUGAUGAG GCCGUUAGGC CGAA AAAUAGUG | 2771 |
| 795 | CGGCUGGU C AAGAGGAU | 71 | AUCCUCUU CUGAUGAG GCCGUUAGGC CGAA ACCAGCCG | 2772 |
| 804 | AAGAGGAU U UCCCAUCU | 72 | AGAUGGGA CUGAUGAG GCCGUUAGGC CGAA AUCCUCUU | 2773 |
| 805 | AGAGGAUU U CCCAUCUG | 73 | CAGAUGGG CUGAUGAG GCCGUUAGGC CGAA AAUCCUCU | 2774 |
| 806 | GAGGAUJU C CCAUCUGU | 74 | ACAGAUGG CUGAUGAG GCCGUUAGGC CGAA AAAUCCUC | 2775 |
| 811 | UUUCCCAU C UGUCCUGC | 75 | GCAGGACA CUGAUGAG GCCGUUAGGC CGAA AUGGGAAA | 2776 |
| 815 | CCAUCUGU C CUGCUUGA | 76 | UCAAGCAG CUGAUGAG GCCGUUAGGC CGAA ACAGAUGG | 2777 |
| 821 | GUCCUGCU U GAAACUGC | 77 | GCAGUUUC CUGAUGAG GCCGUUAGGC CGAA AGCAGGAC | 2778 |
| 834 | CUGCUGCU U CUCUUCCU | 78 | AGGAAGAG CUGAUGAG GCCGUUAGGC CGAA AGCAGCAG | 2779 |
| 835 | UGCUGCUU C UCUUCCUU | 79 | AAGGAAGA CUGAUGAG GCCGUUAGGC CGAA AAGCAGCA | 2780 |
| 837 | CUGCUUCU C UUCCUUCU | 80 | AGAAGGAA CUGAUGAG GCCGUUAGGC CGAA AGAAGCAG | 2781 |
| 839 | GCUUCUCU U CCUUCUCU | 81 | AGAGAAGG CUGAUGAG GCCGUUAGGC CGAA AGAGAAGC | 2782 |
| 840 | CUUCUCUU C CUUCUCUG | 82 | CAGAGAAG CUGAUGAG GCCGUUAGGC CGAA AAGAGAAG | 2783 |
| 843 | CUCUUCCU U CUCUGUCU | 83 | AGACAGAG CUGAUGAG GCCGUUAGGC CGAA AGGAAGAG | 2784 |
| 844 | UCUUCCUU C UCUGUCUC | 84 | GAGACAGA CUGAUGAG GCCGUUAGGC CGAA AAGGAAGA | 2786 |
| 846 | UUCCUUCU C UGUCUCCU | 85 | AGGAGACA CUGAUGAG GCCGUUAGGC CGAA AGAAGGAA | 2786 |
| 850 | UUCUCUGU C UCCUCUCU | 86 | AGAGAGGA CUGAUGAG GCCGUUAGGC CGAA ACAGAGAA | 2787 |
| 852 | CUCUGUCU C CUCUCUCA | 87 | UGAGAGAG CUGAUGAG GCCGUUAGGC CGAA AGACAGAG | 2788 |
| 855 | UGUCUCCU C UCUCAGCC | 88 | GGCUGAGA CUGAUGAG GCCGUUAGGC CGAA AGGAGACA | 2789 |
| 857 | UCUCCUCU C UCAGCCGC | 89 | GCGGCUGA CUGAUGAG GCCGUUAGGC CGAA AGAGGAGA | 2790 |
| 859 | UCCUCUCU C AGCCGCUU | 90 | AAGCGGCU CUGAUGAG GCCGUUAGGC CGAA AGAGAGGA | 2791 |
| 867 | CAGCCGCU U CUUUCAAA | 91 | UUUGAAAG CUGAUGAG GCCGUUAGGC CGAA AGCGGCUG | 2792 |
| 868 | AGCCGCUU C UUUCAAAG | 92 | CUUUGAAA CUGAUGAG GCCGUUAGGC CGAA AAGCGGCU | 2793 |
| 870 | CCGCUUCU U UCAAAGAA | 93 | UUCUUUGA CUGAUGAG GCCGUUAGGC CGAA AGAAGCGG | 2794 |
| 871 | CGCUUCUU U CAAAGAAC | 94 | GUUCUUUG CUGAUGAG GCCGUUAGGC CGAA AAGAAGCG | 2795 |
| 872 | GCUUCUUU C AAAGAACA | 95 | UGUUCUUU CUGAUGAG GCCGUUAGGC CGAA AAAGAAGC | 2796 |
| 886 | ACAUGAAU A CCUUGGUA | 96 | UACCAAGG CUGAUGAG GCCGUUAGGC CGAA AUUCAUGU | 2797 |
| 890 | GAAUACCU U GGUAAUUU | 97 | AAAUUACC CUGAUGAG GCCGUUAGGC CGAA AGGUAUUC | 2798 |
| 894 | ACCUUGGU A ACUUGUCA | 98 | UGACAAAU CUGAUGAG GCCGUUAGGC CGAA ACCAAGGU | 2799 |
| 897 | UUGGUAAU U UGUCAACA | 99 | UGUUGACA CUGAUGAG GCCGUUAGGC CGAA AUUACCAA | 2800 |
| 898 | UGGUAAUU U QUCAACAG | 100 | CUGUUGAC CUGAUGAG GCCGUUAGGC CGAA AAUUACCA | 2801 |
| 901 | UAAUUUGU C AACAGUAU | 101 | AUACUGUU CUGAUGAG GCCGUUAGGC CGAA ACAAAUUA | 2802 |
| 908 | UCAACAGU A UUACCCAC | 102 | GUGGGUAA CUGAUGAG GCCGUUAGGC CGAA ACUGUUGA | 2803 |
| 910 | AACAGUAU U ACCCACUG | 103 | CAGUGGGU CUGAUGAG GCCGUUAGGC CGAA AUACUGUU | 2804 |
| 911 | ACAGUAUU A CCCACUGA | 104 | UCAGUGGG CUGAUGAG GCCGUUAGGC CGAA AAUACUGU | 2805 |
| 929 | GGAACACU U CAAGAAAA | 105 | UUUUCUUG CUGAUGAG GCCGUUAGGC CGAA AGUGUUCC | 2806 |
| 930 | GAACACUU C AAGAAAAU | 106 | AUUUUCUU CUGAUGAG GCCGUUAGGC CGAA AAGUGUUC | 2807 |
| 941 | GAAAAUGU C AGUGAAGC | 107 | GCUUCACU CUGAUGAG GCCGUUAGGC CGAA ACAUUUUC | 2808 |
| 951 | GUGAAGCU U CUAAAGAG | 108 | CUCUUUAG CUGAUGAG GCCGUUAGGC CGAA AGCUUCAC | 2809 |
| 992 | UGAAGCUU C UAAAGAGG | 109 | CCUCUUUA CUGAUGAG GCCGUUAGGC CGAA AAGCUUCA | 2810 |
| 954 | AAGCUUCU A AAGAGGUC | 110 | GACCUCUU CUGAUGAG GCCGUUAGGC CGAA AGAAGCUU | 2811 |
| 962 | AAAGAGGU C UCAGAGAA | 111 | UUCUCUGA CUGAUGAG GCCGUUAGGC CGAA ACCUCUUU | 2812 |
| 964 | AGAGGUCU C AGAGAAGG | 112 | CCUUCUCU CUGAUGAG GCCGUUAGGC CGAA AGACCUCU | 2813 |
| 981 | CAAAAACU C UACUCAUA | 113 | UAUGAGUA CUGAUGAG GCCGUUAGGC CGAA AGUUUUUG | 2814 |
| 983 | AAAACUCU A CUCAUAGA | 114 | UCUAUGAG CUGAUGAG GCCGUUAGGC CGAA AGAGUUUU | 2815 |
| 986 | ACUCUACU C AUAGAUAG | 119 | CUAUCUAU CUGAUGAG GCCGUUAGGC CGAA AGUAGAGU | 2816 |
| 989 | CUACUCAU A GAUAGAGA | 116 | UCUCUAUC CUGAUGAG GCCGUUAGGC CGAA AUGAGUAG | 2817 |
| 993 | UCAUAGAU A GAGAUUUA | 117 | UAAAUCUC CUGAUGAG GCCGUUAGGC CGAA AUCUAUGA | 2818 |
| 999 | AUAGAGAU U UAACAGAG | 118 | CUCUGUUA CUGAUGAG GCCGUUAGGC CGAA AUCUCUAU | 2819 |
| 1000 | UAGAGAUU U AACAGAGU | 119 | ACUCUGUU CUGAUGAG GCCGUUAGGC CGAA AAUCUCUA | 2820 |
| 1001 | AGAGAUUU A ACAGAGUU | 120 | AACUCUGU CUGAUGAG GCCGUUAGGC CGAA AAAUCUCU | 2821 |
| 1009 | AACAGAGU U UUCAGAAU | 121 | AUUCUGAA CUGAUGAG GCCGUUAGGC CGAA ACUCUGUU | 2822 |
| 1010 | ACAGAGUU U UCAGAAUU | 122 | AAUUCUGA CUGAUGAG GCCGUUAGGC CGAA AACUCUGU | 2823 |
| 1011 | CAGAGUUU U CAGAAUUA | 123 | UAAUUCUG CUGAUGAG GCCGUUAGGC CGAA AAACUCUG | 2824 |
| 1012 | AGAGUUUU C AGAAUUAG | 124 | CUAAUUCU CUGAUGAG GCCGUUAGGC CGAA AAAACUCU | 2825 |
| 1018 | UUCAGAAU U AGAAUACU | 125 | AGUAUUCU CUGAUGAG GCCGUUAGGC CGAA AUUCUGAA | 2826 |
| 1019 | UCAGAAUU A GAAUACUC | 126 | GAGUAUUC CUGAUGAG GCCGUUAGGC CGAA AAUUCUGA | 2827 |
| 1024 | AUUAGAAU A CUCAGAAA | 127 | UUUCUGAG CUGAUGAG GCCGUUAGGC CGAA AUUCUAAU | 2828 |
| 1027 | AGAAUACU C AGAAAUGG | 128 | CCAUUUCU CUGAUGAG GCCGUUAGGC CGAA AGUAUUCU | 2829 |
| 1039 | AAUGGGAU C AUCGUUCA | 129 | UGAACGAU CUGAUGAG GCCGUUAGGC CGAA AUCCCAUU | 2830 |
| 1042 | GGGAUCAU C GUUCAGUG | 130 | CACUGAAC CUGAUGAG GCCGUUAGGC CGAA AUGAUCCC | 2831 |
| 1045 | AUCAUCGU U CAGUGUCU | 131 | AGACACUG CUGAUGAG GCCGUUAGGC CGAA ACGAUGAU | 2832 |
| 1046 | UCAUCGUU C AGUGUCUC | 132 | GAGACACU CUGAUGAG GCCGUUAGGC CGAA AACGAUGA | 2833 |
| 1052 | UUCAGUGU C UCUCCAAA | 133 | UUUGGAGA CUGAUGAG GCCGUUAGGC CGAA ACACUGAA | 2834 |
| 1054 | CAGUGUCU C UCCAAAAG | 134 | CUUUUGGA CUGAUGAG GCCGUUAGGC CGAA AGACACUG | 2835 |
| 1056 | GUGUCUCU C CAAAAGCA | 135 | UGCUUUUG CUGAUGAG GCCGUUAGGC CGAA AGAGACAC | 2836 |
| 1069 | AGCAGAAU C UGCCGUAA | 136 | UUACGGCA CUGAUGAG GCCGUUAGGC CGAA AUUCUGCU | 2837 |
| 1076 | UCUGCCGU A AUAGUAGC | 137 | GCUACUAU CUGAUGAG GCCGUUAGGC CGAA ACGGCAGA | 2838 |
| 1079 | GCCGUAAU A GUAGCAAA | 138 | UUUGCUAC CUGAUGAG GCCGUUAGGC CGAA AUUACGGC | 2839 |
| 1082 | GUAAUAGU A GCAAAUCC | 139 | GGAUUUGC CUGAUGAG GCCGUUAGGC CGAA ACUAUUAC | 2840 |
| 1089 | UAGCAAAU C CUAGGGAA | 140 | UUCCCUAG CUGAUGAG GCCGUUAGGC CGAA AUUUGCUA | 2841 |
| 1092 | CAAAUCCU A GGGAAGAA | 141 | UUCUUCCC CUGAUGAG GCCGUUAGGC CGAA AGGAUUUG | 2842 |
| 1103 | GAAGAAAU A AUCGUGAA | 142 | UUCACGAU CUGAUGAG GCCGUUAGGC CGAA AUUUCUUC | 2843 |
| 1106 | GAAAUAAU C GUGAAAAA | 143 | UUUUUCAC CUGAUGAG GCCGUUAGGC CGAA AUUAUUUC | 2844 |
| 1116 | UGAAAAAU A AAGAUGAA | 144 | UUCAUCUU CUGAUGAG GCCGUUAGGC CGAA AUUUUUCA | 2845 |
| 1135 | AGAGAAGU U AGUUAGUA | 145 | UACUAACU CUGAUGAG GCCGUUAGGC CGAA ACUUCUCU | 2846 |
| 1136 | GAGAAGUU A GUUAGUAA | 146 | UUACUAAC CUGAUGAG GCCGUUAGGC CGAA AACUUCUC | 2847 |
| 1139 | AAGUUAGU U AGUAAUAA | 147 | UUAUUACU CUGAUGAG GCCGUUAGGC CGAA ACUAACUU | 2848 |
| 1140 | AGUUAGUU A GUAAUAAC | 148 | GUUAUUAC CUGAUGAG GCCGUUAGGC CGAA AACUAACU | 2849 |
| 1143 | UAGUUAGU A AUAACAUC | 149 | GAUGUUAU CUGAUGAG GCCGUUAGGC CGAA ACUAACUA | 2850 |
| 1146 | UUAGUAAU A ACAUCCUU | 150 | AAGGAUGU CUGAUGAG GCCGUUAGGC CGAA AUUACUAA | 2851 |
| 1151 | AAUAACAU C CUUCAUAA | 191 | UUAUGAAG CUGAUGAG GCCGUUAGGC CGAA AUGUUAUU | 2852 |
| 1154 | AACAUCCU U CAUAAUCA | 152 | UGAUUAUG CUGAUGAG GCCGUUAGGC CGAA AGGAUGUU | 2853 |
| 1155 | ACAUCCUU C AUAAUCAA | 153 | UUGAUUAU CUGAUGAG GCCGUUAGGC CGAA AAGGAUGU | 2854 |
| 1158 | UCCUUCAU A AUCAACAA | 154 | UUGUUGAU CUGAUGAG GCCGUUAGGC CGAA AUGAAGGA | 2855 |
| 1181 | UUCAUAAU C AACAAGAG | 155 | CUCUUGUU CUGAUGAG GCCGUUAGGC CGAA AUUAUGAA | 2856 |
| 1171 | ACAAGAGU U ACCUACAG | 156 | CUGUAGGU CUGAUGAG GCCGUUAGGC CGAA ACUCUUGU | 2857 |
| 1172 | CAAGAGUU A CCUACAGC | 157 | GCUGUAGG CUGAUGAG GCCGUUAGGC CGAA AACUCUUG | 2858 |
| 1176 | AGUUACCU A CAGCUCUU | 158 | AAGAGCUG CUGAUGAG GCCGUUAGGC CGAA AGGUAACU | 2859 |
| 1182 | CUACAGCU C UUACUAAA | 159 | UUUAGUAA CUGAUGAG GCCGUUAGGC CGAA AGCUGUAG | 2860 |
| 1184 | ACAGCUCU U ACUAAAUU | 160 | AAUUUAGU CUGAUGAG GCCGUUAGGC CGAA AGAGCUGU | 2861 |
| 1185 | CAGCUCUU A CUAAAUUG | 161 | CAAUUUAG CUGAUGAG GCCGUUAGGC CGAA AAGAGCUG | 2862 |
| 1188 | CUCUUACU A AAUUGGUU | 162 | AACCAAUU CUGAUGAG GCCGUUAGGC CGAA AGUAAGAG | 2863 |
| 1192 | UACUAAAU U GGUUAAAG | 163 | CUUUAACC CUGAUGAG GCCGUUAGGC CGAA AUUUAGUA | 2864 |
| 1196 | AAAUUGGU U AAAGAGGA | 164 | UCCUCUUU CUGAUGAG GCCGUUAGGC CGAA ACCAAUUU | 2865 |
| 1197 | AAUUGGUU A AAGAGGAU | 165 | AUCCUCUU CUGAUGAG GCCGUUAGGC CGAA AACCAAUU | 2866 |
| 1211 | GAUGAAGU U GUGUCUUC | 166 | GAAGACAC CUGAUGAG GCCGUUAGGC CGAA ACUUCAUC | 2867 |
| 1216 | AGUUGUGU C UUCAGAAA | 167 | UUUCUGAA CUGAUGAG GCCGUUAGGC CGAA ACACAACU | 2868 |
| 1218 | UUGUGUCU U CAGAAAAA | 168 | UUUUUCUG CUGAUGAG GCCGUUAGGC CGAA AGACACAA | 2869 |
| 1219 | UGUGUCUU C AGAAAAAG | 169 | CUUUUUCU CUGAUGAG GCCGUUAGGC CGAA AAGACACA | 2870 |
| 1239 | AAGACAGU U UUAAUGAA | 170 | UUCAUUAA CUGAUGAG GCCGUUAGGC CGAA ACUGUCUU | 2871 |
| 1240 | AGACAGUU U UAAUGAAA | 171 | UUUCAUUA CUGAUGAG GCCGUUAGGC CGAA AACUGUCU | 2872 |
| 1241 | GACAGUUU U AAUGAAAA | 172 | UUUUCAUU CUGAUGAG GCCGUUAGGC CGAA AAACUGUC | 2873 |
| 1242 | ACAGUUUU A AUGAAAAG | 173 | CUUUUCAU CUGAUGAG GCCGUUAGGC CGAA AAAACUGU | 2874 |
| 1256 | AAGAGAGU U GCAGUGGA | 174 | UCCACUGC CUGAUGAG GCCGUUAGGC CGAA ACUCUCUU | 2875 |
| 1269 | UGGAAGCU C CUAUGAGG | 175 | CCUCAUAG CUGAUGAG GCCGUUAGGC CGAA AGCUUCCA | 2876 |
| 1272 | AAGCUCCU A UGAGGGAG | 176 | CUCCCUCA CUGAUGAG GCCGUUAGGC CGAA AGGAGCUU | 2877 |
| 1285 | GGAGGAAU A UGCAGACU | 177 | AGUCUGCA CUGAUGAG GCCGUUAGGC CGAA AUUCCUCC | 2878 |
| 1294 | UGCAGACU U CAAACCAU | 178 | AUGGUUUG CUGAUGAG GCCGUUAGGC CGAA AGUCUGCA | 2879 |
| 1295 | GCAGACUU C AAACCAUU | 179 | AAUGGUUU CUGAUGAG GCCGUUAGGC CGAA AAGUCUGC | 2880 |
| 1303 | CAAACCAU U UGAGCGAG | 180 | CUCGCUCA CUGAUGAG GCCGUUAGGC CGAA AUGGUUUG | 2881 |
| 1304 | AAACCAUU U GAGCGAGU | 181 | ACUCGCUC CUGAUGAG GCCGUUAGGC CGAA AAUGGUUU | 2882 |
| 1313 | GAGCGAGU A UGGGAAGU | 182 | ACUUCCCA CUGAUGAG GCCGUUAGGC CGAA ACUCGCUC | 2883 |
| 1329 | UGAAAGAU A GUAAGGAA | 183 | UUCCUUAC CUGAUGAG GCCGUUAGGC CGAA AUCUUUCA | 2884 |
| 1332 | AAGAUAGU A AGGAAGAU | 184 | AUCUUCCU CUGAUGAG GCCGUUAGGC CGAA ACUAUCUU | 2885 |
| 1341 | AGGAAGAU A GUGAUAUG | 185 | CAUAUCAC CUGAUGAG GCCGUUAGGC CGAA AUCUUCCU | 2886 |
| 1347 | AUAGUGAU A UGUUGGCU | 186 | AGCCAACA CUGAUGAG GCCGUUAGGC CGAA AUCACUAU | 2887 |
| 1351 | UGAUAUGU U GGCUGCUG | 187 | CAGCAGCC CUGAUGAG GCCGUUAGGC CGAA ACAUAUCA | 2888 |
| 1365 | CUGGAGGU A AAAUCGAG | 188 | CUCGAUUU CUGAUGAG GCCGUUAGGC CGAA ACCUCCAG | 2889 |
| 1370 | GGUAAAAU C GAGAGCAA | 189 | UUGCUCUC CUGAUGAG GCCGUUAGGC CGAA AUUUUACC | 2890 |
| 1381 | GAGCAACU U GGAAAGUA | 190 | UACUUUCC CUGAUGAG GCCGUUAGGC CGAA AGUUGCUC | 2891 |
| 1389 | UGGAAAGU A AAGUGGAU | 191 | AUCCACUU CUGAUGAG GCCGUUAGGC CGAA ACUUUCCA | 2892 |
| 1398 | AAGUGGAU A AAAAAUGU | 192 | ACAUUUUU CUGAUGAG GCCGUUAGGC CGAA AUCCACUU | 2893 |
| 1407 | AAAAAUGU U UUGCAGAU | 193 | AUCUGCAA CUGAUGAG GCCGUUAGGC CGAA ACAUUUUU | 2894 |
| 1408 | AAAAUGUU U UGCAGAUA | 194 | UAUCUGCA CUGAUGAG GCCGUUAGGC CGAA AACAUUUU | 2895 |
| 1409 | AAAUGUUU U GCAGAUAG | 195 | CUAUCUGC CUGAUGAG GCCGUUAGGC CGAA AAACAUUU | 2896 |
| 1416 | UUGCAGAU A GCCUUGAG | 196 | CUCAAGGC CUGAUGAG GCCGUUAGGC CGAA AUCUGCAA | 2897 |
| 1421 | GAUAGCCU U GAGCAAAC | 197 | GUUUGCUC CUGAUGAG GCCGUUAGGC CGAA AGGCUAUC | 2898 |
| 1431 | AGCAAACU A AUCACGAA | 198 | UUCGUGAU CUGAUGAG GCCGUUAGGC CGAA AGUUUGCU | 2899 |
| 1434 | AAACUAAU C ACGAAAAA | 199 | UUUUUCGU CUGAUGAG GCCGUUAGGC CGAA AUUAGUUU | 2900 |
| 1446 | AAAAAGAU A GUGAGAGU | 200 | ACUCUCAC CUGAUGAG GCCGUUAGGC CGAA AUCUUUUU | 2901 |
| 1455 | GUGAGAGU A GUAAUGAU | 201 | AUCAUUAC CUGAUGAG GCCGUUAGGC CGAA ACUCUCAC | 2902 |
| 1458 | AGAGUAGU A AUGAUGAU | 202 | AUCAUCAU CUGAUGAG GCCGUUAGGC CGAA ACUACUCU | 2903 |
| 1467 | AUGAUGAU A CUUCUUUC | 203 | GAAAGAAG CUGAUGAG GCCGUUAGGC CGAA AUCAUCAU | 2904 |
| 1470 | AUGAUACU U CUUUCCCC | 204 | GGGGAAAG CUGAUGAG GCCGUUAGGC CGAA AGUAUCAU | 2905 |
| 1471 | UGAUACUU C UUUCCCCA | 205 | UGGGGAAA CUGAUGAG GCCGUUAGGC CGAA AAGUAUCA | 2906 |
| 1473 | AUACUUCU U UCCCCAGU | 206 | ACUGGGGA CUGAUGAG GCCGUUAGGC CGAA AGAAGUAU | 2907 |
| 1474 | UACUUCUU U CCCCAGUA | 207 | UACUGGGG CUGAUGAG GCCGUUAGGC CGAA AAGAAGUA | 2908 |
| 1475 | ACUUCUUU C CCCAGUAC | 208 | GUACUGGG CUGAUGAG GCCGUUAGGC CGAA AAAGAAGU | 2909 |
| 1482 | UCCCCAGU A CGCCAGAA | 209 | UUCUGGCG CUGAUGAG GCCGUUAGGC CGAA ACUGGGGA | 2910 |
| 1494 | CAGAAGGU A UAAAGGAU | 210 | AUCCUUUA CUGAUGAG GCCGUUAGGC CGAA ACCUUCUG | 2911 |
| 1496 | GAAGGUAU A AAGGAUCG | 211 | CGAUCCUU CUGAUGAG GCCGUUAGGC CGAA AUACCUUC | 2912 |
| 1503 | UAAAGGAU C GUUCAGGA | 212 | UCCUGAAC CUGAUGAG GCCGUUAGGC CGAA AUCCUUUA | 2913 |
| 1506 | AGGAUCGU U CAGGAGCA | 213 | UGCUCCUG CUGAUGAG GCCGUUAGGC CGAA ACGAUCCU | 2914 |
| 1507 | GGAUCGUU C AGGAGCAU | 214 | AUGCUCCU CUGAUGAG GCCGUUAGGC CGAA AACGAUCC | 2915 |
| 1516 | AGGAGCAU A UAUCACAU | 215 | AUGUGAUA CUGAUGAG GCCGUUAGGC CGAA AUGCUCCU | 2916 |
| 1518 | GAGCAUAU A UCACAUGU | 216 | ACAUGUGA CUGAUGAG GCCGUUAGGC CGAA AUAUGCUC | 2917 |
| 1520 | GCAUAUAU C ACAUGUGC | 217 | GCACAUGU CUGAUGAG GCCGUUAGGC CGAA AUAUAUGC | 2918 |
| 1530 | CAUGUGCU C CCUUUAAC | 218 | GUUAAAGG CUGAUGAG GCCGUUAGGC CGAA AGCACAUG | 2919 |
| 1534 | UGCUCCCU U UAACCCAG | 219 | CUGGGUUA CUGAUGAG GCCGUUAGGC CGAA AGGGAGCA | 2920 |
| 1535 | GCUCCCUU U AACCCAGC | 220 | GCUGGGUU CUGAUGAG GCCGUUAGGC CGAA AAGGGAGC | 2921 |
| 1536 | CUCCCUUU A ACCCAGCA | 221 | UGCUGGGU CUGAUGAG GCCGUUAGGC CGAA AAAGGGAG | 2922 |
| 1559 | GAGAGCAU U GCAACAAA | 222 | UUUGUUGC CUGAUGAG GCCGUUAGGC CGAA AUGCUCUC | 2923 |
| 1571 | ACAAACAU U UUUCCUUU | 223 | AAAGGAAA CUGAUGAG GCCGUUAGGC CGAA AUGUUUGU | 2924 |
| 1572 | CAAACAUU U UUCCUUUG | 224 | CAAAGGAA CUGAUGAG GCCGUUAGGC CGAA AAUGUUUG | 2925 |
| 1573 | AAACAUUU U UCCUUUGU | 225 | ACAAAGGA CUGAUGAG GCCGUUAGGC CGAA AAAUGUUU | 2926 |
| 1574 | AACAUUUU U CCUUUGUU | 226 | AACAAAGG CUGAUGAG GCCGUUAGGC CGAA AAAAUGUU | 2927 |
| 1575 | ACAUUUUU C CUUUGUUA | 227 | UAACAAAG CUGAUGAG GCCGUUAGGC CGAA AAAAAUGU | 2928 |
| 1578 | UUUUUCCU U UGUUAGGA | 228 | UCCUAACA CUGAUGAG GCCGUUAGGC CGAA AGGAAAAA | 2929 |
| 1579 | UUUUCCUU U GUUAGGAG | 229 | CUCCUAAC CUGAUGAG GCCGUUAGGC CGAA AAGGAAAA | 2930 |
| 1582 | UCCUUUGU U AGGAGAUC | 230 | GAUCUCCU CUGAUGAG GCCGUUAGGC CGAA ACAAAGGA | 2931 |
| 1583 | CCUUUGUU A GGAGAUCC | 231 | GGAUCUCC CUGAUGAG GCCGUUAGGC CGAA AACAAAGG | 2932 |
| 1590 | UAGGAGAU C CUACUUCA | 232 | UGAAGUAG CUGAUGAG GCCGUUAGGC CGAA AUCUCCUA | 2933 |
| 1593 | GAGAUCCU A CUUCAGAA | 233 | UUCUGAAG CUGAUGAG GCCGUUAGGC CGAA AGGAUCUC | 2934 |
| 1596 | AUCCUACU U CAGAAAAU | 234 | AUUUUCUG CUGAUGAG GCCGUUAGGC CGAA AGUAGGAU | 2935 |
| 1597 | UCCUACUU C AGAAAAUA | 235 | UAUUUUCU CUGAUGAG GCCGUUAGGC CGAA AAGUAGGA | 2936 |
| 1605 | CAGAAAAU A AGACCGAU | 236 | AUCGGUCU CUGAUGAG GCCGUUAGGC CGAA AUUUUCUG | 2937 |
| 1625 | AAAAAAAU A GAAGAAAA | 237 | UUUUCUUC CUGAUGAG GCCGUUAGGC CGAA AUUUUUUU | 2938 |
| 1646 | GCCCAAAU A GUAACAGA | 238 | UCUGUUAC CUGAUGAG GCCGUUAGGC CGAA AUUUGGGC | 2939 |
| 1649 | CAAAUAGU A ACAGAGAA | 239 | UUCUCUGU CUGAUGAG GCCGUUAGGC CGAA ACUAUUUG | 2940 |
| 1662 | AGAAGAAU A CUAGCACC | 240 | GGUGCUAG CUGAUGAG GCCGUUAGGC CGAA AUUCUUCU | 2941 |
| 1665 | AGAAUACU A GCACCAAA | 241 | UUUGGUGC CUGAUGAG GCCGUUAGGC CGAA AGUAUUCU | 2942 |
| 1678 | CAAAACAU C AAACCCUU | 242 | AAGGGUUU CUGAUGAG GCCGUUAGGC CGAA AUGUUUUG | 2943 |
| 1686 | CAAACCCU U UUCUUGUA | 243 | UACAAGAA CUGAUGAG GCCGUUAGGC CGAA AGGGUUUG | 2944 |
| 1687 | AAACCCUU U UCUUGUAG | 244 | CUACAAGA CUGAUGAG GCCGUUAGGC CGAA AAGGGUUU | 2945 |
| 1688 | AACCCUUU U CUUGUAGC | 245 | GCUACAAG CUGAUGAG GCCGUUAGGC CGAA AAAGGGUU | 2946 |
| 1589 | ACCCUUUU C UUGUAGCA | 246 | UGCUACAA CUGAUGAG GCCGUUAGGC CGAA AAAAGGGU | 2947 |
| 1691 | CCUUUUCU U GUAGCAGC | 247 | GCUGCUAC CUGAUGAG GCCGUUAGGC CGAA AGAAAAGG | 2948 |
| 1694 | UUUCUUGU A GCAGCACA | 248 | UGUGCUGC CUGAUGAG GCCGUUAGGC CGAA ACAAGAAA | 2949 |
| 1707 | CACAGGAU U CUGAGACA | 249 | UGUCUCAG CUGAUGAG GCCGUUAGGC CGAA AUCCUGUG | 2950 |
| 1705 | ACAGGAUU C UGAGACAG | 250 | CUGUCUCA CUGAUGAG GCCGUUAGGC CGAA AAUCCUGU | 2951 |
| 1719 | AGACAGAU U AUGUCACA | 251 | UGUGACAU CUGAUGAG GCCGUUAGGC CGAA AUCUGUCU | 2952 |
| 1720 | GACAGAUU A UGUCACAA | 252 | UUGUGACA CUGAUGAG GCCGUUAGGC CGAA AAUCUGUC | 2953 |
| 1724 | GAUUAUGU C ACAACAGA | 253 | UCUGUUGU CUGAUGAG GCCGUUAGGC CGAA ACAUAAUC | 2954 |
| 1734 | CAACAGAU A AUUUAACA | 254 | UGUUAAAU CUGAUGAG GCCGUUAGGC CGAA AUCUGUUG | 2955 |
| 1737 | CAGAUAAU U UAACAAAG | 255 | CUUUGUUA CUGAUGAG GCCGUUAGGC CGAA AUUAUCUG | 2956 |
| 1738 | AGAUAAUU U AACAAAGG | 256 | CCUUUGUU CUGAUGAG GCCGUUAGGC CGAA AAUUAUCU | 2957 |
| 1739 | GAUAAUUU A ACAAAGGU | 257 | ACCUUUGU CUGAUGAG GCCGUUAGGC CGAA AAAUUAUC | 2958 |
| 1760 | GAGGAAGU C GUGGCAAA | 258 | UUUGCCAC CUGAUGAG GCCGUUAGGC CGAA ACUUCCUC | 2959 |
| 1788 | GCCUGACU C CACAUUUA | 259 | UAAAUCUG CUGAUGAG GCCGUUAGGC CGAA AGUCAGGC | 2960 |
| 1794 | CUCCAGAG U GAGUACAG | 280 | CUGUACUA CUGAUGAG GCCGUUAGGC CGAA AUCUGGAG | 2961 |
| 1795 | UCCAGAUU U AGUACAGG | 261 | CCUGUACU CUGAUGAG GCCGUUAGGC CGAA AAUCUGGA | 2962 |
| 1796 | CCAGAUUU A GUACAGGA | 262 | UCCUGUAC CUGAUGAG GCCGUUAGGC CGAA AAAUCUGG | 2963 |
| 1799 | GAUUUAGU A CAGGAAGC | 263 | GCUUCCUG CUGAUGAG GCCGUUAGGC CGAA ACUAAAUC | 2964 |
| 1822 | AAGUGAAU U GAAUGAAG | 264 | CUUCAUUC CUGAUGAG GCCGUUAGGC CGAA AUUCACUU | 2965 |
| 1832 | AAUGAAGU U ACUGGUAC | 265 | GUACCAGU CUGAUGAG GCCGUUAGGC CGAA ACUUCAUU | 2966 |
| 1833 | AUGAAGUU A CUGGUACA | 266 | UGUACCAG CUGAUGAG GCCGUUAGGC CGAA AACUUCAU | 2967 |
| 1839 | UUACUGGU A CAAAGAUU | 267 | AAUCUUUG CUGAUGAG GCCGUUAGGC CGAA ACCAGUAA | 2968 |
| 1847 | ACAAAGAU U GCUUAUGA | 268 | UCAUAAGC CUGAUGAG GCCGUUAGGC CGAA AUCUUUGU | 2969 |
| 1851 | AGAUUGCU U AUGAAACA | 269 | UGUUUCAU CUGAUGAG GCCGUUAGGC CGAA AGCAAUCU | 2970 |
| 1852 | GAUUGCUU A UGAAACAA | 270 | UUGUUUCA CUGAUGAG GCCGUUAGGC CGAA AAGCAAUC | 2971 |
| 1870 | AAUGGACU U GGUUCAAA | 271 | UUUGAACC CUGAUGAG GCCGUUAGGC CGAA AGUCCAUU | 2972 |
| 1874 | GACUUGGU U CAAACAUC | 272 | GAUGUUUG CUGAUGAG GCCGUUAGGC CGAA ACCAAGUC | 2973 |
| 1875 | ACUUGGUU C AAACAUCA | 273 | UGAUGUUU CUGAUGAG GCCGUUAGGC CGAA AACCAAGU | 2974 |
| 1882 | UCAAACAU C AGAAGUUA | 274 | UAACUUCU CUGAUGAG GCCGUUAGGC CGAA AUGUUUGA | 2975 |
| 1889 | UCAGAAGU U AUGCAAGA | 275 | UCUUGCAU CUGAUGAG GCCGUUAGGC CGAA ACUUCUGA | 2976 |
| 1890 | CAGAAGUU A UGCAAGAG | 276 | CUCUUGCA CUGAUGAG GCCGUUAGGC CGAA AACUUCUG | 2977 |
| 1900 | GCAAGAGU C ACUCUAUC | 277 | GAUAGAGU CUGAUGAG GCCGUUAGGC CGAA ACUCUUGC | 2878 |
| 1904 | GAGUCACU C UAUCCUGC | 278 | GCAGGAUA CUGAUGAG GCCGUUAGGC CGAA AGUGACUC | 2979 |
| 1906 | GUCACUCU A UCCUGCAG | 279 | CUGCAGGA CUGAUGAG GCCGUUAGGC CGAA AGAGUGAC | 2980 |
| 1908 | CACUCUAU C CUGCAGCA | 280 | UGCUGCAG CUGAUGAG GCCGUUAGGC CGAA AUAGAGUG | 2981 |
| 1922 | GCACAGCU U UGCCCAUC | 281 | GAUGGGCA CUGAUGAG GCCGUUAGGC CGAA AGCUGUGC | 2982 |
| 1923 | CACAGCUU U GCCCAUCA | 282 | UGAUGGGC CUGAUGAG GCCGUUAGGC CGAA AAGCUGUG | 2983 |
| 1930 | UUGCCCAU C AUUUGAAG | 283 | CUUCAAAU CUGAUGAG GCCGUUAGGC CGAA AUGGGCAA | 2984 |
| 1933 | CCCAUCAU U UGAAGAGU | 284 | ACUCUUCA CUGAUGAG GCCGUUAGGC CGAA AUGAUGGG | 2985 |
| 1934 | CCAUCAUU U GAAGAGUC | 285 | GACUCUUC CUGAUGAG GCCGUUAGGC CGAA AAUGAUGG | 2986 |
| 1942 | UGAAGAGU C AGAAGCUA | 286 | UAGCUUCU CUGAUGAG GCCGUUAGGC CGAA ACUCUUCA | 2987 |
| 1950 | CAGAAGCU A CUCCUUCA | 287 | UGAAGGAG CUGAUGAG GCCGUUAGGC CGAA AGCUUCUG | 2988 |
| 1953 | AAGCUACU C CUUCACCA | 288 | UGGUGAAG CUGAUGAG GCCGUUAGGC CGAA AGUAGCUU | 2989 |
| 1956 | CUACUCCU U CACCAGUU | 289 | AACUGGUG CUGAUGAG GCCGUUAGGC CGAA AGGAGUAG | 2990 |
| 1957 | UACUCCUU C ACCAGUUU | 290 | AAACUGGU CUGAUGAG GCCGUUAGGC CGAA AAGGAGUA | 2991 |
| 1964 | UCACCAGU U UUGCCUGA | 291 | UCAGGCAA CUGAUGAG GCCGUUAGGC CGAA ACUGGUGA | 2992 |
| 1965 | CACCAGUU U UGCCUGAC | 292 | GUCAGGCA CUGAUGAG GCCGUUAGGC CGAA AACUGGUG | 2993 |
| 1966 | ACCAGUUU U GCCUGACA | 293 | UGUCAGGC CUGAUGAG GCCGUUAGGC CGAA AAACUGGU | 2994 |
| 1976 | CCUGACAU U GUUAUGGA | 294 | UCCAUAAC CUGAUGAG GCCGUUAGGC CGAA AUGUCAGG | 2995 |
| 1979 | GACAUUGU U AUGGAAGC | 295 | GCUUCCAU CUGAUGAG GCCGUUAGGC CGAA ACAAUGUC | 2996 |
| 1980 | ACAUUGUU A UGGAAGCA | 296 | UGCUUCCA CUGAUGAG GCCGUUAGGC CGAA AACAAUGU | 2997 |
| 1993 | AGCACCAU U GAAUUCUG | 297 | CAGAAUUC CUGAUGAG GCCGUUAGGC CGAA AUGGUGCU | 2998 |
| 1998 | CAUUGAAU U CUGCAGUU | 298 | AACUGCAG CUGAUGAG GCCGUUAGGC CGAA AUUCAAUG | 2999 |
| 1999 | AUUGAAUU C UGCAGUUC | 299 | GAACUGCA CUGAUGAG GCCGUUAGGC CGAA AAUUCAAU | 3000 |
| 2006 | UCUGCAGU U CCUAGUGC | 300 | GCACUAGG CUGAUGAG GCCGUUAGGC CGAA ACUGCAGA | 3001 |
| 2007 | CUGCAGUU C CUAGUGCU | 301 | AGCACUAG CUGAUGAG GCCGUUAGGC CGAA AACUGCAG | 3002 |
| 2010 | CAGUUCCU A GUGCUGGU | 302 | ACCAGCAC CUGAUGAG GCCGUUAGGC CGAA AGGAACUG | 3003 |
| 2022 | CUGGUGCU U CCGUGAUA | 303 | UAUCACGG CUGAUGAG GCCGUUAGGC CGAA AGCACCAG | 3004 |
| 2023 | UGGUGCUU C CGUGAUAC | 304 | GUAUCACG CUGAUGAG GCCGUUAGGC CGAA AAGCACCA | 3005 |
| 2030 | UCCGUGAU A CAGCCCAG | 305 | CUGGGCUG CUGAUGAG GCCGUUAGGC CGAA AUCACGGA | 3006 |
| 2041 | GCCCAGCU C AUCACCAU | 306 | AUGGUGAU CUGAUGAG GCCGUUAGGC CGAA AGCUGGGC | 3007 |
| 2044 | CAGCUCAU C ACCAUUAG | 307 | CUAAUGGU CUGAUGAG GCCGUUAGGC CGAA AUGAGCUG | 3008 |
| 2050 | AUCACCAU U AGAAGCUU | 308 | AAGCUUCU CUGAUGAG GCCGUUAGGC CGAA AUGGUGAU | 3009 |
| 2051 | UCACCAUU A GAAGCUUC | 309 | GAAGCUUC CUGAUGAG GCCGUUAGGC CGAA AAUGGUGA | 3010 |
| 2053 | UAGAAGCU U CUUCAGUU | 310 | AACUGAAG CUGAUGAG GCCGUUAGGC CGAA AGCUUCUA | 3011 |
| 2059 | AGAAGCUU C UUCAGUUA | 311 | UAACUGAA CUGAUGAG GCCGUUAGGC CGAA AAGCUUCU | 3012 |
| 2061 | AAGCUUCU U CAGUUAAU | 312 | AUUAACUG CUGAUGAG GCCGUUAGGC CGAA AGAAGCUU | 3013 |
| 2062 | AGCUUCUU C AGUUAAUU | 313 | AAUUAACU CUGAUGAG GCCGUUAGGC CGAA AAGAAGCU | 3014 |
| 2066 | UCUUCAGU U AAUUAUGA | 314 | UCAUAAUU CUGAUGAG GCCGUUAGGC CGAA ACUGAAGA | 3015 |
| 2067 | CUUCAGUU A AUUAUGAA | 315 | UUCAUAAU CUGAUGAG GCCGUUAGGC CGAA AACUGAAG | 3016 |
| 2070 | CAGUUAAU U AUGAAAGC | 316 | GCUUUCAU CUGAUGAG GCCGUUAGGC CGAA AUUAACUG | 3017 |
| 2071 | AGUUAAUU A UGAAAGCA | 317 | UGCUUUCA CUGAUGAG GCCGUUAGGC CGAA AAUUAACU | 3018 |
| 2081 | GAAAGCAU A AAACAUGA | 318 | UCAUGUUU CUGAUGAG GCCGUUAGGC CGAA AUGCUUUC | 3019 |
| 2110 | CCCACCAU A UGAAGAGG | 319 | CCUCUUCA CUGAUGAG GCCGUUAGGC CGAA AUGGUGGG | 3020 |
| 2129 | AUGAGUGU A UCACUAAA | 320 | UUUAGUGA CUGAUGAG GCCGUUAGGC CGAA ACACUCAG | 3021 |
| 2131 | GAGUGUAU C ACUAAAAA | 321 | UUUUUAGU CUGAUGAG GCCGUUAGGC CGAA AUACACUC | 3022 |
| 2135 | GUAUCACU A AAAAAAGU | 322 | ACUUUUUU CUGAUGAG GCCGUUAGGC CGAA AGUGAUAC | 3023 |
| 2144 | AAAAAAGU A UCAGGAAU | 323 | AUUCCUGA CUGAUGAG GCCGUUAGGC CGAA ACUUUUUU | 3024 |
| 2146 | AAAAGUAU C AGGAAUAA | 324 | UUAUUCCU CUGAUGAG GCCGUUAGGC CGAA AUACUUUU | 3025 |
| 2153 | UCAGGAAU A AAGGAAGA | 325 | UCUUCCUU CUGAUGAG GCCGUUAGGC CGAA AUUCCUGA | 3026 |
| 2165 | GAAGAAAU U AAAGAGCC | 326 | GGCUCUUU CUGAUGAG GCCGUUAGGC CGAA AUUUCUUC | 3027 |
| 2166 | AAGAAAUU A AAGAGCCU | 327 | AGGCUCUU CUGAUGAG GCCGUUAGGC CGAA AAUUUCUU | 3028 |
| 2181 | CUGAAAAU A UUAAUGCA | 328 | UGCAUUAA CUGAUGAG GCCGUUAGGC CGAA AUUUUCAG | 3029 |
| 2183 | GAAAAUAU U AAUGCAGC | 329 | GCUGCAUU CUGAUGAG GCCGUUAGGC CGAA AUAUUUUC | 3030 |
| 2184 | AAAAUAUU A AUGCAGCU | 330 | AGCUGCAU CUGAUGAG GCCGUUAGGC CGAA AAUAUUUU | 3031 |
| 2193 | AUGCAGCU C UUCAAGAA | 331 | UUCUUGAA CUGAUGAG GCCGUUAGGC CGAA AGCUGCAU | 3032 |
| 2195 | GCAGCUCU U CAAGAAAC | 332 | GUUUCUUG CUGAUGAG GCCGUUAGGC CGAA AGAGCUGC | 3033 |
| 2196 | CAGCUCUU C AAGAAACA | 333 | UGUUUCUU CUGAUGAG GCCGUUAGGC CGAA AAGAGCUG | 3034 |
| 2211 | CAGAAGCU C CUUAUAUA | 334 | UAUAUAAG CUGAUGAG GCCGUUAGGC CGAA AGCUUCUG | 3035 |
| 2214 | AAGCUCCU U AUAUAUCU | 335 | AGAUAUAU CUGAUGAG GCCGUUAGGC CGAA AGGAGCUU | 3036 |
| 2215 | AGCUCCUU A UAUAUCUA | 336 | UAGAUAUA CUGAUGAG GCCGUUAGGC CGAA AAGGAGCU | 3037 |
| 2217 | CUCCUUAU A UAUCUAUU | 337 | AAUAGAUA CUGAUGAG GCCGUUAGGC CGAA AUAAGGAG | 3038 |
| 2219 | CCUUAUAU A UCUAUUGC | 338 | GCAAUAGA CUGAUGAG GCCGUUAGGC CGAA AUAUAAGG | 3039 |
| 2221 | UUAUAUAU C UAUUGCAU | 339 | AUGCAAUA CUGAUGAG GCCGUUAGGC CGAA AUAUAUAA | 3040 |
| 2223 | AUAUAUCU A UUGCAUGU | 340 | ACAUGCAA CUGAUGAG GCCGUUAGGC CGAA AGAUAUAU | 3041 |
| 2225 | AUAUCUAU U GCAUGUGA | 341 | UCACAUGC CUGAUGAG GCCGUUAGGC CGAA AUAGAUAU | 3042 |
| 2235 | CAUGUGAU U UAAUUAAA | 342 | UUUAAUUA CUGAUGAG GCCGUUAGGC CGAA AUCACAUG | 3043 |
| 2236 | AUGUGAUU U AAUUAAAG | 343 | CUUUAAUU CUGAUGAG GCCGUUAGGC CGAA AAUCACAU | 3044 |
| 2237 | UGUGAUUU A AUUAAAGA | 344 | UCUUUAAU CUGAUGAG GCCGUUAGGC CGAA AAAUCACA | 3045 |
| 2240 | GAUUUAAU U AAAGAAAC | 345 | GUUUCUUU CUGAUGAG GCCGUUAGGC CGAA AUUAAAUC | 3046 |
| 2241 | AUUUAAUU A AAGAAACA | 346 | UGUUUCUU CUGAUGAG GCCGUUAGGC CGAA AAUUAAAU | 3047 |
| 2255 | ACAAAGCU U UCUGCUGA | 347 | UCAGCAGA CUGAUGAG GCCGUUAGGC CGAA AGCUUUGU | 3048 |
| 2256 | CAAAGCUU U CUGCUGAA | 348 | UUCAGCAG CUGAUGAG GCCGUUAGGC CGAA AAGCUUUG | 3049 |
| 2257 | AAAGCUUU C UGCUGAAC | 349 | GUUCAGCA CUGAUGAG GCCGUUAGGC CGAA AAAGCUUU | 3050 |
| 2271 | AACCAGCU C CGGAUUUC | 350 | GAAAUCCG CUGAUGAG GCCGUUAGGC CGAA AGCUGGUU | 3051 |
| 2277 | CUCCGGAU U UCUCUGAU | 351 | AUCAGAGA CUGAUGAG GCCGUUAGGC CGAA AUCCGGAG | 3052 |
| 2278 | UCCGGAUU U CUCUGAUU | 352 | AAUCAGAG CUGAUGAG GCCGUUAGGC CGAA AAUCCGGA | 3053 |
| 2279 | CCGGAUUU C UCUGAUUA | 353 | UAAUCAGA CUGAUGAG GCCGUUAGGC CGAA AAAUCCGG | 3054 |
| 2281 | GGAUUUCU C UGAUUAUU | 354 | AAUAAUCA CUGAUGAG GCCGUUAGGC CGAA AGAAAUCC | 3055 |
| 2286 | UCUCUGAU U AUUCAGAA | 355 | UUCUGAAU CUGAUGAG GCCGUUAGGC CGAA AUCAGAGA | 3056 |
| 2287 | CUCUGAUU A UUCAGAAA | 356 | UUUCUGAA CUGAUGAG GCCGUUAGGC CGAA AAUCAGAG | 3057 |
| 2289 | CUGAUUAU U CAGAAAUG | 357 | CAUCUCUG CUGAUGAG GCCGUUAGGC CGAA AUAAUCAG | 3058 |
| 2290 | UGAUUAUU C AGAAAUGG | 358 | CCAUUUCU CUGAUGAG GCCGUUAGGC CGAA AAUAAUCA | 3059 |
| 2306 | GCAAAAGU U GAACAGCC | 359 | GGCUGUUC CUGAUGAG GCCGUUAGGC CGAA ACUUUUGC | 3060 |
| 2325 | UGCCUGAU C AUUCUGAG | 360 | CUCAGAAU CUGAUGAG GCCGUUAGGC CGAA AUCAGGCA | 3061 |
| 2328 | CUGAUCAU U CUGAGCUA | 361 | UAGCUCAG CUGAUGAG GCCGUUAGGC CGAA AUGAUCAG | 3062 |
| 2329 | UGAUCAUU C UGAGCUAG | 362 | CUAGCUCA CUGAUGAG GCCGUUAGGC CGAA AAUGAUCA | 3063 |
| 2336 | UCUGAGCU A GUUGAAGA | 363 | UCUUCAAC CUGAUGAG GCCGUUAGGC CGAA AGCUCAGA | 3064 |
| 2339 | GAGCUAGU U GAAGAUUC | 364 | GAAUCUUC CUGAUGAG GCCGUUAGGC CGAA ACUAGGUC | 3065 |
| 2346 | UUGAAGAU U CCUCACCU | 365 | AGGUGAGG CUGAUGAG GCCGUUAGGC CGAA AUCUUCAA | 3066 |
| 2347 | UGAAGAUU C CUCACCUG | 366 | CAGGUGAG CUGAUGAG GCCGUUAGGC CGAA AAUCUUCA | 3067 |
| 2350 | AGAUUCCU C ACCUGAUU | 367 | AAUCAGGU CUGAUGAG GCCGUUAGGC CGAA AGGAAUCU | 3068 |
| 2358 | CACCUGAU U CUGAACCA | 368 | UGGUUCAG CUGAUGAG GCCGUUAGGC CGAA AUCAGGUG | 3069 |
| 2359 | ACCUGAUU C UGAACCAG | 369 | CUGGUUCA CUGAUGAG GCCGUUAGGC CGAA AAUCAGGU | 3070 |
| 2369 | GAACCAGU U GACUUAUU | 370 | AAUAAGUC CUGAUGAG GCCGUUAGGC CGAA ACUGGUUC | 3071 |
| 2374 | AGUUGACU U AUUUAGUG | 371 | CACUAAAU CUGAUGAG GCCGUUAGGC CGAA AGUCAACU | 3072 |
| 2375 | GUUGACUU A UUUAGUGA | 372 | UCACUAAA CUGAUGAG GCCGUUAGGC CGAA AAGUCAAC | 3073 |
| 2377 | UGACUUAU U UAGUGAUG | 373 | CAUCACUA CUGAUGAG GCCGUUAGGC CGPA AUAAGUCA | 3074 |
| 2378 | GACUUAUU U AGUGAUGA | 374 | UCAUCACU CUGAUGAG GCCGUUAGGC CGAA AAUAAGUC | 3075 |
| 2379 | ACUUAUUU A GUGAUGAU | 375 | AUCAUCAC CUGAUGAG GCCGUUAGGC CGAA AAAUAAGU | 3076 |
| 2388 | GUGAUGAU U CAAUACCU | 376 | AGGUAUUG CUGAUGAG GCCGUUAGGC CGAA AUCAUCAC | 3077 |
| 2389 | UGAUGAUU C AAUACCUG | 377 | CAGGUAUU CUGAUGAG GCCGUUAGGC CGAA AAUCAUCA | 3078 |
| 2393 | GAUUCAAU A CCUGACGU | 378 | ACGUCAGG CUGAUGAG GCCGUUAGGC CGAA AUUGAAUC | 3079 |
| 2402 | CCUGACGU U CCACAAAA | 379 | UUUUGUGG CUGAUGAG GCCGUUAGGC CGAA ACGUCAGG | 3080 |
| 2403 | CUGACGUU C CACAAAAA | 380 | UUUUUGUG CUGAUGAG GCCGUUAGGC CGAA AACGUCAG | 3081 |
| 2432 | GUGAUGCU U GUGAAAGA | 381 | UCUUUCAC CUGAUGAG GCCGUUAGGC CGAA AGCAUCAC | 3082 |
| 2445 | AAGAAAGU C UCACUGAG | 382 | CUCAGUGA CUGAUGAG GCCGUUAGGC CGAA ACUUUCUU | 3083 |
| 2447 | GAAAGUCU C ACUGAGAC | 383 | GUCUCAGU CUGAUGAG GCCGUUAGGC CGAA AGACUUUC | 3084 |
| 2457 | CUGAGACU U CAUUUGAG | 384 | CUCAAAUG CUGAUGAG GCCGUUAGGC CGAA AGUCUCAG | 3085 |
| 2458 | UGAGACUU C AUUUGAGU | 385 | ACUCAAAU CUGAUGAG GCCGUUAGGC CGAA AAGUCUCA | 3086 |
| 2461 | GACUUCAU U UGAGUCAA | 386 | UUGACUCA CUGAUGAG GCCGUUAGGC CGAA AUGAAGUC | 3087 |
| 2462 | ACUUCAUU U GAGUCAAU | 387 | AUUGACUC CUGAUGAG GCCGUUAGGC CGAA AAUGAAGU | 3088 |
| 2467 | AUUUGAGU C AAUGAUAG | 388 | CUAUCAUU CUGAUGAG GCCGUUAGGC CGAA ACUCAAAU | 3089 |
| 2474 | UCAAUGAU A GAAUAUGA | 389 | UCAUAUUC CUGAUGAG GCCGUUAGGC CGAA AUCAUUGA | 3090 |
| 2479 | GAUAGAAU A UGAAAAUA | 390 | UAUUUUCA CUGAUGAG GCCGUUAGGC CGAA AUUCUAUC | 3091 |
| 2487 | AUGAAAAU A AGGAAAAA | 391 | UUUUUCCU CUGAUGAG GCCGUUAGGC CGAA AUUUUCAU | 3092 |
| 2498 | GAAAAACU C AGUGCUUU | 392 | AAAGCACU CUGAUGAG GCCGUUAGGC CGAA AGUUUUUC | 3093 |
| 2505 | UCAGUGCU U UGCCACCU | 393 | AGGUGGCA CUGAUGAG GCCGUUAGGC CGAA AGCACUGA | 3094 |
| 2506 | CAGUGCUU U GCCACCUG | 394 | CAGGUGGC CUGAUGAG GCCGUUAGGC CGAA AAGCACUG | 3095 |
| 2530 | AAAGCCAU A UUUGGAAU | 395 | AUUCCAAA CUGAUGAG GCCGUUAGGC CGAA AUGGCUUU | 3096 |
| 2532 | AGCCAUAU U UGGAAUCU | 396 | AGAUUCCA CUGAUGAG GCCGUUAGGC CGAA AUAUGGCU | 3097 |
| 2533 | GCCAUAUU U GGAAUCUU | 397 | AAGAUUCC CUGAUGAG GCCGUUAGGC CGAA AAUAUGGC | 3098 |
| 2539 | UUUGGAAU C UUUUAAGC | 398 | GCUUAAAA CUGAUGAG GCCGUUAGGC CGAA AUUCCAAA | 3099 |
| 2541 | UGGAAUCU U UUAAGCUC | 399 | GAGCUUAA CUGAUGAG GCCGUUAGGC CGAA AGAUUCCA | 3100 |
| 2542 | GGAAUCUU U UAAGCUCA | 400 | UGAGCUUA CUGAUGAG GCCGUUAGGC CGAA AAGAUUCC | 3101 |
| 2543 | GAAUCUUU U AAGCUCAG | 401 | CUGAGCUU CUGAUGAG GCCGUUAGGC CGAA AAAGAUUC | 3102 |
| 2544 | AAUCUUUU A AGCUCAGU | 402 | ACUGAGCU CUGAUGAG GCCGUUAGGC CGAA AAAAGAUU | 3103 |
| 2549 | UUUAAGCU C AGUUUAGA | 403 | UCUAAACU CUGAUGAG GCCGUUAGGC CGAA AGCUUAAA | 3104 |
| 2553 | AGCUCAGU U UAGAUAAC | 404 | GUUAUCUA CUGAUGAG GCCGUUAGGC CGAA ACUGAGCU | 3105 |
| 2554 | GCUCAGUU U AGAUAACA | 405 | UGUUAUCU CUGAUGAG GCCGUUAGGC CGAA AACUGAGC | 3106 |
| 2555 | CUCAGUUU A GAUAACAC | 406 | GUGUUAUC CUGAUGAG GCCGUUAGGC CGAA AAACUGAG | 3107 |
| 2559 | GUUUAGAU A ACACAAAA | 407 | UUUUGUGU CUGAUGAG GCCGUUAGGC CGAA AUCUAAAC | 3108 |
| 2571 | CAPAAGAU A CCCUGUUA | 408 | UAACAGGG CUGAUGAG GCCGUUAGGC CGAA AUCUUUUG | 3109 |
| 2578 | UACCCUGU U ACCUGAUG | 409 | CAUCAGGU CUGAUGAG GCCGUUAGGC CGAA ACAGGGUA | 3110 |
| 2579 | ACCCUGUU A CCUGAUGA | 410 | UCAUCAGG CUGAUGAG GCCGUUAGGC CGAA AACAGGGU | 3111 |
| 2591 | GAUGAAGU U UCAACAUU | 411 | AAUGUUGA CUGAUGAG GCCGUUAGGC CGAA ACUUCAUC | 3112 |
| 2592 | AUGAAGUU U CAACAUUG | 412 | CAAUAUUG CUGAUGAG GCCGUUAGGC CGAA AACUUCAU | 3113 |
| 2593 | UGAAGUUU C AACAUUGA | 413 | UCAAUGUU CUGAUGAG GCCGUUAGGC CGAA AAACUUCA | 3114 |
| 2599 | UUCAACAU U GAGCAAAA | 414 | UUUUGCUC CUGAUGAG GCCGUUAGGC CGAA AUGUUGAA | 3115 |
| 2618 | GAGAAAAU U CCUUUGCA | 415 | UGCAAAGG CUGAUGAG GCCGUUAGGC CGAA AUUUUCUC | 3116 |
| 2619 | AGAAAAUU C CUUUGCAG | 416 | CUGCAAAG CUGAUGAG GCCGUUAGGC CGAA AAUUUUCU | 3117 |
| 2622 | AAAUUCCU U UGCAGAUG | 417 | CAUCUGCA CUGAUGAG GCCGUUAGGC CGAA AGGAAUUU | 3118 |
| 2623 | AAUUCCUU U GCAGAUGG | 418 | CCAUCUGC CUGAUGAG GCCGUUAGGC CGAA AAGGAAUU | 3119 |
| 2639 | GAGGAGCU C AGUACUGC | 419 | GCAGUACU CUGAUGAG GCCGUUAGGC CGAA AGCUCCUC | 3120 |
| 2643 | AGCUCAGU A CUGCAGUU | 420 | AACUGCAG CUGAUGAG GCCGUUAGGC CGAA ACUGAGCU | 3121 |
| 2651 | ACUGCAGU U UAUUCAAA | 421 | UUUGAAUA CUGAUGAG GCCGUUAGGC CGAA ACUGCAGU | 3122 |
| 2652 | CUGCAGUU U AUUCAAAU | 422 | AUUUGAAU CUGAUGAG GCCGUUAGGC CGAA AACUGCAG | 3123 |
| 2653 | UGCAGUUU A UUCAAAUG | 423 | CAUUUGAA CUGAUGAG GCCGUUAGGC CGAA AAACUGCA | 3124 |
| 2655 | CAGUUUAU U CAAAUGAU | 424 | AUCAUUUG CUGAUGAG GCCGUUAGGC CGAA AUAAACUG | 3125 |
| 2656 | AGUUUAUU C AAAUGAUG | 425 | CAUCAUUU CUGAUGAG GCCGUUAGGC CGAA AAUAAACU | 3126 |
| 2668 | UGAUGACU U AUUUAUUU | 426 | AAAUAAAU CUGAUGAG GCCGUUAGGC CGAA AGUCAUCA | 3127 |
| 2669 | GAUGACUU A UUUAUUUC | 427 | GAAAUAAA CUGAUGAG GCCGUUAGGC CGAA AAGUCAUC | 3128 |
| 2671 | UGACUUAU U UAUUUCUA | 428 | UAGAAAUA CUGAUGAG GCCGUUAGGC CGAA AUAAGUCA | 3129 |
| 2672 | GACUUAUU U AUUUCUAA | 429 | UUAGAAAU CUGAUGAG GCCGUUAGGC CGAA AAUAAGUC | 3130 |
| 2673 | ACUUAUUU A UUUCUAAG | 430 | CUUAGAAA CUGAUGAG GCCGUUAGGC CGAA AAAUAAGU | 3131 |
| 2675 | UUAUUUAU U UCUAAGGA | 431 | UCCUUAGA CUGAUGAG GCCGUUAGGC CGAA AUAAAUAA | 3132 |
| 2676 | UAUUUAUU U CUAAGGAA | 432 | UUCCUUAG CUGAUGAG GCCGUUAGGC CGAA AAUAAAUA | 3133 |
| 2677 | AUUUAUUU C UAAGGAAG | 433 | CUUCCUUA CUGAUGAG GCCGUUAGGC CGAA AAAUAAAU | 3134 |
| 2679 | UUAUUUCU A AGGAAGCA | 434 | UGCUUCCU CUGAUGAG GCCGUUAGGC CGAA AGAAAUAA | 3135 |
| 2693 | GCACAGAU A AGAGAAAC | 435 | GUUUCUCU CUGAUGAG GCCGUUAGGC CGAA AUCUGUGC | 3136 |
| 2710 | UGAAACGU U UUCAGAUU | 436 | AAUCUGAA CUGAUGAG GCCGUUAGGC CGAA ACGUUUCA | 3137 |
| 2711 | GAAACGUU U UCAGAUUC | 437 | GAAUCUGA CUGAUGAG GCCGUUAGGC CGAA AACGUUUC | 3138 |
| 2712 | AAACGUUU U CAGAUUCA | 438 | UGAAUCUG CUGAUGAG GCCGUUAGGC CGAA AAACGUUU | 3139 |
| 2713 | AACGUUUU C AGAUUCAU | 439 | AUGAAUCU CUGAUGAG GCCGUUAGGC CGAA AAAACGUU | 3140 |
| 2718 | UUUCAGAU U CAUCUCCA | 440 | UGGAGAUG CUGAUGAG GCCGUUAGGC CGAA AUCUGAAA | 3141 |
| 2719 | UUCAGAUU C AUCUCCAA | 441 | UUGGAGAU CUGAUGAG GCCGUUAGGC CGAA AAUCUGAA | 3142 |
| 2722 | AGAUUCAU C UCCAAUUG | 442 | CAAUUGGA CUGAUGAG GCCGUUAGGC CGAA AUGAAUCU | 3143 |
| 2724 | AUUCAUCU C CAAUUGAA | 443 | UUCAAUUG CUGAUGAG GCCGUUAGGC CGAA AGAUGAAU | 3144 |
| 2729 | UCUCCAAU U GAAAUUAU | 444 | AUAAUUUC CUGAUGAG GCCGUUAGGC CGAA AUUGGAGA | 3145 |
| 2735 | AUUGAAAU U AUAGAUGA | 445 | UCAUCUAU CUGAUGAG GCCGUUAGGC CGAA AUUUCAAU | 3146 |
| 2736 | UUGAAAUU A UAGAUGAG | 446 | CUCAUCUA CUGAUGAG GCCGUUAGGC CGAA AAUUUCAA | 3147 |
| 2738 | GAAAUUAU A GAUGAGUU | 447 | AACUCAUC CUGAUGAG GCCGUUAGGC CGAA AUAAUUUC | 3148 |
| 2746 | AGAUGAGU U CCCUACAU | 448 | AUGUAGGG CUGAUGAG GCCGUUAGGC CGAA ACUCAUCU | 3149 |
| 2747 | GAUGAGUU C CCUACAUU | 449 | AAUGUAGG CUGAUGAG GCCGUUAGGC CGAA AACUCAUC | 3150 |
| 2751 | AGUUCCCU A CAUUGAUC | 450 | GAUCAAUG CUGAUGAG GCCGUUAGGC CGAA AGGGAACU | 3151 |
| 2755 | CCCUACAU U GAUCAGUU | 451 | AACUGAUC CUGAUGAG GCCGUUAGGC CGAA AUGUAGGG | 3152 |
| 2759 | ACAUUGAU C AGUUCUAA | 452 | UUAGAACU CUGAUGAG GCCGUUAGGC CGAA AUCAAUGU | 3153 |
| 2763 | UGAUCAGU U CUAAAACU | 453 | AGUUUUAG CUGAUGAG GCCGUUAGGC CGAA ACUGAUCA | 3154 |
| 2764 | GAUCAGUU C UAAAACUG | 454 | CAGUUUUA CUGAUGAG GCCGUUAGGC CGAA AACUGAUC | 3155 |
| 2766 | UCAGUUCU A AAACUGAU | 455 | AUCAGUUU CUGAUGAG GCCGUUAGGC CGAA AGAACUGA | 3156 |
| 2775 | AAACUGAU U CAUUUUCU | 456 | AGAAAAUG CUGAUGAG GCCGUUAGGC CGAA AUCAGUUU | 3157 |
| 2776 | AACUGAUU C AUUUUCUA | 457 | UAGAAAAU CUGAUGAG GCCGUUAGGC CGAA AAUCAGUU | 3158 |
| 2779 | UGAUUCAU U UUCUAAAU | 458 | AUUUAGAA CUGAUGAG GCCGUUAGGC CGAA AUGAAUCA | 3159 |
| 2780 | GAUUCAUU U UCUAAAUU | 459 | AAUUUAGA CUGAUGAG GCCGUUAGGC CGAA AAUGAAUC | 3160 |
| 2781 | AUUCAUUU U CUAAAUUA | 460 | UAAUUUAG CUGAUGAG GCCGUUAGGC CGAA AAAUGAAU | 3161 |
| 2782 | UUCAUUUU C UAAAUUAG | 461 | CUAAUUUA CUGAUGAG GCCGUUAGGC CGAA AAAAUGAA | 3162 |
| 2784 | CAUUUUCU A AAUUAGCC | 462 | GGCUAAUU CUGAUGAG GCCGUUAGGC CGAA AGAAAAUG | 3163 |
| 2788 | UUCUAAAU U AGCCAGGG | 463 | CCCUGGCU CUGAUGAG GCCGUUAGGC CGAA AUUUAGAA | 3164 |
| 2789 | UCUAAAUU A GCCAGGGA | 464 | UCCCUGGC CUGAUGAG GCCGUUAGGC CGAA AAUUUAGA | 3165 |
| 2800 | CAGGGAAU A UACUGACC | 466 | GGUCAGUA CUGAUGAC GCCGUUAGGC CGAA AUUCCCUG | 3166 |
| 2802 | GGGAAUAU A CUGACCUA | 466 | UAGGUCAG CUGAUGAG GCCGUUAGGC CGAA AUAUUCCC | 3167 |
| 2810 | ACUGACCU A GAAGUAUC | 467 | GAUACUUC CUGAUGAG GCCGUUAGGC CGAA AGGUCAGU | 3168 |
| 2816 | CUAGAAGU A UCCCACAA | 468 | UUGUGGGA CUGAUGAG GCCGUUAGGC CGAA ACUUCUAG | 3169 |
| 2818 | AGAAGUAU C CCACAAAA | 469 | UUUUGUGG CUGAUGAG GCCGUUAGGC CGAA AUACUUCU | 3170 |
| 2834 | AAGUGAAA U GCUAAUGC | 470 | GCAUUAGC CUGAUGAG GCCGUUAGGC CGAA AUUUCACU | 3171 |
| 2838 | AAAUUGCU A AUGCCCCG | 471 | CGGGGCAU CUGAUGAG GCCGUUAGGC CGAA AGCAAUUU | 3172 |
| 2860 | AGCUGGGU C AUUGCCUU | 472 | AAGGCAAU CUGAUGAG GCCGUUAGGC CGAA ACCCAGCU | 3173 |
| 2863 | UGGGUCAU U GCCUUGCA | 473 | UGCAAGGC CUGAUGAG GCCGUUAGGC CGAA AUGACCCA | 3174 |
| 2868 | CAUUGCCU U GCACAGAA | 474 | UUCUGUGC CUGAUGAG GCCGUUAGGC CGAA AGGCAAUG | 3175 |
| 2878 | CACAGAAU U GCCCCAUG | 475 | CAUGGGGC CUGAUGAG GCCGUUAGGC CGAA AUUCUGUG | 3176 |
| 2891 | CAUGACCU U UCUUUGAA | 476 | UUCAAAGA CUGAUGAG GCCGUUAGGC CGAA AGGUCAUG | 3177 |
| 2892 | AUGACCUU U CUUUGAAG | 477 | CUUCAAAG CUGAUGAG GCCGUUAGGC CGAA AAGGUCAU | 3178 |
| 2893 | UGACCUUU C UUUGAAGA | 478 | UCUUCAAA CUGAUGAG GCCGUUAGGC CGAA AAAGGUCA | 3179 |
| 2895 | ACCUUUCU U UGAAGAAC | 479 | GUUCUUCA CUGAUGAG GCCGUUAGGC CGAA AGAAAGGU | 3180 |
| 2896 | CCUUUCUU U GAAGAACA | 480 | UGUUCUUC CUGAUGAG GCCGUUAGGC CGAA AAGAAAGG | 3181 |
| 2906 | AAGAACAU A CAACCCAA | 481 | UUGGGUUG CUGAUGAG GCCGUUAGGC CGAA AUGUUCUU | 3182 |
| 2918 | CCCAAAGU U GAAGAGAA | 482 | UUCUCUUC CUGAUGAG GCCGUUAGGC CGAA ACUUUGGG | 3183 |
| 2930 | GAGAAAAU C AGUUUCUC | 483 | GAGAAACU CUGAUGAG GCCGUUAGGC CGAA AUUUUCUC | 3184 |
| 2934 | AAAUCAGU U UCUCAGAU | 484 | AUCUGAGA CUGAUGAG GCCGUUAGGC CGAA ACUGAGUG | 3185 |
| 2935 | AAUCAGUU U CUCAGAUG | 485 | CAUCUGAG CUGAUGAG GCCGUUAGGC CGAA AACUGAUU | 3186 |
| 2936 | AUCAGUUU C UCAGAUGA | 486 | UCAUCUGA CUGAUGAG GCCGUUAGGC CGAA AAACUGAU | 3187 |
| 2938 | CAGUUUCU C AGAUGACU | 487 | AGUCAUCU CUGAUGAG GCCGUUAGGC CGAA AGAAACUG | 3188 |
| 2947 | AGAUGACU U UUCUAAAA | 488 | UUUUAGAA CUGAUGAG GCCGUUAGGC CGAA AGUCAUCU | 3189 |
| 2948 | GAUGACUU U UCUAAAAA | 489 | UUUUUAGA CUGAUGAG GCCGUUAGGC CGAA AAGUCAUC | 3190 |
| 2949 | AUGACUUU U CUAAAAAU | 490 | AUUUUUAG CUGAUGAG GCCGUUAGGC CGAA AAAGUCAU | 3191 |
| 2950 | UGACUUUU C UAAAAAUG | 491 | CAUUUUUA CUGAUGAG GCCGUUAGGC CGAA AAAAGUCA | 3192 |
| 2952 | ACUUUUCU A AAAAUGGG | 492 | CCCAUUUU CUGAUGAG GCCGUUAGGC CGAA AGAAAAGU | 3193 |
| 2962 | AAAUGGGU C UGCUACAU | 493 | AUGUAGCA CUGAUGAG GCCGUUAGGC CGAA ACCCAUUU | 3194 |
| 2967 | GGUCUGCU A CAUCAAAG | 494 | CUUUGAUG CUGAUGAG GCCGUUAGGC CGAA AGCAGACC | 3195 |
| 2971 | UGCUACAU C AAAGGUGC | 495 | GCACCUUU CUGAUGAG GCCGUUAGGC CGAA AUGUAGCA | 3196 |
| 2981 | AAGGUGCU C UUAUUGCC | 496 | GGCAAUAA CUGAUGAG GCCGUUAGGC CGAA AGCACCUU | 3197 |
| 2983 | GGUGCUCU U AUUGCCUC | 497 | GAGGCAAU CUGAUGAG GCCGUUAGGC CGAA AGAGCACC | 3198 |
| 2984 | GUCCUCUG A UUGCCUCC | 498 | GGAGGCAA CUGAUGAG GCCGUUAGGC CGAA AAGAGCAC | 3199 |
| 2986 | GCUCUUAU U GCCUCCAG | 499 | CUGGAGGC CUGAUGAG GCCGUUAGGC CGAA AUAAGAGC | 3200 |
| 2991 | UAUUGCCU C CAGAUGUU | 500 | AACAUCUG CUGAUGAG GCCGUUAGGC CGAA AGGCAAUA | 3201 |
| 2999 | CCAGAUGU U UCUGCUUU | 501 | AAAGCAGA CUGAUGAG GCCGUUAGGC CGAA ACAUCUGG | 3202 |
| 3000 | CAGAUGUU U CUGCUUUG | 502 | CAAAGCAG CUGAUGAG GCCGUUAGGC CGAA AACAUCUG | 3203 |
| 3001 | AGAUGUUU C UGCUUUGG | 503 | CCAAAGCA CUGAUGAG GCCGUUAGGC CGAA AAACAUCU | 3204 |
| 3006 | UUUCUGCU U UGGCCACU | 504 | AGUGGGCA CUGAUGAG GCCGUUAGGC CGAA AGCAGAAA | 3205 |
| 3007 | UUCUGCUU U GGCCACUC | 505 | GAGUGGCC CUGAUGAG GCCGUUAGGC CGAA AAGCAGAA | 3206 |
| 3015 | UGGCCACU C AAGCAGAG | 506 | CUCUGCUU CUGAUGAG GCCGUUAGGC CGAA AGUGGCCA | 3207 |
| 3026 | GCAGAGAU A GAGAGCAU | 507 | AUGCUCUC CUGAUGAG GCCGUUAGGC CGAA AUCUCUGC | 3208 |
| 3035 | GAGAGCAU A GUUAAACC | 508 | GGUUUAAC CUGAUGAG GCCGUUAGGC CGAA AUGCUCUC | 3209 |
| 3038 | AGCAUAGU U AAACCCAA | 509 | UUGGGUUU CUGAUGAG GCCGUUAGGC CGAA ACUAUGCU | 3210 |
| 3039 | GCAUAGUG A AACCCAAA | 510 | UUUGGGUU CUGAUGAG GCCGUUAGGC CGAA AACUAUGC | 3211 |
| 3050 | CCCAAAGU U CUUGUGAA | 511 | UUCACAAG CUGAUGAG GCCGUUAGGC CGAA ACUUUGGG | 3212 |
| 3051 | CCAAAGUU C UUGUGAAA | 512 | UUUCACAA CUGAUGAG GCCGUUAGGC CGAA AACUUUGG | 3213 |
| 3053 | AAAGUUCU U GUGAAAGA | 513 | UCUUUCAC CUGAUGAG GCCGUUAGGC CGAA AGAACUUU | 3214 |
| 3077 | AAAAAACU U CCUUCCGA | 514 | UCGGAAGG CUGAUGAG GCCGUUAGGC CGAA AGUUUUUU | 3215 |
| 3078 | AAAAACUU C CUUCCGAU | 515 | AUCGGAAG CUGAUGAG GCCGUUAGGC CGAA AAGUUUUU | 3216 |
| 3081 | AACUUCCU U CCGAUACA | 516 | UGUAUCGG CUGAUGAG GCCGUUAGGC CGAA AGGAAGUU | 3217 |
| 3082 | ACUUCCUU C CGAUACAG | 517 | CUGUAUCG CUGAUGAG GCCGUUAGGC CGAA AAGGAAGU | 3218 |
| 3087 | CUUCCGAU A CAGAAAAA | 518 | UUUUUCUG CUGAUGAG GCCGUUAGGC CGAA AUCGGAAG | 3219 |
| 3106 | GGACAGAU C ACCAUCUG | 519 | CAGAUGGU CUGAUGAG GCCGUUAGGC CGAA AUCUGUCC | 3220 |
| 3112 | AUCACCAU C UGCUAUAU | 520 | AUAUAGCA CUGAUGAG GCCGUUAGGC CGAA AUGGUGAU | 3221 |
| 3117 | CAUCUGCU A UAUUUUCA | 521 | UGAAAAUA CUGAUGAG GCCGUUAGGC CGAA AGCAGAUG | 3222 |
| 3119 | UCUGCUAU A UUUUCAGC | 522 | GCUGAAAA CUGAUGAG GCCGUUAGGC CGAA AUAGCAGA | 3223 |
| 3121 | UGCUAUAU U UUCAGCAG | 523 | CUGCUGAA CUGAUGAG GCCGUUAGGC CGAA AUAUAGCA | 3224 |
| 3122 | GCUAUAUU U UCAGCAGA | 524 | UCUGCUGA CUGAUGAG GCCGUUAGGC CGAA AAUAUAGC | 3225 |
| 3123 | CUAUAUUU U CAGCAGAG | 525 | CUCUGCUG CUGAUGAG GCCGUUAGGC CGAA AAAUAUAG | 3226 |
| 3124 | UAUAUUUU C AGCAGAGC | 526 | GCUCUGCU CUGAUGAG GCCGUUAGGC CGAA AAAAUAUA | 3227 |
| 3138 | AGCUGAGU A AAACUUCA | 527 | UGAAGUUU CUGAUGAG GCCGUUAGGC CGAA ACUCAGCU | 3228 |
| 3144 | GUAAAACU U CAGUUGUU | 528 | AACAACUG CUGAUGAG GCCGUUAGGC CGAA AGUUUUAC | 3229 |
| 3145 | UAAAACUU C AGUUGUUG | 529 | CAACAACU CUGAUGAG GCCGUUAGGC CGAA AAGUUUUA | 3230 |
| 3149 | ACUUCAGU U GUUGACCU | 530 | AGGUCAAC CUGAUGAG GCCGUUAGGC CGAA ACUGAAGU | 3231 |
| 3152 | UCAGUUGU U GACCUCCU | 531 | AGGAGGUC CUGAUGAG GCCGUUAGGC CGAA ACAACUGA | 3232 |
| 3158 | GUUGACCU C CUGUACUG | 532 | CAGUACAG CUGAUGAG GCCGUUAGGC CGAA AGGUCAAC | 3233 |
| 3163 | CCUCCUGU A CUGGAGAG | 533 | CUCUCCAG CUGAUGAG GCCGUUAGGC CGAA ACAGGAGG | 3234 |
| 3176 | AGAGACAU U AAGAAGAC | 534 | GUCUUCUU CUGAUGAG GCCGUUAGGC CGAA AUGUCUCU | 3235 |
| 3177 | GAGACAUU A AGAAGACU | 535 | AGUCUUCU CUGAUGAG GCCGUUAGGC CGAA AAUGUCUC | 3236 |
| 3196 | AGUGGUGU U UGGUGCCA | 536 | UGGCACCA CUGAUGAG GCCGUUAGGC CGAA ACACCACU | 3237 |
| 3197 | GUGGUGUU U GGUGCCAG | 537 | CUGGCACC CUGAUGAG GCCGUUAGGC CGAA AACACCAC | 3238 |
| 3209 | GCCAGCCU A UUCCUGCU | 538 | AGCAGGAA CUGAUGAG GCCGUUAGGC CGAA AGGCUGGC | 3239 |
| 3211 | CAGCCUAU U CCUGCUGC | 539 | GCAGCAGG CUGAUGAG GCCGUUAGGC CGAA AUAGGCUG | 3240 |
| 3212 | AGCCUAUU C CUGCUGCU | 540 | AGCAGCAG CUGAUGAG GCCGUUAGGC CGAA AAUAGGCU | 3241 |
| 3221 | CUGCUGCU U UCAUUGAC | 541 | GUCAAUGA CUGAUGAG GCCGUUAGGC CGAA AGCAGCAG | 3242 |
| 3222 | UGCUGCUU U CAUUGACA | 542 | UGUCAAUG CUGAUGAG GCCGUUAGGC CGAA AAGCAGCA | 3243 |
| 3223 | GCUGCUUU C AUUGACAG | 543 | CUGUCAAU CUGAUGAG GCCGUUAGGC CGAA AAAGCAGC | 3244 |
| 3226 | GCUUUCAU U GACAGUAU | 544 | AUACUGUC CUGAUGAG GCCGUUAGGC CGAA AUGAAAGC | 3245 |
| 3233 | UUGACAGU A UUCAGCAU | 545 | AUGCUGAA CUGAUGAG GCCGUUAGGC CGAA ACUGUCAA | 3246 |
| 3235 | GACAGUAU U CAGCAUUG | 546 | CAAUGCUG CUGAUGAG GCCGUUAGGC CGAA AUACUGUC | 3247 |
| 3236 | ACAGUAUU C AGCAUUGU | 547 | ACAAUGCU CUGAUGAG GCCGUUAGGC CGAA AAUACUGU | 3248 |
| 3242 | UUCAGCAU U GUGAGCGU | 548 | ACGCUCAC CUGAUGAG GCCGUUAGGC CGAA AUGCUGAA | 3249 |
| 3251 | GUGAGCGU A ACAGCCUA | 549 | UAGGCUGU CUGAUGAG GCCGUUAGGC CGAA ACGCUCAC | 3250 |
| 3259 | AACAGCCU A CAUUGCCU | 550 | AGGCAAUG CUGAUGAG GCCGUUAGGC CGAA AGGCUGUU | 3251 |
| 3263 | GCCUACAU U GCCUUGGC | 551 | GCCAAGGC CUGAUGAG GCCGUUAGGC CGAA AUGUAGGC | 3252 |
| 3268 | CAUUGCCU U GGCCCUGC | 552 | GCAGGGCC CUGAUGAG GCCGUUAGGC CGAA AGGCAAUG | 3253 |
| 3278 | GCCCUGCU C UCUGUGAC | 553 | GUCACAGA CUGAUGAG GCCGUUAGGC CGAA AGCAGGGC | 3254 |
| 3280 | CCUGCUCU C UGUGACCA | 554 | UGGUCACA CUGAUGAG GCCGUUAGGC CGAA AGAGCAGG | 3255 |
| 3290 | GUGACCAU C AGCUUUAG | 555 | CUAAAGCU CUGAUGAG GCCGUUAGGC CGAA AUGGUCAC | 3256 |
| 3295 | CAUCAGCU U UAGGAUAU | 556 | AUAUCCUA CUGAUGAG GCCGUUAGGC CGAA AGCUGAUG | 3257 |
| 3296 | AUCAGCUU U AGGAUAUA | 557 | UAUAUCCU CUGAUGAG GCCGUUAGGC CGAA AAGCUGAU | 3258 |
| 3297 | UCAGCUUU A GGAUAUAC | 558 | GUAUAUCC CUGAUGAG GCCGUUAGGC CGAA AAAGCUGA | 3259 |
| 3302 | UUUAGGAU A UACAAGGG | 559 | CCCUUGUA CUGAUGAG GCCGUUAGGC CGAA AUCCUAAA | 3260 |
| 3304 | UAGGAUAU A CAAGGGUG | 560 | CACCCUUG CUGAUGAG GCCGUUAGGC CGAA AUAUCCUA | 3261 |
| 3317 | GGUGUGAU C CAAGCUAU | 561 | AUAGCUUG CUGAUGAG GCCGUUAGGC CGAA AUCACACC | 3262 |
| 3324 | UCCAAGCU A UCCAGAAA | 562 | UUUCUGGA CUGAUGAG GCCGUUAGGC CGAA AGCUUGGA | 3263 |
| 3326 | CAAGCUAU C CAGAAAUC | 563 | GAUUUCUG CUGAUGAG GCCGUUAGGC CGAA AUAGCUUG | 3264 |
| 3334 | CCAGAAAU C AGAUGAAG | 564 | CUUCAUCU CUGAUGAG GCCGUUAGGC CGAA AUUUCUGG | 3265 |
| 3352 | CCACCCAU U CAGGGCAU | 565 | AUGCCCUG CUGAUGAG GCCGUUAGGC CGAA AUGGGUGG | 3266 |
| 3353 | CACCCAUU C AGGGCAUA | 566 | UAUGCCCU CUGAUGAG GCCGUUAGGC CGAA AAUGGGUG | 3267 |
| 3361 | CAGGGCAU A UCUGGAAU | 567 | ADUCCAGA CUGAUGAG GCCGUUAGGC CGAA AUGCCCUG | 3268 |
| 3363 | GGGCAUAU C UGGAAUCU | 563 | AGACUCCA CUGAUGAG GCCGUUAGGC CGAA AUAUGCCC | 3269 |
| 3370 | UCUGGAAU C UGAAGUUG | 569 | CAACUUCA CUGAUGAG GCCGUUAGGC CGAA AUUCCAGA | 3270 |
| 3377 | UCUGAAGU U GCUAUAUC | 570 | GAUAUAGC CUGAUGAG GCCGUUAGGC CGAA ACUUCAGA | 3271 |
| 3381 | AAGUUGCU A UAUCUGAG | 571 | CUCAGAUA CUGAUGAG GCCGUUAGGC CGAA AGCAACUU | 3272 |
| 3383 | GUUGCUAU A UCUGAGGA | 572 | UCCUCAGA CUGAUGAG GCCGUUAGGC CGAA AUAGCAAC | 3273 |
| 3385 | UGCUAUAU C UGAGGAGU | 573 | ACUCCUCA CUGAUGAG GCCGUUAGGC CGAA AUAUAGCA | 3274 |
| 3394 | UGAGGAGU U GGUUCAGA | 574 | UCUGAACC CUGAUGAG GCCGUUAGGC CGAA ACUCCUCA | 3275 |
| 3398 | GAGUUGGU U CAGAAGUA | 575 | UACUUCUG CUGAUGAG GCCGUUAGGC CGAA ACCAACUC | 3276 |
| 3399 | AGUUGGUU C AGAAGUAC | 576 | GUACUUCU CUGAUGAG GCCGUUAGGC CGAA AACCAACU | 3277 |
| 3406 | UCAGAAGU A CAGUAAUU | 577 | AAUUACUG CUGAUGAG GCCGUUAGGC CGAA ACUUCUGA | 3278 |
| 3411 | AGUACAGU A AUUCUGCU | 578 | AGCAGAAU CUGAUGAG GCCGUUAGGC CGAA ACUGUACU | 3279 |
| 3414 | ACAGUAAU U CUGCUCUU | 579 | AAGAGCAG CUGAUGAG GCCGUUAGGC CGAA AUUACUGU | 3280 |
| 3415 | CAGUAAUU C UGCUCUUG | 580 | CAAGAGCA CUGAUGAG GCCGUUAGGC CGAA AAUUACUG | 3281 |
| 3420 | AUUCUGCU C UUGGUCAU | 581 | AUGACCAA CUGAUGAG GCCGUUAGGC CGAA AGCAGAAU | 3282 |
| 3422 | UCUGCUCU U GGUCAUGU | 582 | ACAUGACC CUGAUGAG GCCGUUAGGC CGAA AGAGCAGA | 3283 |
| 3426 | CUCUUGGU C AUGUGAAC | 583 | GUUCACAU CUGAUGAG GCCGUUAGGC CGAA ACCAAGAG | 3284 |
| 3443 | UGCACGAU A AAGGAACU | 584 | AGUUCCUU CUGAUGAG GCCGUUAGGC CGAA AUCGUGCA | 3285 |
| 3452 | AAGGAACU C AGGCGCCU | 585 | AGGCGCCU CUGAUGAG GCCGUUAGGC CGAA AGUUCCUU | 3286 |
| 3461 | AGGCGCCU C UUCUUAGU | 586 | ACUAAGAA CUGAUGAG GCCGUUAGGC CGAA AGGCGCCU | 3287 |
| 3463 | GCGCCUCU U CUUAGUUG | 587 | CAACUAAG CUGAUGAG GCCGUUAGGC CGAA AGAGGCGC | 3288 |
| 3464 | CGCCUCUU C UUAGUUGA | 588 | UCAACUAA CUGAUGAG GCCGUUAGGC CGAA AAGAGGCG | 3289 |
| 3466 | CCUCUUCU U AGUUGAUG | 589 | CAUCAACU CUGAUGAG GCCGUUAGGC CGAA AGAAGAGG | 3290 |
| 3467 | CUCUUCUU A GUUGAUGA | 590 | UCAUCAAC CUGAUGAG GCCGUUAGGC CGAA AAGAAGAG | 3291 |
| 3470 | UUCUUAGU U GAUGAUUU | 591 | AAAUCAUC CUGAUGAG GCCGUUAGGC CGAA ACUAAGAA | 3292 |
| 3477 | UUGAUGAU U UAGUUGAU | 592 | AUCAACUA CUGAUGAG GCCGUUAGGC CGAA AUCAUCAA | 3293 |
| 3478 | UGAUGAUU U AGUUGAUU | 593 | AAUCAACU CUGAUGAG GCCGUUAGGC CGAA AAUCAUCA | 3294 |
| 3479 | GAUGAUUU A GUUGAUUC | 594 | GAAUCAAC CUGAUGAG GCCGUUAGGC CGAA AAAUCAUC | 3295 |
| 3482 | GAUUUAGU U GAUUCUCU | 595 | AGAGAAUC CUGAUGAG GCCGUUAGGC CGAA ACUAAAUC | 3296 |
| 3486 | UAGUUGAU U CUCUGAAG | 596 | CUUCAGAG CUGAUGAG GCCGUUAGGC CGAA AUCAACUA | 3297 |
| 3487 | AGUUGAUU C UCUGAAGU | 597 | ACUUCAGA CUGAUGAG GCCGUUAGGC CGAA AAUCAACU | 3298 |
| 3489 | UUGAUUCU C UGAAGUUU | 598 | AAACUUCA CUGAUGAG GCCGUUAGGC CGAA AGAAUCAA | 3299 |
| 3496 | UCUGAAGU U UGCAGUGU | 599 | ACACUGCA CUGAUGAG GCCGUUAGGC CGAA ACUUCAGA | 3300 |
| 3497 | CUGAAGUU U GCAGUGUU | 600 | AACACUGC CUGAUGAG GCCGUUAGGC CGAA AACUUCAG | 3301 |
| 3505 | UGCAGUGU U GAUGUGGG | 601 | CCCACAUC CUGAUGAG GCCGUUAGGC CGAA ACACUGCA | 3302 |
| 3515 | AUGUGGGU A UUUACCUA | 602 | UAGGUAAA CUGAUGAG GCCGUUAGGC CGAA ACCCACAU | 3303 |
| 3517 | GUGGGUAU U UACCUAUG | 603 | CAUAGGUA CUGAUGAG GCCGUUAGGC CGAA AUACCCAC | 3304 |
| 3518 | UGGGUAUU U ACCUAUGU | 604 | ACAUAGGU CUGAUGAG GCCGUUAGGC CGAA AAUACCCA | 3305 |
| 3519 | GGGUAUUU A CCUAUGUU | 605 | AACAUAGG CUGAUGAG GCCGUUAGGC CGAA AAAUACCC | 3306 |
| 3523 | AUUUACCU A UGUUGGUG | 606 | CACCAACA CUGAUGAG GCCGUUAGGC CGAA AGGUAAAU | 3307 |
| 3527 | ACCUAUGU U GGUGCCUU | 607 | AAGGCACC CUGAUGAG GCCGUUAGGC CGAA ACAUAGGU | 3308 |
| 3535 | UGGUGCCU U GUUUAAUG | 608 | CAUUAAAC CUGAUGAG GCCGUUAGGC CGAA AGGCACCA | 3309 |
| 3538 | UGCCUUGU U UAAUGGUC | 609 | GACCAUUA CUGAUGAG GCCGUUAGGC CGAA ACAAGGCA | 3310 |
| 3539 | GCCUUGUU U AAUGGUCU | 610 | AGACCAUU CUGAUGAG GCCGUUAGGC CGAA AACAAGGC | 3311 |
| 3540 | CCUUGUUU A AUGGUCUG | 611 | CAGACCAU CUGAUGAG GCCGUUAGGC CGAA AAACAAGG | 3312 |
| 3546 | UUAAUGGU C UGACACUA | 612 | UAGUGUCA CUGAUGAG GCCGUUAGGC CGAA ACCAUUAA | 3313 |
| 3554 | CUGACACU A CUGAUUUU | 613 | AAAAUCAG CUGAUGAG GCCGUUAGGC CGAA AGUGUCAG | 3314 |
| 3560 | CUACUGAU U UUGGCUCU | 614 | AGAGCCAA CUGAUGAG GCCGUUAGGC CGAA AUCAGUAG | 3315 |
| 3561 | UACUGAUU U UGGCUCUC | 615 | GAGAGCCA CUGAUGAG GCCGUUAGGC CGAA AAUCAGUA | 3316 |
| 3562 | ACUGAUUU U GGCUCUCA | 616 | UGAGAGCC CUGAUGAG GCCGUUAGGC CGAA AAAUCAGU | 3317 |
| 3567 | UUUUGGCU C UCAUUUCA | 617 | UGAAAUGA CUGAUGAG GCCGUUAGGC CGAA AGCCAAAA | 3318 |
| 3569 | UUGGCUCU C AUUUCACU | 618 | AGUGAAAU CUGAUGAG GCCGUUAGGC CGAA AGAGCCAA | 3319 |
| 3572 | GCUCUCAU U UCACUCUU | 619 | AAGAGUGA CUGAUGAG GCCGUUAGGC CGAA AUGAGAGC | 3320 |
| 3573 | CUCUCAUU U CACUCUUC | 620 | GAAGAGUG CUGAUGAG GCCGUUAGGC CGAA AAUGAGAG | 3321 |
| 3574 | UCUCAUUU C ACUCUUCA | 621 | UGAAGAGU CUGAUGAG GCCGUUAGGC CGAA AAAUGAGA | 3322 |
| 3578 | AUUUCACU C UUCAGUGU | 622 | ACACUGAA CUGAUGAG GCCGUUAGGC CGAA AGUGAAAU | 3323 |
| 3580 | UUCACUCU U CAGUGUUC | 623 | GAACACUG CUGAUGAG GCCGUUAGGC CGAA AGAGUGAA | 3324 |
| 3582 | UCACUCUG C AGUGUUCC | 624 | GGAACACU CUGAUGAG GCCGUUAGGC CGAA AAGAGUGA | 3325 |
| 3587 | UUCAGUGU U CCUGUUAU | 625 | AUAACAGG CUGAUGAG GCCGUUAGGC CGAA ACACUGAA | 3326 |
| 3588 | UCAGUGUU C CUGUUAUU | 626 | AAUAACAG CUGAUGAG GCCGUUAGGC CGAA AACACUGA | 3327 |
| 3593 | GUUCCUGU U AUUUAUGA | 627 | UCAUAAAU CUGAUGAG GCCGUUAGGC CGAA ACAGGAAC | 3328 |
| 3594 | UUCCUGUU A UUUAUGAA | 628 | UUCAUAAA CUGAUGAG GCCGUUAGGC CGAA AACAGGAA | 3329 |
| 3596 | CCUGUUAU U UAUGAACG | 629 | CGUUCAUA CUGAUGAG GCCGUUAGGC CGAA AUAACAGG | 3330 |
| 3597 | CUGUUAUU U AUGAACGG | 630 | CCGUUCAU CUGAUGAG GCCGUUAGGC CGAA AAUAACAG | 3331 |
| 3598 | UGUUAUUU A UGAACGGC | 631 | GCCGUUCA CUGAUGAG GCCGUUAGGC CGAA AAAUAACA | 3332 |
| 3609 | AACGGCAU C AGGCACAG | 632 | CUGUGCCU CUGAUGAG GCCGUUAGGC CGAA AUGCCGUU | 3333 |
| 3620 | GCACAGAU A GAUCAUUA | 633 | UAAUGAUC CUGAUGAG GCCGUUAGGC CGAA AUCUGUGC | 3334 |
| 3624 | AGAUAGAU C AGUAUCUA | 634 | UAGAUAAU CUGAUGAG GCCGUUAGGC CGAA AUCUAUCU | 3335 |
| 3627 | UAGAUCAU U AUCUAGGA | 635 | UCCUAGAU CUGAUGAG GCCGUUAGGC CGAA AUGAUCUA | 3336 |
| 3628 | AGAUCAUU A UCUAGGAC | 636 | GUCCUAGA CUGAUGAG GCCGUUAGGC CGAA AAUGAUCU | 3337 |
| 3630 | AUCAUUAU C UAGGACUU | 637 | AAGUCCUA CUGAUGAG GCCGUUAGGC CGAA AUAAUGAU | 3338 |
| 3632 | CAUUAUCU A GGACUUGC | 638 | GCAAGUCC CUGAUGAG GCCGUUAGGC CGAA AGAUAAUG | 3339 |
| 3638 | CUAGGACU U GCAAAUAA | 639 | UUAUUUGC CUGAUGAG GCCGUUAGGC CGAA AGUCCUAG | 3340 |
| 3645 | UUGCAAAU A AGAAUGUU | 640 | AACAUUCU CUGAUGAG GCCGUUAGGC CGAA AUUUGCAA | 3341 |
| 3653 | AAGAAUGU U AAAGAUGC | 641 | GCAUCUUU CUGAUGAG GCCGUUAGGC CGAA ACAUUCUU | 3342 |
| 3654 | AGAAUGUU A AAGAUGCU | 642 | AGCAUCUU CUGAUGAG GCCGUUAGGC CGAA AACAUUCU | 3343 |
| 3663 | AAGAUGCU A UGGCUAAA | 643 | UUUAGCCA CUGAUGAG GCCGUUAGGC CGAA AGCAUCUU | 3344 |
| 3669 | CUAUGGCU A AAAUCCAA | 644 | UUGGAUUU CUGAUGAG GCCGUUAGGC CGAA AGCCAUAG | 3345 |
| 3674 | GCUAAAAU C CAAGCAAA | 645 | UUUGCUUG CUGAUGAG GCCGUUAGGC CGAA AUUUUAGC | 3346 |
| 3586 | GCAAAPAU C CCUGGAUU | 646 | AAUCCAGG CUGAUGAG GCCGUUAGGC CGAA AUUUUUGC | 3347 |
| 3694 | CCCUGGAU U GAAGCGCA | 647 | UGCGCUUC CUGAUGAG GCCGUUAGGC CGAA AUCCAGGG | 3348 |
| 3727 | CCCAAAAU A AUUAGUAG | 648 | CUACUAAU CUGAUGAG GCCGUUAGGC CGAA AUUUUGGG | 3349 |
| 3730 | AAAAUAAU U AGUAGGAG | 649 | CUCCUACU CUGAUGAG GCCGUUAGGC CGAA AUUAUUUU | 3350 |
| 3731 | AAAUAAUU A GUAGGAGU | 650 | ACUCCUAC CUGAUGAG GCCGUUAGGC CGAA AAUUAUUU | 3351 |
| 3734 | UAAUUAGU A GGAGUUCA | 651 | UGAACUCC CUGAUGAG GCCGUUAGGC CGAA ACUAAUUA | 3352 |
| 3740 | GUAGGAGU U CAUCUUUA | 652 | UAAAGAUG CUGAUGAG GCCGUUAGGC CGAA ACUCCUAC | 3353 |
| 3741 | UAGGAGUU C AUCUUUAA | 653 | UUAAAGAU CUGAUGAG GCCGUUAGGC CGAA AACUCCUA | 3354 |
| 3744 | GAGUUCAU C UUUAAAGG | 654 | CCUUUAAA CUGAUGAG GCCGUUAGGC CGAA AUGAACUC | 3355 |
| 3746 | GUUCAUCU U UAAAGGGG | 655 | CCCCUUUA CUGAUGAG GCCGUUAGGC CGAA AGAUGAAC | 3356 |
| 3747 | UUCAUCUU U AAAGGGGA | 656 | UCCCCUUU CUGAUGAG GCCGUUAGGC CGAA AAGAUGAA | 3357 |
| 3748 | UCAUCUUU A AAGGGGAU | 657 | AUCCCCUU CUGAUGAG GCCGUUAGGC CGAA AAAGAUGA | 3358 |
| 3757 | AAGGGGAU A UUCAUUUG | 658 | CAAAUGAA CUGAUGAG GCCGUUAGGC CGAA AUCCCCUU | 3359 |
| 3759 | GGGGAUAU U CAUUUGAU | 659 | AUCAAAUG CUGAUGAG GCCGUUAGGC CGAA AUAUCCCC | 3360 |
| 3760 | GGGAUAUU C AUUUGAUU | 660 | AAUCAAAU CUGAUGAG GCCGUUAGGC CGAA AAUAUCCC | 3361 |
| 3763 | AUAUUCAU U UGAUUAUA | 661 | UAUAAUCA CUGAUGAG GCCGUUAGGC CGAA AUGAAUAU | 3362 |
| 3764 | UAUUCAUU U GAUUAUAC | 662 | GUAUAAUC CUGAUGAG GCCGUUAGGC CGAA AAUGAAUA | 3363 |
| 3768 | CAUUUGAU U AUACGGGG | 663 | CCCCGUAU CUGAUGAG GCCGUUAGGC CGAA AUCAAAUG | 3364 |
| 3769 | AUUUGAUU A UACGGGGG | 664 | CCCCCGUA CUGAUGAG GCCGUUAGGC CGAA AAUCAAAU | 3365 |
| 3771 | UUGAUUAU A CGGGGGAG | 665 | CUCCCCCG CUGAUGAG GCCGUUAGGC CGAA AUAAUCAA | 3366 |
| 3783 | GGGAGGGU C AGGGAAGA | 666 | UCUUCCCU CUGAUGAG GCCGUUAGGC CGAA ACCCUCCC | 3367 |
| 3800 | ACGAACCU U GACGUUGC | 667 | GCAACGUC CUGAUGAG GCCGUUAGGC CGAA AGGUUCGU | 3368 |
| 3806 | CUUGACGU U GCAGUGCA | 668 | UGCACUGC CUGAUGAG GCCGUUAGGC CGAA ACGUCAAG | 3369 |
| 3817 | AGUGCAGU U UCACAGAU | 669 | AUCUGUGA CUGAUGAG GCCGUUAGGC CGAA ACUGCACU | 3370 |
| 3818 | GUGCAGUU U CACAGAUC | 670 | GAUCUGUG CUGAUGAG GCCGUUAGGC CGAA AACUGCAC | 3371 |
| 3819 | UGCAGUUU C ACAGAUCG | 671 | CGAUCUGU CUGAUGAG GCCGUUAGGC CGAA AAACUGCA | 3372 |
| 3826 | UCACAGAU C GUUGUUAG | 672 | CUAACAAC CUGAUGAG GCCGUUAGGC CGAA AUCUGUGA | 3373 |
| 3829 | CAGAUCGU U GUUAGAUC | 673 | GAUCUAAC CUGAUGAG GCCGUUAGGC CGAA ACGAUCUG | 3374 |
| 3832 | AUCGUUGU U AGAUCUUU | 674 | AAAGAUCU CUGAUGAG GCCGUUAGGC CGAA ACAACGAU | 3375 |
| 3833 | UCGUUGUU A GAUCUUUA | 675 | UAAAGAUC CUGAUGAG GCCGUUAGGC CGAA AACAACGA | 3376 |
| 3837 | UGUUAGAU C UUUAUUUU | 676 | AAAAUAAA CUGAUGAG GCCGUUAGGC CGAA AUCUAACA | 3377 |
| 3839 | UUAGAUCU U UAUUUUUA | 677 | UAAAAAUA CUGAUGAG GCCGUUAGGC CGAA AGAUCUAA | 3378 |
| 3840 | UAGAUCUU U AUUUUUAG | 678 | CUAAAAAU CUGAUGAG GCCGUUAGGC CGAA AAGAUCUA | 3379 |
| 3841 | AGAUCUUU A UUUUUAGC | 679 | GCUAAAAA CUGAUGAG GCCGUUAGGC CGAA AAAGAUCU | 3380 |
| 3843 | AUCUUUAU U UUUAGCCA | 680 | UGGCUAAA CUGAUGAG GCCGUUAGGC CGAA AUAAAGAU | 3381 |
| 3844 | UCUUUAUU U UUAGCCAU | 681 | AUGGCUAA CUGAUGAG GCCGUUAGGC CGAA AAUAAAGA | 3382 |
| 3845 | CUUUAUUU U UAGCCAUG | 682 | CAUGGCUA CUGAUGAG GCCGUUAGGC CGAA AAAUAAAG | 3383 |
| 3846 | UUUAUUUU U AGCCAUGC | 683 | GCAUGGCU CUGAUGAG GCCGUUAGGC CGAA AAAAUAAA | 3384 |
| 3847 | UUAUUUUU U GCCAUGCA | 684 | UGCAUGGC CUGAUGAG GCCGUUAGGC CGAA AAAAAUAA | 3385 |
| 3860 | UGCACUGU U GUGAGGAA | 685 | UUCCUCAC CUGAUGAG GCCGUUAGGC CGAA ACAGUGCA | 3386 |
| 3873 | GGAAAAAU U ACCUGUCU | 686 | AGACAGGU CUGAUGAG GCCGUUAGGC CGAA AUUUUUCC | 3387 |
| 3874 | GAAAAAUU A CCUGUCUU | 687 | AAGACAGG CUGAUGAG GCCGUUAGGC CGAA AAUUUUUC | 3388 |
| 3880 | UUACCUGU C UUGACUGC | 688 | GCAGUCAA CUGAUGAG GCCGUUAGGC CGAA ACAGGUAA | 3389 |
| 3882 | ACCUGUCU U GACUGCCA | 689 | UGGCAGUC CUGAUGAG GCCGUUAGGC CGAA AGACAGGU | 3390 |
| 3896 | CCAUGUGU U CAUCAUCU | 690 | AGAUGAUG CUGAUGAG GCCGUUAGGC CGAA ACACAUGG | 3391 |
| 3897 | CAUGUGUU C AUCAUCUU | 691 | AAGAUGAU CUGAUGAG GCCGUUAGGC CGAA AACACAUG | 3392 |
| 3900 | GUGUUCAU C AUCUUAAG | 692 | CUUAAGAU CUGAUGAG GCCGUUAGGC CGAA AUGAACAC | 3393 |
| 3903 | UUCAUCAU C UUAAGUAU | 693 | AUACUUAA CUGAUGAG GCCGUUAGGC CGAA AUGAUGAA | 3394 |
| 3905 | CAUCAUCU U AAGUAUUG | 694 | CAAUACUU CUGAUGAG GCCGUUAGGC CGAA AGAUGAUG | 3395 |
| 3906 | AUCAUCUU A AGUAUUGU | 695 | ACAAUACU CUGAUGAG GCCGUUAGGC CGAA AAGAUGAU | 3396 |
| 3910 | UCUUAAGU A UUGUAAGC | 696 | GCUUACAA CUGAUGAG GCCGUUAGGC CGAA ACUUAAGA | 3397 |
| 3912 | UUAAGUAU U GUAAGCUG | 697 | CAGCUUAC CUGAUGAG GCCGUUAGGC CGAA AUACUUAA | 3398 |
| 3915 | AGUAUUGU A AGCUGCUA | 698 | UAGCAGCU CUGAUGAG GCCGUUAGGC CGAA ACAAUACU | 3399 |
| 3923 | AAGCUGCU A UGUAUGGA | 699 | UCCAUACA CUGAUGAG GCCGUUAGGC CGAA AGCAGCUU | 3400 |
| 3927 | UGCUAUGU A UGGAUUUA | 700 | UAAAUCCA CUGAUGAG GCCGUUAGGC CGAA ACAUAGCA | 3401 |
| 3933 | GUAUGGAU U UAAACCGU | 701 | ACGGUUUA CUGAUGAG GCCGUUAGGC CGAA AUCCAUAC | 3402 |
| 3934 | UAUGGAUU U AAACCGUA | 702 | UACGGUUU CUGAUGAG GCCGUUAGGC CGAA AAUCCAUA | 3403 |
| 3935 | AUGGAUUU A AACCGUAA | 703 | UUACGGUU CUGAUGAG GCCGUUAGGC CGAA AAAUCCAU | 3404 |
| 3942 | UAAACCGU A AUCAUAUC | 704 | GAUAUGAU CUGAUGAG GCCGUUAGGC CGAA ACGGUUUA | 3405 |
| 3945 | ACCGUAAU C AUAUCUUU | 705 | AAAGAUAU CUGAUGAG GCCGUUAGGC CGAA AUUACGGU | 3406 |
| 3948 | GUAAUCAU A UCUUUUUC | 706 | GAAAAAGA CUGAUGAG GCCGUUAGGC CGAA AUGAUGAC | 3407 |
| 3950 | AAUCAUAU C UUUUUCCU | 707 | AGGAAAAA CUGAUGAG GCCGUUAGGC CGAA AUAUGAUU | 3408 |
| 3952 | UCAUAUCU U UUUCCUAU | 708 | AUAGGAAA CUGAUGAG GCCGUUAGGC CGAA AGAUAUGA | 3409 |
| 3953 | CAUAUCUU U UUCCUAUC | 709 | GAUAGGAA CUGAUGAG GCCGUUAGGC CGAA AAGAUAUG | 3410 |
| 3954 | AUAUCUUU U UCCUAUCU | 710 | AGAUAGGA CUGAUGAG GCCGUUAGGC CGAA AAAGAUAU | 3411 |
| 3955 | UAUCUUUU U CCUAUCUG | 711 | CAGAUAGG CUGAUGAG GCCGUUAGGC CGAA AAAAGAUA | 3412 |
| 3956 | AUCUUUUC C CUAUCUGA | 712 | UCAGAUAG CUGAUGAG GCCGUUAGGC CGAA AAAAAGAU | 3413 |
| 3959 | UUUUUCCU A UCUGAGGC | 713 | GCCUCAGA CUGAUGAG GCCGUUAGGC CGAA AGGAAAAA | 3414 |
| 3961 | UUUCCUAU C UGAGGCAC | 714 | GUGCCUCA CUGAUGAG GCCGUUAGGC CGAA AUAGGAAA | 3415 |
| 3979 | GGUGGAAU A AAAAACCU | 715 | AGGUUUUU CUGAUGAG GCCGUUAGGC CGAA AUUCCACC | 3416 |
| 3990 | AAACCUGU A UAUUUUAC | 716 | GUAAAAUA CUGAUGAG GCCGUUAGGC CGAA ACAGGUUU | 3417 |
| 3992 | ACCUGUAU A UUUUACUU | 717 | AAGUAAAA CUGAUGAG GCCGUUAGGC CGAA AUACAGGU | 3418 |
| 3994 | CUGUAUAU U UUACUUUG | 718 | CAAAGUAA CUGAUGAG GCCGUUAGGC CGAA AUAUACAG | 3419 |
| 3995 | UGUAUAUU U UACUUUGU | 719 | ACAAAGUA CUGAUGAG GCCGUUAGGC CGAA AAUAUACA | 3420 |
| 3996 | GUAUAUUU U ACUUUGUU | 720 | AACAAAGU CUGAUGAG GCCGUUAGGC CGAA AAAUAUAC | 3421 |
| 3997 | UAUAUUUU A CUUUGUUG | 721 | CAACAAAG CUGAUGAG GCCGUUAGGC CGAA AAAAUAUA | 3422 |
| 4000 | AUUUUACU U UGUUGCAG | 722 | CUGCAACA CUGAUGAG GCCGUUAGGC CGAA AGUAAAAU | 3423 |
| 4001 | UUUUACUU U GUUGCAGA | 723 | UCUGCAAC CUGAUGAG GCCGUUAGGC CGAA AAGUAAAA | 3424 |
| 4004 | UACUUUGU U GCAGAUAG | 724 | CUAUCUGC CUGAUCAG GCCGUUAGGC CGAA ACAAAGUA | 3425 |
| 4011 | UUGCAGAU A GUCUUGCC | 725 | GGCAAGAC CUGAUGAG GCCGUUAGGC CGAA AUCUGCAA | 3426 |
| 4014 | CAGAUAGU C UUGCCGCA | 726 | UGCGGCAA CUGAUGAG GCCGUUAGGC CGAA ACUAUCUG | 3427 |
| 4016 | GAUAGUCU U GCCGCAUC | 727 | GAUGCGGC CUGAUGAG GCCGUUAGGC CGAA AGACUAUC | 3428 |
| 4024 | UGCCGCAU C UUGGCAAG | 728 | CUUGCCAA CUGAUGAG GCCGUUAGGC CGAA AUGCGGCA | 3429 |
| 4026 | CCGCAUCU U GGCAAGUU | 729 | AACUUGCC CUGAUGAG GCCGUUAGGC CGAA AGAUGCGG | 3430 |
| 4034 | UGGCAAGU U GCAGAGAU | 730 | AUCUCUGC CUGAUGAG GCCGUUAGGC CGAA ACUUGCCA | 3431 |

Input Sequence AB020693. Cut Site = Un/. Stem Length = 8 Core Sequence = CUGAUGAG GCCGUUAGGC CGAA AB020693 (Homo sepiens mRNA for KIAA0886 G protein (Nogo-A); 4053 bp)

Underlined region can be any X sequence or linker, as described herein.

TABLE IV

Human NGGG NCH Ribozyme and Substrate Seqeunce

| | | | | |
|---|---|---|---|---|
| | | | | Rz Seq |
| Pos | Substrate | Seq ID | Ribozyme | ID |

| 15 | AGUAGGUC C CUCGGCUC | 731 | GAGCCGAG CUGAUGAG GCCGUUAGGC CGAA IACCUACU | 3432 | |
|---|---|---|---|---|---|
| 16 | GUAGGUCC C UCGGCUCA | 732 | UGAGCCGA CUGAUGAG GCCGUUAGGC CGAA IGACCUAC | 3433 | |
| 17 | UAGGUCCC U CGGCUCAG | 733 | CUGAGCCG CUGAUGAG GCCGUUAGGC CGAA IGGACCUA | 3434 | |
| 22 | CCCUCCGC U CAGUCGGC | 734 | GCCGACUG CUCAUGAG GCCGUUAGGC CGAA ICCGAGGG | 3435 | |
| 24 | CUCGGCUC A GUCGGCCC | 735 | GGGCCGAC CUGAUGAG GCCGUUAGGC CGAA IAGCCGAG | 3436 | |
| 31 | CAGUCGGC C CAGCCCCU | 736 | AGGGGCUG CUGAUGAG GCCGUUAGGC CGAA ICCGACUG | 3437 | |
| 32 | AGUCGGCC C AGCCCCUC | 737 | GAGGGGCU CUGAUGAG GCCGUUAGGC CGAA IGCCGACU | 3438 | |
| 33 | GUCGGCCC A GCCCCUCU | 738 | AGAGGGGC CUGAUGAG GCCGUUAGGC CGAA IGGCCGAC | 3439 | |
| 36 | GGCCCAGC C UCUCUCAG | 739 | CUGAGAGG CUGAUGAG GCCGUUAGGC CGAA ICUGGGCC | 3440 | |
| 37 | GCCCAGCC C CUCUCAGU | 740 | ACUGAGAG CUGAUGAG GCCGUUAGGC CGAA IGCUGGGC | 3441 | |
| 38 | CCCAGCCC C UCUCAGUC | 741 | GACUGAGA CUGAUGAG GCCGUUAGGC CGAA IGGCUGGG | 3442 | |
| 39 | CCAGCCCC U CUCAGUCC | 742 | GGACUGAG CUGAUGAG GCCGUUAGGC CGAA IGGGCUGG | 3443 | |
| 41 | AGCCCCUC U CAGUCCUC | 743 | GAGGACUG CUGAUGAG GCCGUUAGGC CGAA IAGGGGCU | 3444 | |
| 43 | CGCCUCUC A GUCCUCCC | 744 | GGGAGGAC CUGAUGAG GCCGUUAGGC CGAA IAGAGGGG | 3445 | |
| 47 | UCUCAGUC C UCCCCAAC | 745 | GUUGGGGA CUGAUGAG GCCGUUAGGC CGAA IACUGAGA | 3446 | |
| 48 | CUCAGUCC U CCCCAACC | 746 | GGUUGGGG CUGAUGAG GCCGUUAGGC CGAA IGACUGAG | 3447 | |
| 50 | CAGUCCUC C CCAACCCC | 747 | GGGGUUGG CUGAUGAG GCCGUUAGGC CGAA IAGGACUG | 3448 | |
| 51 | AGUCCUCC C CAACCCCC | 748 | GGGGGUUG CUGAUGAG GCCGUUAGGC CGAA IGAGGACU | 3449 | |
| 52 | GUCCUCCC C AACCCCCA | 749 | UGGGGGUU CUGAUGAG GCCGUUAGGC CGAA IGGAGGAC | 3450 | |
| 53 | UCCUCCCC A ACCCCCAC | 750 | GUGGGGGU CUGAUGAG GCCGUUAGGC CGAA IGGGAGGA | 3451 | |
| 56 | UCCCCAAC C CCCACAAC | 751 | GUUGUGGG CUGAUGAG GCCGUUAGGC CGAA IUUGGGGA | 3452 | |
| 57 | CCCCAACC C CCACAACC | 752 | GGUUGUGG CUGAUGAG GCCGUUAGGC CGAA IGUUGGGG | 3453 | |
| 58 | CCCAACCC C CACAACCG | 753 | CGGUUGUG CUGAUGAG GCCGUUAGGC CGAA IGGUUGGG | 3454 | |
| 59 | CCAACCCC C ACAACCGC | 754 | GCGGUUGU CUGAUGAG GCCGUUAGGC CGAA IGGGUUGG | 3455 | |
| 60 | CAACCCCC A CAACCGCC | 755 | GGCGGUUG CUGAUGAG GCCGUUAGGC CGAA IGGGGUUG | 3456 | |
| 62 | ACCCCCAC A ACCGCCCG | 756 | CGGGCGGU CUGAUGAG GCCGUUAGGC CGAA IUGGGGGU | 3457 | |
| 65 | CCCACAAC C GCCCGCGG | 757 | CCGCGGGC CUGAUGAG GCCGUUAGGC CGAA IUUGUGGG | 3458 | |
| 68 | ACAACCGC C CGCGGCUC | 758 | GAGCCGCG CUGAUGAG GCCGUUAGGC CGAA ICGGUUGU | 3459 | |
| 69 | CAACCGCC C GCGGCUCU | 759 | AGAGCCGC CUGAUGAG GCCGUUAGGC CGAA IGCGGUUG | 3460 | |
| 75 | CCCGCGGC U CUGAGACG | 760 | CGUCUCAG CUGAUGAG GCCGUUAGGC CGAA ICCGCGGG | 3461 | |
| 77 | CGCGGCUC U GAGACGCG | 761 | CGCGUCUC CUGAUGAG GCCGUUAGGC CGAA IAGCCGCG | 3462 | |
| 88 | GACGCGGC C CCGGCGGC | 762 | GCCGCCGG CUGAUGAG GCCGUUAGGC CGAA ICCGCGUC | 3463 | |
| 89 | ACGCGGCC C CGGCGGCG | 763 | CGCCGCCG CUGAUGAG GCCGUUAGGC CGAA IGCCGCGU | 3464 | |
| 90 | CGCGGCCC C GGCGGCGG | 764 | CCGCCGCC CUGAUGAG GCCGUUAGGC CGAA IGGCCGCG | 3465 | |
| 103 | GCGGCGGC A GCAGCUGC | 765 | GCAGCUGC CUGAUGAG GCCGUUAGGC CGAA ICCGCCGC | 3466 | |
| 106 | GCGGCAGC A GCUGCAGC | 766 | GCUGCAGC CUGAUGAG GCCGUUAGGC CGAA ICUGCCGC | 3467 | |
| 109 | GCAGCAGC U GCAGCAUC | 767 | GAUGCUGC CUGAUGAG GCCGUUAGGC CGAA ICUGCUGC | 3468 | |
| 112 | GCAGCUGC A GCAUCAUC | 768 | GAUGAUGC CUGAUGAG GCCGUUAGGC CGAA ICAGCUGC | 3469 | |
| 115 | GCUGCAGC A UCAUCUCC | 769 | GGAGAUGA CUGAUGAG GCCGUUAGGC CGAA ICUGCAGC | 3470 | |
| 118 | GCAGCAUC A UCUCCACC | 770 | GGUGGAGA CUGAUGAG GCCGUUAGGC CGAA IAUGCUGC | 3471 | |
| 121 | GCAUCAUC U CCACCCUC | 771 | GAGGGUGG CUGAUGAG GCCGUUAGGC CGAA IAUGAUGC | 3472 | |
| 123 | AUCAUCUC C ACCCUCCA | 772 | UGGAGGGU CUGAUGAG GCCGUUAGGC CGAA IAGAUGAU | 3473 | |
| 124 | UCAUCUCC A CCCUCCAG | 773 | CUGGAGGG CUGAUGAG GCCGUUAGGC CGAA IGAGAUGA | 3474 | |
| 126 | AUCUCCAC C CUCCAGCC | 774 | GGCUGGAG CUGAUGAG GCCGUUAGGC CGAA IUGGAGAU | 3475 | |
| 127 | UCUCCACC C UCCAGCCA | 775 | UGGCUGGA CUGAUGAG GCCGUUAGGC CGAA IGUGGAGA | 3476 | |
| 128 | CUCCACCC U CCAGCCAU | 776 | AUGGCUGG CUGAUGAG GCCGUUAGGC CGAA IGGUGGAG | 3477 | |
| 130 | CCACCCUC C AGCCAUGG | 777 | CCAUGGCU CUGAUGAG GCCGUUAGGC CGAA IAGGGUGG | 3478 | |
| 131 | CACCCUCC A GCCAUGGA | 778 | UCCAUGGC CUGAUGAG GCCGUUAGGC CGAA IGAGGGUG | 3479 | |
| 134 | CCUCCAGC C AUGGAAGA | 775 | UCUUCCAU CUGAUGAG GCCGUUAGGC CGAA ICUGGAGG | 3480 | |
| 135 | CUCCAGCC A UGGAAGAC | 780 | GUCUUCCA CUGAUGAG GCCGUUAGGC CGAA IGCUGGAG | 3481 | |
| 144 | UGGAAGAC C UGGACCAG | 781 | CUGGUCCA CUGAUGAG GCCGUUAGGC CGAA IUCUUCCA | 3482 | |
| 145 | GGAAGACC U GGACCAGU | 782 | ACUGGUGC CUGAUGAG GCCGUUAGGC CGAA IGUCUUCC | 3483 | |
| 150 | ACCUGGAC C AGUCUCCU | 783 | AGGAGACU CUGAUGAG GCCGUUAGGC CGAA IUCCAGGU | 3484 | |
| 151 | CCUGGACC A GUCUCCUC | 784 | GAGGAGAC CUGAUGAG GCCGUUAGGC CGAA IGUCCAGG | 3485 | |
| 155 | GACCAGUC U CCUCUGGU | 785 | ACCAGAGG CUGAUGAG GCCGUUAGGC CGAA IACUGGUC | 3488 | |
| 157 | CCAGUCUC C UCUGGUCU | 785 | AGACCAGA CUGAUGAG GCCGUUAGGC CGAA IAGACUGG | 3487 | |
| 158 | CAGUCUCC U CUGGUCUC | 787 | GAGACCAG CUGAUGAG GCCGUUAGGC CGAA IGAGACUG | 3488 | |
| 160 | GUCUCCUC U GGUCUCGU | 788 | ACGAGACC CUGAUGAG GCCGUUAGGC CGAA IAGGAGAC | 3489 | |
| 165 | CUCUGGUC U CGUCCUCG | 789 | CGAGGACG CUGAUGAG GCCGUUAGGC CGAA IACCAGAG | 3490 | |
| 170 | GUCUGGUC C UCGGACAG | 790 | CUGUCCGA CUGAUGAG GCCGUUAGGC CGAA IACGAGAC | 3491 | |
| 171 | UCUCGUCC U CGGACAGC | 791 | GCUGUCCG CUGAUGAG GCCGUUAGGC CGAA IGACGAGA | 3492 | |
| 177 | CCUCGGAC A GCCCACCC | 792 | GGGUGGGC CUGAUGAG GCCGUUAGGC CGAA IUCCGAGG | 3493 | |
| 180 | CGGACAGC C CACCCCGG | 793 | CCGGGGUG CUGAUGAG GCCGUUAGGC CGAA ICUGUCCG | 3494 | |
| 181 | GGACAGCC C ACCCCGGC | 794 | GCCGGGGU CUGAUGAG GCCGUUAGGC CGAA IGCUGUCC | 3495 | |
| 182 | GACAGCCC A CCCCGGCC | 795 | GGCCGGGG CUGAUGAG GCCGUUAGGC CGAA IGGCUGUC | 3496 | |
| 184 | CAGCCCAC C CCGGCCGC | 796 | GCGGCCGG CUGAUGAG GCCGUUAGGC CGAA IUGGGCUG | 3497 | |
| 185 | AGCCCACC C CGGCCGCA | 797 | UGCGGCCG CUGAUGAG GCCGUUAGGC CGAA IGUGGGCU | 3498 | |
| 186 | GCCCACCC C GGCCGCAG | 798 | CUGCGGCC CUGAUGAG GCCGUUAGGC CGAA IGGUGGGC | 3499 | |
| 190 | ACCCCGGC C GCAGCCCG | 799 | CGGGCUGC CUGAUGAG GCCGUUAGGC CGAA ICCGGGGU | 3500 | |
| 193 | CCGGCCGC A GCCCGCGU | 800 | ACGCGGGC CUGAUGAG GCCGUUAGGC CGAA ICGGCCGG | 3501 | |
| 196 | GCCGCAGC C CGCGUUCA | 801 | UGAACGCG CUGAUGAG GCCGUUAGGC CGAA ICUGCGGC | 3502 | |
| 197 | CCGCAGCC C GCGUUCAA | 802 | UUGAACGC CUGAUGAG GCCGUUAGGC CGAA IGCUGCGG | 3503 | |
| 204 | CCGCGUUC A AGUACCAG | 803 | CUGGUACU CUGAUGAG GCCGUUAGGC CGAA IAACGCGG | 3504 | |
| 210 | UCAAGUAC C AGUUCGUG | 804 | CACGAACU CUGAUGAG GCCGUUAGGC CGAA IUACUUGA | 3505 | |
| 211 | CAAGUACC A GUUCGUGA | 805 | UCACGAAC CUGAUGAG GCCGUUAGGC CGAA IGUACUUG | 3506 | |
| 226 | GAGGGAGC C CGAGGACG | 806 | CGUCCUCG CUGAUGAG GCCGUUAGGC CGAA ICUCCCUC | 3507 | |
| 227 | AGGGAGCC C GAGGACGA | 807 | UCGUCCUC CUGAUGAG GCCGUUAGGC CGAA IGCUCCCU | 3508 | |
| 279 | ACGAAGAC C UGGAGGAG | 808 | CUCCUCCA CUGAUGAG GCCGUUAGGC CGAA IUCUUCGU | 3509 | |
| 280 | CGAAGACC U GGAGGAGC | 809 | GCUCCUCC CUGAUGAG GCCGUUAGGC CGAA IGUCUUCG | 3510 | |
| 289 | GGAGGAGC U GGAGGUGC | 810 | GCACCUCC CUGAUGAG GCCGUUAGGC CGAA ICUCCUCC | 3511 | |
| 298 | GGAGGUGC U GGAGAGGA | 811 | UCCUCUCC CUGAUGAG GCCGUUAGGC CGAA ICACCUGC | 3512 | |
| 310 | GAGGAAGC C CGCCGCCG | 812 | CGGCGGCG CUGAUGAG GCCGUUAGGC CGAA ICUUCCUC | 3513 | |
| 311 | AGGAAGCC C GCCGCCGG | 813 | CCGGCGGC CUGAUGAG GCCGUUAGGC CGAA IGCUUCCU | 3514 | |
| 314 | AAGCCCGC C GCCGGGCU | 814 | AGCCCGGC CUGAUGAG GCCGUUAGGC CGAA ICGGGCUU | 3515 | |
| 317 | CCCGCCGC C GGGCUGUC | 815 | GACAGCCC CUGAUGAG GCCGUUAGGC CGAA ICGGCGGG | 3516 | |
| 322 | CGCCGGGC U GUCCGCGG | 816 | CCGCGGAC CUGAUGAG GCCGUUAGGC CGAA ICCCGGCG | 3517 | |
| 326 | GGGCUGUC C GCGGCCCC | 817 | GGGGCCGC CUGAUGAG GCCGUUAGGC CGAA IACAGCCC | 3518 | |
| 332 | UCCGCGGC C CCAGUGCC | 818 | GGCACUGG CUGAUGAG GCCGUUAGGC CGAA ICCGCGGA | 3519 | |
| 333 | CCGCGGCC C CAGUGCCC | 819 | GGGCACUG CUGAUGAG GCCGUUAGGC CGAA IGCCGCGG | 3520 | |
| 334 | CGCGGCCC C AGUGCCCA | 820 | UGGGCACU CUGAUGAG GCCGUUAGGC CGAA IGGCCGCG | 3521 | |
| 335 | GCGGCCCC A GUGCCCAC | 821 | GUGGGCAC CUGAUGAG GCCGUUAGGC CGAA IGGGCCGC | 3522 | |
| 340 | CCCAGUGC C CACCGCCC | 822 | GGGCGGUG CUGAUGAG GCCGUUAGGC CGAA ICACUGGG | 3523 | |
| 341 | CCAGUGCC C ACCGCCCC | 823 | GGGGCGGU CUGAUGAG GCCGUUAGGC CGAA IGCACUGG | 3524 | |
| 342 | CAGUGCCC A CCGCCCCU | 824 | AGGGGCGG CUGAUGAG GCCGUUAGGC CGAA IGGCACUG | 3525 | |
| 344 | GUGCCCAC C GCCCCUGC | 825 | GCAGGGGC CUGAUGAG GCCGUUAGGC CGAA IUGGGCAC | 3526 | |
| 347 | CCCACCGC C CCUGCCGC | 826 | GCGGCAGG CUGAUGAG GCCGUUAGGC CGAA ICGGUGGG | 3527 | |
| 348 | CCACCGCC C CUGCCGCC | 827 | GGCGGCAG CUGAUGAG GCCGUUAGGC CGAA IGCGGUGG | 3528 | |
| 349 | CACCGCCC C UGCCGCCG | 828 | CGGCGGCA CUGAUGAG GCCGUUAGGC CGAA IGGCGGUG | 3529 | |
| 350 | ACCGCCCC U GCCGCCGG | 829 | CCGGCGGC CUGAUGAG GCCGUUAGGC CGAA IGGGCGGU | 3530 | |
| 353 | GCCCCUGC C GCCGGCGC | 830 | GCGCCGGC CUGAUGAG GCCGUUAGGC CGAA ICAGGGGC | 3531 | |
| 356 | CCUGCCGC C GGCGCGCC | 831 | GGCGCGCC CUCAUGAG GCCGUUAGGC CGAA ICGGCAGG | 3532 | |
| 364 | CGGCGCGC C CCUGAUGG | 832 | CCAUCAGG CUGAUGAG GCCGUUAGGC CGAA ICGCGCCG | 3533 | |
| 365 | GGCGCGCC C CUGAUGGA | 833 | UCCAUCAG CUGAUGAG GCCGUUAGGC CGAA IGCGCGCC | 3534 | |
| 366 | GCGCGCCC C UGAUGGAC | 834 | GUCCAUCA CUGAUGAG GCCGUUAGGC CGAA IGGCGCGC | 3535 | |
| 367 | CGCGCCCC U GAUGGACU | 835 | AGUCCAUC CUGAUGAG GCCGUUAGGC CGAA IGGGCGCG | 3536 | |
| 376 | UGAUGGAC U UCGGAAAU | 836 | AUUUCCGA CUGAUGAG GCCGUUAGGC CGAA IUCCAUCA | 3537 | |
| 387 | GAAAUGAC U UCGUGCCG | 837 | CGGCACGA CUGAUGAG GCCGUUAGGC CGAA IUCAUUUC | 3538 | |
| 394 | CUUCGUGC C GCCGGCGC | 838 | GCGCCGGC CUGAUGAG GCCGUUAGGC CGAA ICACGAAG | 3539 | |
| 397 | CGUGCCGC C GGCGCCCC | 839 | GGGGCGCC CUGAUGAG GCCGUUAGGC CGAA ICGGCACG | 3540 | |
| 403 | GCCGGCGC C CCGGGGAC | 840 | GUCCCCGG CUGAUGAG GCCGUUAGGC CGAA ICGCCGGC | 3541 | |
| 404 | CCGGCGCC C CGGGGACC | 841 | GGUCCCCG CUGAUGAG GCCGUUAGGC CGAA IGCGCCGG | 3542 | |
| 405 | CGGCGCCC C GGGGACCC | 842 | GGGUCCCC CUGAUGAG GCCGUUAGGC CGAA IGGCGCCG | 3543 | |
| 412 | CCGGGGAC C CCUGCCGG | 843 | CCGGCAGG CUGAUGAG GCCGUUAGGC CGAA IUCCCCGG | 3544 | |
| 413 | CGGGGACC C CUGCCGGC | 844 | GCCGGCAG CUGAUGAG GCCGUUAGGC CGAA IGUCCCCG | 3545 | |
| 414 | GGGGACCC C UGCCGGCC | 845 | GGCCGGCA CUGAUGAG GCCGUUAGGC CGAA IGGUCCCC | 3546 | |
| 415 | GGGACCCC U GCCGGCCG | 846 | CGGCCGGC CUGAUGAG GCCGUUAGGC CGAA IGGGUCCC | 3547 | |
| 418 | ACCCCUGC C GGCCGCUC | 847 | GAGCGGCC CUGAUGAG GCCGUUAGGC CGAA ICAGGGGU | 3548 | |
| 422 | CUGCCGGC C GCUCCCCC | 848 | GGGGGAGC CUGAUGAG GCCGUUAGGC CGAA ICCGGCAG | 3549 | |
| 425 | CCGGCCGC U CCCCCCGU | 849 | ACGGGGGG CUGAUGAG GCCGUUAGGC CGAA ICGGCCGG | 3550 | |
| 427 | GGCCGCUC C CCCCGUCG | 850 | CGACGGGG CUGAUGAG GCCGUUAGGC CGAA IAGCGGCC | 3551 | |
| 428 | GCCGCUCC C CCCGUCGC | 851 | GCGACGGG CUGAUGAG GCCGUUAGGC CGAA IGAGCGGC | 3552 | |
| 429 | CCGCUCCC C CCGUCGCC | 852 | GGCGACGG CUGAUGAG GCCGUUAGGC CGAA IGGAGCGG | 3553 | |
| 430 | CGCUCCCC C CGUCGCCC | 853 | GGGCGACG CUGAUGAG GCCGUUAGGC CGAA IGGGAGCG | 3554 | |
| 431 | GCUCCCCC C GUCGCCCC | 854 | GGGGCGAC CUGAUGAG GCCGUUAGGC CGAA IGGGGAGC | 3555 | |
| 437 | CCCGUCGC C CCGGAGCG | 855 | CGCUCCGG CUGAUGAG GCCGUUAGGC CGAA ICGACGGG | 3556 | |
| 438 | CCGUCGCC C CGGAGCGG | 856 | CCGCUCCG CUGAUGAG GCCGUUAGGC CGAA IGCGACGG | 3557 | |
| 439 | CGUCGCCC C GGAGCGGC | 857 | GCCGCUCC CUGAUGAG GCCGUUAGGC CGAA IGGCGACG | 3558 | |
| 448 | GGAGCGGC A GCCGUCUU | 858 | AAGACGGC CUGAUGAG GCCGUUAGGC CGAA ICCGCUCC | 3559 | |
| 451 | GCGGCAGC C GUCUUGGG | 859 | CCCAAGAC CUGAUGAG GCCGUUAGGC CGAA ICUGCCGC | 3560 | |
| 455 | CAGCCGUC U UGGGACCC | 860 | GGGUCCCA CUGAUGAG GCCGUUAGGC CGAA IACGGCUG | 3561 | |
| 462 | CUUGGGAC C CGAGCCCG | 861 | CGGGCUCG CUGAUGAG GCCGUUAGGC CGAA IUCCCAAG | 3562 | |
| 463 | UUGGGACC C GAGCCCGG | 862 | CCGGGCUC CUGAUGAG GCCGUUAGGC CGAA IGUCCCAA | 3563 | |
| 468 | ACCCGAGC C CGGUGUCG | 863 | CGACACCG CUGAUGAG GCCGUUAGGC CGAA ICUCGGGU | 3564 | |
| 469 | CCCGAGCC C GGUGUCGU | 864 | ACGACACC CUGAUGAG GCCGUUAGGC CGAA IGCUCGGG | 3565 | |
| 482 | UCGUCGAC C GUGCCCGC | 865 | GCGGGCAC CUGAUGAG GCCGUUAGGC CGAA IUCGACGA | 3566 | |
| 487 | GACCGUGC C CGCGCCAU | 866 | AUGGCGCG CUGAUGAG GCCGUUAGGC CGAA ICACCGUC | 3567 | |
| 488 | ACCGUGCC C GCGCCAUC | 867 | GAUGGCGC CUGAUGAG GCCGUUAGGC CGAA UGCACGGU | 3568 | |
| 493 | GCCCGCGC C AUCCCCGC | 868 | GCGGGGAU CUGAUGAG GCCGUUAGGC CGAA ICGCGGGC | 3569 | |
| 494 | CCCGCGCC A UCCCCGCU | 869 | AGCGGGGA CUGAUGAG GCCGUUAGGC CGAA IGCGCGGG | 3570 | |
| 497 | GCGCCAUC C CCGCUGUC | 870 | GACAGCGG CUGAUGAG GCCGUUAGGC CGAA IAUGGCGC | 3571 | |
| 498 | CGCCAUCC C CGCUGUCU | 871 | AGACAGCG CUGAUGAG GCCGUUAGGC CGAA IGAUGGCG | 3572 | |
| 499 | GCCAUCCC C GCUGUCUG | 872 | CAGACAGC CUGAUGAG GCCGUUAGGC CGAA IGGAUGGC | 3573 | |
| 502 | AUCCCCGC U GUCUGCUG | 873 | CAGCAGAC CUGAUGAG GCCGUUAGGC CGAA ICGGGGAU | 3574 | |
| 506 | CCGCUGUC U GCUGCCGC | 874 | GCGGCAGC CUGAUGAG GCCGUUAGGC CGAA IACAGCGG | 3575 | |
| 509 | CUGUCUGC U GCCGCAGU | 875 | ACUGCGGC CUGAUGAG GCCGUUAGGC CGAA ICAGACAG | 3576 | |
| 512 | UCUGCUGC C GCAGUCUC | 876 | GAGACUGC CUGAUGAG GCCGUUAGGC CGAA ICAGCAGA | 3577 | |
| 515 | GCUGCCGC A GUCUCGCC | 877 | GGCGAGAC CUGAUGAG GCCGUUAGGC CGAA ICGGCAGC | 3578 | |
| 519 | CCGCAGUC U CGCCCUCC | 878 | GGAGGGCG CUGAUGAG GCCGUUAGGC CGAA IACUGCGG | 3579 | |
| 523 | AGUCUCGC C CUCCAAGC | 879 | GCUUGGAG CUGAUGAG GCCGUUAGGC CGAA ICGAGACU | 3580 | |
| 524 | GUCUCGCC C UCCAAGCU | 880 | AGCUUGGA CUGAUGAG GCCGUUAGGC CGAA IGCGAGAC | 3581 | |
| 525 | UCUCGCCC U CCAAGCUC | 881 | GAGCUUGG CUGAUGAG GCCGUUAGGC CGAA IGGCGAGA | 3582 | |
| 527 | UCGCCCUC C AAGCUCCC | 882 | GGGAGCUU CUGAUGAG GCCGUUAGGC CGAA IAGGGCGA | 3583 | |
| 528 | CGCCCUCC A AGCUCCCU | 883 | AGGGAGCU CUGAUGAG GCCGUUAGGC CGAA IGAGGGCG | 3584 | |
| 532 | CUCCAAGC U CCCUGAGG | 884 | CCUCAGGG CUGAUGAG GCCGUUAGGC CGAA ICUUGGAG | 3585 | |
| 534 | CCAAGCUC C CUGAGGAC | 885 | GUCCUCAG CUGAUGAG GCCGUUAGGC CGAA IAGCUUGG | 3586 | |
| 535 | CAAGCUCC C UGAGGACG | 886 | CGUCCUCA CUGAUGAG GCCGUUAGGC CGAA IGAGCUUG | 3587 | |
| 536 | AAGCUCCC U GAGGACGA | 887 | UCGUCCUC CUGAUGAG GCCGUUAGGC CGAA IGGAGCUU | 3588 | |
| 550 | CGACGAGC C UCCGGCCC | 888 | GGGCCGGA CUGAUGAG GCCGUUAGGC CGAA ICUCGUCG | 3589 | |
| 551 | GACUAGUC U CCGGCCCG | 889 | CGGGCCGG CUGAUGAG GCCGUUAGGC CGAA IGCUCGUC | 3590 | |
| 553 | CGAGCCUC C GGCCCGGC | 890 | GCCGGGCC CUGAUGAG GCCGUUAGGC CGAA IAGGCUCG | 3591 | |
| 557 | CCUCCGGC C CGGCCUCC | 891 | GGAGGCCG CUGAUGAG GCCGUUAGGC CGAA ICCGGAGG | 3592 | |
| 558 | CUCCGGCC C GGCCUCCC | 892 | GGGAGGCC CUGAUGAG GCCGUUAGGC CGAA IGCCGGAG | 3593 | |
| 562 | GGCCCGGC C UCCCCCUC | 893 | GAGGGGGA CUGAUGAG GCCGUUAGGC CGAA ICCGGGCC | 3594 | |
| 563 | GCCCGGCC U CCCCCUCC | 894 | GGAGGGGG CUGAUGAG GCCGUUAGGC CGAA IGCCGGGC | 3595 | |
| 565 | CCGGCCUC C CCCUCCUC | 895 | GAGGAGGG CUGAUGAG GCCGUUAGGC CGAA IAGGCCGG | 3596 | |
| 566 | CGGCCUCC C CCUCCUCC | 896 | GGAGGAGG CUGAUGAG GCCGUUAGGC CGAA IGAGGCCG | 3597 | |
| 567 | GGCCUCCC C CUCCUCCC | 897 | UGGAGGAG CUGAUGAG GCCGUUAGGC CGAA IGGAGGCC | 3598 | |
| 568 | GCCUCCCC C UCCUCCUC | 898 | GGGGAGGA CUGAUGAG GCCGUUAGGC CGAA IGGGAGGC | 3599 | |
| 569 | CCUCCCCC U CCUCCCCC | 899 | GGGGGAGG CUGAUGAG GCCGUUAGGC CGAA IGGGGAGG | 3600 | |
| 571 | UCCCCCUC C UCCCCCGG | 900 | CCGGGGGA CUGAUGAG GCCGUUAGGC CGAA IAGGGGGA | 3601 | |
| 572 | CCCCCUCC U CCCCCGGC | 901 | GCCGGGGG CUGAUGAG GCCGUUAGGC CGAA IGAGGGGG | 3502 | |
| 574 | CCCUCCUC C CCCGGCCA | 902 | UGGCCGGG CUGAUGAG GCCGUUAGGC CGAA IAGGAGGG | 3503 | |
| 575 | CCUCCUCC C CCGGCCAG | 903 | CUGGCCGG CUGAUGAG GCCGUUAGGC CGAA IGAGGAGG | 3604 | |
| 576 | CUCCUCCC C CGGCCAGC | 904 | GCUGGCCG CUGAUGAG GCCGUUAGGC CGAA IGGAGGAG | 3605 | |
| 577 | UCCUCCCC C GGCCAGCG | 905 | CGCUGGCC CUGAUGAG GCCGUUAGGC CGAA IGGGAGGA | 3606 | |
| 581 | CCCCCGGC C AGCGUGAG | 906 | CUCACGCU CUGAUGAG GCCGUUAGGC CGAA ICCGGGGG | 3607 | |
| 582 | CCCCGGCC A GCGUGAGC | 907 | GCUCACGC CUGAUGAG GCCGUUAGGC CGAA IGCCGGGG | 3608 | |
| 591 | GCGUGAGC C CCCAGGCA | 908 | UGCCUGGG CUGAUGAG GCCGUUAGGC CGAA ICUCACGC | 3509 | |
| 592 | CGUGAGCC C CCAGGCAG | 909 | CUGCCUGG CUGAUGAG GCCGUUAGGC CGAA IGCUCACG | 3510 | |
| 593 | GUGAGCCC C CAGGCAGA | 910 | UCUGCCUG CUGAUGAG GCCGUUAGGC CGAA IGGCUCAC | 3611 | |
| 594 | UGAGCCCC C AGGCAGAG | 911 | CUCUGCCU CUGAUGAG GCCGUUAGGC CGAA IGGGCUCA | 3612 | |
| 595 | GAGCCCCC A GGCAGAGC | 912 | GCUCUGCC CUGAUGAG GCCGUUAGGC CGAA IGGGGCUC | 3613 | |
| 599 | CCCCAGGC A GAGCCCGU | 913 | ACGGGCUC CUGAUGAG GCCGUUAGGC CGAA ICCUGGGG | 3614 | |
| 604 | GGCAGAGC C CGUGUGGA | 914 | UCCACACG CUGAUGAG GCCGUUAGGC CGAA ICUCUGCC | 3615 | |
| 605 | GCAGAGCC C GUGUGGAC | 915 | GUCCACAC CUGAUGAG GCCGUUAGGC CGAA IGCUCUGC | 3616 | |
| 614 | GUGUGGAC C CCGCCAGC | 915 | GCUGGCGG CUGAUGAG GCCGUUAGGC CGAA IUCCACAC | 3517 | |
| 615 | UGUGGACC C CGCCAGCC | 917 | GGCUGGCG CUGAUGAG GCCGUUAGGC CGAA IGUCCACA | 3618 | |
| 616 | GUGGACCC C GCCAGCCC | 918 | GGGCUGGC CUGAUGAG GCCGUUAGGC CGAA IGGUCCAC | 3619 | |
| 519 | GACCCCGC C AGCCCCGG | 919 | CCGGGGCU CUGAUGAG GCCGUUAGGC CGAA ICGGGGUC | 3620 | |
| 620 | ACCCCGCC A GCCCCGGC | 920 | GCCGGGGC CUGAUGAG GCCGUUAGGC CGAA IGCGGGGU | 3521 | |
| 523 | CCGCCAGC C CCGGCUCC | 921 | GGAGCCGG CUGAUGAG GCCGUUAGGC CGAA ICUGGCGG | 3622 | |
| 624 | CGCCAGCC C CGGCUCCC | 922 | GGGAGCCG CUGAUGAG GCCGUUAGGC CGAA IGCUGGCG | 3623 | |
| 525 | GCCAGCCC C GGCUCCCG | 923 | CGGGAGCC CUGAUGAG GCCGUUAGGC CGAA IGGCUGGC | 3624 | |
| 629 | GCCCCGGC U CCCGCCGC | 924 | GCGGCGGG CUGAUGAG GCCGUUAGGC CGAA ICCGGGGC | 3625 | |
| 631 | CCCGGCUC C CGCCGCGC | 925 | GCGCGGCG CUGAUGAG GCCGUUAGGC CGAA IAGCCGGG | 3626 | |
| 632 | CCGGCUCC C GCCGCGCC | 926 | GGCGCGGC CUGAUGAG GCCGUUAGGC CGAA IGAGCCGG | 3627 | |
| 535 | GCUCCCGC C GCGCCCCC | 927 | GGGGGCGC CUGAUGAG GCCGUUAGGC CGAA ICGGGAGC | 3528 | |
| 540 | CGCCGCGC C CCCCUCCA | 928 | UGGAGGGG CUGAUGAG GCCGUUAGGC CGAA ICGCGGCG | 3529 | |
| 541 | GCCGCGCC C CCCUCCAC | 929 | GUGGAGGG CUGAUGAG GCCGUUAGGC CGAA IGCGCGGC | 3530 | |
| 642 | CCGCCCCC C CCUCCACC | 930 | GGUGGAGG CUGAUGAG GCCGUUAGGC CGAA IGGCGCGG | 3631 | |
| 543 | CGCGCCCC C CUCCACCC | 931 | GGGUGGAG CUGAUGAG GCCGUUAGGC CGAA IGGGCGCG | 3532 | |
| 644 | GCGCCCCC C UCCACCCC | 932 | GGGGUGGA CUGAUGAG GCCGUUAGGC CGAA IGGGGCGC | 3533 | |
| 645 | CGCCCCCC U CCACCCCG | 933 | CGGGGUGG CUGAUGAG GCCGUUAGGC CGAA IGGGGGCG | 3534 | |
| 547 | CCCCCCUC C ACCCCGGC | 934 | GCCGGGGU CUGAUGAG GCCGUUAGGC CGAA IAGGGGGG | 3535 | |
| 648 | CCCCCUCC A CCCCGGCC | 935 | GGCCGGGG CUGAUGAG GCCGUUAGGC CGAA IGAGGGGG | 3636 | |
| 650 | CCCUCCAC C CCGGCCCC | 936 | GCGGCCGG CUGAUGAG GCCGUUAGGC CGAA IUGGAGGG | 3637 | |
| 651 | CCUCCACC C CGGCCGCG | 937 | CGCGGCCG CUGAUGAG GCCGUUAGGC CGAA IGUGGAGG | 3638 | |
| 652 | CUCCACCC C GGCCGCGC | 938 | GCGCGGCC CUGAUGAG GCCGUUAGGC CGAA IGGUGGAG | 3639 | |
| 656 | ACCCCGGC C GCGCCCAA | 939 | UUGGGCGC CUGAUGAG GCCGUUAGGC CGAA ICCGGGGU | 3640 | |
| 661 | GGCCGCGC C CAAGCGCA | 940 | UGCGCUUG CUGAUGAG GCCGUUAGGC CGAA ICGCGGCC | 3641 | |
| 662 | GCCACGCC C AAGCGCAG | 941 | CUGCGCUU CUGAUGAG GCCGUUAGGC CGAA IGCGCGGC | 3642 | |
| 663 | CCGCGCCC A AGCGCAGG | 942 | CCUGCGCU CUGAUGAG GCCGUUAGGC CGAA IGGCGCGG | 3643 | |
| 669 | CCAAGCGC A GGGGCUCC | 943 | GGAGCCCC CUGAUGAG GCCGUUAGGC CGAA ICGCUUGG | 3644 | |
| 675 | GCAGGGGC U CCUCGGGC | 944 | GCCCGAGG CUGAUGAG GCCGUUAGGC CGAA ICCCCUGC | 3645 | |
| 677 | AGGGGCUC C UCGGGCUC | 945 | GAGCCCGA CUGAUGAG GCCGUUAGGC CGAA IAGCCCCU | 3646 | |
| 678 | GGGGCUCC U CGGGCUCA | 946 | UGAGCCCG CUGAUGAG GCCGUUAGGC CGAA IGAGCCCC | 3647 | |
| 684 | CCUCGGGC U CAGUGGAU | 947 | AUCCACUG CUGAUGAG GCCGUUAGGC CGAA ICCCGAGG | 3648 | |
| 686 | UCGGGCUC A GUGGAUGA | 948 | UCAUCCAC CUGAUGAG GCCGUUAGGC CGAA IAGCCCGA | 3649 | |
| 698 | GAUGAGAC C CUUUUUGC | 949 | GCAAAAAG CUGAUGAG GCCGUUAGGC CGAA IUCUCAUC | 3650 | |
| 699 | AUGAGACC C UUUUUGCU | 950 | AGCAAAAA CUGAUGAG GCCGUUAGGC CGAA IGUCUCAU | 3651 | |
| 700 | UGAGACCC U UUUUGCUC | 951 | GAGCAAAA CUGAUGAG GCCGUUAGGC CGAA IGGUCUCA | 3652 | |
| 707 | CUUUUUGC U CUUCCUGC | 952 | GCAGGAAG CUGAUGAG GCCGUUAGGC CGAA ICAAAAAG | 3653 | |
| 709 | UUUUGCUC U UCCUGCUG | 953 | CAGCAGGA CUGAUGAG GCCGUUAGGC CGAA IAGCAAAA | 3654 | |
| 712 | UGCUCUUC C UGCUGCAU | 954 | AUGCAGCA CUGAUGAG GCCGUUAGGC CGAA IAAGAGCA | 3655 | |
| 713 | GCUCUUCC U GCUGCAUC | 955 | GAUGCAGC CUGAUGAG GCCGUUAGGC CGAA IGAAGAGC | 3656 | |
| 716 | CUUCCUGC U GCAUCUGA | 956 | UCAGAUGC CUGAUGAG GCCGUUAGGC CGAA ICAGGAAG | 3657 | |
| 719 | CCUGCUGC A UCUGAGCC | 957 | GGCUCAGA CUGAUGAG GCCGUUAGGC CGAA ICAGCAGG | 3658 | |
| 722 | GCUGCAUC U GAGCCUGU | 958 | ACAGGCUC CUGAUGAG GCCGUUAGGC CGAA IAUGCAGC | 3659 | |
| 727 | AUCUGAGC C UGUGAUAC | 959 | GUAUCACA CUGAUGAG GCCGUUAGGC CGAA ICUCAGAU | 3660 | |
| 728 | UCUGAGCC U GUGAUACG | 960 | CGUAUCAC CUGAUGAG GCCGUUAGGC CGAA IGCUCAGA | 3661 | |
| 738 | UGAUACGC U CCUCUGCA | 961 | UGCAGAGG CUGAUGAG GCCGUUAGGC CGAA ICGUAUCA | 3662 | |
| 740 | AUACGCUC C UCUGCAGA | 962 | UCUGCAGA CUGAUGAG GCCGUUAGGC CGAA IAGCGUAU | 3663 | |
| 741 | UACGCUCC U CUGCAGAA | 963 | UUCUGCAG CUGAUGAG GCCGUUAGGC CGAA IGAGCGUA | 3664 | |
| 743 | CGCUCCUC U GCAGAAAA | 964 | UUUUCUGC CUGAUGAG GCCGUUAGGC CGAA IAGGAGCG | 3665 | |
| 746 | UCCUCUGC A GAAAAUAU | 965 | AUAUUUUC CUGAUGAG GCCGUUAGGC CGAA ICAGAGGA | 3666 | |
| 759 | AUAUGGAC U UGAAGGAG | 966 | CUCCUUCA CUGAUGAG GCCGUUAGGC CGAA IUCCAUAU | 3667 | |
| 769 | GAAGGAGC A GCCAGGUA | 967 | UACCUGGC CUGAUGAG GCCGUUAGGC CGAA ICUCCUUC | 3668 | |
| 772 | GGAGCAGC C AGGUAACA | 968 | UGUUACCU CUGAUGAG GCCGUUAGGC CGAA ICUGCUCC | 3669 | |
| 773 | GAGCAGCC A GGUAACAC | 969 | GUGUUACC CUGAUGAG GCCGUUAGGC CGAA IGCUGCUC | 3670 | |
| 780 | CAGGUAAC A CUAUUUCG | 970 | CGAAAUAG CUGAUGAG GCCGUUAGGC CGAA IUGACCUG | 3671 | |
| 782 | GGUAACAC U AUUUCGGC | 971 | GCCGAAAU CUGAUGAG GCCGUUAGGC CGAA IUGUUACC | 3672 | |
| 791 | AUUUCGGC U GGUCAAGA | 972 | UCUUGACC CUGAUGAG GCCGUUAGGC CGAA ICCGAAAU | 3673 | |
| 796 | GGCUGGUC A AGAGGAUG | 973 | AAUCCUCU CUGAUGAG GCCGUUAGGC CGAA IACCAGCC | 3674 | |
| 807 | AGGAUUUC C CAUCUGUC | 974 | GACAGAUG CUGAUGAG GCCGUUAGGC CGAA IAAAUCCU | 3675 | |
| 808 | GGAUUUCC C AUCUGUCC | 975 | GGACAGAU CUGAUGAG GCCGUUAGGC CGAA IGAAAUCC | 3676 | |
| 809 | GAUUUCCC A UCUGUCCU | 976 | AGGACAGA CUGAUGAG GCCGUUAGGC CGAA IGGAAAUC | 3677 | |
| 812 | UGGCCAUC U GUCCUGCU | 977 | AGCAGGAC CUGAUGAG GCCGUUAGGC CGAA IAUGGGAA | 3678 | |
| 816 | CAUCUGUC C UGCUUGAA | 978 | UUCAAGCA CUGAUGAG GCCGUUAGGC CGAA IACAGAUG | 3679 | |
| 817 | AUCUGUCC U GCUUGAAA | 979 | UUUCAAGC CUGAUGAG GCCGUUAGGC CGAA IGACAGAU | 3680 | |
| 820 | UGUCCUGC U UGAAACUG | 980 | CAGUUUCA CUGAUGAG GCCGUUAGGC CGAA ICAGGACA | 3681 | |
| 827 | CUUGAAAC U GCUGCUUC | 981 | GAAGCAGC CUGAUGAG GCCGUUAGGC CGAA IUUUCAAG | 3682 | |
| 830 | GAAACUGC U GCUUCUCU | 982 | AGAGAAGC CUGAUGAG GCCGUUAGGC CGAA ICAGUUUC | 3683 | |
| 833 | ACUGCUGC U UCUCUGGC | 983 | GGAAGAGA CUGAUGAG GCCGUUAGGC CGAA ICAGCAGU | 3684 | |
| 836 | GCUGCUUC U CUUCCUUC | 984 | GAAGGAAG CUGAUGAG GCCGUUAGGC CGAA IAAGCAGC | 3685 | |
| 838 | UGCUGCUC U UCCUUCUC | 985 | GAGAAGGA CUGAUGAG GCCGUUAGGC CGAA IAGAAGCA | 3686 | |
| 841 | UUCUCUUC C UUCUCUGU | 966 | ACAGAGAA CUGAUGAG GCCGUUAGGC CGAA IAAGAGAA | 3687 | |
| 842 | UCUCUUCC U UCUCUGUC | 987 | GACAGAGA CUGAUGAG GCCGUUAGGC CGAA IGAAGAGA | 3688 | |
| 845 | CUUCCUUC U CUGUCUCC | 988 | GUAGACAG CUGAUGAG GCCGUUAGGC CGAA IAAGGAAG | 3689 | |
| 847 | UCGGUCUC U GUCUCCUC | 989 | GAGGAGAC CUGAUGAG GCCGUUAGGC CGAA IAGAAGGA | 3690 | |
| 851 | UCUCUGUC U GCUCUCUC | 990 | GAGAGAGG CUGAUGAG GCCGUUAGGC CGAA IACAGAGA | 3691 | |
| 853 | UCUGUCUC C UCUCUCAG | 991 | CUGAGAGA CUGAUGAG GCCGUUAGGC CGAA IAGACAGA | 3692 | |
| 854 | CUGUCUCC U CUCUCAGC | 992 | GCUGAGAG CUGAUGAG GCCGUUAGGC CGAA IGAGACAG | 3693 | |
| 856 | GUCUCCUC U CUCAGCCG | 993 | CGUCUGAG CUGAUGAG GCCGUUAGGC CGAA IAGGAGAC | 3694 | |
| 858 | CUCCUCUC U CAGCCGCU | 994 | AGCGGCUG CUGAUGAG GCCGUUAGGC CGAA IAGAGGAG | 3695 | |
| 860 | CCUCUCUC A GCCGCUUC | 995 | GAAGCGGC CUGAUGAG GCCGUUAGGC CGAA IAGAGAGG | 3696 | |
| 863 | CUCUCAGC C GCUUCUUU | 996 | AAAGAAGC CUGAUGAG GCCGUUAGGC CGAA ICUGAGAG | 3697 | |
| 866 | UCAGGCUC U UCUUUCAA | 997 | UUGAAAGA CUGAUGAG GCCGUUAGGC CGAA ICGGCUGA | 3698 | |
| 869 | GCCGCUUC U UUCAAAGA | 998 | UCUUUGAA CUGAUGAG GCCGUUAGGC CGAA IAAGCGGC | 3699 | |
| 873 | CUUCUUUC A AAGAACAU | 999 | AUGUUCUU CUGAUGAG GCCGUUAGGC CGAA IAAAGAAG | 3700 | |
| 880 | CAAAGAAC A UGAAUACC | 1000 | GGUAUUCA CUGAUGAG GCCGUUAGGC CGAA IUUCUUUG | 3701 | |
| 888 | AUGAAUAC C UUGGUAAU | 1001 | AUUACCAA CUGAUGAG GCCGUUAGGC CGAA IUAUUCAU | 3702 | |
| 889 | UGAAUACC U UGGUAAUU | 1002 | AAUUACCA CUGAUGAG GCCGUUAGGC CGAA IGUAUUCA | 3703 | |
| 902 | AAUUUGUC A ACAGUAUU | 1003 | AAUACUGU CUGAUGAG GCCGUUAGGC CGAA IACAAAUU | 3704 | |
| 905 | UUGUCAAC A GUAUUACC | 1004 | GGUAAUAC CUGAUGAG GCCGUUAGGC CGAA IUUGACAA | 3705 | |
| 913 | AGUAUUAC C CACUGAAG | 1005 | CUUCAGUG CUGAUGAG GCCGUUAGGC CGAA IUAAUACU | 3706 | |
| 914 | GUAUUACC C ACUGAAGG | 1006 | CCUUCAGU CUGAUGAG GCCGUUAGGC CGAA IGUAAUAC | 3707 | |
| 915 | UAUUACCC A CUGAAGGA | 1007 | UCCUUCAG CUGAUGAG GCCGUUAGGC CGAA IGGUAAUA | 3708 | |
| 917 | UUACCCAC U GAAGGAAC | 1008 | GUUCCUUC CUGAUGAG GCCGUUAGGC CGAA IUGGGUAA | 3709 | |
| 926 | GAAGGAAC A CUUCAAGA | 1009 | UCUUGAAG CUGAUGAG GCCGUUAGGC CGAA IUUCCUUC | 3710 | |
| 928 | AGGAACAC U UCAAGAAA | 1010 | UUUCUUGA CUGAUGAG GCCGUUAGGC CGAA IUGUUCCU | 3711 | |
| 931 | AACACUUC A AGAAAAUG | 1011 | CAUUUUCU CUGAUGAG GCCGUUAGGC CGAA IAAGUGUU | 3712 | |
| 942 | AAAAUGUC A GUGAAGCU | 1012 | AGCUUCAC CUGAUGAG GCCGUUAGGC CGAA IACAULUU | 3713 | |
| 950 | AGUGAAGC U UCUAAAGA | 1013 | UCUUUAGA CUGAUGAG GCCGUUAGGC CGAA ICUUCACU | 3714 | |
| 953 | GAAGCUUC U AAAGAGGU | 1014 | ACCUCUUU CUGAUGAG GCCGUUAGGC CGAA IAAGCUUC | 3715 | |
| 963 | AAGAGGUC U CAGAGAAG | 1015 | CUUCUCUG CUGAUGAG GCCGUUAGGC CGAA IACCUCUU | 3716 | |
| 965 | GAGGUCUC A GAGAAGGC | 1016 | GCCUUCUC CUGAUGAG GCCGUUAGGC CGAA IAGACCUC | 3717 | |
| 974 | GAGAAGGC A AAAACUCU | 1017 | AGAGUUUU CUGAUGAG GCCGUUAGGC CGAA ICCUUCUC | 3718 | |
| 980 | GCAAAAAC U CUACUCAU | 1018 | AUGAGUAG CUGAUGAG GCCGUUAGGC CGAA IUUUUUGC | 3719 | |
| 982 | AAAAACUC U ACUCAUAG | 1019 | CUAUGAGU CUGAUGAG GCCGUUAGGC CGAA IAGUUUUU | 3720 | |
| 985 | AACUCUAC U CAUAGAUA | 1020 | UAUCUAUG CUGAUGAG GCCGUUAGGC CGAA IUAGAGUU | 3721 | |
| 987 | CUCUACUC A UAGAUAGA | 1021 | UCUAUCUA CUGAUGAG GCCGUUAGGC CGAA IAGUAGAG | 3722 | |
| 1004 | GAUUUAAC A GAGUUUUC | 1022 | GAAAACUC CUGAUGAG GCCGUUAGGC CGAA IUUAAAUC | 3723 | |
| 1013 | GAGUUUUC A GAAUUAGA | 1023 | UCUAAUUC CUGAUGAG GCCGUUAGGC CGAA IAAAACUC | 3724 | |
| 1026 | UAGAAUAC U CAGAAAUG | 1024 | CAUUUCUG CUGAUGAG GCCGUUAGGC CGAA IUAUUCUA | 3725 | |
| 1028 | GAAUACUC A GAAAUGGG | 1025 | CCCAUUUC CUGAUGAG GCCGUUAGGC CGAA IAGUAUUC | 3726 | |
| 1040 | AUGGGAUC A UCGUUCAG | 1026 | CUGAACGA CUGAUGAG GCCGUUAGGC CGAA IAUCCCAU | 3727 | |
| 1047 | CAUCGUUC A GUGUCUCU | 1027 | AGAGACAC CUGAUGAG GCCGUUAGGC CGAA IAACGAUG | 3728 | |
| 1053 | UCAGUGUC U CUCCAAAA | 1028 | UUUUGGAG CUGAUGAG GCCGUUAGGC CGAA IACACUGA | 3729 | |
| 1055 | AGUGUCUC U CCAAAAGC | 1029 | GCUUUUGG CUGAUGAG GCCGUUAGGC CGAA IAGACACU | 3730 | |
| 1057 | UGUCUCUC C AAAAGCAG | 1030 | CUGCUUUU CUGAUGAG GCCGUUAGGC CGAA IAGAGACA | 3731 | |
| 1058 | GUCUCUCC A AAAGCAGA | 1031 | UCUGCUUU CUGAUGAG GCCGUUAGGC CGAA IGAGAGAC | 3732 | |
| 1064 | CCAAAAGC A GAAUCUGC | 1032 | GCAGAUUC CUGAUGAG GCCGUUAGGC CGAA ICUUUUGG | 3733 | |
| 1070 | GCAGAAUC U GCCGUAAU | 1033 | AUUACGGC CUGAUGAG GCCGUUAGGC CGAA IAUUCUGC | 3734 | |
| 1073 | GAAUCUGC C GUAAUAGU | 1034 | ACUAUUAC CUGAUGAG GCCGUUAGGC CGAA ICAGAUUC | 3735 | |
| 1085 | AUAGUAGC A AAUCCUAG | 1035 | CUAGGAUU CUGAUGAG GCCGUUAGGC CGAA ICUACUAU | 3736 | |
| 1090 | AGCAAAUC C UAGGGAAG | 1036 | CUUCCCUA CUGAUGAG GCCGUUAGGC CGAA IAUUUGCU | 3737 | |
| 1091 | GCAAAUCC U AGGGAAGA | 1037 | UCUUCCCU CUGAUGAG GCCGUUAGGC CGAA IGAUUUGC | 3738 | |
| 1149 | GUAAUAAC A UCCUUCAU | 1038 | AUGAAGGA CUGAUGAG GCCGUUAGGC CGAA IUUAUUAC | 3739 | |
| 1152 | AUAACAUC C UUCAUAAU | 1039 | AUUAUCAA CUGAUGAG GCCGUUAGGC CGAA IAUGUUAU | 3740 | |
| 1153 | UAACAUCC U UCAUAAUC | 1040 | GAUUAUGA CUGAUGAG GCCGUUAGGC CGAA IGAUGUUA | 3741 | |
| 1156 | CAUCCUUC A UAAUCAAC | 1041 | GUUGAUUA CUGAUGAG GCCGUUAGGC CGAA IAAGGAUG | 3742 | |
| 1162 | UCAUAAUC A ACAAGAGU | 1042 | ACUCUUGU CUGAUGAG GCCGUUAGGC CGAA IAUUAUGA | 3743 | |
| 1165 | UAAUCAAC A AGAGUUAC | 1043 | GUAACUCU CUGAUGAG GCCGUUAGGC CGAA IUUGAUUA | 3744 | |
| 1174 | AGAGUUAC C UACAGCUC | 1044 | GAGCUGUA CUGAUGAG GCCGUUAGGC CGAA IUAACUCU | 3745 | |
| 1175 | GAGUUACC U ACAGCUCU | 1045 | AGAGCUGU CUGAUGAG GCCGUUAGGC CGAA IGUAACUC | 3746 | |
| 1178 | UUACCUAC A GCUCUUAC | 1046 | GUAAGAGC CUGAUGAG GCCGUUAGGC CGAA IUAGGUAA | 3747 | |
| 1181 | CCUACAGC U CUUACUAA | 1047 | UUAGUAAG CUGAUGAG GCCGUUAGGC CGAA ICUGUAGG | 3748 | |
| 1183 | UACAGCUC U UACUAAAU | 1048 | AUUUAGUA CUGAUGAG GCCGUUAGGC CGAA IAGCUGUA | 3749 | |
| 1187 | GCUCUUAC U AAAUUGGU | 1049 | ACCAAUUU CUGAUGAG GCCGUUAGGC CGAA IUAAGAGC | 3750 | |
| 1217 | GUUGUGUC U UCAGAAAA | 1050 | CUUUCUGA CUGAUGAG GCCGUUAGGC CGAA IACACAAC | 3751 | |
| 1220 | GUGUCUUC A GAAAAAGC | 1051 | GCUUUUUC CUGAUGAG GCCGUUAGGC CGAA IAAGACAC | 3752 | |
| 1229 | GAAAAAGC A AAAGACAG | 1052 | CUGUCUUU CUGAUGAG GCCGUUAGGC CGAA ICUUUUUC | 3753 | |
| 1236 | CAAAAGAC A GUUUUAAU | 1053 | AUUAAAAC CUGAUGAG GCCGUUAGGC CGAA IUCUUUUG | 3754 | |
| 1259 | AGAGUUGC A GUGGAAGC | 1054 | GCUUCCAC CUGAUGAG GCCGUUAGGC CGAA ICAACUCU | 3755 | |
| 1268 | GUGGAAGC U CCUAUGAG | 1055 | CUCAUAGG CUGAUGAG GCCGUUAGGC CGAA ICUUCCAC | 3756 | |
| 1270 | GGAAGCUC C UAUGAGGG | 1056 | CCCUCAUA CUGAUGAG GCCGUUAGGC CGAA IAGCUUCC | 3757 | |
| 1271 | GAAGCUCC U AUGAGGGA | 1057 | UCCCUCAU CUGAUGAG GCCGUUAGGC CGAA IGAGCUUC | 3758 | |
| 1289 | GAAUAUGC A GACUUCAA | 1058 | UUGAAGUC CUGAUGAG GCCGUUAGGC CGAA ICAUAUUC | 3759 | |
| 1293 | AUGCAGAC U UCAAACCA | 1059 | UGGUUUGA CUGAUGAG GCCGUUAGGC CGAA IUCUGCAU | 3760 | |
| 1296 | CAGACUUC A AACCAUUU | 1060 | AAAUGGUU CUGAUGAG GCCGUUAGGC CGAA IAAGUCUG | 3761 | |
| 1300 | CUUCAAAC C AUUUGAGC | 1061 | GCUCAAAU CUGAUGAG GCCGUUAGGC CGAA IUUUGAAG | 3762 | |
| 1301 | UUCAAACC A UUUGAGCG | 1062 | CGCUCAAA CUGAUGAG GCCGUUAGGC CGAA IGUUUGAA | 3763 | |
| 1355 | AUGUUGGC U GCUGGAGG | 1063 | CCUCCAGC CUGAUGAG GCCGUUAGGC CGAA ICCAACAU | 3764 | |
| 1358 | UUGGCUGC U GGAGGUAA | 1064 | UUACCUCC CUGAUGAG GCCGUUAGGC CGAA ICAGCCAA | 3765 | |
| 1377 | UCGAGAGC A ACUUGGAA | 1065 | UUCCAAGU CUGAUGAG GCCGUUAGGC CGAA ICUCUCGA | 3766 | |
| 1380 | AGAGCAAC U UGGAAAGU | 1066 | ACUUUCCA CUGAUGAG GCCGUUAGGC CGAA IUUGCUCU | 3767 | |
| 1412 | UGUUUUGC A GAUAGCCU | 1067 | AGGCUAUC CUGAUGAG GCCGUUAGGC CGAA ICAAAACA | 3768 | |
| 1419 | CAGAUAGC C UUGAGCAA | 1068 | UCGCUCAA CUGAUGAG GCCGUUAGGC CGAA ICUAUCUG | 3769 | |
| 1420 | AGAUAGCC U UGAGCAAA | 1069 | UUUGCUCA CUGAUGAG GCCGUUAGGC CGAA IGCUAUCU | 3770 | |
| 1426 | CCUUGAGC A AACUAAUC | 1070 | GAUUAGUU CUGAUGAG GCCGUUAGGC CGAA ICUCAAGG | 3771 | |
| 1430 | GAGCAAAC U AAUCACGA | 1071 | UCGUGAUU CUGAUGAG GCCGUUAGGC CGAA IUUUGCUC | 3772 | |
| 1435 | AACUAAUC A CGAAAAAG | 1072 | CUUUUUCG CUGAUGAG GCCGUUAGGC CGAA IAUUAGUU | 3773 | |
| 1469 | GAUGAUAC U UCUUUCCC | 1073 | GGGAAAGA CUGAUGAG GCCGUUAGGC CGAA IUAUCAUC | 3774 | |
| 1472 | GAUACUUC U UUCCCCAG | 1074 | CUGGGGAA CUGAUGAG GCCGUUAGGC CGAA IAAGUAUC | 3775 | |
| 1476 | CUUCUUUC C CCAGUACG | 1075 | CGUACUGG CUGAUGAG GCCGUUAGGC CGAA IAAAGAAG | 3776 | |
| 1477 | UUCUUUCC C CAGUACGC | 1076 | GCGUACUG CUGAUGAG GCCGUUAGGC CGAA IGAAAGAA | 3777 | |
| 1478 | UCUUUCCC C AGUACGCC | 1077 | GGCGUACU CUGAUGAG GCCGUUAGGC CGAA IGGAAAGA | 3778 | |
| 1479 | CUUUCCCC A GUACGCCA | 1078 | UGGCGUAC CUGAUGAG GCCGUUAGGC CGAA IGGGAAAG | 3779 | |
| 1486 | CAGUACGC C AGAAGGUA | 1079 | UACCUUCU CUGAUGAG GCCGUUAGGC CGAA ICGUACUG | 3780 | |
| 1487 | AGUACGCC A GAAGGUAU | 1080 | AUACCUUC CUGAUGAC GCCGUUAGGC CGAA IGCGUACU | 3781 | |
| 1508 | GAUCGUUC A GGAGCAUA | 1081 | UAUGCUCC CUGAUGAG GCCGUUAGGC CGAA IAACGAUC | 3782 | |
| 1514 | UCAGGAUC A UAUAUCAC | 1082 | GUGAUAUA CUGAUGAG GCCGUUAGGC CGAA ICUCCUGA | 3783 | |
| 1521 | CAUAUAUC A CAUGUGCU | 1083 | AGCACAUG CUGAUGAG GCCGUUAGGC CGAA IAUAUAUG | 3784 | |
| 1523 | UAUAUCAC A UGUGCUCC | 1084 | GGAGCACA CUGAUGAG GCCGUUAGGC CGAA IUGAUAUA | 3785 | |
| 1529 | ACAUGUGC U CCCUUUAA | 1085 | UUAAAGGG CUGAUGAG GCCGUUAGGC CGAA ICACAUGU | 3786 | |
| 1531 | AUGUGCUC C CUUUAACC | 1086 | GGUUAAAG CUGAUGAG GCCGUUAGGC CGAA IAGCACAU | 3787 | |
| 1532 | UGUGCUCC C UUUAACCC | 1087 | GGGUUAAA CUGAUGAG GCCGUUAGGC CGAA IGAGCAAA | 3788 | |
| 1533 | GUGCUCCC U UUAACCCA | 1088 | UGGGUUAA CUGAUGAG GCCGUUAGGC CGAA IGGAGCAC | 3789 | |
| 1539 | CCUUUAAC C CAGCAGCA | 1089 | UGCUGCUG CUGAUGAG GCCGUUAGGC CGAA IUUAAAGG | 3790 | |
| 1540 | CUUUAACC C AGCAGCAA | 1090 | UUGCUGCU CUGAUGAG GCCGUUAGGC CGAA IGUUAAAG | 3791 | |
| 1541 | UUUAACCC A GCAGCAAC | 1091 | GUUGCUGC CUGAUGAG GCCGUUAGGC CGAA IGGUUAAA | 3792 | |
| 1544 | AACCCAGC A GCAACUGA | 1092 | UCAGUUGC CUGAUGAG GCCGUUAGGC CGAA ICUGGGUU | 3793 | |
| 1547 | CCAGCAGC A ACUGAGAG | 1093 | CUCUCAGU CUGAUGAG GCCGUUAGGC CGAA ICUGCUGG | 3794 | |
| 1550 | GCAGCAAC U GAGAGCAU | 1094 | AUGCUCUC CUGAUGAG GCCGUUAGGC CGAA IUUGCUGC | 3795 | |
| 1557 | CUGAGAGC A UUGCAACA | 1095 | UGUUGCAA CUGAUGAG GCCGUUAGGC CGAA ICUCUCAG | 3796 | |
| 1562 | AGCAUUGC A ACAAACAU | 1096 | AUGUUUGU CUGAUGAG GCCGUUAGGC CGAA ICAAUGCU | 3797 | |
| 1565 | AUUGCAAC A AACAUUUU | 1097 | AAAAUGUU CUGAUGAG GCCGUUAGGC CGAA IUUGCAAU | 3798 | |
| 1569 | CAACAAAC A UUUUUCCU | 1098 | AGGAAAAA CUGAUGAG GCCGUUAGGC CGAA IUUUGUUG | 3799 | |
| 1576 | CAUUUUUC C UUUGUUAG | 1099 | CUAACAAA CUGAUGAG GCCGUUAGGC CGAA IAAAAAUG | 3800 | |
| 1577 | AUUUUUCC U UUGUUAGG | 1100 | CCUAACAA CUGAUGAG GCCGUUAGGC CGAA IGAAAAAU | 3801 | |
| 1591 | AGGAGAUC C UACUUCAG | 1101 | CUGAAGUA CUGAUGAG GCCGUUAGGC CGAA IAUCUCCU | 3802 | |
| 1592 | GGAGAUCC U ACCUCAGA | 1102 | UCUGAAGU CUGAUGAG GCCGUUAGGC CGAA IGAUCUCC | 3803 | |
| 1595 | GAUCCUAC U UCAGAAAA | 1103 | UUUUCUGA CUGAUGAG GCCGUUAGGC CGAA IUAGGAUC | 3804 | |
| 1598 | CCUACUUC A GAAAAUAA | 1104 | UUAUUUUC CUGAUGAG GCCGUUAGGC CGAA IAAGUAGG | 3805 | |
| 1610 | AAUAAGAC C GAUGAAAA | 1105 | UUUUCAUC CUGAUGAG GCCGUUAGGC CGAA IUCUUAUU | 3806 | |
| 1640 | AAGAAGGC C CAAAUAGU | 1106 | ACUAUUUG CUGAUGAG GCCGUUAGGC CGAA ICCUUCUU | 3807 | |
| 1641 | AGAAGGCC C AAAUAGUA | 1107 | UACUAUUU CUGAUGAG GCCGUUAGGC CGAA IGCCUUCU | 3808 | |
| 1642 | GAAGGCCC A AAUAGUAA | 1108 | UUACUAUU CUGAUGAG GCCGUUAGGC CGAA IGGCCUUC | 3809 | |
| 1652 | AUAGUAAC A GAGAAGAA | 1109 | UUCUUCUC CUGAUGAG GCCGUUAGGC CGAA IUUACUAU | 3810 | |
| 1664 | AAGAAUAC U AGCACCAA | 1110 | UUGGUGCU CUGAUGAG GCCGUUAGGC CGAA IUAUUCUU | 3811 | |
| 1668 | AUACUAGC A CCAAAACA | 1111 | UGUUUUGG CUGAUGAG GCCGUUAGGC CGAA ICUAGUAU | 3812 | |
| 1670 | ACUAGCAC C AAAACAUC | 1112 | GAUGUUUU CUGAUGAG GCCGUUAGGC CGAA IUGCUAGU | 3813 | |
| 1671 | CUAGCACC A AAACAUCA | 1113 | UGAUGUUU CUGAUGAG GCCGUUAGGC CGAA IGUGCUAG | 3814 | |
| 1676 | ACCAAAAC A UCAAACCC | 1114 | GGGUCUGA CUGAUGAG GCCGUUAGGC CGAA IUUUUGGU | 3815 | |
| 1679 | AAAACAUC A AACCCUUU | 1115 | AAAGGGUU CUGAUGAG GCCGUUAGGC CGAA IAUGUUUU | 3816 | |
| 1683 | CAUCAAAC C CUUUUCUU | 1116 | AAGAAAAG CUGAUGAG GCCGUUAGGC CGAA IUUUGAUG | 3817 | |
| 1684 | AUCAAACC C UUUUCUUG | 1117 | CAAGAAAA CUGAUGAG GCCGUUAGGC CGAA IGUUUGAU | 3818 | |
| 1685 | UCAAACCC U UUUCUUGU | 1118 | ACAAGAAA CUGAUGAG GCCGUUAGGC CGAA IGGUUUGA | 3619 | |
| 1690 | CCCUUUUC U UGUAGCAG | 1119 | CUGCUACA CUGAUGAG GCCGUUAGGC CGAA IAAAAGGG | 3820 | |
| 1697 | CUUGUAGC A GCACAGGA | 1120 | UCCUGUGC CUGAUGAG GCCGUUAGGC CGAA ICUACAAG | 3821 | |
| 1700 | GUAGCAGC A CAGGACUC | 1121 | GAAUCCUG CUGAUGAG GCCGUUAGGC CGAA ICUGCUAC | 3822 | |
| 1702 | AGCAGCAC A GGAUUCUG | 1122 | CAGAAUCC CUGAUGAG GCCGUUAGGC CGAA IUGCUGCU | 3823 | |
| 1709 | CAGGAUUC U GAGACAGA | 1123 | UCUGUCUC CUGAUGAG GCCGUUAGGC CGAA IAAUCCUG | 3824 | |
| 1715 | UCUGAGAC A GAUUAUGU | 1124 | ACAUAAUC CUGAUGAG GCCGUUAGGC CGAA IUCUCAGA | 3825 | |
| 1725 | AUUAUGUC A CAACAGAU | 1125 | AUCUGUUG CUGAUGAG GCCGUUAGGC CGAA IACAUAAU | 3826 | |
| 1727 | UAUGUCAC A ACAGAUAA | 1126 | UUAUCUGU CUGAUGAG GCCGUUAGGC CGAA IUGACAUA | 3827 | |
| 1730 | GUCACAAC A GAUAAUUU | 1127 | AAAUUAUC CUGAUGAG GCCGUUAGGC CGAA IUUGUGAC | 3828 | |
| 1742 | AAUUUAAC A AAGGUGAC | 1128 | GUCACCUU CUGAUGAG GCCGUUAGGC CGAA IUUAAAUU | 3829 | |
| 1751 | AAGGUGAC U GAGGAAGU | 1129 | ACUUCCUC CUGAUGAG GCCGUUAGGC CGAA IUCACCUU | 3830 | |
| 1766 | GUCGUGGC A AACAUGCC | 1130 | GGCAUGUU CUGAUGAG GCCGUUAGGC CGAA ICCACGAC | 3831 | |
| 1770 | UGGCAAAC A UGCCUGAA | 1131 | UUCAGGCA CUGAUGAG GCCGUUAGGC CGAA IUUUGCCA | 3832 | |
| 1774 | AAACAUGC C UGAAGGCC | 1132 | GGCCUUCA CUGAUGAG GCCGUUAGGC CGAA ICAUGUUU | 3833 | |
| 1775 | AACAUGCC U GAAGGCCU | 1133 | AGGCCUUC CUGAUGAG GCCGUUAGGC CGAA IGCAUGUU | 3834 | |
| 1782 | CUGAAGGC C UGACUCCA | 1134 | UGGAGUCA CUGAUGAG GCCGUUAGGC CGAA ICCUUCAG | 3835 | |
| 1783 | UGAAGGCC U GACUCCAG | 1135 | CUGGAGUC CUGAUGAG GCCGUUAGGC CGAA IGCCUUCA | 3836 | |
| 1787 | GGCCUGAC U CCAGAUUU | 1136 | AAAUCUGG CUGAUGAG GCCGUUAGGC CGAA IUCAGGCC | 3837 | |
| 1789 | CCUGACUC C AGAUUUAG | 1137 | CUAAAUCU CUGAUGAG GCCGUUAGGC CGAA IAGUCAGG | 3838 | |
| 1790 | CUGACUCC A GAUUUAGU | 1138 | ACUAAAUC CUGAUGAG GCCGUUAGGC CGAA IGAGUCAG | 3839 | |
| 1801 | UUUAGUAC A GGAAGCAU | 1139 | AUGCUUCC CUGAUGAG GCCGUUAGGC CGAA IUACUAAA | 3840 | |
| 1808 | CAGGAAGC A UGUGAAAG | 1140 | CUUUCACA CUGAUGAG GCCGUUAGGC CGAA ICUUCCUG | 3841 | |
| 1835 | GAAGUUAC U GGUACAAA | 1141 | UUUGUACC CUGAUGAG GCCGUUAGGC CGAA IUAACUUC | 3842 | |
| 1841 | ACUGGUAC A AAGAUUGC | 1142 | GCAAUCUU CUGAUGAG GCCGUUAGGC CGAA IUACCAGU | 3843 | |
| 1350 | AAGAUUGC U UAUGAAAC | 1143 | GUUUCAUA CUGAUGAG GCCGUUAGGC CGAA ICAAUCUU | 3844 | |
| 1859 | UAUGAAAC A AAAAUGGA | 1144 | UCCAUCUC CUGAUGAG GCCGUUAGGC CGAA IUUUCAUA | 3845 | |
| 1869 | AAAUGGAC U UGGUUCAA | 1145 | UUGAACCA CUGAUGAG GCCGUUAGGC CGAA IUCCAUUU | 3846 | |
| 1876 | CUUGGUUC A AACAUCAG | 1146 | CUGAUGUC CUGAUGAG GCCGUUAGGC CGAA IAACCAAG | 3847 | |
| 1880 | GUUCAAAC A UCAGAAGU | 1147 | ACUUCUGA CUGAUGAG GCCGUUAGGC CGAA IUUUGAAC | 3848 | |
| 1883 | CAAACAUC A GAAGUUAU | 1148 | AUAACUUC CUGAUGAG GCCGUUAGGC CGAA IAUGUUUG | 3849 | |
| 1894 | AGUUAUGC A AGAGUCAC | 1149 | GUGACUCU CUGAUGAG GCCGUUAGGC CGAA ICAUAACU | 3850 | |
| 1901 | CAAGAGUC A CUCUAUCC | 1150 | GGAUAGAG CUGAUGAG GCCGUUAGGC CGAA IACUCUUG | 3851 | |
| 1903 | AGAGUCAC U CUAUCCUG | 1151 | CAGGAUAG CUGAUGAG GCCGUUAGGC CGAA IUGACUCU | 3852 | |
| 1905 | AGUCACUC U AUCCUGCA | 1152 | UGCAGGAU CUGAUGAG GCCGUUAGGC CGAA IAGUGACU | 3853 | |
| 1909 | ACUCUAUC C UGCAGCAC | 1153 | GUGCUGCA CUGAUGAG GCCGUUAGGC CGAA IAUAGAGU | 3854 | |
| 1910 | CUCUAUCC U GCAGCACA | 1154 | UGUGCUGC CUUAUGAG GCCGUUAGGC CGAA IGAUAGAG | 3855 | |
| 1913 | UAUCCUGC A GCACAGCU | 1155 | AGCUGUGC CUGAUGAG GCCGUUAGGC CGAA ICAGGAUA | 3856 | |
| 1916 | CCUGCAGC A CAGCUUUG | 1156 | CAAAGCUG CUGAUGAG GCCGUUAGGC CGAA ICUGCAGG | 3857 | |
| 1918 | UGCAGCAC A GCUUUGCC | 1157 | GGCAAAGC CUGAUGAG GCCGUUAGGC CGAA IUGCUGCA | 3858 | |
| 1921 | AGCACAGC U UUGCCCAU | 1158 | AUGGGCAA CUGAUGAG GCCGUUAGGC CGAA ICUGUGCU | 3859 | |
| 1926 | AGCUUUGC C CAUCAUUU | 1159 | AAAUGAUG CUGAUGAG GCCGUUAGGC CGAA ICAAAGCU | 3860 | |
| 1927 | GCUUUGCC C AUCAUUUG | 1160 | CAAAUGAU CUGAUGAG GCCGUUAGGC CGAA IGCAAAGC | 3861 | |
| 1928 | CUUUGCCC A UCAUUUGA | 1161 | UCAAAUGA CUGAUGAG GCCGUUAGGC CGAA IGGCAAAG | 3862 | |
| 1931 | UGCCCAUC A UUUGAAGA | 1162 | UCUUCAAA CUGAUGAG GCCGUUAGGC CGAA IAUGGGCA | 3863 | |
| 1943 | GAAGAGUC A GAAGCUAC | 1163 | GUAGCUUC CUGAUGAG GCCGUUAGGC CGAA IACUCUUC | 3864 | |
| 1949 | UCAGAAGC U ACUCCUUC | 1164 | GAAGGAGU CUGAUGAG GCCGUUAGGC CGAA ICUUCUGA | 3865 | |
| 1952 | GAAGCUAC U CCUUCACC | 1165 | GGUGAAGG CUGAUGAG GCCGUUAGGC CGAA IUAGCUUC | 3866 | |
| 1954 | AGCUACUC C UUCACCAG | 1166 | CUGGUGAA CUGAUGAG GCCGUUAGGC CGAA IAGUAGCU | 3867 | |
| 1955 | GCUACUCC U UCACCAGU | 1167 | ACUGGUGA CUGAUGAG GCCGUUAGGC CGAA IGAGUAGC | 3868 | |
| 1958 | ACUCCUUC A CCAGUUUU | 1168 | AAAACUGG CUGAUGAG GCCGUUAGGC CGAA IAAGGAGU | 3869 | |
| 1960 | UCCUUCAC C AGUUUUGC | 1169 | GCAAAACU CUGAUGAG GCCGUUAGGC CGAA IUGAAGGA | 3870 | |
| 1961 | CCUUCACC A GUUUUGCC | 1170 | GGCAAAAC CUGAUGAG GCCGUUAGGC CGAA IGUGAAGG | 3871 | |
| 1969 | AGUUUUGC C UGACAUUG | 1171 | CAAUGUCA CUGAUGAG GCCGUUAGGC CGAA ICAAAACU | 3872 | |
| 1970 | GUUUUGCC U GACAUUGU | 1172 | ACAAUGUC CUGAUGAG GCCGUUAGGC CGAA IGCAAAAC | 3873 | |
| 1974 | UGCCUGAC A UUGUUAUG | 1173 | CAUAACAA CUGAUGAG GCCGUUAGGC CGAA IUCAGGCA | 3874 | |
| 1988 | AUGGAAGC A CCAUUGAA | 1174 | UUCAAUGG CUGAUGAG GCCGUUAGGC CGAA ICUUCCAU | 3875 | |
| 1990 | GGAAGCAC C AUUGAAUU | 1175 | AAUUCAAU CUGAUGAG GCCGUUAGGC CGAA IUGCUUCC | 3876 | |
| 1991 | GAAGCACC A UUGAAUUC | 1176 | GAAUUCAA CUGAUGAG GCCGUUAGGC CGAA IGUGCUUC | 3877 | |
| 2000 | UUGAAUUC U GCAGUUCC | 1177 | GGAACUGC CUGAUGAG GCCGUUAGGC CGAA IAAUUCAA | 3878 | |
| 2003 | AAUUCUGC A GUUCCUAG | 1178 | CUAGGAAC CUGAUGAG GCCGUUAGGC CGAA ICAGAAUU | 3879 | |
| 2008 | UGCAGUUC C UAGUGCUG | 1179 | CAGCACUA CUGAUGAG GCCGUUAGGC CGAA IAACUGCA | 3880 | |
| 2009 | GCAGUUCC U AGUGCUGG | 1180 | CCAGCACU CUGAUGAG GCCGUUAGGC CGAA IGAACUGC | 3881 | |
| 2015 | CCUAGUGC U GGUGCUUC | 1181 | GAAGCACC CUGAUGAG GCCGUUAGGC CGAA ICACUAGG | 3882 | |
| 2021 | GCUGGUGC U UCCGUGAU | 1182 | AUCACGGA CUGAUGAG GCCGUUAGGC CGAA ICACCAGC | 3883 | |
| 2024 | GGUGCUUC C GUGAUACA | 1183 | UGUAUCAC CUGAUGAG GCCGUUAGGC CGAA IAAGCACC | 3884 | |
| 2032 | CGUGAUAC A GCCCAGCU | 1184 | AGCUGGGC CUGAUGAG GCCGUUAGGC CGAA IUAUCACG | 3885 | |
| 2035 | GAUACAGC C CAGCUCAU | 1185 | AUGAGCUG CUGAUGAG GCCGUUAGGC CGAA ICUGUAUC | 3886 | |
| 2036 | AUACAGCC C AGCUCAUC | 1186 | GAUGAGCU CUGAUGAG GCCGUUAGGC CGAA IGCUGUAU | 3887 | |
| 2037 | UACAGCCC A GCUCAUCA | 1187 | UGAUGAGC CUGAUGAG GCCGUUAGGC CGAA IGGCUGUA | 3888 | |
| 2040 | AGCCCAGC U CAUCACCA | 1188 | UGGUGAUG CUGAUGAG GCCGUUAGGC CGAA ICUGGGCU | 3889 | |
| 2042 | CCCAGCUC A UCACCAUU | 1189 | AAUGGUGA CUGAUGAG GCCGUUAGGC CGAA IAGCUGGG | 3890 | |
| 2045 | AGCUCAUC A CCAUUAGA | 1190 | UCUAAUGG CUGAUGAG GCCGUUAGGC CGAA IAUGAGCU | 3891 | |
| 2047 | CUCAUCAC C AUUAGAAG | 1191 | CUUCUAAU CUGAUGAG GCCGUUAGGC CGAA LUGAUGAG | 3892 | |
| 2048 | UCAUCACC A UUAGAAGC | 1192 | GCUUCUAA CUGAUGAG GCCGUUAGGC CGAA IGUGAUGA | 3893 | |
| 2057 | UUAGAAGC U UCUUCAGU | 1193 | ACUGAAGA CUGAUGAG GCCGUUAGGC CGAA ICUUCUAA | 3894 | |
| 2060 | GAAGCUUC U UCAGUUAA | 1194 | UUAACUGA CUGAUGAG GCCGUUAGGC CGAA IAAGCUUC | 3895 | |
| 2063 | GCUUCUUC A GUUAAUUA | 1195 | UAAUUAAC CUGAUGAG GCCGUUAGGC CGAA IAAGAAGC | 3896 | |
| 2079 | AUGAAAGC A UAAAACAU | 1196 | AUGUUUUA CUGAUGAG GCCGUUAGGC CGAA ICUUUCAU | 3897 | |
| 2086 | CAUAAAAC A UGAGCCUG | 1197 | CAGGCUCA CUGAUGAG GCCGUUAGGC CGAA IUUUUAUG | 3898 | |
| 2092 | ACAUGAGC C UGAAAACC | 1198 | GGUUUUCA CUGAUGAG GCCGUUAGGC CGAA ICUCAUGU | 3899 | |
| 2093 | CAUGAGGC U GAAAACCC | 1199 | GGGUUUUC CUGAUGAG GCCGUUAGGC CGAA IGCUCAUG | 3900 | |
| 2100 | CUGAAAAC C CCCCACCA | 1200 | UGGUGGGG CUGAUGAG GCCGUUAGGC CGAA IUUUUCAG | 3901 | |
| 2101 | UGAAAACC C CCCACCAU | 1201 | AUGGUGGG CUGAUGAG GCCGUUAGGC CGAA IGUUUUCA | 3902 | |
| 2102 | GAAAACCC C CCACCAUA | 1202 | UAUGGUGG CUGAUGAG GCCGUUAGGC CGAA IGGUUUUC | 3903 | |
| 2103 | AAAACCCC C CACCAUAU | 1203 | AUAUGGUG CUGAUGAG GCCGUUAGGC CGAA IGGGUUUU | 3904 | |
| 2104 | AAACCCCC C ACCAUAUG | 1204 | CAUAUGGU CUGAUGAG GCCGUUAGGC CGAA IGGGGUUU | 3905 | |
| 2105 | AACCCCCC A CCAUAUGA | 1205 | UCAUAUGG CUGAUGAG GCCGUUAGGC CGAA IGGGGGUU | 3906 | |
| 2107 | CCCCCCAC C AUAUGAAG | 1206 | CUUCAUAU CUGAUGAG GCCGUUAGGC CGAA IUGGGGGG | 3907 | |
| 2108 | CCCCCACC A UAUGAAGA | 1207 | UCUUCAUA CUGAUGAG GCCGUUAGGC CGAA IGUGGGGG | 3908 | |
| 2120 | GAAGAGGC C AUGAGUGU | 1208 | ACACUCAW CUGAUGAG GCCGUUAGGC CGAA ICCUCUUC | 3909 | |
| 2121 | AAGAGGCC A UGAGUGUA | 1209 | UACACUCA CUGAUGAG GCCGUUAGGC CGAA IGCCUCUU | 3910 | |
| 2132 | AGUGUAUC A CUAAAAAA | 1210 | UUUUUUAG CUGAUGAG GCCGUUAGGC CGAA IAUACACU | 3911 | |
| 2134 | UGUAUCAC U AAAPAAAG | 1211 | CUUUlUUUU CUGAUGAG GCCGUUAGGC CGAA IUGAUACA | 3912 | |
| 2147 | AAAGUAUC A GGAAUAAA | 1212 | UUUAUUCC CUGAUGAG GCCGUUAGGC CGAA IAUACUUU | 3913 | |
| 2173 | UAAAGAGC C UGAAAAUA | 1213 | UAUUUUCA CUGAUGAG GCCGUUAGGC CGAA ICUCUUUA | 3914 | |
| 2174 | AAAGAGCC U GAAAAUAU | 1214 | AUAUUUUC CUGAUGAG GCCGUUAGGC CGAA IGCUCUUU | 3915 | |
| 2189 | AUUAAUGC A GCUCUUCA | 1215 | UGAAGAGC CUGAUGAG GCCGUUAGGC CGAA ICAUUAAU | 3916 | |
| 2192 | AAUGCAGC U CUUCAAGA | 1216 | UCUUGAAG CUGAUGAG GCCGUUAGGC CGAA ICUGCAUU | 3917 | |
| 2194 | UGCAGCUC U UCAAGAAA | 1217 | UUUCUUGA CUGAUGAG GCCGUUAGGC CGAA IAGCUGCA | 3918 | |
| 2197 | AGCUCUUC A AGAAACAG | 1218 | CUGUUUCU CUGAUGAG GCCGUUAGGC CGAA IAAGAGCU | 3919 | |
| 2204 | CAAGAAAC A GAAGCUCC | 1219 | GGAGCUUC CUGAUGAG GCCGUUAGGC CGAA IUUUCUUG | 3920 | |
| 2210 | ACAGAAGC U CCUUAUAU | 1220 | AUAUAAGG CUGAUGAG GCCGUUAGGC CGAA ICUUCUGU | 3921 | |
| 2212 | AGAAGCUC C UUAUAUAU | 1221 | AUAUAUAA CUGAUGAG GCCGUUAGGC CGAA IAGCUUCU | 3922 | |
| 2213 | GAAGCUCC U UAUAUAUC | 1222 | GAUAUAUA CUGAUGAG GCCGUUAGGC CGAA IGAGCUUC | 3923 | |
| 2222 | UAUAUAUC U AUUGCAUG | 1223 | CAUGCAAU CUGAUGAG GCCGUUAGGC CGAA IAUAUAUA | 3924 | |
| 2228 | UCUAUUGC A UGUGAUUU | 1224 | AAAUCACA CUGAUGAG GCCGUUAGGC CGAA ICAAUAGA | 3925 | |
| 2249 | AAAGAAAC A AAGCUUUC | 1225 | GAAAGCUU CUGAUGAG GCCGUUAGGC CGAA IUUUCUUU | 3926 | |
| 2254 | AACAAAGC U UUCUGCUG | 1226 | CAGCAGAA CUGAUGAG GCCGUUAGGC CGAA ICUUUGUU | 3927 | |
| 2258 | AAGCUUUC U GCUGAACC | 1227 | GGUUCAGC CUGAUGAG GCCGUUAGGC CGAA IAAAGCUU | 3928 | |
| 2262 | CUUUCUGC U GAACCAGC | 1228 | CCUGGUUC CUGAUGAG GCCGUUAGGC CGAA ICAGAAAG | 3929 | |
| 2266 | UGCUGAAC C AGCUCCGG | 1229 | CCGGAGCU CUGAUGAG GCCGUUAGGC CGAA IUUCAGCA | 3930 | |
| 2267 | GCUGAACC A GCUCCGGA | 1230 | UCCGGAGC CUGAUGAG GCCGUUAGGC CGAA IGUUCAGC | 3931 | |
| 2270 | GAACCAGC U CCGGAUUU | 1231 | AAAUCCGG CUGAUGAG GCCGUUAGGC CGAA ICUGGUUC | 3932 | |
| 2272 | ACCAGCUC C GGAUUUCU | 1232 | AGAAAUCC CUGAUGAG GCCGUUAGGC CGAA IAGCUGGU | 3933 | |
| 2280 | CGGAUUUC U CUGAUUAU | 1233 | AUAAUCAG CUGAUGAG GCCGUUAGGC CGAA IAAAUCCG | 3934 | |
| 2282 | GAUUUCUC U GAUUAUUC | 1234 | GAAUAAUC CUGAUGAG GCCGUUAGGC CGAA IAGAAAUC | 3935 | |
| 2292 | GAUUAUUC A GAAAUGGC | 1235 | GCCAUUUC CUGAUGAG GCCGUUAGGC CGAA IAAUAAUC | 3936 | |
| 2300 | GAAAUGGC A AAAGUUGA | 1236 | UCAACUUU CUGAUGAG GCCGUUAGGC CGAA ICCAUUUC | 3937 | |
| 2312 | AGUUGAAC A GCCAGUGC | 1237 | GCACUGGC CUGAUGAG GCCGUUAGGC CGAA IUUCAACU | 3938 | |
| 2314 | UGAACAGC C AGUGCCUG | 1238 | CAGGCACU CUGAUGAG GCCGUUAGGC CGAA ICUGUUCA | 3939 | |
| 2315 | GAACAGCC A GUGCCUGA | 1239 | UCAGGCAC CUGAUGAG GCCGUUAGGC CGAA IGCUGUUC | 3940 | |
| 2320 | GCCAGUGC C UGAUCAUU | 1240 | AAUGAUCA CUGAUGAG GCCGUUAGGC CGAA ICACUGGC | 3941 | |
| 2322 | CCAGUGCC U GAUCAUUC | 1241 | GAAUGAUC CUGAUGAG GCCGUUAGGC CGAA IGCACUGG | 3942 | |
| 2326 | GCCUGAUC A UUCUGAGC | 1242 | GCUCAGAA CUGAUGAG GCCGUUAGGC CGAA IAUCAGGC | 3943 | |
| 2330 | GAUCAUUC U GAGCUAGU | 1243 | ACUAGCUC CUGAUGAG GCCGUUAGGC CGAA IAAUGAUC | 3944 | |
| 2335 | UUCUGAGC U AGUUGAAG | 1244 | CUUCAACU CUGAUGAG GCCGUUAGGC CGAA ICUCAGAA | 3945 | |
| 2348 | GAAGAUUC C UCACCUGA | 1245 | UCAGGUGA CUGAUGAG GCCGUUAGGC CGAA IAAUCUUC | 3946 | |
| 2349 | AAGAUUCC U CACCUGAU | 1246 | AUCAGGUG CUGAUGAG GCCGUUAGGC CGAA IGAAUCUU | 3947 | |
| 2351 | GAUUCCUC A CCUGAUUC | 1247 | GAAUCAGG CUGAUGAG GCCGUUAGGC CGAA IAGGAAUC | 3948 | |
| 2353 | UUCCUCAC C UGAUUCUG | 1248 | CAGAAUCA CUGAUGAG GCCGUUAGGC CGAA IUGAGGAA | 3949 | |
| 2354 | UCCUCACC U GAUUCUGA | 1249 | UCAGAAUC CUGAUGAG GCCGUUAGGC CGAA IGUGAGGA | 3950 | |
| 2360 | CCUGAUUC U GAACCAGU | 1250 | ACUGGUUC CUGAUGAG GCCGUUAGGC CGAA IAAUCAGG | 3951 | |
| 2365 | UUCUGAAC C AGUUGACU | 1251 | AGUCAACU CUGAUGAG GCCGUUAGGC CGAA IUUCAGAA | 3952 | |
| 2366 | UCUGAACC A GUUGACUU | 1252 | AAGUCAAC CUGAUGAG GCCGUUAGGC CGAA IGUUCAGA | 3953 | |
| 2373 | CAGUUGAC U UAUUUAGU | 1253 | ACUAAAUA CUGAUGAG GCCGUUAGGC CGAA IUCAACUG | 3954 | |
| 2390 | GAUGAUUC A AUACCUGA | 1254 | UCAGGUAU CUGAUGAG GCCGUUAGGC CGAA IAAUCAUC | 3955 | |
| 2395 | UUCAAUAC C UGACGUUC | 1255 | GAACGUCA CUGAUGAG GCCGUUAGGC CGAA IUAUUGAA | 3956 | |
| 2396 | UCAAUACC U GACGUUCC | 1256 | GGAACGUC CUGAUGAG GCCGUUAGGC CGAA IGUAUUGA | 3957 | |
| 2404 | UGACGUUC C ACAAAAAC | 1257 | GUUUUUGU CUGAUGAG GCCGUUAGGC CGAA IAACGUCA | 3958 | |
| 2405 | GACGUUCC A CAAAAACA | 1258 | UGUUUUUG CUGAUGAG GCCGUUAGGC CGAA IGAACGUC | 3959 | |
| 2407 | CGUUCCAC A AAAACAAG | 1259 | CUUGUUUU CUGAUGAG GCCGUUAGGC CGAA IUGGAACG | 3960 | |
| 2413 | ACAAAAAC A AGAUGAAA | 1260 | UUUCAUCU CUGAUGAG GCCGUUAGGC CGAA IUUUUUGU | 3961 | |
| 2423 | GAUGAAAC U GUGAUGCU | 1261 | ACCAUCAC CUGAUGAG GCCGUUAGGC CGAA IUUUCAUC | 3962 | |
| 2431 | UGUGAUGC U UGUGAAAG | 1262 | CUUUCACA CUGAUGAG GCCGUUAGGC CGAA ICAUCACA | 3963 | |
| 2446 | AGAAAGUC U CACUGAGA | 1263 | UCUCAGUG CUGAUGAG GCCGUUAGGC CGAA IACUUUCU | 3964 | |
| 2448 | AAAGUCUC A CUGAGACU | 1264 | AGUCUCAG CUGAUGAG GCCGUUAGGC CGAA IAGACUUU | 3965 | |
| 2450 | AGUCUCAC U GAGACUUC | 1265 | GAAGUCUC CUGAUGAG GCCGUUAGGC CGAA IUGAGACU | 3966 | |
| 2456 | ACUGAGAC U UCAUUUGA | 1266 | UCAAAUGA CUGAUGAG GCCGUUAGGC CGAA IUCUCAGU | 3967 | |
| 2459 | GAGACUUC A UUUGAGUC | 1267 | GACUCAAA CUGAUGAG GCCGUUAGGC CGAA IAAGUCUC | 3968 | |
| 2468 | UUUGAGUC A AUGAUAGA | 1268 | UCUAUCAU CUGAUGAG GCCGUUAGGC CGAA IACUCAAA | 3969 | |
| 2497 | GGAAAAAC U CAGUGCUIX | 1269 | AAGCACUG CUGAUGAG GCCGUUAGGC CGAA IUUUUUCC | 3970 | |
| 2499 | AAAAACUC A GUGCUUUG | 1270 | CAAAGCAC CUGAUGAG GCCGUUAGGC CGAA IAGUUUUU | 3971 | |
| 2504 | CUCAGUGC U UUGCCACC | 1271 | GGUGGCAA CUGAUGAG GCCGUUAGGC CGAA ICACUGAG | 3972 | |
| 2509 | UGCUUUGC C ACCUGAGG | 1272 | CCUCAGGU CUGAUGAG GCCGUUAGGC CGAA ICAAAGCA | 3973 | |
| 2510 | GCUUUGCC A CCUGAGGG | 1273 | CCCUCAGG CUGAUGAG GCCGUUAGGC CGMA IGCAAAGC | 3974 | |
| 2512 | UUUGCCAC C UGAGGGAG | 1274 | CUCCCUCA CUGAUGAG GCCGUUAGGC CGAA IUGGCAAA | 3975 | |
| 2513 | UUGCCACC U GACGGAGG | 1275 | CCUCCCUC CUGAUGAG GCCGUUAGGC CGAA IGUGGCAA | 3976 | |
| 2527 | AGGAAAGC C AUAUUUGG | 1276 | CCAAAUAU CUGAUGAG GCCGUUAGGC CGAA ICUUUCCU | 3977 | |
| 2528 | GGAAAGCC A UAUUUGGA | 1277 | UCCAAAUA CUGAUGAG GCCGUUAGGC CGAA IGCUUUCC | 3978 | |
| 2540 | UUGGAAUC U UUUAAGCU | 1278 | AGCUUAAA CUGAUGAG GCCGUUAGGC CGAA IAUUCCAA | 3979 | |
| 2548 | UUUUAAGC U CAGUUUAG | 1279 | CUAAACUG CUGAUGAG GCCGUUAGGC CGAA ICUUAAAA | 3980 | |
| 2550 | UUAAGCUC A GUUUAGAU | 1280 | AUCUAAAC CUGAUGAG GCCGUUAGGC CGAA IAGCUUAA | 3981 | |
| 2562 | UAGAUAAC A CAAAAGAU | 1281 | AUCUUUUG CUGAUGAG GCCGUUAGGC CGAA IUUAUCUA | 3982 | |
| 2564 | GAUAACAC A AAAGAUAC | 1282 | GUAUCUUU CUGAUGAG GCCGUUAGGC CGAA IUGUUAUC | 3983 | |
| 2573 | AAAGAUAC C CUGUUACC | 1283 | GGUAACAG CUGAUGAG GCCGUUAGGC CGAA IUAUCUUU | 3984 | |
| 2574 | AAGAUACC C UGUUACCU | 1284 | AGGUAACA CUGAUGAG GCCGUUAGGC CGAA IGUAUCUU | 3985 | |
| 2575 | AGAUACCC U GUUACCUG | 1285 | CAGGUAAC CUGAUGAG GCCGUUAGGC CGAA IGGUAUCU | 3986 | |
| 2581 | CCUGUUAC C UGAUGAAG | 1286 | CUUCAUCA CUGAUGAG GCCGUUAGGC CGAA IUAACAGG | 3987 | |
| 2582 | CUGUUACC U GAUGAAGU | 1287 | ACUUCAUC CUGAUGAG GCCGUUAGGC CGAA IGUAACAG | 3988 | |
| 2594 | GAAGUUUC A ACAUUGAG | 1288 | CUCAAUGU CUGAUGAG GCCGUUAGGC CGAA IAAACUUC | 3989 | |
| 2597 | GUUUCAAC A UUGAGCAA | 1289 | UUGCUCAA CUGAUGAG GCCGUUAGGC CGAA IUUGAAAC | 3990 | |
| 2604 | CAUUGAGC A AAAAGGAG | 1290 | CUCCUUUU CUGAUGAG GCCGUUAGGC CGAA ICUCAAUG | 3991 | |
| 2620 | GAAAAUUC C UUUGCAGA | 1291 | UCUGCAAA CUGAUGAG GCCGUUAGGC CGAA IAAUUUUC | 3992 | |
| 2621 | AAAAUUCC U UUGCAGAU | 1292 | AUCUGCAA CUGAUGAG GCCGUUAGGC CGAA IGAAUUUU | 3993 | |
| 2626 | UCCUUUGC A GAUGGAGG | 1293 | CCUCCAUC CUGAUGAG GCCGUUAGGC CGAA ICAAAGGA | 3994 | |
| 2638 | GGAGGAGC U CAGUACUG | 1294 | CAGUACUG CUGAUGAG GCCGUUAGGC CGAA ICUCCUCC | 3995 | |
| 2640 | AGGAGCUC A GUACUGCA | 1295 | UCCAGUAC CUGAUGAG GCCGUUAGGC CGAA IAGCUCCU | 3996 | |
| 2645 | CUCAGUAC U GCAGUUUA | 1296 | UAAACUGC CUGAUGAG GCCGUUAGGC CGAA LUACUGAG | 3997 | |
| 2648 | AGUACUGC A GUUUAUUC | 1297 | GAAUAAAC CUGAUGAG GCCGUUAGGC CGAA ICAGUACU | 3998 | |
| 2657 | GUUUAUUC A AAUGAUGA | 1298 | UCAUCAUU CUGAUGAG GCCGUUAGGC CGAA IAAUAAAC | 3999 | |
| 2667 | AUGAUCAC U UAUUUAUU | 1229 | AAUAAAUA CUGAUGAG GCCGUUAGGC CGAA IUCAUCAU | 4000 | |
| 2678 | UUUAUUUC U AAGGAAGC | 1300 | GCUUCCUU CUGAUGAG GCCGUUAGGC CGAA IAAAUAAA | 4001 | |
| 2687 | AAGGAAGC A CAGAUAAG | 1301 | CUUAUCUG CUGAUGAG GCCGUUAGGC CGAA ICUUCCUU | 4002 | |
| 2689 | GGAAGCAC A GAUAAGAG | 1302 | CUCUUAUC CUGAUGAG GCCGUUAGGC CGAA IUGCUUCC | 4003 | |
| 2702 | AGAGAAAC U GAAACGUU | 1303 | AACGUUUC CUGAUGAG GCCGUUAGGC CGAA IUUUCUCU | 4004 | |
| 2714 | ACGUUUUC A GAUUCAUC | 1304 | GAUGAAUC CUGAUGAG GCCGUUAGGC CGAA IAAAACGU | 4006 | |
| 2720 | UCAGAUUC A UCUCCAAU | 1306 | AUUGGAGA CUGAUGAG GCCGUUAGGC CGAA IAAUCUGA | 4006 | |
| 2723 | GAUUCAUC U CCAAIIUGA | 1306 | UCAAUUGG CUGAUGAG GCCGUUAGGC CGAA IAUGAAUC | 4007 | |
| 2725 | UUCAUCUC C AAUUGAAA | 1307 | UUUCAAUU CUGAUGAG GCCGUUAGGC CGAA IAGAUGAA | 4008 | |
| 2726 | UCAUCUCC A AUUGAAAU | 1308 | AUUUCAAU CUGAUGAG GCCGUUAGGC CGAA IGAGAUGA | 4009 | |
| 2748 | AUGAGUUC C CUACAUUG | 1309 | CAAUGUAG CUGAUGAG GCCGUUAGGC CGAA IAACUCAU | 4010 | |
| 2749 | UGAGUUCC C UACAUUGA | 1310 | UCAAUGUA CUGAUGAG GCCGUUAGGC CGAA IGAACUCA | 4011 | |
| 2750 | GAGUUCCC U ACAUUGAU | 1311 | AUCAAUGU CUGAUGAG GCCGUUAGGC CGAA IGGAACUC | 4012 | |
| 2753 | UUCCCUAC A UUGAUCAG | 1312 | CUGAUCAA CUGAUGAG GCCGUUAGGC CGAA IUAGGGAA | 4013 | |
| 2760 | CAUUGAUC A GUUCUAAA | 1313 | UUUAGAAC CUGAUGAG GCCGUUAGGC CGAA IAUCAAUG | 4014 | |
| 2765 | AUCAGUUC U AAAACUGA | 1314 | UCAGUUUU CUGAUGAG GCCGUUAGGC CGAA IAACUGAU | 4015 | |
| 2771 | UCUAAAAC U GAUUCAUU | 1315 | AAUGAAUC CUGAUGAG GCCGUUAGGC CGAA IUZJUUAGA | 4016 | |
| 2777 | ACUGAUUC A UUUUCUAA | 1316 | UUAGAAAA CUGAUGAG GCCGUUAGGC CGAA IAAUCAGU | 4017 | |
| 2783 | UCAUUUUC U AAAUUAGC | 1317 | GCUAAUUU CUGAUGAG GCCGUUAGGC CGAA IAAAAUGA | 4018 | |
| 2792 | AAAUUAGC C AGGGAAUA | 1318 | UAUUCCCU CUGAUGAG GCCGUUAGGC CGAA ICUAAUUU | 4019 | |
| 2793 | AAUUAGCC A GGGAAUAU | 1319 | AUAUUCCC CUGAUGAG GCCGUUAGGC CGAA IGCUAAUU | 4020 | |
| 2804 | GAAUAUAC U GACCUAGA | 1320 | UCUAGGUC CUGAUGAG GCCGUUAGGC CGAA IUAUAUUC | 4021 | |
| 2808 | AUACUGAC C UAGAAGUA | 1321 | UACUUCUA CUGAUGAG GCCGUUAGGC CGAA IUCAGUAU | 4022 | |
| 2809 | UACUGACC U AGAAGUAU | 1322 | AUACUUCU CUGAUGAG GCCGUUAGGC CGAA IGUCAGUA | 4023 | |
| 2819 | GAAGUAUC C CACAAAAG | 1323 | CUUUUGUG CUGAUGAG GCCGUUAGGC CGAA IAUACUUC | 4024 | |
| 2820 | AAGUAUCC C ACAAAAGU | 1324 | ACUUUUGU CUGAUGAG GCCGUUAGGC CGAA IGAUACUU | 4025 | |
| 2821 | AGUAUCCC A CAAAAGUG | 1325 | CACUUUUG CUGAUGAG GCCGUUAGGC CGAA IGGAUACU | 4026 | |
| 2823 | UAUCCCAC A AAAGUGAA | 1326 | UUCACUUU CUGAUGAG GCCGUUAGGC CGAA IUGGGAUA | 4027 | |
| 2837 | GAAAUUGC U AAUGCCCC | 1327 | GGGGCAUU CUGAUGAG GCCGUUAGGC CGAA ICAAUUUC | 4028 | |
| 2843 | GCUAAUGC C CCGGAUGG | 1328 | CCAUCCGG CUGAUGAG GCCGUUAGGC CGAA ICAUUAGC | 4029 | |
| 2844 | CUAAUGCC C CGGAUGGA | 1329 | UCCAUCCG CUGAUGAG GCCGUUAGGC CGAA IGCAUUAG | 4030 | |
| 2845 | UAAUGCCC C GGAUGGAG | 1330 | CUCCAUCC CUGAUGAG GCCGUUAGGC CGAA IGGCAUUA | 4031 | |
| 2855 | GAUGGAGC U GGGUCAUU | 1331 | AAUGACCC CUGAUGAG GCCGUUAGGC CGAA ICUCCAUC | 4032 | |
| 2861 | GCUGGGUC A UUGCCUUG | 1332 | CAAGGCAA CUGAUGAG GCCGUUAGGC CGAA IACCCAGC | 4033 | |
| 2866 | GUCAUUGC C UUGCACAG | 1333 | CUGUGCAA CUGAUGAG GCCGUUAGGC CGAA ICAAUGAC | 4034 | |
| 2867 | UCAUUGCC U UGCACAGA | 1334 | UCUGUGCA CUGAUGAG GCCGUUAGGC CGAA IGCAAUGA | 4035 | |
| 2871 | UGCCUUGC A CAGAAUUG | 1335 | CAAUUCUG CUGAUGAG GCCGUUAGGC CGAA ICAAGGCA | 4036 | |
| 2873 | CCUUGCAC A GAAUUGCC | 1336 | GGCAAUUC CUGAUGAG GCCGUUAGGC CGAA IUGCAAGG | 4037 | |
| 2881 | AGAAUUGC C CCAUGACC | 1337 | GGUCAUGG CUGAUGAG GCCGUUAGGC CGAA ICAAUUCU | 4038 | |
| 2882 | GAAUUGCC C CAUGACCU | 1338 | AGGUCAUG CUGAUGAG GCCGUUAGGC CGAA IGCAAUUC | 4039 | |
| 2883 | AAUUGCCC C AUGACCUU | 1339 | AAGGUCAU CUGAUGAG GCCGUUAGGC CGAA IGGCAAUU | 4040 | |
| 2884 | AUUGCCCC A UGACCUUU | 1340 | AAAGGUCA CUGAUGAG GCCGUUAGGC CGAA IGGGCAAU | 4041 | |
| 2889 | CCCAUGAC C UUUCUUUG | 1341 | CAAAGAAA CUGAUGAG GCCGUUAGGC CGAA IUCAUGGG | 4042 | |
| 2890 | CCAUGACC U UUCUUUGA | 1342 | UCAAAGAA CUGAUGAG GCCGUUAGGC CGAA IGUCAUGG | 4043 | |
| 2894 | GACCUUUC U UUGAAGAA | 1343 | UUCUUCAA CUGAUGAG GCCGUUAGGC CGAA IAAAGGUC | 4044 | |
| 2904 | UGAAGAAC A UACAACCC | 1344 | GGGUUGUA CUGAUGAG GCCGUUAGGC CGAA IUUCUUCA | 4045 | |
| 2908 | GAACAUAC A ACCCAAAG | 1345 | CUUUGGGU CUGAUGAG GCCGUUAGGC CGAA IUAUGUUC | 4046 | |
| 2911 | CAUACAAC C CAAAGUUG | 1346 | CAACUUUG CUGAUGAG GCCGUUAGGC CGAA IUUGUAUG | 4047 | |
| 2912 | AUACAACC C AAAGUUGA | 1347 | UCAACUUU CUGAUGAG GCCGUUAGGC CGAA IGUUGUAU | 4048 | |
| 2913 | UACAACCC A AAGUUGAA | 1348 | UUCAACUU CUGAUGAG GCCGUUAGGC CGAA IGGUUGUA | 4049 | |
| 2931 | AGAAAAUC A GUUUCUCA | 1349 | UGAGAAAC CUGAUGAG GCCGUUAGGC CGAA IAUUUUCU | 4050 | |
| 2937 | UCAGUCUC U CAGAUGAC | 1350 | GUCAUCUG CUGAUGAG GCCGUUAGGC CGAA IAAACUGA | 4051 | |
| 2939 | AGUUUCUC A GAUUACUU | 1351 | AAGUCAUC CUGAUGAG GCCGUUAGGC CGAA IAGAAACU | 4052 | |
| 2946 | CAGAUGAC U UUUCUAAA | 1352 | UUUAGAAA CUGAUGAG GCCGUUAGGC CGAA IUCAUCUG | 4053 | |
| 2951 | GACUUUUC U AAAAAUGG | 1353 | CCAUUUUU CUGAUGAG GCCGUUAGGC CGAA IAAAAGUC | 4054 | |
| 2963 | AAUGGGUC U GCUACAUC | 1354 | GAUGUAGC CUGAUGAG GCCGUUAGGC CGAA IACCCAUU | 4055 | |
| 2966 | GGGUCUGC U ACAUCAAA | 1355 | UUUGAUGU CUGAUGAG GCCGUUAGGC CGAA ICAGACCC | 4056 | |
| 2969 | UCUCCUAC A UCAAAGGU | 1356 | ACCUUUGA CUGAUGAG GCCGUUAGGC CGAA IUAGCAGA | 4057 | |
| 2972 | GCUACAUC A AAGGUGCU | 1357 | AGCACCUU CUGAUGAG GCCGUUAGGC CGAA IAUGUAGC | 4058 | |
| 2980 | AAAGGUGC U CUUAUUGC | 1358 | GCAAUAAG CUGAUGAG GCCGUUAGGC CGAA ICACCUUU | 4059 | |
| 2982 | AGGUGCUC U UAUUGCCU | 1359 | AGGCAAUA CUGAUGAG GCCGUUAGGC CGAA IAGCACCU | 4060 | |
| 2989 | CUUAUUGC C UCCAGAUG | 1360 | CAUCUGGA CUGAUGAG GCCGUUAGGC CGAA ICAAUAAG | 4061 | |
| 2990 | UUAUUGCC U CCAGAUGU | 1361 | ACAUCUGG CUGAUGAG GCCGUUAGGC CGAA IGCAAUAA | 4062 | |
| 2992 | AUUGCCUC C AGAUGUUU | 1362 | AAACAUCU CUGAUGAG GCCGUUAGGC CGAA IAGGCAAU | 4063 | |
| 2993 | UUGCCUCC A GAUGUUUC | 1363 | GAAACAUC CUGAUGAG GCCGUUAGGC CGAA IGAGGCAA | 4064 | |
| 3002 | GAUGUUUC U GCUUUGGC | 1364 | GCCAAAGC CUGAUGAG GCCGUUAGGC CGAA IAAACAUC | 4065 | |
| 3005 | GUUUCUGC U UUGGCCAC | 1365 | GUGGCCAA CUGAUGAG GCCGUUAGGC CGAA ICAGAAAC | 4066 | |
| 3011 | GCUUUGGC C ACUCAAGC | 1366 | GCUUGAGU CUGAUGAG GCCGUUAGGC CGAA ICCAAAGC | 4067 | |
| 3012 | CUUUGGCC A CUCAAGCA | 1367 | UCCUGGAG CUGAUGAG GCCGUUAGGC CGAA IGCCAAAG | 4068 | |
| 3014 | UUGGCCAC U CAAGCAGA | 1368 | UCUGCUUG CUGAUGAG GCCGUUAGGC CGAA IUGGCCAA | 4069 | |
| 3016 | GGCCACUC A AGCAGAGA | 1369 | UCUCUGCU CUGAUGAG GCCGUUAGGC CGAA IAGUGGCC | 4070 | |
| 3020 | ACUCAAGC A GAGAUAGA | 1370 | UCUAUCUC CUGAUGAG GCCGUUAGGC CGAA ICUUGAGU | 4071 | |
| 3033 | UAGAGAGC A UAGUUAAA | 1371 | UUUAACUA CUGAUGAG GCCGUUAGGC CGAA ICUCUCUA | 4072 | |
| 3043 | AGUUAAAC C CAAAGUUC | 1372 | GAACUUUG CUGAUGAG GCCGUUAGGC CGAA IUUUAACU | 4073 | |
| 3044 | GUUAAACC C AAAGUUCU | 1373 | AGAACUUU CUGAUGAG GCCGUUAGGC CGAA IGUUUAAC | 4074 | |
| 3045 | UUAAACCC A AAGUUCUU | 1374 | AAGAACUU CUGAUGAG GCCGUUAGGC CGAA IGGUUUAA | 4075 | |
| 3052 | CAAAGUUC U UGUGAAAG | 1375 | CUUUCACA CUGAUGAG GCCGUUAGGC CGAA IAACUUUG | 4076 | |
| 3065 | AAAGAAGC U GAGAAAAA | 1376 | UUUUUCUC CUGAUGAG GCCGUUAGGC CGAA ICUUCUUU | 4077 | |
| 3076 | GAAAAAAC U UCCUUCCG | 1377 | CGGAAGGA CUGAUGAG GCCGUUAGGC CGAA IUUUUUUC | 4078 | |
| 3079 | AAAACUUC C UUCCGAUA | 1378 | UAUCGGAA CUGAUGAG GCCGUUAGGC CGAA IAAGUUUU | 4079 | |
| 3080 | AAACUUCC U UCCGAUAC | 1379 | GUAUCGGA CUGAUGAG GCCGUUAGGC CGAA IGAAGUUU | 4080 | |
| 3083 | CUUCCUUC C GAUACAGA | 1380 | UCUGUAUC CUGAUGAG GCCGUUAGGC CGAA IAAGGAAG | 4081 | |
| 3089 | UCCGAUAC A GAAAAAGA | 1381 | UCUUUUUC CUGAUGAG GCCGUUAGGC CGAA IUAUCGGA | 4082 | |
| 3102 | AAGAGGAC A GAUCACCA | 1382 | UGGUGAUC CUGAUGAG GCCGUUAGGC CGAA IUCCUCUU | 4083 | |
| 3107 | GACAGAUC A CCAUCUGC | 1383 | GCAGAUGG CUGAUGAG GCCGUUAGGC CGAA IAUCUGUC | 4084 | |
| 3109 | CAGAUCAC C AUCUGCUA | 1384 | UAGCAGAU CUGAUGAG GCCGUUAGGC CGAA LUGAUCUG | 4085 | |
| 3110 | AGAUCACC A UCUGCUAU | 1385 | AUAGCAGA CUGAUGAG GCCGUUAGGC CGAA IGUGAUCU | 4086 | |
| 3113 | UCACCAUC U GCUAUAUU | 1386 | AAUAUAGC CUGAUGAG GCCGUUAGGC CGAA IAUGGUGA | 4087 | |
| 3116 | CCAUCUGC U AUAUUUUC | 1387 | GAAAAUAU CUGAUGAG GCCGUUAGGC CGAA ICAGAUGG | 4088 | |
| 3125 | AUAUUUUC A GCAGAGCU | 1388 | AGCUCUGC CUGAUGAG GCCGUUAGGC CGAA IAAAAUAU | 4089 | |
| 3128 | UUUUCAGC A GAGCUGAG | 1389 | CUCAGCUC CUGAUGAG GCCGUUAGGC CGAA ICUGAAAA | 4090 | |
| 3133 | AGCAGAGC U GAGUAAAA | 1390 | UUGUACUC CUGAUGAG GCCGUUAGGC CGAA ICUCUGCU | 4091 | |
| 3143 | AGUAAAAC U UCAGUUGU | 1391 | ACAACUGA CUGAUGAG GCCGUUAGGC CGAA IUUUUACU | 4092 | |
| 3146 | AAAACUUC A GUUGUUGA | 1392 | UCAACAAC CUGAUGAG GCCGUUAGGC CGAA IAAGUUUU | 4093 | |
| 3156 | UUGUUGAC C UCCUGUAC | 1393 | GUACAGGA CUGAUGAG GCCGUUAGGC CGAA IUCAACAA | 4094 | |
| 3157 | UGUUGACC U CCUGUACU | 1394 | AGUACAGG CUGAUGAG GCCGUUAGGC CGAA IGUCAACA | 4095 | |
| 3159 | UUGACCUC C UGUACUGG | 1395 | CCAGUACA CUGAUGAG GCCGUUAGGC CGAA IAGGUCAA | 4096 | |
| 3160 | UGACCUCC U GUACUGGA | 1396 | UCCAGUAC CUGAUGAG GCCGUUAGGC CGAA IGAGGUCA | 4097 | |
| 3165 | UCCUGUAC U GGAGAGAC | 1397 | GUCUCUCC VJGAUGAG GCCGUUAGGC CGAA IUACAGGA | 4098 | |
| 3174 | GGAGAGAC A UUAAGAAG | 1398 | CUUCUUAA CUGAUGAG GCCGUUAGGC CGAA IUCUCUCC | 4099 | |
| 3185 | AAGAAGAC U GGAGUGGU | 1399 | ACCACUCC CUGAUGAG GCCGUUAGGC CGAA IUCUUCUU | 4100 | |
| 3203 | UUUGGUGC C AGCCUAUU | 1400 | AAUAGGCU CUGAUGAG GCCGUUAGGC CGAA ICACCAAA | 4101 | |
| 3204 | UUGGUGCC A GCCUAUUC | 1401 | GAAUAGGC CUGAUGAG GCCGUUAGGC CGAA IGCACCAA | 4102 | |
| 3207 | GUGCCAGC C UAUUCCUG | 1402 | CAGGAAUA CUGAUGAG GCCGUUAGGC CGAA ICUGGCAC | 4103 | |
| 3208 | UGCCAGCC U AUUCCUGC | 1403 | GCAGGAAU CUGAUGAG GCCGUUAGGC CGAA IGCUGGCA | 4104 | |
| 3213 | GCCUAUUC C UGCUGCUU | 1404 | AAGCAGCA CUGAUGAG GCCGUUAGGC CGAA IAAUAGGC | 4105 | |
| 3214 | CCUAUUCC U GCUGCUUU | 1405 | AAAGCAGC CUGAUGAG GCCGUUAGGC CGAA IGAAUAGG | 4106 | |
| 3217 | AUUCCUGC U GCUUUCAU | 1406 | AUGAAAGC CUGAUGAG GCCGUUAGGC CGAA ICAGGAAU | 4107 | |
| 3220 | CCUGCUGC U UUCAUUGA | 1407 | UCAAUGAA CUGAUGAG GCCGUUAGGC CGAA ICAGCAGG | 4108 | |
| 3224 | CUGCUUUC A UUGACAGU | 1408 | ACUGUCAA CUGAUGAG GCCGUUAGGC CGAA IAAAGCAG | 4109 | |
| 3230 | UCAUUGAC A GUAUUCAG | 1409 | CUGAAUAC CUGAUGAG GCCGUUAGGC CGAA IUCAAUGA | 4110 | |
| 3237 | CAGUAUUC A GCAUUGUG | 1410 | CACAAUGC CUGAUGAG GCCGUUAGGC CGAA IAAUACUG | 4111 | |
| 3240 | UAUUCAGC A UUGUGAGC | 1411 | GCUCACAA CUGAUGAG GCCGUUAGGC CGAA ICUGAAUA | 4112 | |
| 3254 | AGCGUAAC A GCCUACAU | 1412 | AUGUAGGC CUGAUGAG GCCGUUAGGC CGAA IUUACGCU | 4113 | |
| 3257 | GUAACAGC C UACAUUGC | 1413 | GCAAUGUA CUGAUGAG GCCGUIUAGGC CGAA ICUGUUAC | 4114 | |
| 3258 | UAACAGCC U ACAUUGCC | 1414 | GGCAAUGU CUGAUGAG GCCGUUAGGC CGAA IGCUGUUA | 4115 | |
| 3261 | CAGCCUAC A UUGCCUUG | 1415 | CAAGGCAA CUGAUGAG GCCGUUAGGC CGAA IUAGGCUG | 4116 | |
| 3266 | UACAUUGC C UUGGCCCU | 1416 | AGGGCCAA CUGAUGAG GCCGUUAGGC CGAA ICAAUGUA | 4117 | |
| 3267 | ACAUUGCC U UGGCCCUG | 1417 | CAGGGCCA CUGAUGAG GCCGUUAGGC CGAA IGCAAUGU | 4118 | |
| 3272 | GCCUUGGC C CUGCUCUC | 1418 | GAGAGCAG CUGAUGAG GCCGUUAGGC CGAA ICCAAGGC | 4119 | |
| 3273 | CCUUGGCC C UGCUCUCU | 1419 | AGAGAGCA CUGAUGAG GCCGUUAGGC CGAA IGCCAAGG | 4120 | |
| 3274 | CUUGGCCC U GCUCUCUG | 1420 | CAGAGAGC CUGAUGAG GCCGUUAGGC CGAA IGGCCAAG | 4121 | |
| 3277 | GGCCCUGC U CUCUGUGA | 1421 | UCACAGAG CUGAUGAG GCCGUUAGGC CGAA ICAGGGCC | 4122 | |
| 3279 | CCCUGCUC U CUGUGACC | 1422 | GGUCACAG CUGAUGAG GCCGUUAGGC CGAA IAGCAGGG | 4123 | |
| 3281 | CUGCUCUC U GUGACCAU | 1423 | AUGGUCAC CUGAUGAG GCCGUUAGGC CGAA IAGAGCAG | 4124 | |
| 3287 | UCUGUGAC C AUCAGCUU | 1424 | AAGCUGAU CUGAUGAG GCCGUUAGGC CGAA IUCACAGA | 4125 | |
| 3288 | CUGUGACC A UCAGCUUU | 1425 | AAAGCUGA CUGAUGAG GCCGUUAGGC CGAA IGUCACAG | 4126 | |
| 3291 | UGACCAUC A GCUUUAGG | 1426 | CCUAAAGC CUGAUGAG GCCGUUAGGC CGAA IAUGGUCA | 4127 | |
| 3294 | CCAUCAGC U UUAGGAUA | 1427 | UAUCCUAA CUGAUGAG GCCGUUAGGC CGAA ICUGAUGG | 4128 | |
| 3306 | GGAUAUAC A AGGGUGUG | 1428 | CACACCCU CUGAUGAG GCCGUUAGGC CGAA IUAUAUCC | 4129 | |
| 3318 | GUGUGAUC C AAGCUAUC | 1429 | GAUAGCUU CUGAUGAG GCCGUUAGGC CGAA IAUCACAC | 4130 | |
| 3319 | UGUGAUCC A AGCUAUCC | 1430 | GGAUAGCU CUGAUGAG GCCGUUAGGC CGAA IGAUCACA | 4131 | |
| 3323 | AUCCAAGC U AUCCAGAA | 1431 | UUCUGGAU CUGAUGAG GCCGUUAGGC CGAA ICUUGGAU | 4132 | |
| 3327 | AAGCUAUC C AGAAAUCA | 1432 | UGAUUUCU CUGAUGAG GCCGUUAGGC CGAA IAUAGCUU | 4133 | |
| 3328 | AGCUAUCC A GAAAUCAG | 1433 | CUGAUUUC CUGAUGAG GCCGUUAGGC CGAA IGAUAGCU | 4134 | |
| 3335 | CAGAAAUC A GAUGAAGG | 1434 | CCUUCAUC CUGAUGAG GCCGUUAGGC CGAA IAUUUCUG | 4135 | |
| 3345 | AUGAAGGC C ACCCAUUC | 1435 | GAAUGGGU CUGAUGAG GCCGUUAGGC CGAA ICCUUCAU | 4136 | |
| 3346 | UGAAGGCC A CCCAUUCA | 1436 | UGAAUGGG CUGAUGAG GCCGUUAGGC CGAA IGCCUUCA | 4137 | |
| 3348 | AAGGCCAC C CAUUCAGG | 1437 | CCUGAAUG CUGAUGAG GCCGUUAGGC CGAA IUGGCCUU | 4138 | |
| 3348 | AGGCCACC C AUUCAGGG | 1438 | CCCUGAAU CUGAUGAG GCCGUUAGGC CGAA IGUGGCCU | 4139 | |
| 3350 | GGCCACCC A UUCAGGGC | 1439 | GCCCUGAA CUGAUGAG GCCGUUAGGC CGAA IGGUGGCC | 4140 | |
| 3354 | ACCCAUUC A GGGCAUAU | 1440 | AUAUGCCC CUGAUGAG GCCGUUAGGC CGAA IAAUGGGU | 4141 | |
| 3359 | UUCAGGGC A UAUCUGGA | 1441 | UCCAGAUA CUGAUGAG GCCGUUAGGC CGAA ICCCUGAA | 4142 | |
| 3364 | GGCAUAUC U GGAAUCUG | 1442 | CAGAUUCC CUGAUGAG GCCGUUAGGC CGAA IAUAUGCC | 4143 | |
| 3371 | CUGGAAUC U GAAGUUGC | 1443 | GCAACUUC CUGAUGAG GCCGUUAGGC CGAA IAUUCCAG | 4144 | |
| 3380 | GAAGUUGC U AUAUCUGA | 1444 | UCAGAUAU CUGAUGAG GCCGUUAGGC CGAA ICAACUUC | 4145 | |
| 3386 | GCUAUAUC U GAGGAGUU | 1445 | AACUCCUC CUGAUGAG GCCGUUAGGC CGAA IAUAUAGC | 4146 | |
| 3400 | GUUGGUUC A GAAGUACA | 1446 | UGUACUUC CUGAUGAG GCCGUUAGGC CGAA IAACCAAC | 4147 | |
| 3408 | AGAAGUAC A GUAAUUCU | 1447 | AGAAUUAC CUGAUGAG GCCGUUAGGC CGAA IUACUUCU | 4148 | |
| 3416 | AGUAAUUC U GCUCUUGG | 1448 | CCAAGAGC CUGAUGAG GCCGUUAGGC CGAA IAAUUACU | 4149 | |
| 3419 | AAUUCUGC U CUUGGUCA | 1449 | UGACCAAG CUGAUGAG GCCGUUAGGC CGAA ICAGAAUU | 4150 | |
| 3421 | UUCUGCUC U UGGUCAUG | 1450 | CAUGACCA CUGAUGAG GCCGUUAGGC CGAA IAGCAGAA | 4151 | |
| 3427 | UCUUGGUC A UGUGAACU | 1451 | AGUUCACA CUGAUGAG GCCGUUAGGC CGAA IACCAAGA | 4152 | |
| 3435 | AUGUGAAC U GCACGAUA | 1452 | UAUCGUGC CUGAUGAG GCCGUUAGGC CGAA IUUCACAU | 4153 | |
| 3438 | UGAACUGC A CGAUAAAG | 1453 | CUUUAUCG CUGAUGAG GCCGUUAGGC CGAA ICAGUUCA | 4154 | |
| 3451 | AAAGGAAC U CAGGCGCC | 1454 | GGCGCCUG CUGAUGAG GCCGUUAGGC CGAA IUUCCUUU | 4155 | |
| 3453 | AGGAACUC A GGCGCCUC | 1455 | GAGGCGCC CUGAUGAG GCCGUUAGGC CGAA IAGUUCCU | 4156 | |
| 3459 | UCAGGCGC C UCUUCUUA | 1456 | UAAGAAGA CUGAUGAG GCCGUUAGGC CGAA ICGCCUGA | 4157 | |
| 3460 | CAGGCGCC U CUUCUUAG | 1457 | CUAAGAAG CUGAUGAG GCCGUUAGGC CGAA IGCGCCUG | 4158 | |
| 3462 | UCCUCCUC U UCUCAGUC | 1458 | AACUAAGA CUGAUGAG GCCGUUAGGC CGAA IAGGCGCC | 4159 | |
| 3465 | GCCUCUUC U UAGUUGAU | 1459 | AUCAACUA CUGAUGAG GCCGUUAGGC CGAA IAAGAGGC | 4160 | |
| 3488 | GUUGAUUC U CUGAAGUU | 1460 | AACUUCAG CUGAUGAG GCCGUUAGGC CGAA IAAUCAAC | 4161 | |
| 3490 | UGAUUCUC U GAAGUUUG | 1461 | CAAACUUC CUGAUGAG GCCGUUAGGC CGAA IAGAAUCA | 4162 | |
| 3500 | AAGUUUGC A GUGUUGAU | 1462 | AUCAACAC CUGAUGAG GCCGUUAGGC CGAA ICAAACUU | 4163 | |
| 3521 | GUAUUUAC C UAUGUUGG | 1463 | CCAACAUA CUGAUGAG GCCGUUAGGC CGAA IUAAAUAC | 4164 | |
| 3522 | UAUUUACC U AUGUUGGU | 1464 | ACCAACAU CUGAUGAG GCCGUUAGGC CGAA IGUAAAUA | 4165 | |
| 3533 | GUUGGUGC C UUGUUUAA | 1465 | UUAAACAA CUGAUGAG GCCGUUAGGC CGAA ICACCAAC | 4166 | |
| 3534 | UUGGUGCC U UGUUUAAU | 1466 | AUUAAACA CUGAUGAG GCCGUUAGGC CGAA IGCACCAA | 4167 | |
| 3547 | UAAUGGUC U GACACUAC | 1467 | GUAGUGUC CUGAUGAG GCCGUUAGGC CGAA IACCAUUA | 4168 | |
| 3551 | GGUCUGAC A CUACUGAU | 1468 | AUCAGUAG CUGAUGAG GCCGUUAGGC CGAA IUCAGACC | 4169 | |
| 3553 | UCUGACAC U ACUGAUUU | 1469 | AAAUCAGU CUGAUGAG GCCGUUAGGC CGAA IUGUCAGA | 4170 | |
| 3556 | GACACUAC U GAUUUUGG | 1470 | CCAAAAUC CUGAUGAG GCCGUUAGGC CGAA IUAGUGUC | 4171 | |
| 3566 | AUUUUGGC U CUCAUCUC | 1471 | GAAAUGAG CUGAUGAG GCCGUUAGGC CGAA ICCAAAAU | 4172 | |
| 3568 | UUUGGCUC U CAUUUCAC | 1472 | GUGAAAUG CUGAUGAG GCCGUUAGGC CGAA IAGCCAAA | 4173 | |
| 3570 | UGGCUCUC A UUUCACUC | 1473 | GAGUGAAA CUGAUGAG GCCGUUAGGC CGAA IAGAGCCA | 4174 | |
| 3575 | CUCAUUUC A CUCUUCAG | 1474 | CUGAAGAG CUGAUGAG GCCGUUAGGC CGAA IAAAUGAG | 4175 | |
| 3577 | CAUCUCAC U CUUCAGUG | 1475 | CACUGAAG CUGAUGAG GCCGUUAGGC CGAA IUGAAAUG | 4176 | |
| 3579 | UUUCACUC U UCAGUGUC | 1476 | AACACUGA CUGAUGAG GCCGUUAGGC CGAA IAGUGAAA | 4177 | |
| 3582 | CACUCUUC A GUGUUCCU | 1477 | AGGAACAC CUGAUGAG GCCGUUAGGC CGAA IAAGAGUG | 4178 | |
| 3589 | CAGUGUUC C UGUUAUUU | 1478 | AAAUAACA CUGAUGAG GCCGUUAGGC CGAA IAACACUG | 4179 | |
| 3590 | AGUGUUCC U GUUAUUUA | 1479 | UAAAUAAC CUGAUGAG GCCGUUAGGC CGAA IGAACACU | 4180 | |
| 3607 | UGAACGGC A UCAGGCAC | 1480 | GUGCCUGA CUGAUGAG GCCGUUAGGC CGAA ICCGUUCA | 4181 | |
| 3610 | ACGGCAUC A GGCACAGA | 1481 | UCUGUGCC CUGAUGAG GCCGUUAGGC CGAA IAUGCCGU | 4182 | |
| 3614 | CAUCAGGC A CAGAUAGA | 1482 | UCUAUCUG CUGAUGAG GCCGUUAGGC CGAA ICCUGAUG | 4183 | |
| 3616 | UCAGGCAC A GAUAGAUC | 1483 | GAUCUAUC CUGAUGAG GCCGUUAGGC CGAA IUGCCUGA | 4184 | |
| 3625 | GAUAGAUC A UUAUCUAG | 1484 | CUAGAUAA CUGAUGAG GCCGUUAGGC CGAA IAUCUAUC | 4185 | |
| 3631 | UCAUCAUC U AGGACCUG | 1485 | CAAGUCCU CUGAUGAG GCCGUUAGGC CGAA IAUAAUGA | 4186 | |
| 3637 | UCUAGGAC U UGCAAAUA | 1486 | UAUUUGCA CUGAUGAG GCCGUUAGGC CGAA IUCCUAGA | 4187 | |
| 3641 | GGACUUGC A AAUAAGAA | 1487 | UCCUUAUU CUGAUGAG GCCGUUAGGC CGAA ICAAGUCC | 4188 | |
| 3662 | AAAGAUGC U AUGGCUAA | 1488 | UUAGCCAU CUGAUGAG GCCGUUAGGC CGAA ICAUCUCU | 4189 | |
| 3668 | GCUAUGGC U AAAAUCCA | 1489 | UGGAUUUU CUGAUGAG GCCGUUAGGC CGAA ICCAUAGC | 4190 | |
| 3675 | CUAAAUC C AAGCAAAA | 1490 | UUUUGCUU CUGAUGAG GCCGUUAGGC CGAA IAUUUUAG | 4191 | |
| 3676 | UAAAAUCC A AGCAAAAA | 1491 | UUUUUGCU CUGAUGAG GCCGUUAGGC CGAA IGAUUUUA | 4192 | |
| 3680 | AUCCAAGC A AAAAUCCC | 1492 | GGGAUUUU CUGAUGAG GCCGUUAGGC CGAA ICUUGGAU | 4193 | |
| 3687 | CAAAAAUC C CUGGAUUG | 1493 | CAAUCCAG CUGAUGAG GCCGUUAGGC CGAA IAUUUUUG | 4194 | |
| 3688 | AAAAAUCC C UGGAUUGA | 1494 | UCAAUCCA CUGAUGAG GCCGUUAGGC CGAA IGAUUUUU | 4195 | |
| 3689 | AAAAUCCC U GGAUUGAA | 1495 | UUCAAUCC CUGAUGAG GCCGUUAGGC CGAA IGGAUUUU | 4196 | |
| 3702 | UGAAGCGC A AAGCUGAA | 1496 | UUCAGCUU CUGAUGAG GCCGUUAGGC CGAA ICGCUCCA | 4197 | |
| 3707 | CGCAAAGC U GAAUGAAA | 1497 | UUUCAUUC CUGAUGAG GCCGUUAGGC CGAA ICUUUGCG | 4198 | |
| 3720 | GAAAACGC C CAAAAUAA | 1498 | UUAUUUUG CUGAUGAG GCCGUUAGGC CGAA ICGUUUUC | 4199 | |
| 3721 | AAAACGCC C AAAAUAAU | 1499 | AUUAUUUC CUGAUGAG GCCGUUAGGC CGAA IGCGUUUU | 4200 | |
| 3722 | AAACGCCC A AAAUAAUU | 1500 | AAUUAUUU CUGAUGAG GCCGUUAGGC CGAA IGGCGUUU | 4201 | |
| 3742 | AGGAGUUC A UCUUUAAA | 1501 | UUUAAAGA CUGAUGAG GCCGUUAGGC CGAA IAACUCCU | 4202 | |
| 3745 | AGUUCAUC U UCAAAGGG | 1502 | CCCUUUAA CUGAUGAG GCCGUUAGGC CGAA IAUGAACU | 4203 | |
| 3761 | GGAUAUUC A UUUGAUUA | 1503 | UAAUCAAA CUGAUGAG GCCGUUAGGC CGAA IAAUAUCC | 4204 | |
| 3784 | GGAGGGUC A GGGAAGAA | 1504 | UUCUUCCC CUGAUGAG GCCGUUAGGC CGAA IACCCUCC | 4205 | |
| 3798 | GAACGAAC C UUGACGUU | 1505 | AACGUCAA CUGAUGAG GCCGUUAGGC CGAA IUUCGUUC | 4206 | |
| 3799 | AACGAACC U UGACGUUG | 1506 | CAACGUCA CUGAUGAG GCCGUUAGGC CGAA IGUUCGUU | 4207 | |
| 3809 | GACGUUGC A GUGCAGUU | 1507 | AACUGCAC CUGAUGAG GCCGUUAGGC CGAA ICAACGUC | 4208 | |
| 3814 | UGCAGUGC A GUUUCACA | 1508 | UGUGAAAC CUGAUGAG GCCGUUAGGC CGAA ICACUGCA | 4209 | |
| 3820 | UCAGUUUC A CAGAUCGU | 1509 | ACCAUCUG CUGAUGAG GCCGUUAGGC CGAA IAAACUGC | 4210 | |
| 3822 | AGUCUCAC A GAUCGUUG | 1510 | CAACGAUC CUGAUGAG GCCGUUAGGC CGAA IUGAAACU | 4211 | |
| 3838 | GUCAGAUC U UUAUUUUU | 1511 | AAAAAUAA CUGAUGAG GCCGUUAGGC CGAA IAUCUAAC | 4212 | |
| 3850 | UUUUUAGC C AUGCACUG | 1512 | CAGUGCAU CUGAUGAG GCCGUUAGGC CGAA ICUAAAAA | 4213 | |
| 3851 | UUUUAGCC A UGCACUGU | 1513 | ACAGUGCA CUGAUGAG GCCGUUAGGC CGAA IGCUAAAA | 4214 | |
| 3855 | AGCCAUGC A CUGUUGUG | 1514 | CACAACAG CUGAUGAG GCCGUUAGGC CGAA ICAUGGCU | 4215 | |
| 3857 | CCAUGCAC U UCUGUGAG | 1515 | CUCACAAC CUGAUGAG GCCGUUAGGC CGAA IUGCAUGG | 4216 | |
| 3876 | AAAAUUAC C UGUCUUGA | 1518 | UCAAGACA CUGAUGAG GCCGUUAGGC CGAA IUAAUUUU | 4217 | |
| 3877 | AAAUUACC U GUCUUGAC | 1517 | GUCAAGAC CUGAUGAG GCCGUUAGGC CGAA IGUAAUUU | 4218 | |
| 3881 | UACCUGUC U UGACUGCC | 1518 | GGCAGUCA CUGAUGAG GCCGUUAGGC CGAA IACAGGUA | 4219 | |
| 3886 | GUCUUGAC U GCCAUGUG | 1519 | CACAUGGC CUGAUGAG GCCGUUAGGC CGAA IUCAAGAC | 4220 | |
| 3889 | UUGACUGC C AUGUGUUC | 1520 | GAACACAU CUGAUGAG GCCGUUAGGC CGAA ICAGUCAA | 4221 | |
| 3890 | UGACUGCC A UGUGUCCA | 1521 | UGAACACA CUGAUGAG GCCGUUAGGC CGAA IGCAGUCA | 4222 | |
| 3898 | AUGUGUUC A UCAUCUCA | 1522 | UAAGAUGA CUGAUGAG GCCGUUAGGC CGAA IAACACAU | 4223 | |
| 3901 | UGUUCAUC A UCUUAAGU | 1523 | ACUUAAGA CUGAUGAG GCCGUUAGGC CGAA IAUGAACA | 4224 | |
| 3904 | UCAUCAUC U UAAGUAUU | 1524 | AAUACUUA CUGAUGAG GCCGUUAGGC CGAA IAUGAUGA | 4225 | |
| 3919 | UUGUAAGC U GCUAUGUA | 1525 | UACAUAGC CUGAUGAG GCCGUUAGGC CGAA ICUUACAA | 4226 | |
| 3922 | UAAGCUGC U AUGUAUGG | 1526 | CCAUACAU CUGAUGAG GCCGUUAGGC CGAA ICAGCUCA | 4227 | |
| 3939 | AUGUAAAC C GUAAUCAU | 1527 | AUGAUCAC CUGAUGAG GCCGUCAGGC CGAA IUUUAAAU | 4228 | |
| 3946 | CCGUAAUC A UAUCUUUU | 1528 | AAAAGAUA CUGAUGAG GCCGUUAGGC CGAA IAUUACGG | 4229 | |
| 3951 | AUCAUAUC U UUUUCCUA | 1529 | UAGGAAAA CUGAUGAG GCCGUUAGGC CGAA IAUAUGAU | 4230 | |
| 3957 | UCUUUUUC C UAUCUGAG | 1530 | CUCAGAUA CUGAUGAG GCCGUCAGGC CGAA IAAAAAGA | 4231 | |
| 3958 | CUUUUUCC U AUCUGAGG | 1531 | CCUCAGAU CUGAUGAG GCCGUUAGGC CGAA IGAAAAAG | 4232 | |
| 3962 | UCCCUAUC U GAGGCACU | 1532 | AGUGCCUC CUGAUGAG GCCGUUAGGC CGAA IAUAGGAA | 4233 | |
| 3968 | UCUGAGGC A CUGGUGGA | 1533 | UCCACCAG CUGAUGAG GCCGUUAGGC CGAA ICCUCAGA | 4234 | |
| 3970 | UGAGGCAC U GGUGGAAU | 1534 | AUCCCACC CUGAUGAG GCCGUUAGGC CGAA IUGCCUCA | 4235 | |
| 3986 | UAAAAAAC C UGUAUAUC | 1535 | AAUAUACA CUGAUGAG GCCGUUAGGC CGAA IUUUUUUA | 4236 | |
| 3987 | AAAAAACC U GUAUAUUU | 1536 | AAAUAUAC CUGAUGAG GCCGUUAGGC CGAA IGUUUUUU | 4237 | |
| 3999 | UAUCUUAC U UUGUUGCA | 1537 | UGCAACAA CUGAUGAG GCCGUUAGGC CGAA IUAAAAUA | 4238 | |
| 4007 | UUUGUCGC A GAUAGUCU | 1538 | AGACUAUC CUGAUGAG GCCGUCAGGC CGAA ICAACAAA | 4239 | |
| 4015 | AGAUAGUC U UGCCGCAU | 1539 | AUGCGGCA CUGAUGAG GCCGUUAGGC CGAA IACUAUCU | 4240 | |
| 4019 | AGUCUCUC C GCAUCUUG | 1540 | CAAGAUGC CUGAUGAG GCCGUCAGGC CGAA ICAAGACU | 4241 | |
| 4022 | CUUGCCGC A UCUCGGCA | 1541 | UGCCAAGA CUGAUGAG GCCGUCAGGC CGAA ICGGCAAG | 4242 | |
| 4025 | GCCGCAUC U UGGCAAGU | 1542 | ACUCGCCA CUGAUGAG GCCGUUAGGC CGAA IAUGCGGC | 4243 | |
| 4030 | AUCUUGGC A AGUCGCAG | 1543 | CUGCAACU CUGAUGAG GCCGUCAGGC CGAA ICCAAGAU | 4244 | |
| 4037 | CAAGUUGC A GAGAUGGU | 1544 | ACCAUCUC CUGAUGAG GCCGUUAGGC CGAA ICAACUUG | 4245 | |
| Input Sequence AB020693. Cut Site | | | | | CH/. Stem Length = 8 Core Sequence = CUGAUGAG GCCGUUAGGC CGAA AB020693 (Homo sapiens rnRNA for K1 AA 0886 protein (Nogo-A); 4053 bp) |

Underlined region can be any X sequence or linker, as described herein. I=Inosine

TABLE V

Human NOGO G-cleaver Ribozyme and Substrate Sequence

| | | | | |
|---|---|---|---|---|
| | | | | Rz Seq |
| Pos | Substrate | Seq ID | Ribozyme | ID |

| 66 | CCACAACC G CCCGCGGC | 1545 | GCCGCGGG UGAUG GCAUGCACUAUGC GCG GGUUGUGG | 4246 | |
|---|---|---|---|---|---|
| 70 | AACCGCCC G CGGCUCUG | 1546 | CAGAGCCG UGAUG GCAUGCACUAUGC GCG GGGCGGUU | 4247 | |
| 78 | GCGGCUCU G AGACGCGG | 1547 | CCGCGUCU UGAUG GCAUGCACUAUGC GCG AGAGCCGC | 4248 | |
| 83 | UCUGAGAC G CGGCCCCG | 1548 | CGGGGCCG UGAUG GCAUGCACUAUGC GCG GUCUCAGA | 4249 | |
| 110 | CAGCAGCU G CAGCAUCA | 1549 | UGAUGCUG UGAUG GCAUGCACUAUGC GCG AGCUGCUG | 4250 | |
| 191 | CCCCGGCC G CAGCCCGC | 1550 | GCGGGCUG UGAUG GCAUGCACUAUGC GCG GGCCGGGG | 4251 | |
| 198 | CGCAGCCC G CGUUCAAG | 1551 | CUUGAACG UGAUG GCAUGCACUAUGC GCG GGGCUGCG | 4252 | |
| 218 | CAGUUCGU G AGGGAGCC | 1552 | GGCUCCCU UGAUG GCAUGCACUAUGC GCG ACGAACUG | 4253 | |
| 228 | GGGAGCCC G AGGACGAG | 1553 | CUCGUCCU UGAUG GCAUGCACUAUGC GCG GGGCUCCC | 4254 | |
| 234 | CCGAGGAC G AGGAGGAA | 1554 | UUCCUCCU UGAUG GCAUGCACUAUGC GCG GUCCUCGG | 4255 | |
| 267 | AGGAGGAC G AGGACGAA | 1555 | UUCGUCCU UGAUG GCAUGCACUAUGC GCG GUCCUCCU | 4256 | |
| 273 | ACGAGGAC G AAGACCUG | 1556 | CAGGUCUG UGAUG GCAUGCACUAUGC GCG GUCCUCGU | 4257 | |
| 296 | CUGGAGGU G CUGGAGAG | 1557 | CUCUCCAG UGAUG GCAUGCACUAUGC GCG ACCUCCAG | 4258 | |
| 312 | GGAAGCCC G CCGCCGGG | 1558 | CCCGGCGG UGAUG GCAUGCACUAUGC GCG GGGCUUCC | 4259 | |
| 315 | AGCCCGCC G CCGGGCUG | 1559 | CAGCCCGG UGAUG GCAUGCACUAUGC GCG GGCGGGCU | 4260 | |
| 327 | GGCUGUCC G CGGCCCCA | 1560 | UGGGGCCG UGAUG GCAUGCACUAUGC GCG GGACAGCC | 4261 | |
| 338 | GCCCCAGU G CCCACCGC | 1561 | GCGGUGGG UGAUG GCAUGCACUAUGC GCG ACUGGGGC | 4262 | |
| 345 | UGCCCACC G CCCCUGCC | 1562 | GGCAGGGG UGAUG GCAUGCACUAUGC GCG GGUGGGCA | 4263 | |
| 351 | CCGCCCCU G CCGCCGGC | 1563 | GCCGGCGG UGAUG GCAUGCACUAUGC GCG AGGGGCGG | 4264 | |
| 354 | CCCCUGCC G CCGGCGCG | 1564 | CGCGCCGG UGAUG GCAUGCACUAUGC GCG GGCAGGGG | 4265 | |
| 360 | CCGCCGGC G CGCCCCUG | 1565 | CAGGGGCG UGAUG GCAUGCACUAUGC GCG GCCGGCGG | 4266 | |
| 362 | GCCGGCGC G CCCCUGAU | 1566 | AUCAGGGG UGAUG GCAUGCACUAUGC GCG CCGCCGGC | 4267 | |
| 368 | GCGCCCCU G AUGCACUU | 1567 | AAGUCCAU UGAUG GCAUGCACUAUGC GCG AGGGGCGC | 4268 | |
| 384 | UCGGAAAU G ACUUCGUG | 1568 | CACGAAGU UGAUG GCAUGCACUAUGC GCG AUUUCCGA | 4269 | |
| 392 | GACUUCGU G CCGCCGGC | 1569 | GCCGGCCG UGAUG GCAUGCACUAUGC GCG ACGAAGUC | 4270 | |
| 395 | UUCGUGCC G CCGGCGCC | 1570 | GGCGCCGG UGAUG GCAUGCACUAUGC GCG CGCACGAA | 4271 | |
| 401 | CCGCCGGC G CCCCGGGG | 1571 | CCCCGGGG UGAUG GCAUGCACUAUGC GCG GCCGGCGG | 4272 | |
| 416 | GGACCCCU G CCGGCCGC | 1572 | GCGGCCGG UGAUG GCAUGCACUAUGC GCG AGGGGUCC | 4273 | |
| 423 | UGCCGGCC G CUCCCCCC | 1573 | GGGGGGAG UGAUG GCAUGCACUAUGC GCG GGCCGGCA | 4274 | |
| 435 | CCCCCGUC G CCCCGCAG | 1574 | CUCCGGGG UGAUG GCAUGCACUAUGC GCG CACGGGGG | 4275 | |
| 464 | UGGCACCC G AGCCCGGU | 1575 | ACCGGGCU UGAUG GCAUGCACUAUGC GCG GGGUCCCA | 4276 | |
| 479 | GUGUCGUC G ACCGUGCC | 1576 | GGCACGGU UGAUG GCAUGCACUAUGC GCG GACGACAC | 4277 | |
| 485 | UCGACCGU G CCCGCGCC | 1577 | GGCGCGGG UGAUG GCAUGCACUAUGC GCG ACGGUCGA | 4278 | |
| 489 | CCGUGCCC G CGCCAUCC | 1578 | GGAUGGCG UGAUG GCAUGCACUAUGC GCG GGGCACGG | 4279 | |
| 491 | GUGCCCGC G CCAUCCCC | 1579 | GGGGAUGG UGAUG GCAUGCACUAUGC GCG GCGGGCAC | 4280 | |
| 500 | CCAUCCCC G CUGUCUGC | 1580 | GCAGACAG UGAUG GCAUGCACUAUGC GCG GGGGAUGC | 4281 | |
| 507 | CGCUGUCU G CUGCCGCA | 1581 | UGCGGCAG UGAUG GCAUGCACUAUGC GCG AGACAGCG | 4282 | |
| 510 | UGUCUGCU G CCGCAGUC | 1582 | GACUGCGG UGAUG GCAUGCACUAUGC GCG AGCAGACA | 4283 | |
| 513 | CUGCUGCC G CAGUCUCG | 1583 | CGAGACUC UGAUG GCAUGCACUAUGC GCG GGCAGCAG | 4284 | |
| 521 | GCAGUCUC G CCCUCCAA | 1584 | UUGGAGGG UGAUG GCAUGCACUAUGC GCG GAGACUGC | 4285 | |
| 537 | AGCUCCCU G AGGACGAC | 1585 | GUCGUCCU UGAUG GCAUGCACUAUGC GCG AGGGAGCU | 4286 | |
| 543 | CUGAGGAC G ACGAGCCU | 1586 | AGGCUCGU UGAUG GCAUGCACUAUGC GCG CUCCUCAG | 4287 | |
| 546 | AGGACGAC G AGCCUCCG | 1587 | CGGAGGCU UGAUG GCAUGCACUAUGC GCG GUCGUCCU | 4288 | |
| 587 | GCCAGCGU G AGCCCCCA | 1588 | UGGGGGCU UGAUG GCAUGCACUAUGC GCG ACGCUGGC | 4289 | |
| 617 | UGGACCCC G CCAGCCCC | 1589 | GGGGCUGG UGAUG GCAUGCACUAUGC GCG GGGGUCCA | 4290 | |
| 633 | CGGCUCCC G CCGCGCCC | 1590 | GGGCGCGG UGAUG GCAUGCACUAUGC GCG GGGAGCCG | 4291 | |
| 636 | CUCCCGCC G CGCCCCCC | 1591 | GGGGGGCG UGAUG GCAUGCACUAUGC GCG GGCGGGAG | 4292 | |
| 638 | CCCGCCGC G CCCCCCUC | 1592 | CAGGGGGG UGAUG GCAUGCACUAUGC GCG GCGCCGGG | 4293 | |
| 657 | CCCCGGCC G CGCCCAAG | 1593 | CUUGGGCG UGAUG GCAUGCACUAUGC GCG GGCCGGGG | 4294 | |
| 659 | CCGCCCGC G CCCAAGCC | 1594 | CGCUUGGG UGAUG GCAUGCACUAUGC GCG GCGGCCGG | 4295 | |
| 667 | GCCCAAGC G CAGGGGCU | 1595 | AGCCCCUG UGAUG GCAUGCACUAUGC GCG GCUUGGGC | 4296 | |
| 693 | CAGUGGAU G AGACCCUU | 1596 | AAGGGUCU UGAUG GCAUGCACUAUGC GCG AUCCACUG | 4297 | |
| 705 | CCCUUUUU G CUCUUCCU | 1597 | AGGAACAG UGAUG GCAUGCACUAUGC GCG AAAAAGGG | 4298 | |
| 714 | CUCUUCCU G CUGCAUCU | 1598 | AGAUCCAG UGAUG GCAUGCACUAUGC GCG AGGAAGAG | 4299 | |
| 717 | UUCCUGCU G CAUCUGAG | 1599 | CUCAGAUG UGAUG GCAUGCACUAUGC GCG AGCAGGAA | 4300 | |
| 723 | CUGCAUCU G AGCCUGUG | 1600 | CACAGGCU UGAUG GCAUGCACUAUGC GCG AGAUGCAG | 4301 | |
| 731 | GAGCCUGU G AUACGCUC | 1601 | GAGCCUAU UGAUG GCAUGCACUAUGC GCG ACAGGCUC | 4302 | |
| 736 | UGUGAUAC G CUCCUCUG | 1602 | CAGAGGAG UGAUG GCAUGCACUAUGC GCG GUAUCACA | 4303 | |
| 744 | GCUCCUCU G CAGAAAAU | 1603 | AUUUUCUG UGAUG GCAUGCACUAUGC GCG AGAGGAGC | 4304 | |
| 761 | AUGGACUU G AAGGAGCA | 1604 | UGCUCCUU UGAUG GCAUGCACUAUGC GCG AAGUCCAU | 4305 | |
| 818 | UCUGUCCU G CUUCAAAC | 1605 | GUUUCAAG UGAUG GCAUGCACUAUGC GCG AGGACAGA | 4306 | |
| 822 | UCCUGCUU G AAACUGCU | 1606 | AGCAGUUU UGAUG GCAUGCACUAUGC GCG AAGCAGGA | 4307 | |
| 828 | UUGAAACU G CUGCUUCU | 1607 | AGAGCCAG UGAUG GCAUGCACUAUGC GCG ACUUUCAA | 4308 | |
| 831 | AAACUGCU G CUUCUCUU | 1608 | AAGAGAAG UGAUG GCAUGCACUAUGC GCG AGCAGUUU | 4309 | |
| 864 | UCUCACCC G CUUCUUUC | 1609 | GAAACAAG UGAUG GCAUGCACUAUGC GCG GGCUGAGA | 4310 | |
| 882 | AAGAACAU G AAUACCUU | 1610 | AAGGUAUU UGAUG GCAUGCACUAUGC GCG AUCUUCUU | 4311 | |
| 918 | UACCCACU G AAGGAACA | 1611 | UGUUCCUU UGAUG GCAUGCACUAUGC GCG AGUGGGUA | 4312 | |
| 945 | AUGUCAGU G AAGCUUCU | 1612 | AGAAGCUU UGAUG GCAUGCACUAUGC GCG ACUGACAU | 4313 | |
| 1071 | CAGAAUCU G CCGUAAUA | 1613 | UAUUACCG UGAUG GCAUGCACUAUGC GCG AGAUCCUG | 4314 | |
| 1109 | AUAAUCGU G AAAAAUAA | 1614 | UUAUUUUU UGAUG GCAUGCACUAUGC GCG ACGAUUAU | 4315 | |
| 1122 | AUAAAGAU G AAGAAGAC | 1615 | CUCUUCUG UGAUG GCAUGCACUAUGC GCG AUCUUUAU | 4316 | |
| 1206 | AAGAGGAU G AAGUUGUG | 1616 | CACAACUU UGAUG GCAUGCACUAUGC GCG AUCCUCUU | 4317 | |
| 1245 | GUUUUAAU G AAAAGAGA | 1617 | UCUCUUUU UGAUG GCAUGCACUAUGC GCG AUUAAAAC | 4318 | |
| 1257 | AGAGAGUU G CAGUGGAA | 1618 | UUCCACUG UGAUG GCAUGCACUAUGC GCG AACUCUCU | 4319 | |
| 1274 | GCUCCUAU G AGGGAGGA | 1619 | UCCUCCCU UGAUG GCAUGCACUAUGC GCG AUAGGAGC | 4320 | |
| 1287 | AGGAAUAU G CAGACUUC | 1620 | GAAGUCUG UGAUG GCAUGCACUAUGC GCG AUAUUCCU | 4321 | |
| 1305 | AACCAUUU G AGCGAGUA | 1621 | UACUCGCU UGAUG GCAUGCACUAUGC GCG AAAUCCUU | 4322 | |
| 1309 | AUUUGAGC G AGUAUGGG | 1622 | CCCAUACU UGAUG GCAUGCACUAUGC GCG GCUCAAAU | 4323 | |
| 1322 | UGGGAAGU G AAAGAUAG | 1623 | CUAUCUUU UGAUG GCAUGCACUAUGC GCG ACUUCCCA | 4324 | |
| 1344 | AAGAUAGU G AUAUGUUG | 1624 | CAACAUAU UGAUG GCAUGCACUAUGC GCG ACUAUCUU | 4325 | |
| 1356 | UGUUGGCU G CUGGAGGU | 1625 | ACCUCCAG UGAUG GCAUGCACUAUGC GCG AGCCAACA | 4326 | |
| 1371 | GUAAAAUC G AGAGCAAC | 1626 | GUUGCUCU UGAUG GCAUGCACUAUGC GCG GAUUUUAC | 4327 | |
| 1410 | AAUGUUUU G CAGAUAGC | 1627 | GCUAUCUG UGAUG GCAUGCACUAUGC GCG AAAACAUU | 4328 | |
| 1422 | AUAGCCUU G AGCAAACU | 1628 | AGUUUGCU UGAUG GCAUGCACUAUGC GCG AAGGCUAU | 4329 | |
| 1437 | CUAAUCAC G AAAAAGAU | 1629 | AUCUUUUU UGAUG GCAUGCACUAUGC GCG CUGAUGAC | 4330 | |
| 1449 | AAGAUAGU G AGAGUAGU | 1630 | ACUACUCU UGAUG GCAUGCACUAUGC GCG ACUAUCUU | 4331 | |
| 1461 | GUAGUAAU G AUGAUACU | 1631 | AGUAUCAU UGAUG GCAUGCACUAUGC GCG AUUACUAC | 4332 | |
| 1464 | GUAAUGAU G AUACUUCU | 1632 | AGAAGUAU UGAUG GCAUGCACUAUGC GCG AUCAUUAC | 4333 | |
| 1484 | CCCAGUAC G CCAGAAGG | 1633 | CCUUCUGG UGAUG GCAUGCACUAUGC GCG CUACUGGG | 4334 | |
| 1527 | UCACAUGU G CUCCCUUU | 1634 | AAAGGGAG UGAUG GCAUGCACUAUGC GCG ACAUCUCA | 4335 | |
| 1551 | CAGCAACU G AGAGCAUC | 1635 | AAUGCUCU UGAUG GCAUGCACUAUGC GCG AGUUGCUG | 4336 | |
| 1560 | AGAGCAUU G CAACAAAC | 1636 | GUUUGUUG UGAUG GCAUGCACUAUGC GCG AAUGCUCU | 4337 | |
| 1611 | AUAAGACC G AUGAAAAA | 1637 | UUUUUCAU UGAUG GCAUGCACUAUGC GCG GGUCUUAU | 4338 | |
| 1614 | AGACCGAU G AAAAAAAA | 1638 | UUUUUUUU UGAUG GCAUGCACUAUGC GCG AUCGGUCU | 4339 | |
| 1710 | AGGAUUCU G AGACAGAU | 1639 | AUCUGUCU UGAUG GCAUGCACUAUGC GCG AGAAUCCU | 4340 | |
| 1748 | ACAAAGGU G ACUGAGGA | 1640 | UCCUCAGU UGAUG GCAUGCACUAUGC GCG ACCUUUGU | 4341 | |
| 1752 | AGGUGACU G AGGAAGUC | 1641 | GACUUCCU UGAUG GCAUGCACUAUGC GCG AGUCACCU | 4342 | |
| 1772 | GCAAACAU G CCUGAAGG | 1642 | CCUUCAGG UGAUG GCAUGCACUAUGC GCG AUGUUUGC | 4343 | |
| 1776 | ACAUGCCU G AAGGCCUC | 1643 | CAGGCCUU UGAUG GCAUGCACUAUGC GCG AGGCAUGU | 4344 | |
| 1784 | GAAGGCCU G ACUCCAGA | 1644 | UCUGGAGU UGAUG GCAUGCACUAUGC GCG AGGCCUUC | 4345 | |
| 1818 | GUGAAAGU G AAUUGAAU | 1646 | AUUCAAUU UGAUG GCAUGCACUAUGC GCG ACUUUCAC | 4347 | |
| 1812 | AAGCAUGU G AAAGUGAA | 1645 | UUCACUUU UGAUG GCAUGCACUAUGC GCG ACAUGCUU | 4346 | |
| 1823 | AGUGAAUU G AAUGAAGU | 1647 | ACUUCAUU UGAUG GCAUGCACUAUGC GCG AAUUCACU | 4348 | |
| 1827 | AAUGCAAU G AAGUUACU | 1648 | AGUAACUU UGAUG GCAUGCACUAUGC GCG AUUCAAUU | 4349 | |
| 1848 | CAAAGAUU G CUUAUGAA | 1649 | UUCAUAAG UGAUG GCAUGCACUAUGC GCG AAUCUUUG | 4350 | |
| 1854 | UUGCUUAU G AAACAAAA | 1650 | UUUUGUUU UGAUG GCAUGCACUAUGC GCG AUAAGCAA | 4351 | |
| 1892 | GAAGUUAU G CAAGAGUC | 1651 | GACUCUUG UGAUG GCAUGCACUAUGC GCG AUAACUUC | 4352 | |
| 1911 | UCUAUCCU G CAGCACAG | 1652 | CUGUCCUG UGAUG GCAUGCACUAUGC GCG AGGAUAGA | 4353 | |
| 1924 | ACAGCUUU G CCCAUCAU | 1653 | AUGAUGGG UGAUG GCAUGCACUAUGC GCG AAAGCUGU | 4354 | |
| 1935 | CAUCAUUU G AAGAGUCA | 1654 | UGACUCUU UGAUG GCAUGCACUAUGC GCG AAAUGAUG | 4355 | |
| 1967 | CCAGUUUU G CCUGACAU | 1655 | AUGUCAGG UGAUG GCAUGCACUAUGC GCG AAAACUGG | 4356 | |
| 1971 | UUUUGCCU G ACAUUGUU | 1656 | AACAAUGU UGAUG GCAUGCACUAUGC GCG AGGCAAAA | 4357 | |
| 1994 | GCACCAUU G AAUUCUGC | 1657 | GCAGAAUU UGAUG GCAUGCACUAUGC GCG AAUGGUGC | 4358 | |
| 2001 | UGAAUUCU G CAGUUCCU | 1658 | AGGAACUG UGAUG GCAUGCACUAUGC GCG AGAAUUCA | 4359 | |
| 2013 | UUCCUAGU G CUGGUGCU | 1659 | AGCACCAG UGAUG GCAUGCACUAUGC GCG ACUAGGAA | 4360 | |
| 2019 | CUGCUGGU G CUUCCGUG | 1660 | CACGCAAG UGAUG GCAUGCACUAUGC GCG ACCAGCAC | 4361 | |
| 2027 | GCUUCCGU G AUACAGCC | 1661 | GCCUGUAU UGAUG GCAUGCACUAUGC GCG ACGGAAGC | 4362 | |
| 2073 | UUAAUUAU G AAAGCAUA | 1662 | UAUGCUUU UGAUG GCAUGCACUAUGC GCG AUAAUUAA | 4363 | |
| 2088 | UAAAACAU G AGCCUGAA | 1663 | UUCAGGCU UGAUG GCAUGCACUAUGC GCG AUCUUUUA | 4364 | |
| 2094 | AUGAGCCU G AAAACCCC | 1664 | GGGGUUUU UGAUG GCAUGCACUAUGC GCG AGGCUCAU | 4365 | |
| 2112 | CACCAUAU G AAGAGGCC | 1665 | GGCCUCUU UGAUG GCAUGCACUAUGC GCG AUAUGGUG | 4366 | |
| 2123 | GAGGCCAU G AGUGUAUC | 1666 | GAUACACU UGAUG GCAUGCACUAUGC GCG AUGGCCUC | 4367 | |
| 2175 | AAGAGCCU G AAAAUAUU | 1667 | AAUAUUUU UGAUG GCAUGCACUAUGC GCG AGGCUCUU | 4368 | |
| 2187 | AUAUUAAU G CAGCUCUU | 1668 | AAGAGCUC UGAUG GCAUGCACUAUGC GCG AUUAAUAU | 4369 | |
| 2226 | UAUCUAUU G CAUGUGAU | 1669 | AUCACAUG UGAUG GCAUGCACUAUGC GCG AAUAGAUA | 4370 | |
| 2232 | UUGCAUGU G AUUUAAUU | 1670 | AAUUAAAU UGAUG GCAUGCACUAUGC GCG ACAUCCAA | 4371 | |
| 2259 | AGCUUUCU G CUGAACCA | 1671 | UGGUUCAG UGAUG GCAUGCACUAUGC GCG AGAAAGCU | 4372 | |
| 2262 | UUUCUGCU G AACCAGCU | 1672 | AGCUGGUC UGAUG GCAUGCACUAUGC GCG AGCAGAAA | 4373 | |
| 2283 | AUUUCUCU G AUUAUUCA | 1673 | UGAAUAAU UGAUG GCAUGCACUAUGC GCG AGAGAAAU | 4374 | |
| 2307 | CAAAAGUU G AACACCCA | 1674 | UGGCUGUU UGAUG GCAUGCACUAUGC GCG AACUUUUG | 4375 | |
| 2318 | CAGCCAGU G CCUGAUGA | 1675 | UGAUGAGG UGAUG GCAUGCACUAUGC GCG ACUGGCUC | 4376 | |
| 2322 | CAGUGCCU G AUCAUUCU | 1676 | AGAAUCAU UGAUG GCAUGCACUAUGC GCG AGGCACUG | 4377 | |
| 2331 | AUCAUUCU G AGCUAGUU | 1677 | AACUAGCU UGAUG GCAUGCACUAUGC GCG AGAAUCAU | 4378 | |
| 2340 | AGCUAGUU G AAGAUUCC | 1678 | GGAAUCUU UGAUG GCAUGCACUAUGC GCG AACUAGCU | 4379 | |
| 2355 | CCUCACCU G AUUCUGAA | 1679 | UUCACAAU UGAUG GCAUGCACUAUGC GCG AGGUGACC | 4380 | |
| 2361 | CUGAUUCU G AACCAGUU | 1680 | AACUGGUU UGAUG GCAUGCACUAUGC GCG AGAAUCAG | 4381 | |
| 2370 | AACCACUU G ACUUAUUU | 1681 | AAAUAAGU UGAUG GCAUGCACUAUGC GCG AACUCGUU | 4382 | |
| 2382 | UAUUUACU G AUGAUUCA | 1682 | UCAAUCAU UGAUG GCAUGCACUAUGC GCG ACUAAAUA | 4383 | |
| 2385 | UUAGUGAU G AUUCAAUA | 1683 | UAUUCAAU UGAUG GCAUGCACUAUGC GCG AUCACUAA | 4384 | |
| 2397 | CAAUACCU G ACGUUCCA | 1684 | UGGAACGU UGAUG GCAUGCACUAUGC GCG AGGUAUUG | 4385 | |
| 2418 | AACAAGAU G AAACUGUG | 1685 | CACAGUUU UGAUG GCAUGCACUAUGC GCG AUCUUCUU | 4386 | |
| 2426 | GAAACUGU G AUGCUUGU | 1686 | ACAAGCAU UGAUG GCAUGCACUAUGC GCG ACAGUUUC | 4387 | |
| 2429 | ACUGUCAU G CUUGUGAA | 1687 | UUCACAAG UGAUG GCAUGCACUAUGC GCG AUCACAGU | 4388 | |
| 2435 | AUGCUUGU G AAAGAAAG | 1688 | CUUUCUUU UGAUG GCAUGCACUAUGC GCG ACAAGCAU | 4389 | |
| 2451 | GUCUCACU G AGACUUCA | 1689 | UGAAGUCU UGAUG GCAUGCACUAUGC GCG AGUGACAC | 4390 | |
| 2463 | CUUCAUUU G AGUCAAUG | 1690 | CAUUCACU UGAUG GCAUGCACUAUGC GCG AAAUGAAC | 4391 | |
| 2471 | GAGUCAAU G AUAGAAUA | 1691 | UAUUCUAU UGAUG GCAUGCACUAUGC GCG AUUGACUC | 4392 | |
| 2481 | UACAAUAU G AAAAUAAC | 1692 | CUUAUUUU UGAUG GCAUGCACUAUGC GCG AUAUUCUA | 4393 | |
| 2502 | AACUCACU G CUUUGCCA | 1693 | UGCCAAAG UGAUG GCAUGCACUAUGC GCG ACUCACUG | 4394 | |
| 2507 | AGUGCUUU G CCACCUCA | 1694 | UCACCUGC UGAUG GCAUGCACUAUGC GCG AAAGCACU | 4395 | |
| 2514 | UCCCACCU G ACCGAGCA | 1695 | UCCUCCCU UGAUG GCAUGCACUAUGC GCG ACGUGCCA | 4396 | |
| 2583 | UCUUACCU G AUGAAGUU | 1696 | AACUUCAU UGAUG GCAUGCACUAUGC GCG AGGUAACA | 4397 | |
| 2586 | UACCUGAU G AAGUUUCA | 1697 | UGAAACUU UGAUG GCAUGCACUAUGC GCG AUCAGGUA | 4398 | |
| 2600 | UCAACAUU G AGCAAAAA | 1698 | UUUUUGCU UGAUG GCAUGCACUAUGC GCG AAUGUUGA | 4399 | |
| 2624 | AUUCCUUU G CAGAUGGA | 1699 | UCCAUCUG UGAUG GCAUGCACUAUGC GCG AAAGGAAU | 4400 | |
| 2646 | UCAGUACU G CAGUUUAU | 1700 | AUAAACUG UGAUG GCAUGCACUAUGC GCG AGUACUGA | 4401 | |
| 2661 | AUUCAAAU G AUGACUUA | 1701 | UAAGUCAU UGAUG GCAUGCACUAUGC GCG AUUUCAAU | 4402 | |
| 2664 | CAAAUGAU G ACUUAUUU | 1702 | AAAUAAGU UGAUG GCAUGCACUAUGC GCG AUCAUUUG | 4403 | |
| 2703 | GAGAAACU G AAACGUUU | 1703 | AAACGUUU UGAUG GCAUGCACUAUGC GCG AGUUUCUC | 4404 | |
| 2730 | CUCCAAUU G AAAUUAUA | 1704 | UAUAAUUU UGAUG GCAUGCACUAUGC CCG AAUUGGAG | 4405 | |
| 2742 | UUAUAGAU G AGUUCCCU | 1705 | AGGGAACU UGAUG GCAUGCACUAUGC GCG AUCUAUAA | 4406 | |
| 2756 | CCUACAUU G AUCAGUUC | 1706 | GAACUGAU UGAUG GCAUGCACUAUGC GCG AAUGUAGG | 4407 | |
| 2772 | CUAAAACU G AUUCAUUU | 1707 | AAAUGAAU UGAUG GCAUGCACUAUGC GCG AGUUUUAG | 4408 | |
| 2805 | AAUAUACU G ACCUAGAA | 1708 | UUCUAGGU UGAUG GCAUGCACUAUGC GCG AGUAUAUU | 4409 | |
| 2829 | ACAAAAGU G AAAUUGCU | 1709 | AGCAAUUU UGAUG GCAUGCACUAUGC GCG ACUUUUGU | 4410 | |
| 2835 | GUGAAAUU G CUAAUGCC | 1710 | GGCAUUAG UGAUG GCAUGCACUAUGC GCG AAUUUCAC | 4411 | |
| 2841 | UUGCUAAU G CCCCGGAU | 1711 | AUCCGGGG UGAUG GCAUGCACUAUGC GCG AUUAGCAA | 4412 | |
| 2864 | GGGUCAUU G CCUUGCAC | 1712 | GUGCAAGG UGAUG GCAUGCACUAUGC GCG AAUGACCC | 4413 | |
| 2869 | AUUGCCUU G CACAGAAU | 1713 | AUUCUGUG UGAUG GCAUGCACUAUGC GCG AAGGCAAU | 4414 | |
| 2879 | ACAGAAUU G CCCCAUGA | 1714 | UCAUGGGG UGAUG GCAUGCACUAUGC GCG AAUUCUGU | 4415 | |
| 2886 | UGCCCCAU G ACCUUUCU | 1715 | AGAAACGU UGAUG GCAUGCACUAUGC GCG AUGCGGCA | 4416 | |
| 2897 | CUUUCUUU G AAGAACAU | 1716 | AUGUUCUU UGAUG GCAUGCACUAUGC GCG AAAGAAAG | 4417 | |
| 2919 | CCAAAGUU G AAGAGAAA | 1717 | UUUCUCUU UGAUG GCAUGCACUAUGC GCG AACUUUGC | 4418 | |
| 2943 | UCUCAGAU G ACUUUUCU | 1718 | AGAAAAGU UGAUG GCAUGCACUAUGC GCG AUCUGAGA | 4419 | |
| 2964 | AUGGGUCU G CUACAUCA | 1719 | UGAUGUAG UGAUG GCAUGCACUAUGC GCG AGACCCAU | 4420 | |
| 2978 | UCAAAGGU G CUCUUAUU | 1720 | AAUAAGAG UGAUG GCAUGCACUAUGC GCG ACCUUUGA | 4421 | |
| 2987 | CUCUUAUU G CCUCCAGA | 1721 | UCUGGAGG UGAUG GCAUGCACUAUGC GCG AAUAAGAG | 4422 | |
| 3003 | AUGUUUCU G CUUUGGCC | 1722 | GGCCAAAG UGAUG GCAUGCACUAUGC GCG AGAAACAU | 4423 | |
| 3056 | GUUCUUGU G AAAGAAGC | 1723 | GCUUCUUU UGAUG GCAUGCACUAUGC GCG ACAAGAAC | 4424 | |
| 3066 | AAGAAGCU G AGAAAAAA | 1724 | UUUUUUCU UGAUG GCAUGCACUAUGC GCG AGCUUCUU | 4425 | |
| 3084 | UUCCUUCC G AUACAGAA | 1725 | UUCUGUAU UGAUG GCAUGCACUAUGC GCG GGAAGGAA | 4426 | |
| 3114 | CACCAUCU G CUAUAUUU | 1726 | AAAUAUAG UGAUG GCAUGCACUAUGC GCG AGAUGGUG | 4427 | |
| 3134 | GCAGAGCU G AGUAAAAC | 1727 | GUUUUACU UGAUG GCAUGCACUAUGC GCG AGCUCUGC | 4428 | |
| 3153 | CAGUUCUU G ACCUCCUG | 1728 | CAGGAGGU UGAUG GCAUGCACUAUGC GCG AACAACUG | 4429 | |
| 3201 | UGUUUGGU G CCAGCCUA | 1729 | UAGGCUGG UGAUG GCAUGCACUAUGC GCG ACCAAACA | 4430 | |
| 3215 | CUAUUCCU G CUGCUUUC | 1730 | GAAAGCAG UGAUG GCAUGCACUAUGC GCG AGCAAUAC | 4431 | |
| 3218 | UUCCUGCU G CUUUCAUU | 1731 | AAUGAAAG UGAUG GCAUGCACUAUGC GCG AGCAGCAA | 4432 | |
| 3227 | CUUUCAUU G ACAGUAUU | 1732 | AAUACUGU UGAUG GCAUGCACUAUGC GCG AAUGAAAG | 4433 | |
| 3245 | AGCAUUGU G AGCGUAAC | 1733 | GUUACGCU UGAUG GCAUGCACUAUGC GCG ACAAUGCU | 4434 | |
| 3264 | CCUACAUU G CCUUGGCC | 1734 | GGCCAAGG UGAUG GCAUGCACUAUGC GCG AAUGUAGG | 4435 | |
| 3275 | UUGGCCCU G CUCUCUGU | 1735 | ACAGAGAG UGAUG GCAUGCACUAUGC GCG AGGGCCAA | 4436 | |
| 3284 | CUCUCUGU G ACCAUCAG | 1736 | CUGAUGGU UGAUG GCAUGCACUAUGC GCG ACAGAGAG | 4437 | |
| 3314 | AAGGGUGU G AUCCAAGC | 1737 | GCUUGGAU UGAUG GCAUGCACUAUGC GCG ACACCCUU | 4438 | |
| 3339 | AAUCAGAU G AAGGCCAC | 1738 | GUGGCCUU UGAUG GCAUGCACUAUGC GCG AUCUCAUU | 4439 | |
| 3372 | UGGAAUCU G AAGUUGCU | 1739 | AGCAACUU UGAUG GCAUGCACUAUGC GCG AGAUUCCA | 4440 | |
| 3378 | CUGAAGUU G CUAUAUCU | 1740 | AGAUAUAG UGAUG GCAUGCACUAUGC GCG AACUUCAG | 4441 | |
| 3387 | CUAUAUCU G AGGAGUUG | 1741 | CAACUCCU UGAUG GCAUGCACUAUGC GCG ACAUAUAC | 4442 | |
| 3417 | GUAAUUCU G CUCUUGGU | 1742 | ACCAAGAG UGAUG GCAUGCACUAUGC GCG ACAAUUAC | 4443 | |
| 3431 | GGUCAUGU G AACUGCAC | 1743 | GUGCACUU UGAUG GCAUGCACUAUGC GCG ACAUGACC | 4444 | |
| 3436 | UGUGAACU G CACGAUAA | 1744 | UUAUCGUC UGAUG GCAUGCACUAUGC GCG ACUUCACA | 4445 | |
| 3440 | AACUGCAC G AUAAAGGA | 1745 | UCCUUUAU UGAUG GCAUGCACUAUGC GCG GUGCAGUU | 4446 | |
| 3457 | ACUCAGGC G CCUCUUCU | 1746 | AGAAGAGG UGAUG GCAUGCACUAUGC GCG GCCUGAGU | 4447 | |
| 3471 | UCUUAGUU G AUGAUUUA | 1747 | UAAAUCAU UGAUG GCAUGCACUAUGC GCG AACUAAGA | 4448 | |
| 3474 | UAGUUGAU G AUUUAGUU | 1748 | AACUAAAU UGAUG GCAUGCACUAUGC GCG AUCAACUA | 4449 | |
| 3483 | AUUUAGUU G AUUCUCUG | 1749 | CAGAGAAU UGAUG GCAUGCACUAUGC GCG AACUAAAU | 4450 | |
| 3491 | GAUUCUCU G AAGUUUGC | 1750 | GCAAACUU UGAUG GCAUGCACUAUGC GCG AGAGAAUC | 4451 | |
| 3498 | UGAAGUUU G CAGUGUUG | 1751 | CAACACUG UGAUG GCAUGCACUAUGC GCG AAACUUCA | 4452 | |
| 3506 | GCAGUGUU G AUGUGGGU | 1752 | ACCCACAU UGAUG GCAUGCACUAUGC GCG AACACUGC | 4453 | |
| 3531 | AUGUUGGU G CCUUGUUU | 1753 | AAACAAGG UGAUG GCAUGCACUAUGC GCG ACCAACAU | 4454 | |
| 3548 | AAUGGUCU G ACACUACU | 1754 | AGUAGUGU UGAUG GCAUGCACUAUGC GCG AGACCAUU | 4455 | |
| 3557 | ACACUACU G AUUUUGGC | 1755 | GCCAAAAU UGAUG GCAUGCACUAUGC GCG AGUAGUGU | 4456 | |
| 3600 | UUAUUUAU G AACGGCAU | 1756 | AUGCCGUU UGAUG GCAUGCACUAUGC GCG AUAAAUAA | 4457 | |
| 3639 | UAGGACUU G CAAAUAAG | 1757 | CUUAUUUG UGAUG GCAUGCACUAUGC GCG AAGUCCUA | 4458 | |
| 3660 | UUAAAGAU G CUAUGGCU | 1758 | AGCCAUAG UGAUG GCAUGCACUAUGC GCG AUCUUUAA | 4459 | |
| 3695 | CCUGGAUU G AAGCGCAA | 1759 | UUGCGCUU UGAUG GCAUGCACUAUGC GCG AAUCCAGG | 4460 | |
| 3700 | AUUGAAGC G CAAAGCUG | 1760 | CAGCUUUG UGAUG GCAUGCACUAUGC GCG GCUUCAAU | 4461 | |
| 3708 | GCAAAGCU G AAUGAAAA | 1761 | UUUUCAUU UGAUG GCAUGCACUAUGC GCG AGCUUUGC | 4462 | |
| 3712 | AGCUGAAU G AAAACGCC | 1762 | GGCGUUUU UGAUG GCAUGCACUAUGC GCG AUUCAGCU | 4463 | |
| 3718 | AUGAAAAC G CCCAAAAU | 1763 | AUUUUGGG UGAUG GCAUGCACUAUGC GCG GUUUUCAU | 4464 | |
| 3765 | AUUCAUUU G AUUAUACG | 1764 | CGUAUAAU UGAUG GCAUGCACUAUGC GCG AAAUGAAU | 4465 | |
| 3794 | GGAAGAAC G AACCUUGA | 1765 | UCAAGGUU UGAUG GCAUGCACUAUGC GCG GUUCUUCC | 4466 | |
| 3801 | CGAACCUU G ACGUUGCA | 1766 | UGCAACGU UGAUG GCAUGCACUAUGC GCG AAGGUUCG | 4467 | |
| 3807 | UUGACGUU G CAGUGCAG | 1767 | CUGCACUG UGAUG GCAUGCACUAUGC GCG AACGUCAA | 4468 | |
| 3812 | GUUGCAGU G CAGUUUCA | 1768 | UGAAACUG UGAUG GCAUGCACUAUGC GCG ACUGCAAC | 4469 | |
| 3853 | UUAGCCAU G CACUGUUG | 1769 | CAACAGUG UGAUG GCAUGCACUAUGC GCG AUGGCUAA | 4470 | |
| 3863 | ACUGUUGU G AGGAAAAA | 1770 | UUUUUCCU UGAUG GCAUGCACUAUGC GCG ACAACAGU | 4471 | |
| 3883 | CCUGUCUU G ACUGCCAU | 1771 | AUGGCAGU UGAUG GCAUGCACUAUGC GCG AAGACAGG | 4472 | |
| 3887 | UCUUGACU G CCAUGUGU | 1772 | ACACAUGG UGAUG GCAUGCACUAUGC GCG AGUCAAGA | 4473 | |
| 3920 | UGUAAGCU G CUAUGUAU | 1773 | AUACAUAG UGAUG GCAUGCACUAUGC GCG AGCUUACA | 4474 | |
| 3963 | UCCUAUCU G AGGCACUG | 1774 | CAGUGCCU UGAUG GCAUGCACUAUGC GCG AGAUAGGA | 4475 | |
| 4005 | ACUUUGUU G CAGAUAGU | 1775 | ACUAUCUG UGAUG GCAUGCACUAUGC GCG AACAAAGU | 4476 | |
| 4017 | AUAGUCUU G CCGCAUCU | 1776 | AGAUGCGG UGAUG GCAUGCACUAUGC GCG AAGACUAU | 4477 | |
| 4020 | GUCUUGCC G CAUCUUGG | 1777 | CCAAGAUG UGAUG GCAUGCACUAUGC GCG GGCAAGAC | 4478 | |
| 4035 | GGCAAGUU G CAGAGAUG | 1778 | CAUCUCUG UGAUG GCAUGCACUAUGC GCG AACUUGCC | 4479 | |
| Input Sequence = A3020693. Cut Site = YG/M or UG/U. Stem Length = 8. Core Sequence = UGAUG GCAUGCACUAUGC GCG AB020693 (Homo sapiens mRNA for KIAA0886 protein (Nogo-A); 4053 | | | | | bp) |

TABLE VI

Human NCGC Zinzyme Ribozyme and Substrate Sequence

| | | | | |
|---|---|---|---|---|
| | | | | Rz Seq |
| Pos | Substrate | Seq ID | Ribozyme | ID |

| 66 | CCACAACC G CCCGCGGC | 1545 | GCCGCGGG GCCGAAAGGCGAGUCAAGGUCU GGUUGUGG | 4480 | | |
|---|---|---|---|---|---|---|
| 70 | AACCGCCC G CGGCUCUG | 1546 | CAGAGCCG GCCGAAAGGCGAGUCAAGGUCU GGGCGGUU | 4481 | | |
| 83 | UCUGAGAC G CGGCCCCG | 1548 | CGGGGCCG GCCGAAAGGCGAGUCAAGGUCU GUCUCAGA | 4482 | | |
| 110 | CAGCAGCU G CAGCAUCA | 1549 | UGAUGCUG GCCGAAAGGCGAGUCAAGGUCU AGGUGCUG | 4483 | | |
| 191 | CCCCGGCC G CAGCCCGC | 1550 | GCGGGCUG GCCGAAAGGCGAGUCAAGGUCU GGCCGGGG | 4484 | | |
| 198 | CGCAGCCC G CGUUCAAG | 1551 | CUUGAACG GCCGAAAGGCGAGUCAAGGUCU GGGCUGCG | 4485 | | |
| 296 | CUGGAGGU G CUGGAGAG | 1557 | CUCUCCAG GCCGAAAGGCGAGUCAAGGUCU ACCUCCAG | 4486 | | |
| 312 | GGAAGCCC G CCGCCGGG | 1558 | CCCGGCGG GCCGAAAGGCGAGUCAAGGUCU GGGCUUCC | 4487 | | |
| 315 | AGCCCGCC G CCGGGCUG | 1559 | CAGCCCGG GCCGAAAGGCGAGUCAAGGUCU GGCGGGCU | 4488 | | |
| 327 | GGCUGUCC G CGGCCCCA | 1560 | UGGGGCCG GCCGAAAGGCGAGUCAAGGUCU GGACAGCC | 4489 | | |
| 338 | GCCCCAGU G CCCACCGC | 1561 | GCGGUGGG GCCGAAAGGCGAGUCAAGGUCU ACUGGGGC | 4490 | | |
| 345 | UGCCCACC G CCCCUGCC | 1562 | GGCAGGGG GCCGAAAGGCGAGUCAAGGUCU GGUGGGCA | 4491 | | |
| 351 | CCGCCCCU G CCGCCGGC | 1563 | GCCGGCGG GCCGAAAGGCGAGUCAAGGUCU AGGGGCGG | 4492 | | |
| 354 | CCCCUGCC G CCGGCGCG | 1564 | CGCGCCGG GCCGAAAGGCGAGUCAAGGUCU GGCAGGGG | 4493 | | |
| 360 | CCGCCGGC G CGCCCCUG | 1565 | CAGGGGCG GCCGAAAGGCGAGUCAAGGUCU GCCGGCGG | 4494 | | |
| 362 | GCCGGCGC G CCCCUGAU | 1566 | AUCAGGGG GCCGAAAGGCGAGUCAAGGUCU GCGCCGGC | 4495 | | |
| 392 | GACUUCGU G CCGCCGGC | 1569 | GCCGGCGG GCCGAAAGGCGAGUCAAGGUCU ACGAAGUC | 4496 | | |
| 395 | UUCGUGCC G CCGGCGCC | 1570 | GGCGCCGG GCCGAAAGGCGAGUCAAGGUCU GGCACGAA | 4497 | | |
| 401 | CCGCCGGC G CCCCGGGG | 1571 | CCCCGGGG GCCGAAAGGCGAGUCAAGGUCU GCCGGCGG | 4498 | | |
| 416 | GGACCCCU G CCGGCCGC | 1572 | GCGGCCGG GCCGAAAGGCGAGUCAAGGUCU AGGGGUCC | 4499 | | |
| 423 | UGCCGGCC G CUCCCCCC | 1573 | GGGGGGAG GCCGAAAGGCGAGUCAAGGUCU GGCCGGCA | 4500 | | |
| 435 | CCCCCGUC G CCCCGGAG | 1574 | CUCCGGGG GCCGAAAGGCGAGUCAAGGUCU GACGGGGG | 4501 | | |
| 485 | UCGACCGU G CCCGCGCC | 1577 | GGCGCGUG GCCGAAAGGCGAGUCAAGGUCU ACGGUCCA | 4502 | | |
| 489 | CCGUGCCC G CGCCAUCC | 1578 | GGAUGGCG GCCGAAAGGCGAGUCAAGGUCU GGGCACGG | 4503 | | |
| 491 | GUGCCCUC G CCAUCCCC | 1579 | GGGGAUGG GCCGAAAGGCGAGUCAAGGUCU GCGGGCAC | 4504 | | |
| 500 | CCAUCCCC G CUGUCUGC | 1580 | GCAGACAG GCCGAAAGGCGAGUCAAGGUCU GGGGAUGU | 4505 | | |
| 507 | CGCUGUCU G CUGCCGCA | 1581 | UGCGGCAG GCCGAAAGGCGAGUCAAGGUCU AGACAGCG | 4506 | | |
| 510 | UGUCUGCU G CCGCAGUC | 1582 | GACUGCGG GCCGAAAGGCGAGUCAAGGUCU AGCAGACA | 4507 | | |
| 513 | CUGCUGCC G CAGUCUCG | 1583 | CGAGACUG GCCGAAAGGCGAGUCAAGGUCU GGCAGCAG | 4508 | | |
| 521 | GCAGUCUC G CCCUCCAA | 1584 | UUGGAGGG GCCGAAAGGCGAGUCAAGGUCU GAGACUGC | 4509 | | |
| 617 | UGGACCCC G CCAGCCCC | 1589 | GGGGCUGG GCCGAAAGGCGAGUCAAGGUCU GGGGUCCA | 4510 | | |
| 633 | CGGCUCCC G CCUCUCCC | 1590 | GGGCGCGG GCCGAAAGGCGAGUCAAGGUCU GGGAGCCG | 4511 | | |
| 636 | CUCCCGCC G CGCCCCCC | 1591 | GGGGGGCG GCCGAAAGGCGAGUCAAGGUCU GGCGGGAG | 4512 | | |
| 638 | CCCGCCGC G CCCCCCUC | 1592 | GAGGGGGG GCCGAAAGGCGAGUCAAGGUCU GCGGCGGG | 4513 | | |
| 657 | CCCCGGCC G CGCCCAAG | 1593 | CUUGGGCG GCCGAAAGGCGAGUCAAGGUCU GGCCGGGG | 4514 | | |
| 659 | CCGGCCGC G CCCAAGCG | 1594 | CGCUUGGG GCCGAAAGGCGAGUCAAGGUCU GCGGCCGG | 4515 | | |
| 667 | GCCCAAGC G CAGGGGCU | 1595 | AGCCCCUG GCCGAAAGGCGAGUCAAGGUCU GCUUGGGC | 4516 | | |
| 705 | CCCUUUUU G CUCCUUCU | 1597 | AGGAAGAG GCCGAAAGGCGAGUCAAGGUCU AAAAAGGG | 4517 | | |
| 714 | CUCUUCCU G CUGCAUCU | 1598 | AGAUGCAG GCCGAAAGGCGAGUCAAGGUCU AGGAAGAG | 4518 | | |
| 717 | UUCCUGCU G CAUCUGAG | 1599 | CUCAGAUG GCCGAAAGGCGAGUCAAGGUCU AGCAGGAA | 4519 | | |
| 736 | UGUGAUAC G CUCCUCUG | 1602 | CAGAGGAG GCCGAAAGGCGAGUCAAGGUCU GUAUCACA | 4520 | | |
| 744 | UCUCCUCU G CAGAAAAU | 1603 | AUUUUCUG GCCGAAAGGCGAGUCAAGGUCU AGAUGAUG | 4521 | | |
| 818 | UCUGUCCU G CUUGAAAC | 1605 | GUUUCAAG GCCGAAAGGCGAGUCAAGGUCU AGGACAGA | 4522 | | |
| 828 | UUGAAACU G CUGCUUCU | 1607 | AGAAGCAG GCCGAAAGGCGAGUCAAGGUCU AGUUUCAA | 4523 | | |
| 831 | AAACUGCU G CUUCUCUU | 1608 | AAGAGAAG GCCGAAAGGCGAGUCAAGGUCU AGCAGUUU | 4524 | | |
| 864 | UCUCAGCC G CUUCUUUC | 1609 | GAAAGAAG GCCGAAAGGCGAGUCAAGGUCU GGCUGAGA | 4525 | | |
| 1071 | CAGAAUCU G CCGUAAUA | 1613 | UAUUACGG GCCGAAAGGCGAGUCAAGGUCU AGAUUCUG | 4526 | | |
| 1257 | AGAGAGUU G CAGUGGAA | 1618 | UUCCACUG GCCGAAAGGCGAGUCAAGGUCU AACUCUCU | 4527 | | |
| 1287 | AGGAAUAU G CAGACUUC | 1620 | GAAGUCUG GCCGAAAGGCGAGUCAAGGUCU AUAUUCCU | 4528 | | |
| 1356 | UGUUGGCU G CUGGAGGU | 1625 | ACCUCCAG GCCGAAAGGCGAGUCAAGGUCU AGCCAACA | 4529 | | |
| 1410 | AAUGUUUU G CAGAUAGC | 1627 | GCUAUCUG GCCGAAAGGCGAGUCAAGGUCU AAAACAUU | 4530 | | |
| 1484 | CCCAGUAC G CCAGAAGG | 1633 | CCUUCUGG GCCGAAAGGCGAGUCAAGGUCU GUACUGGG | 4531 | | |
| 1527 | UCACAUGU G CUCCCUUU | 1634 | AAAGGGAG GCCGAAAGGCGAGUCAAGGUCU ACAUGUGA | 4532 | | |
| 1560 | AGAGCAUU G CAACAAAC | 1636 | GUUUGUUG GCCGAAAGGCGAGUCAAGGUCU AAUGCUCU | 4533 | | |
| 1772 | GCAAACAU G CCUGAAGG | 1642 | CCUUCAGG GCCGAAAGGCGAGUCAAGGUCU AUGUUUGC | 4534 | | |
| 1848 | CAAAGAUU G CUUAUGAA | 1649 | UUCAUAAG GCCGAAAGGCGAGUCAAGGUCU AAUCUUUG | 4535 | | |
| 1892 | GAAGUUAU G CAAGAGUC | 1651 | GACUCUUG GCCGAAAGGCGAGUCAAGGUCU AUAACUUC | 4536 | | |
| 1911 | UCUAUCCU G CAGCACAG | 1652 | CUGUGCUG GCCGAAAGGCGAGUCAAGGUCU AGGAUAGA | 4537 | | |
| 1924 | ACAGCUUU G CCCAUCAU | 1653 | AUGAUGGG GCCGAAAGGCGAGUCAAGGUCU AAAGCUGU | 4538 | | |
| 1967 | CCAGUUUU G CCUGACAU | 1655 | AUGUCAGG GCCGAAAGGCGAGUCAAGGUCU AAAACUGG | 4539 | | |
| 2001 | UGAAUUCU G CAGUUCCU | 1658 | AGGAACUG GCCGAAAGGCGAGUCAAGGUCU AGAAUUCA | 4540 | | |
| 2013 | UUCCUAGU G CUGGUGCU | 1659 | AGCACCAG GCCGAAAGGCGAGUCAAGGUCU ACUAGGAA | 4541 | | |
| 2019 | GUGCUGGU G CUUCCGUG | 1660 | CACGGAAG GCCGAAAGGCGAGUCAAGGUCU ACCAGCAC | 4542 | | |
| 2187 | AUAUUAAU G CAGCUCUU | 1668 | AAGAGCUG GCCGAAAGGCGAGUCAAGGUCU AUUAAUAU | 4543 | | |
| 2226 | UAUCUAUU G CAUGUGAU | 1669 | AUCACAUG GCCGAAAGGCGAGUCAAGGUCU AAUACAUA | 4544 | | |
| 2259 | AGCUUUCU G CUGAACCA | 1671 | UGGUUCAG GCCGAAAGGCGAGUCAAGGUCU AGAAAGCU | 4545 | | |
| 2318 | CAGCCAGU G CCUGAUCA | 1675 | UGAUCAGG GCCGAAAGGCGAGUCAAGGUCU ACUGGCUG | 4546 | | |
| 2429 | ACUGUGAU G CUUGUGAA | 1687 | UUCACAAG GCCGAAAGGCGAGUCAAGGUCU AUCACAGU | 4547 | | |
| 2502 | AACUCAGU G CUUUGCCA | 1693 | UGGCAAAG GCCGAAAGGCGAGUCAAGGUCU ACUGAGUU | 4548 | | |
| 2507 | AGUGCUUU G CCACCUGA | 1694 | UCAGGUGG GCCGAAAGGCGAGUCAAGGUCU AAAGCACU | 4549 | | |
| 2624 | AUUCCUUU G CAGAUGGA | 1699 | UCCAUCUG GCCGAAAGGCGAGUCAAGGUCU AAAGGAAU | 4550 | | |
| 2646 | UCAGUACU G CAGUUUAU | 1700 | AUAAACUG GCCGAAAGGCGAGUCAAGGUCU AGUACUGA | 4551 | | |
| 2835 | GUGAAAUU G CUAAUGCC | 1710 | GCCAUUAG GCCGAAAGGCGAGUCAAGGUCU AAUUUCAC | 4552 | | |
| 2841 | UUGCUAAU G CCCCGGAU | 1711 | AUCCGGGG GCCGAAAGGCGAGUCAAGGUCU AUUAGCAA | 4553 | | |
| 2864 | GGGUCAUU G CCUUCCAC | 1712 | GUGCAAGG GCCGAAAGGCGAGUCAAGGUCU AAUGACCC | 4554 | | |
| 2869 | AUUGCCUU G CACACAAU | 1713 | AUUCUCUC GCCGAAAGGCGAGUCAAGGUCU AAGGCAAU | 4555 | | |
| 2879 | ACAGAAUU G CCCCAUGA | 1714 | UCAUGGGG GCCGAAAGGCGAGUCAAGGUCU AAUUCUGU | 4556 | | |
| 2964 | AUGGGUCU G CUACAUCA | 1719 | UGAUGUAG GCCGAAAGGCGAGUCAAGGUCU AGACCCAU | 4557 | | |
| 2978 | UCAAAGGU G CUCUUAUU | 1720 | AAUAAGAG GCCGAAAGGCGAGUCAAGGUCU ACCUUUGA | 4558 | | |
| 2987 | CUCUUAUU G CCUCCAGA | 1721 | UCUGGAGG GCCGAAAGGCGAGUCAAGGUCU AAUAAGAG | 4559 | | |
| 3003 | AUCUUUCU G CUUUGGCC | 1722 | GGCCAAAG GCCGAAAGGCGAGUCAAGGUCU ACAAACAU | 4560 | | |
| 3114 | CACCAUCU G CUAUAUUU | 1726 | AAAUAUAG GCCGAAAGGCGAGUCAAGGUCU AGAUGGUG | 4561 | | |
| 3201 | UGUUUGGU G CCAGCCUA | 1729 | UAGGCUGG GCCGAAAGGCGAGUCAAGGUCU ACCAAACA | 4562 | | |
| 3215 | CUAUUCCU G CUGCUUUC | 1730 | GAAAGCAC GCCGAAAGGCGAGUCAAGGUCU AGGAAUAG | 4563 | | |
| 3218 | UUCCUGCU G CUUUCAUU | 1731 | AAUGAAAG GCCGAAAGGCGAGUCAAGGUCU AGCAGGAA | 4564 | | |
| 3264 | CCUACAUU G CCUUGGCC | 1734 | GGCCAAGG GCCGAAAGGCGAGUCAAGGUCU AAUGUAGG | 4565 | | |
| 3275 | UUGGCCCU G CUCUCUGU | 1735 | ACAGAGAG GCCGAAAGGCGAGUCAAGGUCU AGGGCCAA | 4566 | | |
| 3378 | CUGAAGUU G CUAUAUCU | 1740 | AGAUAUAG GCCGAAAGGCGAGUCAAGGUCU AACUUCAG | 4567 | | |
| 3417 | GUAAUUCU G CUCUUGGU | 1742 | ACCAAGAG GCCGAAAGGCGAGUCAAGGUCU AGAAUUAC | 4568 | | |
| 3436 | UGUGAACU G CACGAUAA | 1744 | UUAUGCUG GCCGAAAGGCGAGUCAAGGUCU AGUUCACA | 4569 | | |
| 3457 | ACUCAGGC G CCUCUUCU | 1746 | AGAAGAGG GCCGAAAGGCGAGUCAAGGUCU GCCUGAGU | 4570 | | |
| 3498 | UGAAGUUU G CAGUGUUG | 1751 | CAACACUG GCCGAAAGGCGAGUCAAGGUCU AAACUUCA | 4571 | | |
| 3531 | AUGUUGGU G CCUUGUUU | 1753 | AAACAAGG GCCGAAAGGCGAGUCAAGGUCU ACCAACAU | 4572 | | |
| 3639 | UAGGACUU G CAAAUAAG | 1757 | CUUAUUUC GCCGAAAGGCGAGUCAAGGUCU AAGUCCUA | 4573 | | |
| 3660 | UUAAAGAU G CUAUGGCU | 1758 | AGCCAUAG GCCGAAAGGCGAGUCAAGGUCU AUCUUUAA | 4574 | | |
| 3700 | AUUGAAGC G CAAAGCUG | 1760 | CAGCUUUG GCCGAAAGGCGAGUCAAGGUCU GCUUCAAU | 4575 | | |
| 3718 | AUGAAAAC G CCCAAAAU | 1763 | AUUUUGGG GCCGAAAGGCGAGUCAAGGUCU CUUUUCAU | 4576 | | |
| 3807 | UUGACGUU G CAGUGCAG | 1767 | CUGCACUC GCCGAAAGGCGAGUCAAGGUCU AACGUCAA | 4577 | | |
| 3812 | GUUGCAGU G CAGUUUCA | 1768 | UGAAACUG GCCGAAAGGCGAGUCAAGGUCU ACUGCAAC | 4578 | | |
| 3853 | UUAGCCAU G CACUGUUG | 1769 | CAACAGUG GCCGAAAGGCGAGUCAAGGUCU AUGGCUAA | 4579 | | |
| 3887 | UCUUGACU G CCAUGUGU | 1772 | ACACAUGG GCCGAAAGGCGAGUCAAGGUCU AGUCAAGA | 4580 | | |
| 3920 | UGUAAGCU G CUAUGUAU | 1773 | AUACAUAG GCCGAAAGGCGAGUCAAGGUCU AGCUUACA | 4581 | | |
| 4005 | ACUUUGUU G CAGAUAGU | 1775 | ACUAUCUG GCCGAAAGGCGAGUCAAGGUCU AACAAAGU | 4582 | | |
| 4017 | AUAGUCUU G CCGCAUCU | 1776 | AGAUCCGG GCCGAAAGGCGAGUCAAGGUCU AAGACUAU | 4583 | | |
| 4020 | GUCUUGCC G CAUCUUGG | 1777 | CCAAGAUG GCCGAAAGGCGAGUCAAGGUCU GGCAAGAC | 4584 | | |
| 4035 | GGCAAGUU G CAGAGAUG | 1778 | CAUCUCUG GCCGAAAGGCGAGUCAAGGUCU AACUUGCC | 4585 | | |
| 12 | CACAGUAG G UCCCUCGG | 1779 | CCGAGGGA GCCGAAAGGCGAGUCAAGGUCU CUACUGUG | 4586 | | |
| 20 | GUCCCUCG G CUCAGUCG | 1780 | CGACUGAG GCCGAAAGGCGAGUCAAGGUCU CGAGGGAC | 4587 | | |
| 25 | UCGGCUCA G UCGGCCCA | 1781 | UGGGCCGA GCCGAAAGGCGAGUCAAGGUCU UGAGCCGA | 4588 | | |
| 29 | CUCAGUCG G CCCAGCCC | 1782 | GGGCUGGG GCCGAAAGGCGAGUCAAGGUCU CGACUGAG | 4589 | | |
| 34 | UCGGCCCA G CCCCUCUC | 1783 | GAGAGGGG GCCGAAAGGCGAGUCAAGGUCU UGGGCCGA | 4590 | | |
| 44 | CCCUCUCA G UCCUCCCC | 1784 | GGGGAGGA GCCGAAAGGCGAGUCAAGGUCU UGAGAGGG | 4591 | | |
| 73 | CGCCCGCG G CUCUGAGA | 1785 | UCUCAGAG GCCGAAAGGCGAGUCAAGGUCU CGCGGGCG | 4592 | | |
| 86 | GAGACGCG G CCCCGGCG | 1786 | CGCCGGGG GCCGAAAGGCGAGUCAAGGUCU CGCGUCUC | 4593 | | |
| 92 | CGGCCCCG G CGGCGGCG | 1787 | CGCCGCCG GCCGAAAGGCGAGUCAAGGUCU CGGGGCCG | 4594 | | |
| 95 | CCCCGGCG G CGGCGGCA | 1788 | UGCCGCCG GCCGAAAGGCGAGUCAAGGUCU CGCCGGGG | 4595 | | |
| 98 | CGGCGGCG G CGGCAGCA | 1789 | UGCUGCCG GCCGAAAGGCGAGUCAAGGUCU CGCCGCCG | 4596 | | |
| 101 | CGGCGGCG G CAGCAGCU | 1790 | AGCUGCUG GCCGAAAGGCGAGUCAAGGUCU CGCCGCCG | 4597 | | |
| 104 | CGGCGGCA G CAGCUGCA | 1791 | UGCAGCUG GCCGAAAGGCGAGUCAAGGUCU UGCCGCCG | 4598 | | |
| 107 | CGGCAGCA G CUGCAGCA | 1792 | UGCUGCAG GCCGAAAGGCGAGUCAAGGUCU UGCUGCCG | 4599 | | |
| 113 | CAGCUGCA G CAUCAUCU | 1793 | AGAUGAUG GCCGAAAGGCGAGUCAAGGUCU UGCAGCUG | 4600 | | |
| 132 | ACCCUCCA G CCAUGGAA | 1794 | UUCCAUGG GCCGAAAGGCGAGUCAAGGUCU UGGAGGGU | 4601 | | |
| 152 | CUGGACCA G UCUCCUCU | 1795 | AGAGGAGA GCCGAAAGGCGAGUCAAGGUCU UGGUCCAG | 4602 | | |
| 162 | CUCCUCUG G UCUCGUCC | 1796 | GGACGAGA GCCGAAAGGCGAGUCAAGGUCU CAGAGGAG | 4603 | | |
| 167 | CUGGUCUC G UCCUCGGA | 1797 | UCCGAGGA GCCGAAAGGCGAGUCAAGGUCU GAGACCAG | 4604 | | |
| 178 | CUCGGACA G CCCACCCC | 1798 | GGGGUGGG GCCGAAAGGCGAGUCAAGGUCU UGUCCGAG | 4605 | | |
| 188 | CCACCCCG G CCGCAGCC | 1799 | GGCUGCGG GCCGAAAGGCGAGUCAAGGUCU CGGGGUGG | 4606 | | |
| 194 | CGGCCGCA G CCCGCGUU | 1800 | AACGCGGG GCCGAAAGGCGAGUCAAGGUCU UGCGGCCG | 4607 | | |
| 200 | CAGCCCGC G UUCAAGUA | 1801 | UACUUGAA GCCGAAAGGCGAGUCAAGGUCU GCGGGCUG | 4608 | | |
| 206 | GCGUUCAA G UACCAGUU | 1802 | AACUGGUA GCCGAAAGGCGAGUCAAGGUCU UUGAACGC | 4609 | | |
| 212 | AAGUACCA G UUCGUGAG | 1803 | CUCACGAA GCCGAAAGGCGAGUCAAGGUCU UGGUACUU | 4610 | | |
| 216 | ACCAGUUC G UGAGGGAG | 1804 | CUCCCUCA GCCGAAAGGCGAGUCAAGGUCU GAACUGGU | 4611 | | |
| 224 | GUGAGGGA G CCCGAGGA | 1805 | UCCUCGGG GCCGAAAGGCGAGUCAAGGUCU UCCCUCAC | 4612 | | |
| 287 | CUGGAGGA G CUGGAGGU | 1806 | ACCUCCAC GCCGAAAGGCGAGUCAAGGUCU UCCUCCAG | 4613 | | |
| 294 | AGCUGGAG G UCCUGGAC | 1807 | CUCCAGCA GCCGAAAGGCGAGUCAAGGUCU CUCCAGCU | 4614 | | |
| 308 | GAGAGGAA G CCCGCCGC | 1808 | GCGGCGGG GCCGAAAGGCGAGUCAAGGUCU UUCCUCUC | 4615 | | |
| 320 | GCCGCCGG G CUGUCCGC | 1809 | GCGGACAG GCCGAAAGGCGAGUCAAGGUCU CCGGCGCC | 4616 | | |
| 323 | GCCGGGCU G UCCGCGGC | 1810 | GCCGCGGA GCCGAAAGGCGAGUCAAGGUCU ACCCCGGC | 4617 | | |
| 330 | UGUCCCCC G CCCCAGUG | 1811 | CACUGGGG GCCGAAAGGCGAGUCAAGGUCU CGCGGACA | 4618 | | |
| 336 | CGGCCCCA G UGCCCACC | 1812 | GGUGGGCA GCCGAAAGGCGAGUCAAGGUCU UGGGGCCC | 4619 | | |
| 358 | UGCCGCCG G CGCGCCCC | 1813 | GGGGCGCG GCCGAAAGGCGAGUCAAGGUCU CGGCGGCA | 4620 | | |
| 390 | AUGACUUC G UGCCGCCG | 1814 | CGGCGGCA GCCGAAAGGCGAGUCAAGGUCU GAAGUCAU | 4621 | | |
| 399 | UGCCGCCG G CGCCCCGG | 1815 | CCGGGGCG GCCGAAAGGCGAGUCAAGGUCU CGGCGCCA | 4622 | | |
| 420 | CCCUGCCG G CCGCUCCC | 1816 | GGGAGCGG GCCGAAAGGCGAGUCAAGGUCU CGGCAGGG | 4623 | | |
| 432 | CUCCCCCC G UCGCCCCG | 1817 | CGGGGCGA GCCGAAAGGCGAGUCAAGGUCU GGGGGGAG | 4624 | | |
| 443 | GCCCCGGA G CGGCAGCC | 1818 | GGCUGCCG GCCGAAAGGCGAGUCAAGGUCU UCCGGGGC | 4625 | | |
| 446 | CCGGAGCG G CAGCCGUC | 1819 | GACGGCUG GCCGAAAGGCGAGUCAAGGUCU CGCUCCGG | 4626 | | |
| 449 | GAGCGGCA G CCGUCUUG | 1820 | CAAGACGG GCCGAAAGGCGAGUCAAGGUCU UGCCCCUC | 4627 | | |
| 452 | CGGCAGCC G UCUUGGGA | 1821 | UCCCAAGA GCCGAAAGGCGAGUCAAGGUCU CGCUGCCC | 4628 | | |
| 466 | GGACCCGA G CCCGGUGU | 1822 | ACACCGGG GCCGAAAGGCGAGUCAAGGUCU UCGGGUCC | 4629 | | |
| 471 | CGAGCCCG G UGUCGUCG | 1823 | CGACGACA GCCGAAAGGCGAGUCAAGGUCU CGGGCUCG | 4630 | | |
| 473 | AGCCCGGU G UCGUCGAC | 1824 | GUCGACGA GCCGAAAGGCGAGUCAAGGUCU ACCGGGCU | 4631 | | |
| 476 | CCGGUGUC G UCGACCGU | 1825 | ACGGUCGA GCCGAAAGGCGAGUCAAGGUCU GACACCGG | 4632 | | |
| 483 | CGUCGACC G UGCCCGCG | 1826 | CGCGGGCA GCCGAAAGGCGAGUCAAGGUCU GGUCGACG | 4633 | | |
| 503 | UCCCCGCU G UCUGCUGC | 1827 | GCAGCAGA GCCGAAAGGCGAGUCAAGGUCU AGCGGGGA | 4634 | | |
| 516 | CUGCCGCA G UCUCGCCC | 1828 | GGGCGAGA GCCGAAAGGCGAGUCAAGGUCU UGCGGCAG | 4635 | | |
| 530 | CCCUCCAA G CUCCCUGA | 1829 | UCAGGGAG GCCGAAAGGCGAGUCAAGGUCU UUGGAGGG | 4636 | | |
| 548 | GACGACGA G CCUCCGGC | 1830 | GCCGGAGG GCCGAAAGGCGAGUCAAGGUCU UCGUCGUC | 4637 | | |
| 555 | AGCCUCCG G CCCGGCCU | 1831 | AGGCCGGG GCCGAAAGGCGAGUCAAGGUCU CGGAGGCU | 4638 | | |
| 560 | CCGGCCCG G CCUCCCCC | 1832 | GGGGGAGG GCCGAAAGGCGAGUCAAGGUCU CGGGCCGG | 4639 | | |
| 579 | CUCCCCCG G CCAGCGUG | 1833 | CACGCUGG GCCGAAAGGCGAGUCAAGGUCU CGGGGGAG | 4640 | | |
| 583 | CCCGGCCA G CGUGAGCC | 1834 | GGCUCACG GCCGAAAGGCGAGUCAAGGUCU UGGCCGGG | 4641 | | |
| 585 | CGGCCAGC G UGAGCCCC | 1835 | GGGGCUCA GCCGAAAGGCGAGUCAAGGUCU GCUGCCCC | 4642 | | |
| 589 | CAGCGUGA G CCCCCAGG | 1836 | CCUGGGGG GCCGAAAGGCGAGUCAACGUCU UCACGCUG | 4643 | | |
| 597 | GCCCCCAC G CAGAGCCC | 1837 | GGGCUCUG GCCGAAAGGCGAGUCAAGGUCU CUGGGGGC | 4644 | | |
| 602 | CAGGCAGA G CCCGUGUG | 1838 | CACACGGG GCCGAAAGGCGAGUCAAGGUCU UCUGCCUG | 4645 | | |
| 606 | CAGAGCCC G UGUGGACC | 1839 | GGUCCACA GCCGAAAGGCGAGUCAAGGUCU GGGCUCUG | 4646 | | |
| 608 | GAGCCCGU G UGGACCCC | 1840 | GGGGUCCA GCCGAAAGGCGAGUCAAGGUCU ACGGGCUC | 4647 | | |
| 621 | CCCCGCCA G CCCCCGGU | 1841 | AGCCGGGG GCCGAAAGGCGAGUCAAGGUCU UGGCCCGG | 4648 | | |
| 627 | CAGCCCCG G CUCCCGCC | 1842 | GGCGGGAG GCCGAAAGGCGAGUCAAGGUCU CGGGGCUC | 4649 | | |
| 654 | CCACCCCG G CCGCGCCC | 1843 | GGGCGCGG GCCGAAAGGCGAGUCAAGGUCU CGGGGUGG | 4650 | | |
| 665 | GCGCCCAA G CGCAGGGG | 1844 | CCCCUCCG GCCGAAAGGCGAGUCAAGGUCU UUGGGCGC | 4651 | | |
| 673 | GCGCAGGG G CUCCUCGG | 1845 | CCGAGGAG GCCGAAAGGCGAGUCAAGGUCU CCCUGCGC | 4652 | | |
| 682 | CUCCUCGG G CUCACUGG | 1846 | CCACUGAG GCCGAAAGGCGAGUCAAGGUCU CCGAGGAG | 4653 | | |
| 687 | CGGGCUCA G UGGAUGAG | 1847 | CUCAUCCA GCCGAAAGGCGAGUCAAGGUCU UGACCCCG | 4654 | | |
| 725 | GCAUCUCA G CCUGUGAU | 1848 | AUCACAGG GCCGAAAGGCGAGUCAAGGUCU UCAGAUGC | 4655 | | |
| 729 | CUGACCCU G UGAUACGC | 1849 | GCGUAUCA GCCGAAAGGCGAGUCAAGGUCU AGGCUCAC | 4656 | | |
| 767 | UUGAAGGA G CAGCCAGG | 1850 | CCUGGCUG GCCGAAAGGCGAGUCAAGGUCU UCCUUCAA | 4657 | | |
| 770 | AAGGAGCA G CCAGGUAA | 1851 | UUACCUGG GCCGAAAGGCGAGUCAAGGUCU UGCUCCUU | 4658 | | |
| 775 | GCAGCCAC G UAACACUA | 1852 | UAGUCUUA GCCGAAAGGCGAGUCAAGGUCU CUGGCUGC | 4659 | | |
| 789 | CUAUUUCG G CUGGUCAA | 1853 | UUGACCAG GCCGAAAGGCGAGUCAAGGUCU CGAAAUAG | 4660 | | |
| 793 | UUCGGCUG G UCAAGAGG | 1854 | CCUCUUGA GCCGAAAGGCGAGUCAAGGUCU CAGCCGAA | 4661 | | |
| 813 | UCCCAUCU G UCCUGCUU | 1855 | AAGCAGGA GCCGAAAGGCGAGUCAAGGUCU AGAUGGGA | 4662 | | |
| 848 | CCUUCUCU G UCUCCUCU | 1856 | AGAGGAGA GCCGAAAGGCGAGUCAAGGUCU AGAGAAGG | 4663 | | |
| 861 | CUCUCUCA G CCGCUUCU | 1857 | AGAAGCGG GCCGAAAGGCGAGUCAAGGUCU UGAGAGAG | 4664 | | |
| 892 | AUACCUUG G UAAUUUCU | 1858 | ACAAAUUA GCCGAAAGGCGAGUCAAGGUCU CAAGGUAU | 4665 | | |
| 899 | GGUAAUUU G UCAACAGU | 1859 | ACUGUUGA GCCGAAAGGCGAGUCAAGGUCU AAAUUACC | 4666 | | |
| 906 | UGUCAACA G UAUUACCC | 1860 | GGGUAAUA GCCGAAAGGCGAGUCAAGGUCU UGUUGACA | 4667 | | |
| 939 | AAGAAAAU G UCAGUGAA | 1861 | UUCACUGA GCCGAAAGGCGAGUCAAGGUCU AUUUUCUU | 4668 | | |
| 943 | AAAUGUCA G UGAAGCUU | 1862 | AAGCUUCA GCCGAAAGGCGAGUCAAGGUCU UGACAUUU | 4669 | | |
| 948 | UCAGUGAA G CUUCUAAA | 1863 | UUUAGAAG GCCGAAAGGCGAGUCAAGGUCU UUCACUCA | 4670 | | |
| 960 | CUAAAGAG G UCUCAGAG | 1864 | CUCUCAGA GCCGAAAGGCGAGUCAAGGUCU CUCUUUAG | 4671 | | |
| 972 | CAGAGAAG G CAAAAACU | 1865 | AGUUUUUG GCCGAAAGGCGAGUCAAGGUCU CUUCUCUC | 4672 | | |
| 1007 | UUAACAGA G UUUUCAGA | 1866 | UCUGAAAA GCCGAAAGGCGAGUCAAGGUCU UCUCUUAA | 4673 | | |
| 1043 | GGAUCAUC G UUCAGUGU | 1867 | ACACUGAA GCCGAAAGGCGAGUCAAGGUCU GAUGAUCC | 4674 | | |
| 1048 | AUCCUUCA G UGUCUCUC | 1868 | GAGAGACA GCCGAAAGGCGAGUCAAGGUCU UGAACGAU | 4675 | | |
| 1050 | CGCUCAGU G UCUCUCCA | 1869 | UGGAGAGA GCCGAAAGGCGAGUCAAGGUCU ACUGAACG | 4676 | | |
| 1062 | CUCCAAAA G CAGAAUCU | 1870 | AGAUUCUG GCCGAAAGGCGAGUCAAGGUCU UUUUGGAC | 4677 | | |
| 1074 | AAUCUGCC G UAAUAGUA | 1871 | UACUAUUA GCCGAAAGGCGAGUCAAGGUCU GGCAGAUU | 4678 | | |
| 1080 | CCGUAAUA G UAGCAAAU | 1872 | AUUUGCUA GCCGAAAGGCGAGUCAAGGUCU UAUUACGG | 4679 | | |
| 1083 | UAAUAGUA G CAAAUCCU | 1873 | AGGAUUUG GCCGAAAGGCGAGUCAAGGUCU UACUAUUA | 4680 | | |
| 1107 | AAAUAAUC G UGAAAAAU | 1874 | AUUUUUCA GCCGAAAGGCGAGUCAAGGUCU GAUUAUUU | 4681 | | |
| 1133 | GAAGAGAA G UUAGUUAG | 1875 | CUAACUAA GCCGAAAGGCGAGUCAAGGUCU UUCUCUUC | 4682 | | |
| 1137 | AGAAGUUA G UUAGUAAU | 1876 | AUUACUAA GCCGAAAGGCGAGUCAAGGUCU UAACUUCU | 4683 | | |
| 1141 | GUUAGUUA G UAAUAACA | 1877 | UGUUAUUA GCCGAAAGGCGAGUCAAGGUCU UAACUAAC | 4684 | | |
| 1169 | CAACAAGA G UUACCUAC | 1878 | GUAGGUAA GCCGAAAGGCGAGUCAAGGUCU UCUUGUUG | 4685 | | |
| 1179 | UACCUACA G CUCUUACU | 1879 | AGUAAGAG GCCGAAAGGCGAGUCAAGGUCU UGUAGGUA | 4686 | | |
| 1194 | CUAAAUUC G UUAAAGAG | 1880 | CUCUUUAA GCCGAAAGGCGAGUCAAGGUCU CAAUUUAC | 4687 | | |
| 1209 | AGGAUGAA G UUGUGUCU | 1881 | AGACACAA GCCGAAAGGCGAGUCAAGGUCU UUCAUCCU | 4688 | | |
| 1212 | AUGAAGUU G UGUCUUCA | 1882 | UGAAGACA GCCGAAAGGCGAGUCAAGGUCU AACUUCAU | 4689 | | |
| 1214 | GAAGUUGU G UCUUCAGA | 1883 | UCUGAAGA GCCGAAAGGCGAGUCAAGGUCU ACAACUUC | 4690 | | |
| 1227 | CAGAAAAA G CAAAAGAC | 1884 | GUCUUUUG GCCGAAAGGCGAGUCAAGGUCU UUUUUCUG | 4691 | | |
| 1237 | AAAAGACA G UUUUAAUG | 1885 | CAUUAAAA GCCGAAAGGCGAGUCAAGGUCU UGUCUUUU | 4692 | | |
| 1254 | AAAAGAGA G UUGCAGUG | 1886 | CACUGCAA GCCGAAAGGCGAGUCAAGGUCU UCUCUUUU | 4693 | | |
| 1260 | GAGUUGCA G UGGAAGCU | 1887 | AGCUUCCA GCCGAAAGGCGAGUCAAGGUCU UGCAACUC | 4694 | | |
| 1266 | CAGUGGAA G CUCCUAUG | 1888 | CAUAGGAG GCCGAAAGGCGAGUCAAGGUCU UUCCACUG | 4695 | | |
| 1307 | CCAUUUGA G CGAGUAUG | 1889 | CAUACUCG GCCGAAAGGCGAGUCAAGGUCU UCAAAUGG | 4696 | | |
| 1311 | UUGAGCGA G UAUGGGAA | 1890 | UUCCCAUA GCCGAAAGGCGAGUCAAGGUCU UCGCUCAA | 4697 | | |
| 1320 | UAUGGGAA G UGAAAGAU | 1891 | AUCUUUCA GCCGAAAGGCGAGUCAAGGUCU UUCCCAUA | 4698 | | |
| 1330 | GAAAGAUA G UAAGGAAG | 1892 | CUUCCUUA GCCGAAAGGCGAGUCAAGGUCU UAUCUUUC | 4699 | | |
| 1342 | GGAAGAUA G UGAUAUGU | 1893 | ACAUAUCA GCCGAAAGGCGAGUCAAGGUCU UAUCUUCC | 4700 | | |
| 1349 | AGUGAUAU G UUGGCUGC | 1894 | GCAGCCAA GCCGAAAGGCGAGUCAAGGUCU AUAUCACU | 4701 | | |
| 1353 | AUAUGUUG G CUGCUGGA | 1895 | UCCAGCAG GCCGAAAGGCGAGUCAAGGUCU CAACAUAU | 4702 | | |
| 1363 | UGCUGGAG G UAAAAUCC | 1896 | CCAUUUUA GCCGAAAGGCGAGUCAAGGUCU CUCCACCA | 4703 | | |
| 1375 | AAUCCAGA G CAACUUGG | 1897 | CCAAGUUG GCCGAAAGGCGAGUCAAGGUCU UCUCGAUU | 4704 | | |
| 1387 | CUUGGAAA G UAAAGUGG | 1898 | CCACUUUA GCCGAAAGGCGAGUCAAGGUCU UUUCCAAG | 4709 | | |
| 1392 | AAAGUAAA G UGGAUAAA | 1899 | UUUAUCCA GCCGAAAGGCGAGUCAAGGUCU UUUACUUU | 4706 | | |
| 1405 | UAAAAAAU G UUUUGCAG | 1900 | CUGCAAAA GCCGAAAGGCGAGUCAAGGUCU AUUUUUUA | 4707 | | |
| 1417 | UGCAGAUA G CCUUGAGC | 1901 | GCUCAAGG GCCGAAAGGCGAGUCAAGGUCU UAUCUGCA | 4708 | | |
| 1424 | AGCCUUGA G CAAACUAA | 1902 | UUAGUUUG GCCGAAAGGCGAGUCAAGGUCU UCAAGGCU | 4709 | | |
| 1447 | AAAAGAUA G UGAGAGUA | 1903 | UACUCUCA GCCGAAAGGCGAGUCAAGGUCU UAUCUUUU | 4710 | | |
| 1453 | UAGUGAGA G UAGUAAUG | 1904 | CAUUACUA GCCGAAAGGCGAGUCAAGGUCU UCUCACUA | 4711 | | |
| 1456 | UGAGAGUA G UAAUGAUG | 1905 | CAUCAUUA GCCGAAAGGCGAGUCAAGGUCU UACUCUCA | 4712 | | |
| 1480 | UUUCCCCA G UACCCCAG | 1906 | CUGGCGUA GCCGAAAGGCGAGUCAAGGUCU UGGGGAAA | 4713 | | |
| 1492 | GCCAGAAG G UAUAAAGG | 1907 | CCUUUAUA GCCGAAAGGCGAGUCAAGGUCU CUUCUGGC | 4714 | | |
| 1504 | AAAGGAUC G UUCAGGAG | 1908 | CUCCUGAA GCCGAAAGGCGAGUCAAGGUCU GAUCCUUU | 4715 | | |
| 1512 | GUUCAGGA G GAUAUAUC | 1909 | CAUAUAUG GCCGAAAGGCGAGUCAAGGUCU UCCUGAAC | 4716 | | |
| 1525 | UAUCACAU G UGCUCCCU | 1910 | AGGGAGCA GCCGAAAGGCGAGUCAAGGUCU AUGUGAUA | 4717 | | |
| 1542 | UUAACCCA G CAGCAACU | 1911 | AGUUGCUG GCCGAAAGGCGAGUCAAGGUCU UGGGUUAA | 4718 | | |
| 1545 | ACCCAGCA G CAACUGAG | 1912 | CUCAGUUG GCCGAAAGGCGAGUCAAGGUCU UGCUGGGU | 4719 | | |
| 1555 | AACUGAGA G CAUUGCAA | 1913 | UUGCAAUG GCCGAAAGGCGAGUCAAGGUCU UCUCACUC | 4720 | | |
| 1580 | UUUCCUUU G UUAGGACA | 1914 | UCUCCUAA GCCGAAAGGCGAGUCAAGGUCU AAAGGAAA | 4721 | | |
| 1638 | AAAAGAAG G CCCAAAUA | 1915 | UAUUUGGG GCCGAAAGGCGAGUCAAGGUCU CUUCUUUC | 4722 | | |
| 1647 | CCCAAAUA G UAACAGAG | 1916 | CUCUGUUA GCCGAAAGGCGAGUCAAGGUCU UAUUUGGG | 4723 | | |
| 1666 | GAAUACUA G CACCAAAA | 1917 | UUUUGGUG GCCGAAAGGCGAGUCAAGGUCU UAGUAUUC | 4724 | | |
| 1692 | CUUUUCUU G UAGCAGCA | 1918 | UGCUGCUA GCCGAAAGGCGAGUCAAGGUCU AAGAAAAG | 4729 | | |
| 1695 | UUCUUGUA G CAGCACAG | 1919 | CUGUGCUG GCCGAAAGGCGAGUCAAGGUCU UACAAGAA | 4726 | | |
| 1698 | UUGUAGCA G CACAGGAU | 1920 | AUCCUGUG GCCGAAAGGCGAGUCAAGGUCU UGCUACAA | 4727 | | |
| 1722 | CAGAUUAU G UCACAACA | 1921 | UGUUGUGA GCCGAAAGGCGAGUCAAGGUCU AUAAUCUG | 4728 | | |
| 1746 | UAACAAAG G UGACUGAG | 1922 | CUCAGUCA GCCGAAAGGCGAGUCAAGGUCU CUUUCUUA | 4729 | | |
| 1758 | CUGAGGAA G UCGUGGCA | 1923 | UGCCACGA GCCGAAAGGCGAGUCAAGGUCU UUCCUCAC | 4730 | | |
| 1761 | AGGAAGUC G UGGCAAAC | 1924 | GUUUGCCA GCCGAAAGGCGAGUCAAGGUCU GACUUCCU | 4731 | | |
| 1764 | AAGUCGUG G CAAACAUG | 1925 | CAUGUUUG GCCGAAAGGCGAGUCAAGGUCU CACGACUU | 4732 | | |
| 1780 | GCCUGAAG G CCUGACUC | 1926 | GAGUCAGG GCCGAAAGGCGAGUCAAGGUCU CUUCAGGC | 4733 | | |
| 1797 | CAGAUUUA G UACAGGAA | 1927 | UUCCUCUA GCCGAAAGGCGAGUCAAGGUCU UAAAUCUG | 4734 | | |
| 1806 | UACAGGAA G CAUGUGAA | 1928 | UUCACAUG GCCGAAAGGCGAGUCAAGGUCU UUCCUCUA | 4735 | | |
| 1810 | GGAAGCAU G UGAAAGUG | 1929 | CACUUUCA GCCGAAAGGCGAGUCAAGGUCU AUGCUUCC | 4736 | | |
| 1816 | AUGUGAAA G UGAAUUGA | 1930 | UCAAUUCA GCCGAAAGGCGAGUCAAGGUCU UUUCACAU | 4737 | | |
| 1830 | UGAAUGAA G UUACUGGU | 1931 | ACCAGUAA GCCGAAAGGCGAGUCAAGGUCU UUCAUUCA | 4738 | | |
| 1837 | AGUUACUG G UACAAAGA | 1932 | UCUUUGUA GCCGAAAGGCGAGUCAAGGUCU CAGUAACU | 4739 | | |
| 1872 | UGGACUUG G UUCAAACA | 1933 | UGUUUCAA GCCGAAAGGCGAGUCAAGGUCU CAAGUCCA | 4740 | | |
| 1887 | CAUCAGAA G UUAUGCAA | 1934 | UUGCAUAA GCCGAAAGGCGAGUCAAGGUCU UUCUCAUG | 4741 | | |
| 1898 | AUGCAAGA G UCACUCUA | 1935 | UAGAGUGA GCCGAAAGGCGAGUCAAGGUCU UCUUGCAU | 4742 | | |
| 1914 | AUCCUGCA G CACAGCUU | 1936 | AAGCUGUG GCCGAAAGGCGAGUCAAGGUCU UGCAGGAU | 4743 | | |
| 1919 | CCAGCACA G CUUUGCCC | 1937 | GGGCAAAG GCCGAAAGGCGAGUCAAGGUCU UGUGCUGC | 4744 | | |
| 1940 | UUUGAAGA G UCAGAAGC | 1938 | GCUUCUGA GCCGAAAGGCGAGUCAAGGUCU UCUUCAAA | 4745 | | |
| 1947 | AGUCAGAA G CUACUCCU | 1939 | AGGAGUAG GCCGAAAGGCGAGUCAAGGUCU UUCUGACU | 4746 | | |
| 1962 | CUUCACCA G UUUUGCCU | 1940 | AGCCAAAA GCCGAAAGGCGAGUCAAGGUCU UGGUGAAG | 4747 | | |
| 1977 | CUGACAUU G UUAUGGAA | 1941 | UUCCAUAA GCCGAAAGGCGAGUCAAGGUCU AAUGUCAG | 4748 | | |
| 1986 | UUAUGGAA G CACCAUUC | 1942 | CAAUGGUG GCCGAAAGGCGAGUCAAGGUCU UUCCAUAA | 4749 | | |
| 2004 | AUUCUGCA G UUCCUAGU | 1943 | ACUAGGAA GCCGAAAGGCGAGUCAAGGUCU UGCAGAAU | 4750 | | |
| 2011 | AGUUCCUA G UGCUGGUC | 1944 | CACCAGCA GCCGAAAGGCGAGUCAAGGUCU UAGGAACU | 4751 | | |
| 2017 | UAGUGCUG G UGCUUCCG | 1945 | CGGAAGCA GCCGAAAGGCGAGUCAAGGUCU CAGCACUA | 4752 | | |
| 2025 | GUGCUUCC G UGAUACAG | 1946 | CUGUAUCA GCCGAAAGGCGAGUCAAGGUCU GGAAGCAC | 4753 | | |
| 2033 | GUGAUACA G CCCAGCUC | 1947 | GAGCUGGG GCCGAAAGGCGAGUCAAGGUCU UGUAUCAC | 4754 | | |
| 2038 | ACAGCCCA G CUCAUCAC | 1948 | GUGAUGAG GCCGAAAGGCGAGUCAAGGUCU UGGGCUGU | 4755 | | |
| 2055 | CAUUAGAA G CUUCUUCA | 1949 | UGAAGAAG GCCGAAAGGCGAGUCAAGGUCU UUCUAAUG | 4756 | | |
| 2064 | CUUCUUCA G UUAAUUAU | 1950 | AUAAUUAA GCCGAAAGGCGAGUCAAGGUCU UGAAGAAG | 4757 | | |
| 2077 | UUAUGAAA G CAUAAAAC | 1951 | GUUUUAUG GCCGAAAGGCGAGUCAAGGUCU UUUCAUAA | 4758 | | |
| 2090 | AAACAUGA G CCUGAAAA | 1952 | UUUUCAGG GCCGAAAGGCGAGUCAAGGUCU UGAUGUUU | 4759 | | |
| 2118 | AUGAAGAG G CCAUGAGU | 1953 | ACUCAUGG GCCGAAAGGCGAGUCAAGGUCU CUCUUCAU | 4760 | | |
| 2125 | GGCCAUGA G UGUAUCAC | 1954 | GUGAUACA GCCGAAAGGCGAGUCAAGGUCU UCAUGGCC | 4761 | | |
| 2127 | CCAUGAGU G UAUCACUA | 1955 | UAGUGAUA GCCGAAAGGCGAGUCAAGGUCU ACUCAUGG | 4762 | | |
| 2142 | UAAAAAAA G UAUCAGGA | 1956 | UCCUGAUA GCCGAAAGGCGAGUCAAGGUCU UUUUUUUA | 4763 | | |
| 2171 | AUUAAAGA G CCUGAAAA | 1957 | UUUUCAGG GCCGAAAGGCGAGUCAAGGUCU UCUUUUAAU | 4764 | | |
| 2190 | UUAAUGCA G CUCUUCAA | 1958 | UUGAAGAG GCCGAAAGGCGAGUCAAGGUCU UGCAUUAA | 4765 | | |
| 2208 | AAACAGAA G CUCCUUAU | 1959 | AUAAGGAG GCCGAAAGGCGAGUCAAGGUCU UUCUGUUU | 4766 | | |
| 2230 | UAUUGCAU G UGAUUUAA | 1960 | UUAAAUCA GCCGAAAGGCGAGUCAAGGUCU AUGCAAUA | 4767 | | |
| 2252 | GAAACAAA G CUUUCUGC | 1961 | GCAGAAAG GCCGAAAGGCGAGUCAAGGUCU UUUGUUUC | 4768 | | |
| 2268 | CUGAACCA G CUCCGGAU | 1962 | AUCCGGAG GCCGAAAGGCGAGUCAAGGUCU UGGUUCAG | 4769 | | |
| 2298 | CAGAAAUG G CAAAAGUU | 1963 | AACUUUUG GCCGAAAGGCGAGUCAAGGUCU CAUUUCUG | 4770 | | |
| 2304 | UGGCAAAA G UUGAACAG | 1964 | CUGUUCAA GCCGAAAGGCGAGUCAAGGUCU UUUUGCCA | 4771 | | |
| 2312 | GUUGAACA G CCAGUGCC | 1965 | GGCACUGG GCCGAAAGGCGAGUCAAGGUCU UGUUCAAC | 4772 | | |
| 2316 | AACAGCCA G UGCCUGAU | 1966 | AUCAGGCA GCCGAAAGGCGAGUCAAGGUCU UGGCUGUU | 4773 | | |
| 2333 | CAUUCUGA G CUAGUUGA | 1967 | UCAACUAG GCCGAAAGGCGAGUCAAGGUCU UCACAAUG | 4774 | | |
| 2337 | CUGAGCUA G UUGAAGAU | 1968 | AUCUUCAA GCCGAAAGGCGAGUCAAGGUCU UAGCUCAC | 4775 | | |
| 2367 | CUGAACCA G UUGACUUA | 1969 | UAAGUCAA GCCGAAAGGCGAGUCAAGGUCU UGGUUCAG | 4776 | | |
| 2380 | CUUAUUUA G UGAUGAUU | 1970 | AAUCAUCA GCCGAAAGGCGAGUCAAGGUCU UAAAUAAG | 4777 | | |
| 2400 | UACCUGAC G UUCCACAA | 1971 | UUGUGGAA GCCGAAAGGCGAGUCAAGGUCU CUCACGUA | 4778 | | |
| 2424 | AUGAAACU G UGAUGCUU | 1972 | AAGCAUCA GCCGAAAGGCGAGUCAAGGUCU AGUUUCAU | 4779 | | |
| 2433 | UGAUGCUU G UGAAAGAA | 1973 | UUCUUUCA GCCGAAAGGCGAGUCAAGGUCU AAGCAUCA | 4780 | | |
| 2443 | GAAAGAAA G UCUCACUG | 1974 | CAGUGAGA GCCGAAAGGCGAGUCAAGGUCU UUUCUUUC | 4781 | | |
| 2465 | UCAUUUGA G UCAAUGAU | 1975 | AUCAUUGA GCCGAAAGGCGAGUCAAGGUCU UCAAAUGA | 4782 | | |
| 2500 | AAAACUCA G UGCUUUGC | 1976 | GCAAAGCA GCCGAAAGGCGAGUCAAGGUCU UGAGUUUU | 4783 | | |
| 2525 | GGAGGAAA G CCAUAUUU | 1977 | AAAUAUGC GCCGAAAGGCGAGUCAAGGUCU UUUCCUCC | 4784 | | |
| 2546 | UCUUUUAA G CUCAGUUU | 1978 | AAACUGAG GCCGAAAGGCGAGUCAAGGUCU UUAAAAGA | 4785 | | |
| 2551 | UAAGCUCA G UUUAGAUA | 1979 | UAUCUAAA GCCGAAAGGCGAGUCAAGGUCU UGAGCUUA | 4786 | | |
| 2576 | GAUACCCU G UUACCUGA | 1980 | UCAGGUAA GCCGAAAGGCGAGUCAAGGUCU AGGGUAUC | 4787 | | |
| 2589 | CUGAUGAA G UUUCAACA | 1981 | UGUUGAAA GCCGAAAGGCGAGUCAAGGUCU UUCAUCAG | 4788 | | |
| 2602 | AACAUUGA G CAAAAAGG | 1982 | CCUUUUUG GCCGAAAGGCGAGUCAAGGUCU UCAAUGUU | 4789 | | |
| 2636 | AUGGAGGA G CUCAGUAC | 1983 | GUACUGAG GCCGAAAGGCGAGUCAAGGUCU UCCUCCAU | 4790 | | |
| 2641 | GGAGCUCA G UACUGCAG | 1984 | CUGCAGUA GCCGAAAGGCGAGUCAAGGUCU UGAGCUCC | 4791 | | |
| 2649 | GUACUGCA G UUUAUUCA | 1985 | UGAAUAAA GCCGAAAGGCGAGUCAAGGUCU UGCAGUAC | 4792 | | |
| 2685 | CUAAGGAA G CACAGAUA | 1986 | UAUCUGUG GCCGAAAGGCGAGUCAAGGUCU UUCCUUAG | 4793 | | |
| 2708 | ACUGAAAC G UUUUCAGA | 1987 | UCUGAAAA GCCGAAAGGCGAGUCAAGGUCU GUUUCAGU | 4794 | | |
| 2744 | AUAGAUGA G UUCCCUAC | 1988 | GUAGGGAA GCCGAAAGGCGAGUCAAGGUCU UCAUCUAU | 4795 | | |
| 2761 | AUUGAUCA G UUCUAAAA | 1989 | UUUUAGAA GCCGAAAGGCGAGUCAAGGUCU UGAUGAAU | 4796 | | |
| 2790 | CUAAAUUA G CCAGGGAA | 1990 | UUCCCUGG GCCGAAAGGCGAGUCAAGGUCU UAAUUUAG | 4797 | | |
| 2814 | ACCUAGAA G UAUCCCAC | 1991 | GUGGGAUA GCCGAAAGGCGAGUCAAGGUCU UUCUAGGU | 4798 | | |
| 2827 | CCACAAAA G UGAAAUUG | 1992 | CAAUUUCA GCCGAAAGGCGAGUCAAGGUCU UUUUGUGG | 4799 | | |
| 2853 | CCGAUGGA G CUGGCUCA | 1993 | UGACCCAG GCCGAAAGGCGAGUCAAGGUCU UCCAUCCG | 4800 | | |
| 2858 | GGAGCUGG G UCAUUGCC | 1994 | GGCAAUGA GCCGAAAGGCGAGUCAAGGUCU CCAGCUCC | 4801 | | |
| 2916 | AACCCAAA G UUGAAGAG | 1995 | CUCUUCAA GCCGAAAGGCGAGUCAAGGUCU UUUGGGUU | 4802 | | |
| 2932 | GAAAAUCA G UUUCUCAG | 1996 | CUGAGAAA GCCGAAAGGCGAGUCAAGGUCU UGAUUUUC | 4803 | | |
| 2960 | AAAAAUGG G UCUGCUAC | 1997 | GUAGCAGA GCCGAAAGGCGAGUCAAGGUCU CCAUUUUU | 4804 | | |
| 2976 | CAUCAAAG G UGCUCUUA | 1998 | UAAGAGCA GCCGAAAGGCGAGUCAAGGUCU CUUUGAUG | 4805 | | |
| 2997 | CUCCAGAU G UUUCUGCU | 1999 | AGCAGAAA GCCGAAAGGCGAGUCAAGGUCU AUCUGGAG | 4806 | | |
| 3009 | CUGCUUUG G GGAGUCAA | 2000 | UUGAGUGG GCCGAAAGGCGAGUCAAGGUCU CAAAGCAG | 4807 | | |
| 3018 | CCACUCAA G CAGAGAUA | 2001 | UAUCUCUG GCCGAAAGGCGAGUCAAGGUCU UUGAGUGG | 4808 | | |
| 3031 | GAUAGAGA G CAUAGUUA | 2002 | UAACUAUG GCCGAAAGGCGAGUCAAGGUCU UCUCUAUC | 4809 | | |
| 3036 | AGAGCAUA G UUAAACCC | 2003 | GGGUUUAA GCCGAAAGGCGAGUCAAGGUCU UAUGCUCU | 4810 | | |
| 3048 | AACCCAAA G UUCUUGUG | 2004 | CACAAGAA GCCGAAAGGCGAGUCAAGGUCU UUUGGGUU | 4811 | | |
| 3054 | AAGUUCUU G UGAAAGAA | 2005 | UUCUUUCA GCCGAAAGGCGAGUCAAGGUCU AAGAACUU | 4812 | | |
| 3063 | UGAAAGAA G CUGAGAAA | 2006 | UUUCUCAG GCCGAAAGGCGAGUCAAGGUCU UUCUUUCA | 4813 | | |
| 3126 | UAUUUUCA G CAGAGCUG | 2007 | CAGCUCUG GCCGAAAGGCGAGUCAAGGUCU UGAAAAUA | 4814 | | |
| 3131 | UCAGCAGA G CUGAGUAA | 2008 | UUACUCAG GCCGAAAGGCGAGUCAAGGUCU UCUCCUGA | 4815 | | |
| 3136 | AGAGCUGA G UAAAACUU | 2009 | AAGUUUUA GCCGAAAGGCGAGUCAAGGUCU UCAGCUCU | 4816 | | |
| 3147 | AAACUUCA G UUGUUGAC | 2010 | GUCAACAA GCCGAAAGGCGAGUCAAGGUCU UGAAGUUU | 4817 | | |
| 3150 | CUUCAGUU G UUGACCUC | 2011 | GAGGUCAA GCCGAAAGGCGAGUCAAGGUCU AACUGAAG | 4818 | | |
| 3161 | GACCUCCU G UACUGGAG | 2012 | CUCCAGUA GCCGAAAGGCGAGUCAAGGUCU AGGAGGUC | 4819 | | |
| 3189 | AGACUGGA G UGGUGUUU | 2013 | AAACACCA GCCGAAAGGCGAGUCAAGGUCU UCCACUCU | 4820 | | |
| 3192 | CUGGAGUG G UGUUUGGU | 2014 | ACCAAACA GCCGAAAGGCGAGUCAAGGUCU CACUCCAG | 4821 | | |
| 3194 | GGAGUGGU G UUUGGUGC | 2015 | GCACCAAA GCCGAAAGGCGAGUCAAGGUCU AGCACUCC | 4822 | | |
| 3199 | GGUGUUUG G UGCCACCC | 2016 | GGCUGGCA GCCGAAAGGCGAGUCAAGGUCU CAAACACC | 4823 | | |
| 3205 | UGGUGCCA G CCUAUUCC | 2017 | GGAAUAGG GCCGAAAGGCGAGUCAAGGUCU UGGCACCA | 4824 | | |
| 3231 | CAUUGACA G UAUUCAGC | 2018 | GCUGAAUA GCCGAAAGGCGAGUCAAGGUCU UGUCAAUG | 4825 | | |
| 3238 | AGUAUUCA G CADUGUGA | 2019 | UCACAAUG GCCGAAAGGCGAGUCAAGGUCU UGAAUACU | 4826 | | |
| 3243 | UCAGCAUU G UGAGCGUA | 2020 | UACGCUCA GCCGAAAGGCGAGUCAAGGUCU AAUGCUCA | 4827 | | |
| 3247 | CAUUGUGA G CGUAACAG | 2021 | CUGUUACG GCCGAAAGGCGAGUCAAGGUCU UCACAAUG | 4828 | | |
| 3249 | UUGUGAGC G UAACAGCC | 2022 | GGCUGUUA GCCGAAAGGCGAGUCAAGGUCU GCUCACAA | 4829 | | |
| 3255 | GCGUAACA G CCUACAUU | 2023 | AAUGUAGG GCCGAAAGGCGAGUCAAGGUCU UGUUACGC | 4830 | | |
| 3270 | UUGCCUUG G CCCUGCUC | 2024 | GAGCAGGG GCCGAAAGGCGAGUCAAGGUCU CAAGGCAA | 4831 | | |
| 3282 | UGCUCUCU G UGACCAUC | 2025 | GAUGGUCA GCCGAAAGGCGAGUCAAGGUCU AGAGAGCA | 4832 | | |
| 3292 | GACCAUCA G CUUUAGGA | 2026 | GCCGAAAG GCCGAAAGGCGAGUCAAGGUCU UCAUGGUC | 4833 | | |
| 3310 | AUACAAGG G UGUGAUGG | 2027 | GGAUCACA GCCGAAAGGCGAGUCAAGGUCU CCUUGUAU | 4834 | | |
| 3312 | ACAAGGGU G UGAUGCAA | 2028 | UUGGAUCA GCCGAAAGGCGAGUCAAGGUCU ACCCUUCU | 4835 | | |
| 3321 | UGAUCCAA G CUAUCCAG | 2029 | CUGGAUAG GCCGAAAGGCGAGUCAAGGUCU UUGGAUCA | 4836 | | |
| 3343 | AGAUGAAG G CCACCCAU | 2030 | AUGGGUGG GCCGAAAGGCGAGUCAAGGUCU CUUCAUCU | 4837 | | |
| 3357 | CAUUCAGG G CAUAUCUG | 2031 | CAGAUAUG GCCGAAAGGCGAGUCAAGGUCU CCUGAAUG | 4838 | | |
| 3375 | AAUCUGAA G UUGCUAUA | 2032 | UAUAGCAA GCCGAAAGGCGAGUCAAGGUCU UUCAGAUU | 4839 | | |
| 3392 | UCUGAGGA G UUGGUUCA | 2033 | UGAACCAA GCCGAAAGGCGAGUCAAGGUCU UCCUCAGA | 4840 | | |
| 3396 | AGGAGUUG G UUCAGAAG | 2034 | CUUCUGAA GCCGAAAGGCGAGUCAAGGUCU CAACUCCU | 4841 | | |
| 3404 | GUUCAGAA G UACAGUAA | 2035 | UUACUGUA GCCGAAAGGCGAGUCAAGGUCU UUCUGAAC | 4842 | | |
| 3409 | GAAGUACA G UAAUUCUG | 2036 | CAGAAUUA GCCGAAAGGCGAGUCAAGGUCU UGUACUUC | 4843 | | |
| 3424 | UGCUCUUG G UCAUGUGA | 2037 | UCACAUGA GCCGAAAGGCGAGUCAAGGUCU CAAGAGCA | 4844 | | |
| 3429 | UUGGUCAU G UGAACUGC | 2038 | GCAGUUCA GCCGAAAGGCGAGUCAAGGUCU AUGACCAA | 4845 | | |
| 3455 | GAACUCAG G CGCCUCUU | 2039 | AAGAGGCG GCCGAAAGGCGAGUCAAGGUCU CUGAGUUC | 4846 | | |
| 3468 | UCUUCUUA G UUGAUGAU | 2040 | AUCAUCAA GCCGAAAGGCGAGUCAAGGUCU UAAGAAAGA | 4847 | | |
| 3480 | AUGAUUUA G UUGAUUCU | 2041 | AGAAUCAA GCCGAAAGGCGAGUCAAGGUCU UAAAUCAU | 4848 | | |
| 3494 | UCUCUGAA G UUUGCAGU | 2042 | ACUGCAAA GCCGAAAGGCGAGUCAAGGUCU UCCAGAGA | 4849 | | |
| 3501 | AGUUUGCA G UGUUGAUG | 2043 | CAUCAACA GCCGAAAGGCGAGUCAAGGUCU UGCAAACU | 4850 | | |
| 3503 | UUUGCAGU G UUGAUGUG | 2044 | CACAUCAA GCCGAAAGGCGAGUCAAGGUCU ACUGCAAA | 4851 | | |
| 3509 | GUGUUGAU G UGGGUAUU | 2045 | AAUACCCA GCCGAAAGGCGAGUCAAGGUCU AUCAACAC | 4852 | | |
| 3513 | UGAUGUGG G UAUUUACC | 2046 | GGUAAAUA GCCGAAAGGCGAGUCAAGGUCU CCACAUCA | 4853 | | |
| 3525 | UUACCUAU G UUGGUGCC | 2047 | GGCACCAA GCCGAAAGGCGAGUCAAGGUCU AUAGGUAA | 4854 | | |
| 3529 | CUAUGUUG G UGCCUUGU | 2048 | ACAAGGCA GCCGAAAGGCGAGUCAAGGUCU CAACAUAG | 4855 | | |
| 3536 | GGUGCCUU G UUUAAUGG | 2049 | CCAUUAAA GCCGAAAGGCGAGUCAAGGUCU AAGGCACC | 4856 | | |
| 3544 | GUUUAAUG G UCUGACAC | 2050 | GUGUCAGA GCCGAAAGGCGAGUCAAGGUCU CAUUAAAC | 4857 | | |
| 3564 | UGAUUUUG G CUCUCAUU | 2051 | AAUGAGAG GCCGAAAGGCGAGUCAAGGUCU CAAAAUCA | 4858 | | |
| 3583 | ACUCUUCA G UGUUCCUG | 2052 | CAGGAACA GCCGAAAGGCGAGUCAAGGUCU UGAAGAGU | 4859 | | |
| 3585 | UCUUCAGU G UUCCUGUU | 2053 | AACAGGAA GCCGAAAGGCGAGUCAAGGUCU ACUGAAGA | 4860 | | |
| 3591 | GUGUUCCU G UUAUUUAU | 2054 | AUAAAUAA GCCGAAAGGCGAGUCAAGGUCU AGGAACAC | 4861 | | |
| 3605 | UAUGAACG G CAUCAGGC | 2055 | GCCUGAUG GCCGAAAGGCGAGUCAAGGUCU CGUUCAUA | 4862 | | |
| 3612 | GGCAUCAG G CACAGAUA | 2056 | UAUCUGUG GCCGAAAGGCGAGUCAAGGUCU CUGAUGCC | 4863 | | |
| 3651 | AUAAGAAU G UUAAAGAU | 2057 | AUCUUUAA GCCGAAAGGCGAGUCAAGGUCU AUUCUUAU | 4864 | | |
| 3666 | AUGCUAUG G CUAAAAUC | 2058 | GAUUUUAG GCCGAAAGGCGAGUCAAGGUCU CAUAGCAU | 4865 | | |
| 3678 | AAAUCCAA G CAAAAAUC | 2059 | GAUUUUUG GCCGAAAGGCGAGUCAAGGUCU UUGGAUUU | 4866 | | |
| 3698 | GGAUUGAA G CGCAAAGC | 2060 | GCUUUGCG GCCGAAAGGCGAGUCAAGGUCU UUCAAUCC | 4867 | | |
| 3705 | AGCGCAAA G CUGAAUGA | 2061 | UCAUUCAG GCCGAAAGGCGAGUCAAGGUCU UUUGCGCU | 4868 | | |
| 3732 | AAUAAUUA G UAGGAGUU | 2062 | AACUCCUA GCCGAAAGGCGAGUCAAGGUCU UAAUUAUU | 4869 | | |
| 3738 | UAGUAGGA G UUCAUCUU | 2063 | AAGAUGAA GCCGAAAGGCGAGUCAAGGUCU UCCUACUA | 4870 | | |
| 3781 | GGGGGAGG G UCAGGGAA | 2064 | UUCCCUGA GCCGAAAGGCGAGUCAAGGUCU CCUCCCCC | 4871 | | |
| 3804 | ACCUUGAC G UUGCAGUG | 2065 | CACUGCAA GCCGAAAGGCGAGUCAAGGUCU GUCAAGGU | 4872 | | |
| 3810 | ACGUUGCA G UGCAGUUU | 2066 | AAACUGCA GCCGAAAGGCGAGUCAAGGUCU UGCAACGU | 4873 | | |
| 3815 | GCAGUGCA G UUUCACAG | 2067 | CUGUGAAA GCCGAAAGGCGAGUCAAGGUCU UGCACUGC | 4874 | | |
| 3827 | CACAGAUC G UUGUUAGA | 2068 | UCUAACAA GCCGAAAGGCGAGUCAAGGUCU GAUCUGUG | 4875 | | |
| 3830 | AGAUCGUU G UUAGAUCU | 2069 | AGAUCUAA GCCGAAAGGCGAGUCAAGGUCU AACGAUCU | 4876 | | |
| 3848 | UAUUUUUA G GCAUGCAC | 2070 | GUGCAUGG GCCGAAAGGCGAGUCAAGGUCU UAAAAAUA | 4877 | | |
| 3858 | CAUGCACU G UUGUGAGG | 2071 | CCUCACAA GCCGAAAGGCGAGUCAAGGUCU AGUGCAUG | 4878 | | |
| 3861 | GCACUGUU G UGAGGAAA | 2072 | UUUCCUCA GCCGAAAGGCGAGUCAAGGUCU AACAGUGC | 4879 | | |
| 3878 | AAUCACCU G UCUUGACU | 2073 | AGUCAAGA GCCGAAAGGCGAGUCAAGGUCU AGGUAAUU | 4880 | | |
| 3892 | ACUGCCAU G UGUUGAUG | 2074 | GAUGAACA GCCGAAAGGCGAGUCAAGGUCU AUGGCAGU | 4881 | | |
| 3894 | UGCCAUGU G UUGAUGAU | 2075 | AUGAUGAA GCCGAAAGGCGAGUCAAGGUCU ACAUGGCA | 4882 | | |
| 3908 | CAUCUUAA G UAUUGUAA | 2076 | UUACAAUA GCCGAAAGGCGAGUCAAGGUCU UUAAGAUG | 4883 | | |
| 3913 | UAAGUAUU G UAAGCUGC | 2077 | GCAGCUUA GCCGAAAGGCGAGUCAAGGUCU AAUACUUA | 4884 | | |
| 3917 | UAUUGUAA G CUGCUAUG | 2078 | CAUAGCAG GCCGAAAGGCGAGUCAAGGUCU UUACAAUA | 4885 | | |
| 3925 | GCUGCUAU G UAUGGAUU | 2079 | AAUCCAUA GCCGAAAGGCGAGUCAAGGUCU AUAGCAGC | 4886 | | |
| 3940 | UUUAAACC G UAAUCAUA | 2080 | UAUGAUUA GCCGAAAGGCGAGUCAAGGUCU GGUUUAAA | 4887 | | |
| 3966 | UAUCUGAG G CACUGGUG | 2081 | CACCAGUG GCCGAAAGGCGAGUCAAGGUCU CUCAGAUA | 4888 | | |
| 3972 | AGGCACUG G UGGAAUAA | 2082 | UUAUUCCA GCCGAAAGGCGAGUCAAGGUCU CAGUGCCU | 4889 | | |
| 3988 | AAAAACCU G UAUAUUUU | 2083 | AAAAUAUA GCCGAAAGGCGAGUCAAGGUCU AGGUUUUU | 4890 | | |
| 4002 | UUUACUUU G UUGCAGAU | 2084 | AUCUGCAA GCCGAAAGGCGAGUCAAGGUCU AAAGUAAA | 4891 | | |
| 4012 | UGCAGAUA G UCUUGCCG | 2085 | CGGCAAGA GCCGAAAGGCGAGUCAAGGUCU UAUCUGCA | 4892 | | |
| 4028 | GCAUCUUG G CAAGUUGC | 2086 | GCAACUUG GCCGAAAGGCGAGUCAAGGUCU CAAGAUGC | 4893 | | |
| 4032 | CUUGGCAA G UUGCAGAG | 2087 | CUCUGCAA GCCGAAAGGCGAGUCAAGGUCU UUGCCAAG | 4894 | | |
| 4044 | CAGAGAUG G UGGAGCUA | 2088 | UAGCUCCA GCCGAAAGGCGAGUCAAGGUCU CAUCUCUG | 4895 | | |
| Input Seqence = AB020693. Cut Site = G/Y Stern Length = 8 | | | | | Core Sequence = GCcgaaagGCGaGuCaaGGuCU AB020593 (Homo sapiens mRNA for KIAA0886 protein (Nogo-A); 4053 | bp) |

TABLE VII
Human NOGO DNAzyme and Substrate Sequence

| Pos | Substrate | Seq ID | DNAzyme | Seq ID |
|---|---|---|---|---|
| 208 | GUUCAAGU A CCAGUUCG | 24 | CGAACTGG GGCTAGCTACAACGA ACTTGAAC | 4896 |
| 734 | CCUGUGAU A CGCUCCUC | 61 | GAGGAGCG GGCTAGCTACAACGA ATCACAGG | 4897 |
| 753 | CAGAAAAU A UGGACUUG | 64 | CAAGTCCA GGCTAGCTACAACGA ATTTTCTG | 4898 |
| 783 | GUAACACU A UUUCGGCU | 67 | AGCCGAAA GGCTAGCTACAACGA AGTGTTAC | 4899 |
| 886 | ACAUGAAU A CCUUGGUA | 96 | TACCAAGG GGCTAGCTACAACGA ATTCATGT | 4900 |
| 908 | UCAACAGU A UUACCCAC | 102 | GTGGGTAA GGCTAGCTACAACGA ACTGTTGA | 4901 |
| 911 | ACAGUAUU A CCCACUGA | 104 | TCAGTGGG GGCTAGCTACAACGA AATACTGT | 4902 |
| 983 | AAAACUCU A CUCAUAGA | 114 | TCTATGAG GGCTAGCTACAACGA AGAGTTTT | 4903 |
| 1024 | AUUAGAAU A CUCAGAAA | 127 | TTTCTGAG GGCTAGCTACAACGA ATTCTAAT | 4904 |
| 1172 | CAAGAGUU A CCUACAGC | 157 | GCTGTAGG GGCTAGCTACAACGA AACTCTTG | 4905 |
| 1176 | AGUUACCU A CAGCUCUU | 158 | AAGAGCTG GGCTAGCTACAACGA AGGTAACT | 4906 |
| 1185 | CAGCUCUU A CUAAAUUG | 161 | CAATTTAG GGCTAGCTACAACGA AAGAGCTG | 4907 |
| 1272 | AAGCUCCU A UGAGGGAG | 176 | CTCCCTCA GGCTAGCTACAACGA AGGAGCTT | 4908 |
| 1285 | GGAGGAAU A UGCAGACU | 177 | AGTCTGCA GGCTAGCTACAACGA ATTCCTCC | 4909 |
| 1313 | GAGCGAGU A UGGGAAGU | 182 | ACTTCCCA GGCTAGCTACAACGA ACTCGCTC | 4910 |
| 1347 | AUAGUGAU A UGUUGGCU | 186 | AGCCAACA GGCTAGCTACAACGA ATCACTAT | 4911 |
| 1467 | AUGAUGAU A CUUCUUUC | 203 | GAAAGAAG GGCTAGCTACAACGA ATCATCAT | 4912 |
| 1482 | UCCCCAGU A CGCCAGAA | 209 | TTCTGGCG GGCTAGCTACAACGA ACTGGGGA | 4913 |
| 1494 | CAGAAGGU A UAAAGGAU | 210 | ATCCTTTA GGCTAGCTACAACGA ACCTTCTG | 4914 |
| 1516 | AGGAGCAU A UAUCACAU | 215 | ATGTGATA GGCTAGCTACAACGA ATGCTCCT | 4915 |
| 1518 | GAGCAUAU A UCACAUGU | 216 | ACATGTGA GGCTAGCTACAACGA ATATGCTC | 4916 |
| 1593 | GAGAUCCU A CUUCAGAA | 233 | TTCTGAAG GGCTAGCTACAACGA AGGATCTC | 4917 |
| 1662 | AGAAGAAU A CUAGCACC | 240 | GGTGCTAG GGCTAGCTACAACGA ATTCTTCT | 4918 |
| 1720 | GACAGAUU A UGUCACAA | 252 | TTGTGACA GGCTAGCTACAACGA AATCTGTC | 4919 |
| 1799 | GAUUUAGU A CAGGAAGC | 263 | GCTTCCTG GGCTAGCTACAACGA ACTAAATC | 4920 |
| 1833 | AUGAAGUU A CUGGUACA | 266 | TGTACCAG GGCTAGCTACAACGA AACTTCAT | 4921 |
| 1839 | UUACUGGU A CAAAGAUU | 267 | AATCTTTG GGCTAGCTACAACGA ACCAGTAA | 4922 |
| 1852 | GAUUGCUU A UGAAACAA | 270 | TTGTTTCA GGCTAGCTACAACGA AAGCAATC | 4923 |
| 1890 | CAGAAGUU A UGCAAGAG | 276 | CTCTTGCA GGCTAGCTACAACGA AACTTCTG | 4924 |
| 1906 | GUCACUCU A UCCUGCAG | 279 | CTGCAGGA GGCTAGCTACAACGA AGAGTGAC | 4925 |
| 1950 | CAGAAGCU A CUCCUUCA | 287 | TGAAGGAG GGCTAGCTACAACGA AGCTTCTG | 4926 |
| 1980 | ACAUUGUU A UGGAAGCA | 296 | TGCTTCCA GGCTAGCTACAACGA AACAATGT | 4927 |
| 2030 | UCCGUGAU A CAGCCCAG | 305 | CTGGGCTG GGCTAGCTACAACGA ATCACGGA | 4928 |
| 2071 | AGUUAAUU A UGAAAGCA | 317 | TGCTTTCA GGCTAGCTACAACGA AATTAACT | 4929 |
| 2110 | CCCACCAU A UGAAGAGG | 319 | CCTCTTCA GGCTAGCTACAACGA ATGGTGGG | 4930 |
| 2129 | AUGAGUGU A UCACUAAA | 320 | TTTAGTGA GGCTAGCTACAACGA ACACTCAT | 4931 |
| 2144 | AAAAAAGU A UCAGGAAU | 323 | ATTCCTGA GGCTAGCTACAACGA ACTTTTTT | 4932 |
| 2181 | CUGAAAAU A UUAAUGCA | 328 | TGCATTAA GGCTAGCTACAACGA ATTTTCAG | 4933 |
| 2215 | AGCUCCUU A UAUAUCUA | 336 | TAGATATA GGCTAGCTACAACGA AAGGAGCT | 4934 |
| 2217 | CUCCUUAU A UAUCUAUU | 337 | AATAGATA GGCTAGCTACAACGA ATAAGGAG | 4935 |
| 2219 | CCUUAUAU A UCUAUUGC | 338 | GCAATAGA GGCTAGCTACAACGA ATATAAGG | 4936 |
| 2223 | AUAUAUCU A UUGCAUGU | 340 | ACATGCAA GGCTAGCTACAACGA AGATATAT | 4937 |
| 2287 | CUCUGAUU A UUCAGAAA | 356 | TTTCTGAA GGCTAGCTACAACGA AATCAGAG | 4938 |
| 2375 | GUUGACUU A UUUAGUGA | 372 | TCACTAAA GGCTAGCTACAACGA AAGTCAAC | 4939 |
| 2393 | GAUUCAAU A CCUGACGU | 378 | ACGTCAGG GGCTAGCTACAACGA ATTGAATC | 4940 |
| 2479 | GAUAGAAU A UGAAAAUA | 390 | TATTTTCA GGCTAGCTACAACGA ATTCTATC | 4941 |
| 2530 | AAAGCCAU A UUUGGAAU | 395 | ATTCCAAA GGCTAGCTACAACGA ATGGCTTT | 4942 |
| 2571 | CAAAAGAU A CCCUGUUA | 408 | TAACAGGG GGCTAGCTACAACGA ATCTTTTG | 4943 |
| 2579 | ACCCUGUU A CCUGAUGA | 410 | TCATCAGG GGCTAGCTACAACGA AACAGGGT | 4944 |
| 2643 | AGCUCAGU A CUGCAGUU | 420 | AACTGCAG GGCTAGCTACAACGA ACTGAGCT | 4945 |
| 2653 | UGCAGUUU A UUCAAAUG | 423 | CATTTGAA GGCTAGCTACAACGA AAACTGCA | 4946 |
| 2669 | GAUGACUU A UUUAUUUC | 427 | GAAATAAA GGCTAGCTACAACGA AAGTCATC | 4947 |
| 2673 | ACUUAUUU A UUUCUAAG | 430 | CTTAGAAA GGCTAGCTACAACGA AAATAAGT | 4948 |
| 2736 | UUGAAAUU A UAGAUGAG | 446 | CTCATCTA GGCTAGCTACAACGA AATTTCAA | 4949 |
| 2751 | AGUUCCCU A CAUUGAUC | 450 | GATCAATG GGCTAGCTACAACGA AGGGAACT | 4950 |
| 2800 | CAGGGAAU A UACUGACC | 465 | GGTCAGTA GGCTAGCTACAACGA ATTCCCTG | 4951 |
| 2802 | GGGAAUAU A CUGACCUA | 466 | TAGGTCAG GGCTAGCTACAACGA ATATTCCC | 4952 |
| 2816 | CUAGAAGU A UCCCACAA | 468 | TTGTGGGA GGCTAGCTACAACGA ACTTCTAG | 4953 |
| 2906 | AAGAACAU A CAACCCAA | 481 | TTGGGTTG GGCTAGCTACAACGA ATGTTCTT | 4954 |
| 2967 | GGUCUGCU A CAUCAAAG | 494 | CTTTGATG GGCTAGCTACAACGA AGCAGACC | 4955 |
| 2984 | GUGCUCUU A UUGCCUCC | 498 | GGAGGCAA GGCTAGCTACAACGA AAGAGCAC | 4956 |
| 3087 | CUUCCGAU A CAGAAAAA | 518 | TTTTTCTG GGCTAGCTACAACGA ATCGGAAG | 4957 |
| 3117 | CAUCUGCU A UAUUUUCA | 521 | TGAAAATA GGCTAGCTACAACGA AGCAGATG | 4958 |
| 3119 | UCUGCUAU A UUUUCAGC | 522 | GCTGAAAA GGCTAGCTACAACGA ATAGCAGA | 4959 |
| 3163 | CCUCCUGU A CUGGAGAG | 533 | CTCTCCAG GGCTAGCTACAACGA ACAGGAGG | 4960 |
| 3209 | GCCAGCCU A UUCCUGCU | 538 | AGCAGGAA GGCTAGCTACAACGA AGGCTGGC | 4961 |
| 3233 | UUGACAGU A UUCAGCAU | 545 | ATGCTGGA GGCTAGCTACAACGA ACTGTCAA | 4962 |
| 3259 | AACAGCCU A CAUUGCCU | 550 | AGGCAATG GGCTAGCTACAACGA AGGCTGTT | 4963 |
| 3302 | UUUAGGAU A UACAAGGG | 559 | CCCTTGTA GGCTAGCTACAACGA ATCCTAAA | 4964 |
| 3304 | UAGGAUAU A CAAGGGUG | 560 | CACCCTTG GGCTAGCTACAACGA ATATCCTA | 4965 |
| 3324 | UCCAAGCU A UCCAGAAA | 562 | TTTCTGGA GGCTAGCTACAACGA AGCTTGGA | 4966 |
| 3361 | CAGGGCAU A UCUGGAAU | 567 | ATTCCAGA GGCTAGCTACAACGA ATGCCCTG | 4967 |
| 3381 | AAGUUGCU A UAUCUGAG | 571 | CTCAGATA GGCTAGCTACAACGA AGCAACTT | 4968 |
| 3383 | GUUGCUAU A UCUGAGGA | 572 | TCCTCAGA GGCTAGCTACAACGA ATAGCAAC | 4969 |
| 3406 | UCAGAAGU A CAGUAAUU | 577 | AATTACTG GGCTAGCTACAACGA ACTTCTGA | 4970 |
| 3515 | AUGUGGGU A UUUACCUA | 602 | TAGGTAAA GGCTAGCTACAACGA ACCCACAT | 4971 |
| 3519 | GGGUAUUU A CCUAUGUU | 605 | AACATAGG GGCTAGCTACAACGA AAATACCC | 4972 |
| 3523 | AUUUACCU A UGUUGGUG | 606 | CACCAACA GGCTAGCTACAACGA AGGTAAAT | 4973 |
| 3554 | CUGACACU A CUGAUUUU | 613 | AAAATCAG GGCTAGCTACAACGA AGTGTCAG | 4974 |
| 3594 | UUCCUGUU A UUUAUGAA | 628 | TTCATAAA GGCTAGCTACAACGA AACAGGAA | 4975 |
| 3598 | UGUUAUUU A UGAACGGC | 631 | GCCGTTCA GGCTAGCTACAACGA AAATAACA | 4976 |
| 3628 | AGAUCAUU A UCUAGGAC | 636 | GTCCTAGA GGCTAGCTACAACGA AATGATCT | 4977 |
| 3663 | AAGAUGCU A UGGCUAAA | 643 | TTTAGCCA GGCTAGCTACAACGA AGCATCTT | 4978 |
| 3757 | AAGGGGAU A UUCAUUUG | 658 | CAAATGAA GGCTAGCTACAACGA ATCCCCTT | 4979 |
| 3769 | AUUUGAUU A UACGGGGG | 664 | CCCCCGTA GGCTAGCTACAACGA AATCAAAT | 4980 |
| 3771 | UUGAUUAU A CGGGGGAG | 665 | CTCCCCCG GGCTAGCTACAACGA ATAATCAA | 4981 |
| 3841 | AGAUCUUU A UUUUUAGC | 679 | GCTAAAAA GGCTAGCTACAACGA AAAGATCT | 4982 |
| 3874 | GAAAAAUU A CCUGUCUU | 687 | AAGACAGG GGCTAGCTACAACGA AATTTTTC | 4983 |
| 3910 | UCUUAAGU A UUGUAAGC | 696 | GCTTACAA GGCTAGCTACAACGA ACTTAAGA | 4984 |
| 3923 | AAGCUGCU A UGUAUGGA | 699 | TCCATACA GGCTAGCTACAACGA AGCAGCTT | 4985 |
| 3927 | UGCUAUGU A UGGAUUUA | 700 | TAAATCCA GGCTAGCTACAACGA ACATAGCA | 4986 |
| 3948 | GUAAUCAU A UCUUUUUC | 706 | GAAAAAGA GGCTAGCTACAACGA ATGATTAC | 4987 |
| 3959 | UUUUUCCU A UCUGAGGC | 713 | GCCTCAGA GGCTAGCTACAACGA AGGAAAAA | 4988 |
| 3990 | AAACCUGU A UAUUUUAC | 716 | GTAAAATA GGCTAGCTACAACGA ACAGGTTT | 4989 |
| 3992 | ACCUGUAU A UUUUACUU | 717 | AAGTAAAA GGCTAGCTACAACGA ATACAGGT | 4990 |
| 3997 | UAUAUUUU A CUUUGUUG | 721 | CAACAAAG GGCTAGCTACAACGA AAAATATA | 4991 |
| 60 | CAACCCCC A CAACCGCC | 755 | GGCGGTTG GGCTAGCTACAACGA GGGGGTTG | 4992 |
| 115 | GCUGCAGC A UCAUCUCC | 769 | GGAGATGA GGCTAGCTACAACGA GCTGCAGC | 4993 |
| 118 | GCAGCAUC A UCUCCACC | 770 | GGTGGAGA GGCTAGCTACAACGA GATGCTGC | 4994 |
| 124 | UCAUCUCC A CCCUCCAG | 773 | CTGGAGGG GGCTAGCTACAACGA GGAGATGA | 4995 |
| 135 | CUCCAGCC A UGGAAGAC | 780 | GTCTTCCA GGCTAGCTACAACGA GGCTGGAG | 4996 |
| 182 | GACAGCCC A CCCCGGCC | 795 | GGCCGGGG GGCTAGCTACAACGA GGGCTGTC | 4997 |
| 342 | CAGUGCCC A CCGCCCCU | 824 | AGGGGCGG GGCTAGCTACAACGA GGGCACTG | 4998 |
| 494 | CCCGCGCC A UCCCCGCU | 869 | AGCGGGGA GGCTAGCTACAACGA GGCGCGGG | 4999 |
| 648 | CCCCCUCC A CCCCGGCC | 935 | GGCCGGGG GGCTAGCTACAACGA GGAGGGGG | 5000 |
| 719 | CCUGCUGC A UCUGAGCC | 957 | GGCTCAGA GGCTAGCTACAACGA GCAGCAGG | 5001 |
| 780 | CAGGUAAC A CUAUUUCG | 970 | CGAAATAG GGCTAGCTACAACGA GTTACCTG | 5002 |
| 809 | GAUUUCCC A UCUGUCCU | 976 | AGGACAGA GGCTAGCTACAACGA GGGAAATC | 5003 |
| 880 | CAAAGAAC A UGAAUACC | 1000 | GGTATTCA GGCTAGCTACAACGA GTTCTTTG | 5004 |
| 915 | UAUUACCC A CUGAAGGA | 1007 | TCCTTCAG GGCTAGCTACAACGA GGGTAATA | 5005 |
| 926 | GAAGGAAC A CUUCAAGA | 1009 | TCTTGAAG GGCTAGCTACAACGA GTTCCTTC | 5006 |
| 987 | CUCUACUC A UAGAUAGA | 1021 | TCTATCTA GGCTAGCTACAACGA GAGTAGAG | 5007 |
| 1040 | AUGGGAUC A UCGUUCAG | 1026 | CTGAACGA GGCTAGCTACAACGA GATCCCAT | 5008 |
| 1149 | GUAAUAAC A UCCUUCAU | 1038 | ATGAAGGA GGCTAGCTACAACGA GTTATTAC | 5009 |
| 1156 | CAUCCUUC A UAAUCAAC | 1041 | GTTGATTA GGCTAGCTACAACGA GAAGGATG | 5010 |
| 1301 | UUCAAACC A UUUGAGCG | 1062 | CGCTCAAA GGCTAGCTACAACGA GGTTTGAA | 5011 |
| 1435 | AACUAAUC A CGAAAAAG | 1072 | CTTTTTCG GGCTAGCTACAACGA GATTAGTT | 5012 |
| 1514 | UCAGGAGC A UAUAUCAC | 1082 | GTGATATA GGCTAGCTACAACGA GCTCCTGA | 5013 |
| 1521 | CAUAUAUC A CAUGUGCU | 1083 | AGCACATG GGCTAGCTACAACGA GATATATG | 5014 |
| 1523 | UAUAUCAC A UGUGCUCC | 1084 | GGAGCACA GGCTAGCTACAACGA GTGATATA | 5015 |
| 1557 | CUGAGAGC A UUGCAACA | 1095 | TGTTGCAA GGCTAGCTACAACGA GCTCTCAG | 5016 |
| 1569 | CAACAAAC A UUUUUCCU | 1098 | AGGAAAAA GGCTAGCTACAACGA GTTTGTTG | 5017 |
| 1668 | AUACUAGC A CCAAAACA | 1111 | TGTTTTGG GGCTAGCTACAACGA GCTAGTAT | 5018 |
| 1676 | ACCAAAAC A UCAAAGGC | 1114 | GGGTTTGA GGCTAGCTACAACGA GTTTTGGT | 5019 |
| 1700 | GUAGCAGC A CAGGAUUC | 1121 | GAATCCTG GGCTAGCTACAACGA GCTGCTAC | 5020 |
| 1725 | AUUAUGUC A CAACAGAU | 1125 | ATCTGTTG GGCTAGCTACAACGA GACATAAT | 5021 |
| 1770 | UGGCAAAC A UGCCUGAA | 1131 | TTCAGGCA GGCTAGCTACAACGA GTTTGCCA | 5022 |
| 1808 | CAGGAAGC A UGUGAAAG | 1140 | CTTTCACA GGCTAGCTACAACGA GCTTCCTG | 5023 |
| 1880 | GUUCAAAC A UCAGAAGU | 1147 | ACTTCTGA GGCTAGCTACAACGA GTTTGAAC | 5024 |
| 1901 | CAAGAGUC A CUCUAUGC | 1150 | GGATAGAG GGCTAGCTACAACGA GACTCTTG | 5025 |
| 1916 | CCUGCAGC A CAGCUUUG | 1156 | CAAAGCTG GGCTAGCTACAACGA GCTGCAGG | 5026 |
| 1928 | CUUUGCCC A UCAUUUGA | 1161 | TCAAATGA GGCTAGCTACAACGA GGGCAAAG | 5027 |
| 1931 | UGCCCAUC A UUUGAAGA | 1162 | TCTTCAAA GGCTAGCTACAACGA GATGGGCA | 5028 |
| 1958 | ACUCCUUC A CCAGUUUU | 1168 | AAAACTGG GGCTAGCTACAACGA GAAGGAGT | 5029 |
| 1974 | UGCCUGAC A UUGUUAUG | 1173 | CATAACAA GGCTAGCTACAACGA GTCAGGCA | 5030 |
| 1988 | AUGGAAGC A CCAUUGAA | 1174 | TTCAATGG GGCTAGCTACAACGA GCTTCCAT | 5031 |
| 1991 | GAAGCACC A UUGAAUUC | 1176 | GAATTCAA GGCTAGCTACAACGA GGTGCTTC | 5032 |
| 2042 | CCCAGCUC A UCACCAUU | 1189 | AATGGTGA GGCTAGCTACAACGA GAGCTGGG | 5033 |
| 2045 | AGCUCAUC A CCAUUAGA | 1190 | TCTAATGG GGCTAGCTACAACGA GATGAGCT | 5034 |
| 2048 | UGAUGACC A UUAGAAGC | 1192 | GCTTCTAA GGCTAGCTACAACGA GGTGATGA | 5035 |
| 2079 | AUGAAAGC A UAAAACAU | 1196 | ATGTTTTA GGCTAGCTACAACGA GCTTTCAT | 5036 |
| 2086 | CAUAAAAC A UGAGCCUG | 1197 | CAGGCTCA GGCTAGCTACAACGA GTTTTATG | 5037 |
| 2105 | AACCCCCC A CCAUAUGA | 1205 | TCATATGG GGCTAGCTACAACGA GGGGGGTT | 5038 |
| 2108 | CCCCCACC A UAUGAAGA | 1207 | TCTTCATA GGCTAGCTACAACGA GGTGGGGG | 5039 |
| 2121 | AAGAGGCC A UGAGUGUA | 1209 | TACACTCA GGCTAGCTACAACGA GGCCTCTT | 5040 |
| 2132 | AGUGUAUC A CUAAAAAA | 1210 | TTTTTTAG GGCTAGCTACAACGA GATACACT | 5041 |
| 2228 | UCUAUUGC A UGUGAUUU | 1224 | AAATCACA GGCTAGCTACAACGA GCAATAGA | 5042 |
| 2326 | GCCUGAUG A UUCUGAGC | 1242 | GCTCAGAA GGCTAGCTACAACGA GATCAGGC | 5043 |
| 2351 | GAUUCCUC A CCUGAUUC | 1247 | GAATCAGG GGCTAGCTACAACGA GAGGAATC | 5044 |
| 2405 | GACGUUCC A CAAAAACA | 1258 | TGTTTTTG GGCTAGCTACAACGA GGAACGTC | 5045 |
| 2448 | AAAGUCUC A CUGAGACU | 1264 | AGTCTCAG GGCTAGCTACAACGA GAGACTTT | 5046 |
| 2459 | GAGACUUC A UUUGAGUC | 1267 | GACTCAAA GGCTAGCTACAACGA GAAGTCTC | 5047 |
| 2510 | GCUUUGCC A CCUGAGGG | 1273 | CCCTCAGG GGCTAGCTACAACGA GGCAAAGC | 5048 |
| 2528 | GGAAAGGC A UAUUUGGA | 1277 | TCCAAATA GGCTAGCTACAACGA GGCTTTCC | 5049 |
| 2562 | UAGAUAAC A CAAAAGAU | 1281 | ATCTTTTG GGCTAGCTACAACGA GTTATCTA | 5050 |
| 2597 | GUUUCAAC A UUGAGCAA | 1289 | TTGCTCAA GGCTAGCTACAACGA GTTGAAAC | 5051 |
| 2687 | AAGGAAGC A CAGAUAAG | 1301 | CTTATCTG GGCTAGCTACAACGA GCTTCCTT | 5052 |
| 2720 | UCAGAUUC A UCUCCAAU | 1305 | ATTGGAGA GGCTAGCTACAACGA GAATCTGA | 5053 |
| 2753 | UUCCCUAC A UUGAUGAG | 1312 | CTGATCAA GGCTAGCTACAACGA GTAGGGAA | 5054 |
| 2777 | ACUGAUUC A UUUUCUAA | 1316 | TTAGAAAA GGCTAGCTACAACGA GAATCAGT | 5055 |
| 2821 | AGUAUCCC A CAAAAGUG | 1325 | CACTTTTG GGCTAGCTACAACGA GGGATACT | 5056 |
| 2861 | GCUGGGUC A UUGCCUUG | 1332 | CAAGGCAA GGCTAGCTACAACGA GACCCAGC | 5057 |
| 2871 | UGCCUUGC A CAGAAUUG | 1335 | CAATTCTG GGCTAGCTACAACGA GCAAGGCA | 5058 |
| 2884 | AUUGCCCC A UGACCUUU | 1340 | AAAGGTCA GGCTAGCTACAACGA GGGGCAAT | 5059 |
| 2904 | UGAAGAAC A UACAACCC | 1344 | GGGTTGTA GGCTAGCTACAACGA GTTCTTCA | 5060 |
| 2969 | UCUGCUAC A UCAAAGGU | 1356 | ACCTTTGA GGCTAGCTACAACGA GTAGCAGA | 5061 |
| 3012 | CUUUGGCC A CUCAAGCA | 1367 | TGCTTGAG GGCTAGCTACAACGA GGCCAAAG | 5062 |
| 3033 | UAGAGAGC A UAGUUAAA | 1371 | TTTAACTA GGCTAGCTACAACGA GCTCTCTA | 5063 |
| 3107 | GACAGAUC A CCAUCUGC | 1383 | GCAGATGG GGCTAGCTACAACGA GATCTGTC | 5064 |
| 3110 | AGAUCACC A UCUGCUAU | 1385 | ATAGCAGA GGCTAGCTACAACGA GGTGATCT | 5065 |
| 3174 | GGAGAGAC A UUAAGAAG | 1398 | CTTCTTAA GGCTAGCTACAACGA GTCTCTCC | 5066 |
| 3224 | CUGCUUUC A UUGACAGU | 1408 | ACTGTCAA GGCTAGCTACAACGA GAAAGCAG | 5067 |
| 3240 | UAUUCAGC A UUGUGAGC | 1411 | GCTCACAA GGCTAGCTACAACGA GCTGAATA | 5068 |
| 3261 | CAGCCUAC A UUGCCUUG | 1415 | CAAGGCAA GGCTAGCTACAACGA GTAGGCTG | 5069 |
| 3288 | CUGUGACC A UCAGCUUU | 1425 | AAAGCTGA GGCTAGCTACAACGA GGTCACAG | 5070 |
| 3346 | UGAAGGCC A CCCAGUCA | 1436 | TGAATGGG GGCTAGCTACAACGA GGCCTTCA | 5071 |
| 3350 | GGCCACCC A UUCAGGGC | 1439 | GCCCTGAA GGCTAGCTACAACGA GGGTGGCC | 5072 |
| 3359 | UUCAGGGC A UAUCUGGA | 1441 | TCCAGATA GGCTAGCTACAACGA GCCCTGAA | 5073 |
| 3427 | UCUUGGUC A UGUGAACU | 1451 | AGTTCACA GGCTAGCTACAACGA GACCAAGA | 5074 |
| 3438 | UGAACUGC A CGAUAAAG | 1453 | CTTTATCG GGCTAGCTACAACGA GCAGTTCA | 5075 |
| 3551 | CGUCUGAC A CUACUGAU | 1468 | ATCAGTAG GGCTAGCTACAACGA GTCAGACC | 5076 |
| 3570 | UGGCUCUC A UUUCACUC | 1473 | GAGTGAAA GGCTAGCTACAACGA GAGAGCCA | 5077 |
| 3575 | CUCAUUUC A CUCUUCAG | 1474 | CTGAAGAG GGCTAGCTACAACGA GAAATGAG | 5078 |
| 3607 | UGAACGGC A UCAGGCAC | 1480 | GTGCCTGA GGCTAGCTACAACGA GCCGTTCA | 5079 |
| 3614 | CAUCAGGC A CAGAUAGA | 1482 | TCTATCTG GGCTAGCTACAACGA GCCTGATG | 5080 |
| 3625 | GAGAGAUC A UUAUCUAG | 1484 | CTAGATAA GGCTAGCTACAACGA GATCTATC | 5081 |
| 3742 | AGGAGUUC A UCUUUAAA | 1501 | TTTAAAGA GGCTAGCTACAACGA GAACTCCT | 5082 |
| 3761 | GGAUAUUC A UUUGAUUA | 1503 | TAATCAAA GGCTAGCTACAACGA GAATATCC | 5083 |
| 3820 | GCAGUUUC A CAGAUCGU | 1509 | ACGATCTG GGCTAGCTACAACGA GAAACTGC | 5084 |
| 3851 | UUUUAGCC A UGCACUGU | 1513 | ACAGTGCA GGCTAGCTACAACGA GGCTAAAA | 5085 |
| 3855 | AGGCAUGC A CUGUGGUG | 1514 | CACAACAG GGCTAGCTACAACGA GCATGGCT | 5086 |
| 3890 | UGACUGCC A UGUGUUCA | 1521 | TGAACACA GGCTAGCTACAACGA GGCAGTCA | 5087 |
| 3898 | AUGUGGUC A UCAUCUUA | 1522 | TAAGATGA GGCTAGCTACAACGA GAACACAT | 5088 |
| 3901 | UGUUCAUC A UCUUAAGU | 1523 | ACTTAAGA GGCTAGCTACAACGA GATGAACA | 5089 |
| 3946 | CCGUAAUC A UAUCUUUU | 1528 | AAAAGATA GGCTAGCTACAACGA GATTACGG | 5090 |
| 3968 | UCUGAGGC A CUGGUGGA | 1533 | TCCACCAG GGCTAGCTACAACGA GCCTCAGA | 5091 |
| 4022 | CUUGCCGC A UCUUGGCA | 1541 | TGCCAAGA GGCTAGCTACAACGA GCGGCAAG | 5092 |
| 66 | CCACAACC G CCCGCGGC | 1545 | GCCGCGGG GGCTAGCTACAACGA GGTTGTGG | 5093 |
| 70 | AACCGCCC G CGGCUCUG | 1546 | CAGAGCCG GGCTAGCTACAACGA GGGCGGTT | 5094 |
| 83 | UCUGAGAC G CGGCCCCG | 1548 | CGGGGCCG GGCTAGCTACAACGA GTCTCAGA | 5095 |
| 110 | CAGCAGCU G CAGCAUCA | 1549 | TGATGCTG GGCTAGCTACAACGA AGCTGCTG | 5096 |
| 191 | CCCCGGCC G CAGCCCGC | 1556 | GCGGGCTG GGCTAGCTACAACGA GGCCGGGG | 5097 |
| 198 | CGCAGCCC G CGUUCAAG | 1551 | CTTGAACG GGCTAGCTACAACGA GGGCTGCG | 5098 |
| 296 | CUGGAGGU G CUGGAGAG | 1557 | CTCTCCAG GGCTAGCTACAACGA ACCTCCAG | 5099 |
| 312 | GGAAGCCC G CCGCCGGG | 1558 | CCCGGCGG GGCTAGCTACAACGA GGGCTTCC | 5100 |
| 315 | AGCCCGCC G CCGGGCUG | 1559 | CAGCCCGG GGCTAGCTACAACGA GGCGGGCT | 5101 |
| 327 | GGCUGUCC G CGGCCCCA | 1550 | TGGGGCCG GGCTAGCTACAACGA GGACAGCC | 5102 |
| 338 | GCCCCAGU G CCCACCGC | 1551 | GCGGTGGG GGCTAGCTACAACGA ACTGGGGC | 5103 |
| 345 | UGCCCACC G CCCCUGCC | 1562 | GGCACGGG GGCTAGCTACAACGA GGTGGGCA | 5104 |
| 351 | CCGCCCCU G CCGCCGGC | 1563 | GCCGGCGG GGCTAGCTACAACGA AGGGGCGG | 5105 |
| 354 | CCCCUGCC G CCGGCGCG | 1564 | CGCGCCGG GGCTAGCTACAACGA GGCAGGGG | 5106 |
| 360 | CCGCCGGC G CGCCCCUG | 1565 | CAGGGGCG GGCTAGCTACAACGA GCCGGCGG | 5107 |
| 362 | GCCGGCGC G CCCCUGAU | 1566 | ATCAGGGG GGCTAGCTACAACGA GCGCCGGC | 5108 |
| 392 | GACUUCGU G CCGCCGGC | 1569 | GCCGGCGG GGCTAGCTACAACGA ACGAAGTC | 5109 |
| 395 | UUCGUGCC G CCGGCGCC | 1570 | GGCGCCGG GGCTAGCTACAACGA GGCACGAA | 5110 |
| 401 | CCGCCGGC G CCCCGGGG | 1571 | CCCCGGGG GGCTAGCTACAACGA GCCGGCGG | 5111 |
| 416 | GGACCCCU G CCGGCCGC | 1572 | GCGGCCGG GGCTAGCTACAACGA AGGGGTCC | 5112 |
| 423 | UGCCGGCC G CUCCCCCC | 1573 | GGGGGGAG GGCTAGCTACAACGA GGCCGGCA | 5113 |
| 435 | CCCCCGUC G CCCCGGAG | 1574 | CTCCGGGG GGCTAGCTACAACGA GACGGGGG | 5114 |
| 485 | UCGACCGU G CCCGCGCC | 1577 | GGCGCGGG GGCTAGCTACAACGA ACGGTCGA | 5115 |
| 489 | CCGUGCCC G CGCCAUCC | 1578 | GGATGGCG GGCTAGCTACAACGA GGGCACGG | 5116 |
| 491 | GUGCCCGC G CCAUCCCC | 1579 | GGGGATGG GGCTAGCTACAACGA GCGGGCAC | 5117 |
| 500 | CCAUCCCC G CUGUCUGC | 1580 | GCAGACAG GGCTAGCTACAACGA GGGGATGG | 5118 |
| 507 | CGCUGUCU G CUGCCGCA | 1581 | TGCGGCAG GGCTAGCTACAACGA AGACAGCG | 5119 |
| 510 | UGUCUGCU G CCGCAGUC | 1582 | GACTGCGG GGCTAGCTACAACGA AGCAGACA | 5120 |
| 513 | CUGCUGCC G CAGUCUCG | 1583 | CGAGACTG GGCTAGCTACAACGA GGCAGCAG | 5121 |
| 521 | GCAGUCUC G CCCUCCAA | 1584 | TTGGAGGG GGCTAGCTACAACGA GAGACTGC | 5122 |
| 617 | UGGACCCC G CCAGCCCC | 1589 | GGGGCTGG GGCTAGCTACAACGA GGGGTCCA | 5123 |
| 633 | CGGCUCCC G CCGCGCCC | 1590 | CGCCCCGG GGCTAGCTACAACGA GGGAGCCG | 5124 |
| 636 | CUCCCGCC G CGCCCCCC | 1591 | GGGGGGCG GGCTAGCTACAACGA GGCGGGAG | 5125 |
| 638 | CCCGCCGC G CCCCCCUC | 1592 | GAGGGGGG GGCTAGCTACAACGA GCGGCGGG | 5126 |
| 657 | CCCCGGCC G CGCCCAAG | 1593 | CTTGGGCG GGCTAGCTACAACGA GGCCGGGG | 5127 |
| 659 | CCGGCCGC G CCCAAGCG | 1594 | CGCTTGGG GGCTAGCTACAACGA GCGGCCGG | 5128 |
| 667 | GCCCAAGC G CAGGGGCU | 1595 | AGCCCCTG GGCTAGCTACAACGA GCTTGGGC | 5129 |
| 705 | CCCUUUUU G CUCUUCCU | 1597 | AGGAAGAG GGCTAGCTACAACGA AAAAAGGG | 5130 |
| 714 | CUCUUCCU G CUGCAUCU | 1598 | AGATGCAG GGCTAGCTACAACGA AGGAAGAG | 5131 |
| 717 | UUCCUGCU G CAUCUGAG | 1599 | CTCAGATG GGCTAGCTACAACGA AGCAGGAA | 5132 |
| 736 | UGUGAUAC G CUCCUCUG | 1602 | CAGAGGAG GGCTAGCTACAACGA GTATCACA | 5133 |
| 744 | GCUCCUCU G CAGAAAAU | 1603 | ATTTTCTG GGCTAGCTACAACGA AGAGGAGC | 5134 |
| 818 | UCUGUCCU G CUUGAAAC | 1605 | GTTTCAAG GGCTAGCTACAACGA AGGACAGA | 5135 |
| 828 | UUGAAACU G CUGCUUCU | 1607 | AGAAGCAG GGCTAGCTACAACGA AGTTTCAA | 5136 |
| 831 | AAACUGCU G CUUCUCUU | 1608 | AAGAGAAG GGCTAGCTACAACGA AGCAGTTT | 5137 |
| 864 | UCUCAGCC G CUUCUUUC | 1609 | GAAAGAAG GGCTAGCTACAACGA GCCTCAGA | 5138 |
| 1071 | CAGAAUCU G CCGUAAUA | 1613 | TATTACGG GGCTAGCTACAACGA AGATTCTG | 5139 |
| 1257 | AGAGAGUU G CAGUGGAA | 1618 | TTCCACTG GGCTAGCTACAACGA AACTCTCT | 5140 |
| 1287 | AGGAAUAU G CAGACUUC | 1620 | GAAGTCTG GGCTAGCTACAACGA ATATTCCT | 5141 |
| 1356 | UGUUGGCU G CUGGAGGU | 1625 | ACCTCCAG GGCTAGCTACAACGA AGCCAACA | 5142 |
| 1410 | AAUGUUUU G CAGAUAGC | 1627 | GCTATCTG GGCTAGCTACAACGA AAAACATT | 5143 |
| 1484 | CCCAGUAC G CCACAAGG | 1633 | CCTTCTGG GGCTAGCTACAACGA GTACTGGG | 5144 |
| 1527 | UCACAUGU G CUCCCUUU | 1634 | AAAGGGAG GGCTAGCTACAACGA ACATGTGA | 5145 |
| 1560 | AGAGCAUU G CAACAAAC | 1636 | GTTTGTTG GGCTAGCTACAACGA AATGCTCT | 5146 |
| 1772 | GCAAACAU G CCUGAAGG | 1642 | CCTTCAGG GGCTAGCTACAACGA ATGTTTGC | 5147 |
| 1848 | GAAAGAUU G CUUAUGAA | 1649 | TTCATAAG GGCTAGCTACAACGA AATCTTTG | 5148 |
| 1892 | GAAGUUAU G CAAGAGUC | 1651 | GACTCTTG GGCTAGCTACAACGA ATAACTTC | 5149 |
| 1911 | UCUAUCCU G CAGCACAG | 1652 | CTGTGCTG GGCTAGCTACAACGA AGGATAGA | 5150 |
| 1924 | ACAGCUUU G CCCAUCAU | 1653 | ATGATGGG GGCTAGCTACAACGA AAAGCTGT | 5151 |
| 1967 | CCAGUUUU G CCUCACAU | 1655 | ATGTCAGG GGCTAGCTACAACGA AAAACTGG | 5152 |
| 2001 | UGAAUUCU G CAGUUCCU | 1658 | AGGAACTG GGCTAGCTACAACGA AGAATTCA | 5153 |
| 2013 | UUCCUAGU G CUGGUGCU | 1659 | AGCACCAG GGCTAGCTACAACGA ACTAGGAA | 5154 |
| 2019 | GUGCUGGU G CUUCCGUG | 1660 | CACGGAAG GGCTAGCTACAACGA ACCAGCAC | 5155 |
| 2187 | AUAUUAAU G CAGCUCUU | 1668 | AAGAGCTG GGCTAGCTACAACGA ATTAATAT | 5156 |
| 2226 | UAUCUAUU G CAUGUGAU | 1669 | ATCACATG GGCTAGCTACAACGA AATAGATA | 5157 |
| 2259 | AGCUUUCU G CUGAACCA | 1671 | TGGTTCAG GGCTAGCTACAACGA AGAAAGCT | 5158 |
| 2318 | CAGCCAGU G CCUGAUGA | 1675 | TGATCAGG GGCTAGCTACAACGA ACTGGCTG | 5159 |
| 2429 | ACUGUGAU G CUUGUGAA | 1687 | TTCACAAG GGCTAGCTACAACGA ATCACAGT | 5160 |
| 2502 | AACUCAGU G CUUUGCCA | 1693 | TGGCAAAG GGCTAGCTACAACGA ACTGAGTT | 5161 |
| 2507 | AGUGCUUU G CCACCUGA | 1694 | TCAGGTGG GGCTAGCTACAACGA AAAGCACT | 5162 |
| 2624 | AUUCCUUU G CAGAUGGA | 1699 | TCCATCTG GGCTAGCTACAACGA AAAGGAAT | 5163 |
| 2646 | UCAGUACU G CAGUUUAU | 1700 | ATAAACTG GGCTAGCTACAACGA AGTACTGA | 5164 |
| 2835 | GUGAAAUU G CUAAUGCC | 1710 | GGCATTAG GGCTAGCTACAACGA AATTTCAC | 5165 |
| 2841 | UUGCUAAU G CCCCGGAU | 1711 | ATCCGGGG GGCTAGCTACAACGA ATTAGCAA | 5166 |
| 2864 | GGGUCAUU G CCUUGCAC | 1712 | GTGCAAGG GGCTAGCTACAACGA AATGACCC | 5167 |
| 2869 | AUUGCCUU G CACAGAAU | 1713 | ATTCTGTG GGCTAGCTACAACGA AAGGCAAT | 5168 |
| 2879 | ACAGAAUU G CCCCAUGA | 1714 | TCATGGGG GGCTAGCTACAACGA AATTCTGT | 5169 |
| 2964 | AUGGGUCU G CUACAUCA | 1719 | TGATGTAG GGCTAGCTACAACGA AGACCCAT | 5170 |
| 2978 | UCAAAGGU G CUCUUAUU | 1720 | AATAAGAG GGCTAGCTACAACGA ACCTTTGA | 5171 |
| 2987 | CUCUUAUU G CCUCCAGA | 1721 | TCTGGAGG GGCTAGCTACAACGA AATAAGAG | 5172 |
| 3003 | AUGGGUCU G CUUUGGCC | 1722 | GGCCAAAG GGCTAGCTACAACGA AGAAACAT | 5173 |
| 3114 | CACCAUCU G CUAUAUUU | 1726 | AAATATAG GGCTAGCTACAACGA AGATGGTG | 5174 |
| 3201 | UGUUUGGU G CCAGCCUA | 1729 | TAGGCTGG GGCTAGCTACAACGA ACCAAACA | 5175 |
| 3215 | CUAUUCCU G CUCCUUUC | 1730 | CAAAGCAC GGCTAGCTACAACGA AGGAATAG | 5176 |
| 3218 | UUCCUGCU G CUUUCAUU | 1731 | AATGAAAG GGCTAGCTACAACGA AGCAGGAA | 5177 |
| 3264 | CCUACAUU G CCUUGGCC | 1734 | CGCCAAGC GGCTAGCTACAACGA AATGTAGG | 5178 |
| 3275 | UUGGCCCU G CUCUCUGU | 1735 | ACAGAGAG GGCTAGCTACAACGA AGGGCCAA | 5179 |
| 3378 | CUGAAGUU G CUAUAUCU | 1740 | AGATATAG GGCTAGCTACAACGA AACTTCAG | 5180 |
| 3417 | GUAAUUCU G CUCUUGGU | 1742 | ACCAAGAG GGCTAGCTACAACGA AGAATTAC | 5181 |
| 3436 | UGUGAACU G CACGAUAA | 1744 | TTATCGTG GGCTAGCTACAACGA AGTTCACA | 5182 |
| 3457 | ACUCAGGC G CCUCUUCU | 1746 | AGAAGAGG GGCTAGCTACAACGA GCCTGAGT | 5183 |
| 3498 | UGAAGUUU G CAGUGUUG | 1751 | CAACACTG GGCTAGCTACAACGA AAACTTCA | 5184 |
| 3531 | AUGUUGGU G CCUUGUUU | 1753 | AAACAAGG GGCTAGCTACAACGA ACCAACAT | 5185 |
| 3639 | UAGGACUU G CAAAUAAG | 1757 | CTTATTTG GGCTAGCTACAACGA AAGTCCTA | 5186 |
| 3660 | UUAAAGAU G CUAUGGCU | 1758 | AGCCATAG GGCTAGCTACAACGA ATCTTTAA | 5187 |
| 3700 | AUUGAAGC G CAAAGCUG | 1760 | CAGCTTTC GGCTAGCTACAACGA GCTTCAAT | 5188 |
| 3718 | AUGAAAAC G CCCAAAAU | 1763 | ATTTTGGG GGCTAGCTACAACGA GTTTTCAT | 5189 |
| 3807 | UUGACGUU G CACUGCAC | 1767 | CTGCACTG GGCTAGCTACAACGA AACGTCAA | 5190 |
| 3812 | GUUGCAGU G CAGUUUCA | 1768 | TCAAACTC GGCTAGCTACAACGA ACTGCAAC | 5191 |
| 3853 | UUAGCCAU G CACUCUUG | 1769 | CAACAGTG GGCTAGCTACAACGA ATGGCTAA | 5192 |
| 3887 | UCUUGACU G CCAUGUGU | 1772 | ACACATCG GGCTAGCTACAACGA AGTCAAGA | 5193 |
| 3920 | UGUAAGCU G CUAUGUAU | 1773 | ATACATAC GGCTAGCTACAACGA AGCTTACA | 5194 |
| 4005 | ACUUUGUU G CAGAUAGU | 1775 | ACTATCTG GGCTAGCTACAACGA AACAAAGT | 5195 |
| 4017 | AUAGUCUU G CCGCAUCU | 1776 | ACATCCCC GGCTAGCTACAACGA AAGACTAT | 5196 |
| 4020 | CUCUUgCC G CAUCUUGG | 1777 | CCAAGATG GGCTAGCTACAACGA GGCAAGAC | 5197 |
| 4035 | GGCAAGUU G CAGAGAUG | 1778 | CATCTCTG GGCTAGCTACAACGA AACTTGCC | 5198 |
| 12 | CACAGUAG G UCCCUCGG | 1779 | CCGAGGGA GGCTAGCTACAACGA CTACTCTC | 5199 |
| 20 | GUCCCUCG G CUCAGUCG | 1780 | CGACTGAG GGCTAGCTACAACGA CGAGGGAC | 5200 |
| 25 | UGGCCUCA G UCGGCCCA | 1781 | TGGGCCGA GGCTAGCTACAACGA TGAGCCCA | 5201 |
| 29 | CUCACUGG G CCCAGCCC | 1782 | GGGCTGGG GGCTAGCTACAACGA CGACTGAG | 5202 |
| 34 | UCGGCCCA G CCCCUCUC | 1783 | GAGAGGGG GGCTAGCTACAACGA TGGGCCGA | 5203 |
| 44 | CCCUCUCA G UCCUCCCC | 1784 | GGGGAGGA GGCTAGCTACAACGA TGAGAGGG | 5204 |
| 73 | CGCCCGCG G CUCUGAGA | 1785 | TCTCAGAG GGCTAGCTACAACGA CGCGGGCG | 5205 |
| 86 | GAGACGCG G CCCCGGCG | 1786 | CGCCGGGG GGCTAGCTACAACGA CGCGTCTC | 5206 |
| 92 | CGGCCCCG G CGGCGGCG | 1787 | CGCCGCCG GGCTAGCTACAACGA CGGGGCCG | 5207 |
| 95 | CCCCGGCG G CGGCGGCA | 1788 | TGCCGCCG GGCTAGCTACAACGA CGCCGGGG | 5208 |
| 98 | CGGCGGCG G CGGCAGCA | 1789 | TGCTGCCG GGCTAGCTACAACGA CGCCGCCG | 5209 |
| 101 | CGGCGGCG G CAGCAGCU | 1790 | AGCTGCTG GGCTAGCTACAACGA CGCCGCCG | 5210 |
| 104 | CGGCGGCA G CAGCUGCA | 1791 | TGCAGCTG GGCTAGCTACAACGA TGCCGCCG | 5211 |
| 107 | CGGCAGCA G CUGCAGCA | 1792 | TGCTGCAG GGCTAGCTACAACGA TGCTGCCG | 5212 |
| 113 | CAGCUGCA G CAUCAUCU | 1793 | AGATGATG GGCTAGCTACAACGA TGCAGCTG | 5213 |
| 132 | ACCCUCCA G CCAUGGAA | 1794 | TTCCATGG GGCTAGCTACAACGA TGGAGGGT | 5214 |
| 152 | CUGGACCA G UCUCCUCU | 1795 | AGAGGAGA GGCTAGCTACAACGA TGGTCCAG | 5215 |
| 162 | CUCCUCUG G UCUCGUCC | 1796 | GGACGAGA GGCTAGCTACAACGA CAGAGGAG | 5216 |
| 167 | CUGGUCUC G UCCUCGGA | 1797 | TCCGAGGA GGCTAGCTACAACGA GAGACCAG | 5217 |
| 178 | CUCGGACA G CCCACCCC | 1798 | GGGGTGGG GGCTAGCTACAACGA TGTCCGAG | 5218 |
| 188 | CCACCCCG G CCGCAGCC | 1799 | GGCTGCGG GGCTAGCTACAACGA CGGGGTGG | 5219 |
| 194 | CGGCCGCA G CCCGCGUU | 1800 | AACGCGGG GGCTAGCTACAACGA TGCGGCCG | 5220 |
| 200 | CAGCCCGC G UUCAAGUA | 1801 | TACTTGAA GGCTAGCTACAACGA GCGGGCTG | 5221 |
| 206 | GCGUUCAA G UACCAGUU | 1802 | AACTGGTA GGCTAGCTACAACGA TTGAACGC | 5222 |
| 212 | AAGUACCA G UUCGUGAG | 1803 | CTCACGAA GGCTAGCTACAACGA TGGTACTT | 5223 |
| 216 | ACCAGUUC G UGAGGGAG | 1804 | CTCCCTCA GGCTAGCTACAACGA GAACTGGT | 5224 |
| 224 | GUGAGGGA G CCCGAGGA | 1805 | TCCTCGGG GGCTAGCTACAACGA TCCCTCAC | 5225 |
| 287 | CUGGAGGA G CUGGAGGU | 1806 | ACCTCCAG GGCTAGCTACAACGA TCCTCCAG | 5226 |
| 294 | AGCUGGAG G UGCUGGAG | 1807 | CTCCAGCA GGCTAGCTACAACGA CTCCAGCT | 5227 |
| 308 | GAGAGGAA G CCCGCCGC | 1808 | GCGGCGGG GGCTAGCTACAACGA TTCCTCTC | 5228 |
| 320 | GCCGCCGG G CUGUCCGC | 1809 | GCGGACAG GGCTAGCTACAACGA CCGGCGGC | 5229 |
| 323 | GCCGGGCU G UCCGCGGC | 1810 | GCCGCGGA GGCTAGCTACAACGA AGCCCGGC | 5230 |
| 330 | UGUCCGCG G CCCCAGUG | 1811 | CACTGGGG GGCTAGCTACAACGA CGCGGACA | 5231 |
| 336 | CGGCCCCA G UGCCCACC | 1812 | GGTGGGCA GGCTAGCTACAACGA TGGGGCCG | 5232 |
| 358 | UGCCGCCG G CGCGCCCC | 1813 | GGGGCGCG GGCTAGCTACAACGA CGGCGGCA | 5233 |
| 390 | AUGACUUC G UGCCGCCG | 1814 | CGGCGGCA GGCTAGCTACAACGA GAAGTCAT | 5234 |
| 399 | UGCCGCCG G CGCCCCGG | 1815 | CCGGGGCG GGCTAGCTACAACGA CGGCGGCA | 5235 |
| 420 | CCCUGCCG G CCGCUCCC | 1816 | GGGAGCGG GGCTAGCTACAACGA CGGCAGGG | 5236 |
| 432 | CUCCCCCC G UCGCCCCG | 1817 | CGGGGCGA GGCTAGCTACAACGA GGGGGGAG | 5237 |
| 443 | GCCCCGGA G CGGCAGCC | 1818 | GGCTGCCG GGCTAGCTACAACGA TCCGGGGC | 5238 |
| 446 | CCGGAGCG G CAGCCGUC | 1819 | GACGGCTG GGCTAGCTACAACGA CGCTCCGG | 5239 |
| 449 | GAGCGGCA G CCGUCUUG | 1820 | CAAGACGG GGCTAGCTACAACGA TGCCGCTC | 5240 |
| 452 | CGGCAGCC G UCUUGGGA | 1821 | TCCCAAGA GGCTAGCTACAACGA GGCTGCCG | 5241 |
| 466 | GGACCCGA G CCCGGUGU | 1822 | ACACCGGG GGCTAGCTACAACGA TCGGGTCC | 5242 |
| 471 | CGAGCCCG G UGUCGUCG | 1823 | CGACGACA GGCTAGCTACAACGA CGGGCTCG | 5243 |
| 473 | AGCCCGGU G UCGUCGAC | 1824 | GTCGACGA GGCTAGCTACAACGA ACCGGGCT | 5244 |
| 476 | CCGGUGUC G UCGACCGU | 1825 | ACGGTCGA GGCTAGCTACAACGA GACACCGG | 5245 |
| 483 | CGUCGACC G UGCCCGCG | 1826 | CGCGGGCA GGCTAGCTACAACGA GGTCGACG | 5246 |
| 503 | UCCCCGCU G UCUGCUGC | 1827 | GCAGCAGA GGCTAGCTACAACGA AGCGGGGA | 5247 |
| 516 | CUGCCGCA G UCUCGCCC | 1828 | GGGCGAGA GGCTAGCTACAACGA TGCGGCAG | 5248 |
| 530 | CCCUCCAA G CUCCCUGA | 1829 | TCAGGGAG GGCTAGCTACAACGA TTGGAGGG | 5249 |
| 548 | GACGACGA G CCUCCGGC | 1830 | GCCGGAGG GGCTAGCTACAACGA TCGTCGTC | 5250 |
| 555 | AGCCUCCG G CCCGGCCU | 1831 | AGGCCGGG GGCTAGCTACAACGA CGGAGGCT | 5251 |
| 560 | CCGGCCCG G CCUCCCCC | 1832 | GGGGGAGG GGCTAGCTACAACGA CGGGCCGG | 5252 |
| 579 | CUCCCCCG G CCAGCGUG | 1833 | CACGCTGG GGCTAGCTACAACGA CGGGGGAG | 5253 |
| 583 | CCCGGCCA G CGUGAGCC | 1834 | GGCTCACG GGCTAGCTACAACGA TGGCCGGG | 5254 |
| 585 | CGGCCAGC G UGAGCCCC | 1835 | GGGGCTCA GGCTAGCTACAACGA GCTGGCCG | 5255 |
| 589 | CAGCGUGA G CCCCCAGG | 1836 | CCTGGGGG GGCTAGCTACAACGA TCACGCTG | 5256 |
| 597 | GCCCCCAG G CAGAGCCC | 1837 | GGGCTCTG GGCTAGCTACAACGA CTGGGGGC | 5257 |
| 602 | CAGGCAGA G CCCGUGUG | 1838 | CACACGGG GGCTAGCTACAACGA TCTGCCTG | 5258 |
| 606 | CAGAGCCC G UGUGGACC | 1839 | GGTCCACA GGCTAGCTACAACGA GGGCTCTG | 5259 |
| 608 | GAGCCCGU G UGGACCCC | 1840 | GGGGTCCA GGCTAGCTACAACGA ACGGGCTC | 5260 |
| 621 | CCCCGCCA G CCCCGGCU | 1841 | AGCCGGGG GGCTAGCTACAACGA TGGCGGGG | 5261 |
| 627 | CAGCCCCG G CUCCCGCC | 1842 | GGCGGGAG GGCTAGCTACAACGA CGGGGCTG | 5262 |
| 654 | CCACCCCG G CCGCGCCC | 1843 | GGGCGCGG GGCTAGCTACAACGA CGGGGTGG | 5263 |
| 665 | GCGCCCAA G CGCAGGGG | 1844 | CCCCTGCG GGCTAGCTACAACGA TTGGGCGC | 5264 |
| 673 | GCGCAGGG G CUCCUCGG | 1845 | CCGAGGAG GGCTAGCTACAACGA CCCTGCGC | 5265 |
| 682 | CUCCUCGG G CUCAGUGG | 1846 | CCACTGAG GGCTAGCTACAACGA CCGAGGAG | 5266 |
| 687 | CGGGCUCA G UGGAUGAG | 1847 | CTCATCCA GGCTAGCTACAACGA TGAGCCCG | 5267 |
| 725 | GCAUCUGA G CCUGUGAU | 1848 | ATCACAGG GGCTAGCTACAACGA TCAGATGC | 5268 |
| 729 | CUGAGCCU G UGAUACGC | 1849 | GCGTATCA GGCTAGCTACAACGA AGGCTCAG | 5269 |
| 767 | UUGAAGGA G CAGCCAGG | 1850 | CCTCCCTG GGCTAGCTACAACGA TCCTTCAA | 5270 |
| 770 | AAGGAGCA G CCAGGUAA | 1851 | TTACCTGG GGCTAGCTACAACGA TGCTCCTT | 5271 |
| 775 | GCAGCCAG G UAACACUA | 1852 | TAGTGTTA GGCTAGCTACAACGA CTGGCTGC | 5272 |
| 789 | CUAUUUCG G CUGGUCAA | 1853 | TTGACCAG GGCTAGCTACAACGA CGAAATAG | 5273 |
| 793 | UUCGGCUG G UCAAGAGG | 1854 | CCTCTTGA GGCTAGCTACAACGA CAGCCGAA | 5274 |
| 813 | UCCCAUCU G UCCUGCUU | 1855 | AAGCAGGA GGCTAGCTACAACGA AGATGGGA | 5275 |
| 848 | CCUUCUCU G UCUCCUCU | 1856 | AGAGGAGA GGCTAGCTACAACGA AGAGAAGG | 5276 |
| 861 | CUCUCUCA G CCGCUUCU | 1857 | AGAAGCGG GGCTAGCTACAACGA TGAGAGAG | 5277 |
| 892 | AUACCUUG G UAAUUUGU | 1858 | ACAAATTA GGCTAGCTACAACGA CAAGGTAT | 5278 |
| 899 | GGUAAUUU G UCAACAGU | 1859 | ACTGTTGA GGCTAGCTACAACGA AAATTACC | 5279 |
| 906 | UCUCAACA G UAUUACCC | 1860 | GGGTAATA GGCTAGCTACAACGA TGTTGACA | 5280 |
| 939 | AAGAAAAU G UCAGUGAA | 1861 | TTCACTGA GGCTAGCTACAACGA ATTTTCTT | 5281 |
| 943 | AAAUGUCA G UGAAGCUU | 1862 | AAGCTTCA GGCTAGCTACAACGA TGACATTT | 5282 |
| 948 | UCAGUGAA G CUUCUAAA | 1863 | TTTAGAAG GGCTAGCTACAACGA TTCACTGA | 5283 |
| 960 | CUAAAGAG G UCUCAGAG | 1864 | CTCTGAGA GGCTAGCTACAACGA CTCTTTAG | 5284 |
| 972 | CAGAGAAG G CAAAAACU | 1865 | GCTTTTTG GGCTAGCTACAACGA CTTCTCTG | 5285 |
| 1007 | UUAACAGA G UUUUCAGA | 1866 | TCTGAAAA GGCTAGCTACAACGA TCTCTTAA | 5286 |
| 1043 | GGAUCAUC G UUCAGUGU | 1867 | ACACTGAA GGCTAGCTACAACGA GATGATCC | 5287 |
| 1048 | AUCGUUCA G UGUCUCUC | 1868 | GAGAGACA GGCTAGCTACAACGA TGAACGAT | 5288 |
| 1050 | GCUUCAGU G UCUCUCCA | 1869 | TGGAGAGA GGCTAGCTACAACGA ACTGAACG | 5289 |
| 1062 | CUCCAAAA G CAGAAUCU | 1870 | AGATTCTG GGCTAGCTACAACGA TTTTGGAC | 5290 |
| 1074 | AAUCUGCC G UAAUAGUA | 1871 | TACTATTA GGCTAGCTACAACGA GGCAGATT | 5291 |
| 1080 | CCGUAAUA G UAGCAAAU | 1872 | ATTTGCTA GGCTAGCTACAACGA TATTACGG | 5292 |
| 1083 | UAAUAGUA G CAAAUCCU | 1873 | AGGATTTG GGCTAGCTACAACGA TACTATTA | 5293 |
| 1107 | AAAUAAUC G UGAAAAAU | 1874 | ATTTTTCA GGCTAGCTACAACGA GATTATTT | 5294 |
| 1133 | GAAGAGAA G UUAGUUAG | 1875 | CTAACTAA GGCTAGCTACAACGA TTCTCTTC | 5295 |
| 1137 | AGAAGUUA G UUAGUAAU | 1876 | ATTACTAA GGCTAGCTACAACGA TAACTTCT | 5296 |
| 1141 | GUUAGUUA G UAAUAACA | 1877 | TCTTATTA GGCTAGCTACAACGA TAACTAAC | 5297 |
| 1169 | CAACAAGA G UUACCUAC | 1878 | GTAGGTAA GGCTAGCTACAACGA TCTTGTTG | 5298 |
| 1179 | UACCUACA G CUCUUACU | 1879 | AGTAAGAG GGCTAGCTACAACGA TGTAGGTA | 5299 |
| 1194 | CUAAAUUG G UUAAAGAG | 1880 | CTCTTTAA GGCTAGCTACAACGA CAATTTAG | 5300 |
| 1209 | AGGAUCAA G UUGUGUCU | 1881 | AGACACAA GGCTAGCTACAACGA TTCATCCT | 5301 |
| 1212 | AUGAAGUU G UGUCUUCA | 1882 | TGAAGACA GGCTAGCTACAACGA AACTTCAT | 5302 |
| 1214 | GAAGUUGU G UCUUCAGA | 1883 | TCTGAAGA GGCTAGCTACAACGA ACAACTTG | 5303 |
| 1227 | CAGAAAAA G CAAAAGAC | 1884 | GTCTTTTG GGCTAGCTACAACGA TTTTTCTG | 5304 |
| 1237 | AAAAGACA G UUUUAAUG | 1885 | CATTAAAA GGCTAGCTACAACGA TGTCTTTT | 5305 |
| 1254 | AAAAGAGA G UUGCAGUG | 1886 | CACTGCAA GGCTAGCTACAACGA TCTCTTTT | 5306 |
| 1260 | GAGUUGCA G UGGAAGCU | 1887 | AGCTTCCA GGCTAGCTACAACGA TGCAACTC | 5307 |
| 1266 | CAGUGGAA G CUCCUAUG | 1888 | CATAGGAG GGCTAGCTACAACGA TTCCACTG | 5308 |
| 1307 | CCAUUUGA G CGAGUAUG | 1889 | CATACTCG GGCTAGCTACAACGA TCAAATGG | 5309 |
| 1311 | UUGAGCGA G UAUGGGAA | 1890 | TTCCCATA GGCTAGCTACAACGA TCGCTCAA | 5310 |
| 1320 | UAUGGGAA G UGAAAGAU | 1891 | ATCTTTCA GGCTAGCTACAACGA TTCCCATA | 5311 |
| 1330 | GAAAGAUA G UAAGGAAG | 1892 | CTTCCTTA GGCTAGCTACAACGA TATCTTTC | 5312 |
| 1342 | GGAAGAUA G UGAUAUGU | 1893 | ACATATCA GGCTAGCTACAACGA TATCTTCC | 5313 |
| 1349 | AGUGAUAU G UUGGCUGC | 1894 | GCAGCCAA GGCTAGCTACAACGA ATATCACT | 5314 |
| 1353 | AUAUGUUG G CUGCUGGA | 1895 | TCCAGCAG GGCTAGCTACAACGA CAACATAT | 5315 |
| 1363 | UGCUGGAG G UAAAAUCG | 1896 | CGATTTTA GGCTAGCTACAACGA CTCCAGCA | 5316 |
| 1375 | AAUCGAGA G CAACUUGG | 1897 | CCAAGTTG GGCTAGCTACAACGA TCTCGATT | 5317 |
| 1387 | CUUGGAAA G UAAAGUGG | 1898 | CCACTTTA GGCTAGCTACAACGA TTTCCAAG | 5318 |
| 1392 | AAAGUAAA G UGGAUAAA | 1899 | TTTATCCA GGCTAGCTACAACGA TTTACTTT | 5319 |
| 1405 | UAAAAAAU G UUUUGCAG | 1900 | CTGCAAAA GGCTAGCTACAACGA ATTTTTTA | 5320 |
| 1417 | UGCAGAUA G CCUUGAGC | 1901 | GCTCAAGG GGCTAGCTACAACGA TATCTGCA | 5321 |
| 1424 | AGCCUUGA G CAAACUAA | 1902 | TTAGTTTG GGCTAGCTACAACGA TCAAGGCT | 5322 |
| 1447 | AAAAGAUA G UGAGAGUA | 1903 | TACTCTCA GGCTAGCTACAACGA TATCTTTT | 5323 |
| 1453 | UAGUGAGA G UAGUAAUG | 1904 | CATTACTA GGCTAGCTACAACGA TCTCACTA | 5324 |
| 1456 | UGAGAGUA G UAAUGAUG | 1905 | CATCATTA GGCTAGCTACAACGA TACTCTCA | 5325 |
| 1480 | UUUCCCCA G UACGCCAG | 1906 | CTGGCGTA GGCTAGCTACAACGA TGGGGAAA | 5326 |
| 1492 | GCCACAAG G UAUAAAGG | 1907 | CCTTTATA GGCTAGCTACAACGA CTTCTGGC | 5327 |
| 1504 | AAAGGAUC G UUCAGGAG | 1908 | CTCCTGAA GGCTAGCTACAACGA GATCCTTT | 5328 |
| 1512 | GUUCAGGA G CAUAUAUC | 1909 | GATATATG GGCTAGCTACAACGA TCCTGAAC | 5329 |
| 1525 | UAUCACAU G UGCUCCCU | 1910 | AGGGAGCA GGCTAGCTACAACGA ATGTGATA | 5330 |
| 1542 | UUAACCCA G CAGCAACU | 1911 | AGTTGCTG GGCTAGCTACAACGA TGGGTTAA | 5331 |
| 1545 | ACCCAGCA G CAACUGAG | 1912 | CTCAGTTG GGCTAGCTACAACGA TGCTGGGT | 5332 |
| 1555 | AACUGAGA G CAUUGCAA | 1913 | TTGCAATG GGCTAGCTACAACGA TCTCAGTT | 5333 |
| 1580 | UUUCCUUU G UUAGGAGA | 1914 | TCTCCTAA GGCTAGCTACAACGA AAAGGAAA | 5334 |
| 1638 | AAAAGAAG G CCCAAAUA | 1915 | TATTTGGG GGCTAGCTACAACGA CTTCTTTT | 5335 |
| 1647 | CCCAAAUA G UAACAGAG | 1916 | CTCTGTTA GGCTAGCTACAACGA TATTTGGG | 5336 |
| 1666 | GAAUACUA G CACCAAAA | 1917 | TTTTGGTG GGCTAGCTACAACGA TAGTATTC | 5337 |
| 1692 | CUUUUCUU G UAGCAGCA | 1918 | TGCTGCTA GGCTAGCTACAACGA AAGAAAAG | 5338 |
| 1695 | UUCUUGUA G CAGCACAG | 1919 | CTGTGCTG GGCTAGCTACAACGA TACAAGAA | 5339 |
| 1698 | UUGUAGCA G CACAGGAU | 1920 | ATCCTGTG GGCTAGCTACAACGA TGCTACAA | 5340 |
| 1722 | CAGAUUAU G UCACAACA | 1921 | TCTTGTGA GGCTAGCTACAACGA ATAATCTG | 5341 |
| 1746 | UAACAAAG G UGACUGAG | 1922 | CTCAGTCA GGCTAGCTACAACGA CTTTGTTA | 5342 |
| 1758 | CUGAGGAA G UCGUGGCA | 1923 | TGCCACGA GGCTAGCTACAACGA TTCCTCAG | 5343 |
| 1761 | AGGAAGUC G UGGCAAAC | 1924 | GTTTGCCA GGCTAGCTACAACGA GACTTCCT | 5344 |
| 1764 | AAGUCGUG G CAAACAUG | 1925 | CATGTTTG GGCTAGCTACAACGA CACGACTT | 5345 |
| 1780 | GCCUGAAG G CCUGAGUC | 1926 | GAGTCAGG GGCTAGCTACAACGA CTTCAGGC | 5346 |
| 1797 | CAGAUUUA G UACAGGAA | 1927 | TTCCTGTA GGCTAGCTACAACGA TAAATCTG | 5347 |
| 1806 | UACAGGAA G CAUGUGAA | 1928 | TTCACATG GGCTAGCTACAACGA TTCCTGTA | 5348 |
| 1810 | GGAAGCAU G UGAAAGUG | 1929 | CACTTTCA GGCTAGCTACAACGA ATGCTTCC | 5349 |
| 1816 | AUGUCAAA G UGAAUUGA | 1930 | TCAATTCA GGCTAGCTACAACGA TTTCACAT | 5350 |
| 1830 | UGAAUCAA G UUACUGGU | 1931 | ACCAGTAA GGCTAGCTACAACGA TTCATTCA | 5351 |
| 1837 | AGUUACUG G UACAAAGA | 1932 | TCTTTGTA GGCTAGCTACAACGA CAGTAACT | 5352 |
| 1872 | UGGACUUG G UUCAAACA | 1933 | TGTTTGAA GGCTAGCTACAACGA CAAGTCCA | 5353 |
| 1887 | CAUCAGAA G UUAUGCAA | 1934 | TTGCATAA GGCTAGCTACAACGA TTCTGATG | 5354 |
| 1898 | AUGCAAGA G UCACUCUA | 1935 | TAGACTGA GGCTAGCTACAACGA TCTTGCAT | 5355 |
| 1914 | AUCCUGCA G CACAAGUU | 1936 | AAGCTGTG GGCTAGCTACAACGA TGCAGGAT | 5356 |
| 1919 | GCAGCACA G CUUUGCCC | 1937 | GGGCAAAG GGCTAGCTACAACGA TGTGCTGC | 5357 |
| 1940 | UUUGAAGA G UCAGAAGC | 1938 | GCTTCTGA GGCTAGCTACAACGA TCTTCAAA | 5358 |
| 1947 | AGUCAGAA G CUACUCCU | 1939 | AGGACTAC GGCTAGCTACAACGA TTCTGACT | 5359 |
| 1962 | CUUCACCA G UUUUGCCU | 1940 | AGGCAAAA GGCTAGCTACAACGA TGGTGAAG | 5360 |
| 1977 | CUGACAUU G UUAUGGAA | 1941 | TTCCATAA GGCTAGCTACAACGA AATGTCAG | 5361 |
| 1986 | UUAUGGAA G CACCAUUG | 1942 | CAATGGTG GGCTAGCTACAACGA TTCCATAA | 5362 |
| 2004 | AUUCUGCA G UUCCUAGU | 1943 | ACTAGGAA GGCTAGCTACAACGA TGCAGAAT | 5363 |
| 2011 | AGUUCCUA G UGCUGGUG | 1944 | CACCACCA GGCTAGCTACAACGA TAGGAACT | 5364 |
| 2017 | UAGUGCUG G UGCUUCCG | 1945 | CGGAAGCA GGCTAGCTACAACGA CAGCACTA | 5365 |
| 2025 | GUGCUUCC G UGAUACAG | 1946 | CTGTATCA GGCTAGCTACAACGA GGAAGCAC | 5366 |
| 2033 | GUGAUACA G CCCAGCUC | 1947 | GAGCTGGG GGCTAGCTACAACGA TGTATCAC | 5367 |
| 2038 | ACAGCCCA G CUCAUCAC | 1948 | GTGATGAG GGCTAGCTACAACGA TGGGCTGT | 5368 |
| 2055 | CAUUAGAA G CUUCUUCA | 1949 | TGAAGAAG GGCTAGCTACAACGA TTCTAATC | 5369 |
| 2064 | CUUCUUCA G UUAAUUAU | 1950 | ATAATTAA GGCTAGCTACAACGA TGAAGAAG | 5370 |
| 2077 | UUAUGAAA G CAUAAAAC | 1951 | GTTTTATC GGCTAGCTACAACGA TTTCATAA | 5371 |
| 2090 | AAACAUGA G CCUGAAAA | 1952 | TTTTCAGG GGCTAGCTACAACGA TCATGTTT | 5372 |
| 2118 | AUGAAGAG G CCAUGAGU | 1953 | ACTCATCG GGCTAGCTACAACGA CTCTTCAT | 5373 |
| 2125 | GGCCAUGA G UGUAUCAC | 1954 | GTGATACA GGCTAGCTACAACGA TCATGGCC | 5374 |
| 2127 | CCAUGAGU G UAUCACUA | 1955 | TAGTGATA GGCTAGCTACAACGA ACTCATGG | 5375 |
| 2142 | UAAAAAAA G UAUCAGGA | 1956 | TCCTGATA GGCTAGCTACAACGA TTTTTTTA | 5376 |
| 2171 | AUUAAAGA G CCUGAAAA | 1957 | TTTTCAGG GGCTAGCTACAACGA TCTTTAAT | 5377 |
| 2190 | UUAAUGCA G CUCUUCAA | 1958 | TTGAAGAG GGCTAGCTACAACGA TGCATTAA | 5378 |
| 2208 | AAACAGAA G CUCCUCAU | 1959 | ATAAGGAG GGCTAGCTACAACGA TTCTGTTT | 5379 |
| 2230 | UAUUGCAU G UGAUUUAA | 1960 | TTAAATCA GGCTAGCTACAACGA ATGCAATA | 5380 |
| 2252 | GAAACAAA G CUUUCUGC | 1961 | GCAGAAAG GGCTAGCTACAACGA TTTGTTTC | 5381 |
| 2268 | CUGAACGA G CUCCGGAU | 1962 | ATCCGGAG GGCTAGCTACAACGA TGGTTCAG | 5382 |
| 2298 | CAGAAAUC G CAAAAGUU | 1963 | AACTTTTG GGCTAGCTACAACGA CATTTCTG | 5383 |
| 2304 | UGGCAAAA G UUGAACAG | 1964 | CTGTTCAA GGCTAGCTACAACGA TTTTGCCA | 5384 |
| 2312 | GUUGAACA G CCAGUGCC | 1965 | GGCACTGG GGCTAGCTACAACGA TGTTCAAC | 5385 |
| 2316 | AACAGCCA G UGCCUGAU | 1966 | ATCAGGCA GGCTAGCTACAACGA TGGCTGTT | 5386 |
| 2333 | CAUUCUGA G CUAGUCGA | 1967 | TCAACTAG GGCTAGCTACAACGA TCAGAATG | 5387 |
| 2337 | CUGAGCUA G UUGAAGAU | 1968 | ATCTTCAA GGCTAGCTACAACGA TAGCTCAC | 5388 |
| 2367 | CUGAACCA G UUGACUUA | 1969 | TAAGTCAA GGCTAGCTACAACGA TGGTTCAG | 5389 |
| 2380 | CUUAUUUA G UGAUGAUU | 1970 | AATCATCA GGCTAGCTACAACGA TAAATAAC | 5390 |
| 2400 | UACCUGAC G UUCCACAA | 1971 | TTGTCGAA GGCTAGCTACAACGA GTCAGGTA | 5391 |
| 2424 | AUGAAACU G UGAUGCUU | 1972 | AAGCATCA GGCTAGCTACAACGA AGTTTCAT | 5392 |
| 2433 | UGAUGCUU G UGAAAGAA | 1973 | TTCTTTCA GGCTAGCTACAACGA AAGCATCA | 5393 |
| 2443 | GAAAGAAA G UCUCACUG | 1974 | CAGTGAGA GGCTAGCTACAACGA TTTCTTTC | 5394 |
| 2465 | UCAUUUGA G UCAAUGAU | 1975 | ATCATTGA GGCTAGCTACAACGA TCAAATGA | 5395 |
| 2500 | AAAACUCA G UGCUUUGC | 1976 | GCAAAGCA GGCTAGCTACAACGA TGAGTTTT | 5396 |
| 2525 | GGAGGAAA G CCAUAUUU | 1977 | AAATATGG GGCTAGCTACAACGA TTTCCTCC | 5397 |
| 2546 | UCUUUUAA G CUCAGUUU | 1978 | AAACTGAG GGCTAGCTACAACGA TTAAAAGA | 5398 |
| 2551 | UAAGCUCA G UUUAGAUA | 1979 | TATCTAAA GGCTAGCTACAACGA TGAGCTTA | 5399 |
| 2576 | GAUACCCU G UUACCUGA | 1980 | TCAGCTAA GGCTAGCTACAACGA AGGGTATC | 5400 |
| 2589 | CUGAUGAA G UUUCAACA | 1981 | TGTTGAAA GGCTAGCTACAACGA TTCATCAG | 5401 |
| 2602 | AACAUUGA G CAAAAAGG | 1982 | CCTTTTTG GGCTAGCTACAACGA TCAATGTT | 5402 |
| 2636 | AUGGAGGA G CUCAGUAC | 1983 | GTACTGAG GGCTAGCTACAACGA TCCTCCAT | 5403 |
| 2641 | GGAGCUCA G UACUGCAG | 1984 | CTGGAGTA GGCTAGCTACAACGA TGAGCTCC | 5404 |
| 2649 | GUACUGCA G UUUAUUCA | 1985 | TGAATAAA GGCTAGCTACAACGA TGCAGTAC | 5405 |
| 2685 | CUAAGGAA G CACAGAUA | 1986 | TATCTGTG GGCTAGCTACAACGA TTCCTTAG | 5406 |
| 2708 | ACUGAAAC G UUUUCAGA | 1987 | TCTGAAAA GGCTAGCTACAACGA CTTTCAGT | 5407 |
| 2744 | AUAGAUGA G UUCCCUAC | 1988 | GTAGGGAA GGCTAGCTACAACGA TCATCTAT | 5408 |
| 2761 | AUUGAUGA G UUCUAAAA | 1989 | TTTTAGAA GGCTAGCTACAACGA TGATCAAT | 5409 |
| 2790 | CUAAAUUA G CCAGGGAA | 1990 | TTCCCTGG GGCTAGCTACAACGA TAATTTAG | 5410 |
| 2814 | ACCUAGAA G UAUCCCAC | 1991 | GTGGGATA GGCTAGCTACAACGA TTCTAGGT | 5411 |
| 2827 | CCACAAAA G UGAAAUUG | 1992 | CAATTTCA GGCTAGCTACAACGA TTTTGTGG | 5412 |
| 2853 | CGGAUGGA G CUGGGUCA | 1993 | TGACCCAG GGCTAGCTACAACGA TCCATCCG | 5413 |
| 2858 | GGAGCUGG G UCAUUGCC | 1994 | GGCAATGA GGCTAGCTACAACGA CCAGCTCC | 5414 |
| 2916 | AACCCAAA G UUGAAGAG | 1995 | CTCTTCAA GGCTAGCTACAACGA TTTGGGTT | 5415 |
| 2932 | GAAAAUCA G UUUCUCAG | 1996 | CTGAGAAA GGCTAGCTACAACGA TGATTTTC | 5416 |
| 2960 | AAAAAUGG G UCUGCUAC | 1997 | GTAGCAGA GGCTAGCTACAACGA CCATTTTT | 5417 |
| 2976 | CAUCAAAG G UGCUCUUA | 1998 | TAAGAGCA GGCTAGCTACAACGA CTTTGATG | 5418 |
| 2997 | CUCCAGAU G UUUCUGCU | 1999 | AGCAGAAA GGCTAGCTACAACGA ATCTGGAG | 5419 |
| 3009 | CUGCUUUG G CCACUCAA | 2000 | TTGAGTGG GGCTAGCTACAACGA CAAAGCAG | 5420 |
| 3018 | CCACUCAA G CAGAGAUA | 2001 | TATCTCTG GGCTAGCTACAACGA TTGAGTGG | 5421 |
| 3031 | GAUAGAGA G CAUAGUUA | 2002 | TAACTATG GGCTAGCTACAACGA TCTCTATC | 5422 |
| 3036 | AGAGCAUA G UUAAACCC | 2003 | GGGTTTAA GGCTAGCTACAACGA TATGCTCT | 5423 |
| 3048 | AACCCAAA G UUCUUGUG | 2004 | CACAAGAA GGCTAGCTACAACGA TTTGGGTT | 5424 |
| 3054 | AAGUUCUU G UGAAAGAA | 2005 | TTCTTTCA GGCTAGCTACAACGA AAGAACTT | 5425 |
| 3063 | UGAAAGAA G CUGAGAAA | 2006 | TTTCTCAG GGCTAGCTACAACGA TTCTTTCA | 5426 |
| 3126 | UAUUUUCA G CAGAGCUG | 2007 | CAGCTCTG GGCTAGCTACAACGA TCAAAATA | 5427 |
| 3131 | UCAGCAGA G CUGACGAA | 2008 | TTACTCAG GGCTAGCTACAACGA TCTGCTGA | 5428 |
| 3136 | AGAGCUGA G UAAAACUU | 2009 | AAGTTTTA GGCTAGCTACAACGA TCAGCTCT | 5429 |
| 3147 | AAACUUCA G UUGUUGAC | 2010 | GTCAACAA GGCTAGCTACAACGA TGAAGTTT | 5430 |
| 3150 | CUUCAGUU G UUGACCUC | 2011 | GAGGTGAA GGCTAGCTACAACGA AACTCAAG | 5431 |
| 3161 | GACCUCCU G UACUGGAG | 2012 | CTCCAGTA GGCTAGCTACAACGA AGGAGGTC | 5432 |
| 3189 | AGACUGGA G UGGUGUUU | 2013 | AAACACCA GGCTAGCTACAACGA TCCAGTCT | 5433 |
| 3192 | CUGGAGUG G UGUUUGGU | 2014 | ACCAAACA GGCTAGCTACAACGA CACTCCAG | 5434 |
| 3194 | GGAGUGGU G UUUGGUGC | 2015 | GCACCAAA GGCTAGCTACAACGA ACCACTCC | 5435 |
| 3199 | GGUGUUUG G UGCCAGCC | 2016 | GGCTGGCA GGCTAGCTACAACGA CAAACACC | 5436 |
| 3205 | UGGUGCCA G CCUAUUCC | 2017 | CGGATAGG GGCTAGCTACAACGA TGGCACCA | 5437 |
| 3231 | CAUUCACA G UAUUCAGC | 2018 | GCTGAATA GGCTAGCTACAACGA TGTCAATG | 5438 |
| 3238 | AGUAUUCA G CAUUGUGA | 2019 | TCACAATG GGCTAGCTACAACGA TGAATACT | 5439 |
| 3243 | UCAGCAUU G UGAGCCUA | 2020 | TACGCTCA GGCTAGCTACAACGA AATGCTGA | 5440 |
| 3247 | CAUUGUGA G CGUAACAG | 2021 | CTGTTACG GGCTAGCTACAACGA TCACAATC | 5441 |
| 3249 | UUGUGAGC G UAACAGCC | 2022 | GGCTGTTA GGCTAGCTACAACGA GCTCACAA | 5442 |
| 3255 | GCGUAACA G CCUACAUU | 2023 | AATGTACG GGCTAGCTACAACGA TGTTACGC | 5443 |
| 3270 | UUGCCUUG G CCCUGCUC | 2024 | CAGCAGGG GGCTAGCTACAACGA CAAGGCAA | 5444 |
| 3282 | UGCUCUCU G UGACCAUC | 2025 | GATGGTCA GGCTAGCTACAACGA AGACAGCA | 5445 |
| 3292 | GACCAUCA G CUUUAGGA | 2026 | TCCTAAAG GGCTAGCTACAACGA TGATGGTC | 5446 |
| 3310 | AUACAAGG G UGUGAUGC | 2027 | GGATCACA GGCTAGCTACAACGA CCTTGTAT | 5447 |
| 3312 | ACAAGGGU G UGAUGCAA | 2028 | TTGGATCA GGCTAGCTACAACGA ACCCTTGT | 5448 |
| 3321 | UGAUGCAA G CUAUCCAC | 2029 | CTGGATAG GGCTAGCTACAACGA TTGGATCA | 5449 |
| 3343 | AGAUGAAG G CCACCCAU | 2030 | ATGGGTCC GGCTAGCTACAACGA CTTCATCT | 5450 |
| 3357 | CAUUCAGG G CAUAUCUG | 2031 | CAGATATG GGCTAGCTACAACGA CCTGAATG | 5451 |
| 3375 | AAUCUGAA G UUGCUAUA | 2032 | TATAGCAA GGCTAGCTACAACGA TTCAGATT | 5452 |
| 3392 | UCUGAGGA G UUGGUUCA | 2033 | TGAACCAA GGCTAGCTACAACGA TCCTCAGA | 5453 |
| 3396 | AGGAGUUG G UUCAGAAG | 2034 | CTTCTGAA GGCTAGCTACAACGA CAACTCCT | 5454 |
| 3404 | GUUCAGAA G UACAGUAA | 2035 | TTACTGTA GGCTAGCTACAACGA TTCTGAAC | 5455 |
| 3409 | GAAGUACA G UAAUUCUG | 2036 | CACAATTA GGCTAGCTACAACGA TGTACTTC | 5456 |
| 3424 | UGCUCUUG G UCAUGUGA | 2037 | TCACATGA GGCTAGCTACAACGA CAAGAGCA | 5457 |
| 3429 | UUGGUCAU G UGAACUGC | 2038 | GCACTTCA GGCTAGCTACAACGA ATGACCAA | 5458 |
| 3455 | GAACUCAG G CGCCUCUU | 2039 | AAGAGGCC GGCTAGCTACAACGA CTGAGTTC | 5459 |
| 3468 | UCUUCUUA G UUGAUGAU | 2040 | ATCATCAA GGCTAGCTACAACGA TAAGAAGA | 5460 |
| 3480 | AUGAUUUA G UUGAUUCU | 2041 | AGAATCAA GGCTAGCTACAACGA TAAATCAT | 5461 |
| 3494 | UCUCUGAA G UUUGCAGU | 2042 | ACTGCAAA GGCTAGCTACAACGA TTCAGAGA | 5462 |
| 3501 | AGUUUGCA G UGUUGAUG | 2043 | CATCAACA GGCTAGCTACAACGA TGCAAACT | 5463 |
| 3503 | UUUGCAGU G UUGAUGUG | 2044 | CACATCAA GGCTAGCTACAACGA ACTGCAAA | 5464 |
| 3509 | GUGUUGAU G UGGGUAUU | 2045 | AATACCCA GGCTAGCTACAACGA ATCAACAC | 5465 |
| 3513 | UGAUGUGG G UAUUUACC | 2046 | GGTAAATA GGCTAGCTACAACGA CCACATCA | 5466 |
| 3525 | UUACCUAU G UUGGUGCC | 2047 | GGCACCAA GGCTAGCTACAACGA ATAGGTAA | 5467 |
| 3529 | CUAUGUUG G UGCCUUGU | 2048 | ACAAGGCA GGCTAGCTACAACGA CAACATAG | 5468 |
| 3536 | GGUGCCUU G UUUAAUGG | 2049 | CCATTAAA GGCTAGCTACAACGA AAGGCACC | 5469 |
| 3544 | GUUUAAUG G UCUGACAC | 2050 | GTGTCAGA GGCTAGCTACAACGA CATTAAAC | 5470 |
| 3564 | UGAUUUUG G CUCUCAUU | 2051 | AATGAGAG GGCTAGCTACAACGA CAAAATCA | 5471 |
| 3583 | ACUCUUCA G UGUUCCUG | 2052 | CAGGAACA GGCTAGCTACAACGA TGAAGAGT | 5472 |
| 3585 | UCUUCAGU G UUCCUGUU | 2053 | AACAGGAA GGCTAGCTACAACGA ACTGAAGA | 5373 |
| 3591 | GUGUUCCU G UUAUUUAU | 2054 | ATAAATAA GGCTAGCTACAACGA AGGAACAC | 5474 |
| 3605 | UAUGAACG G CAUCAGGC | 2055 | GCCTGATG GGCTAGCTACAACGA CGTTCATA | 5475 |
| 3612 | GGCAUCAG G CACAGAUA | 2056 | TATCTGTG GGCTAGCTACAACGA CTGATGCC | 5476 |
| 3651 | AUAAGAAU G UUAAAGAU | 2057 | ATCTTTAA GGCTAGCTACAACGA ATTCTTAT | 5477 |
| 3666 | AUGCUAUG G CUAAAAUC | 2058 | GATTTTAG GGCTAGCTACAACGA CATAGCAT | 5478 |
| 3678 | AAAUCCAA G CAAAAAUC | 2059 | GATTTTTG GGCTAGCTACAACGA TTGGATTT | 5479 |
| 3698 | GGAUUGAA G CGCAAAGC | 2060 | GCTTTGCG GGCTAGCTACAACGA TTCAATCC | 5480 |
| 3705 | AGCGCAAA G CUGAAUGA | 2061 | TCATTCAG GGCTAGCTACAACGA TTTGCGCT | 5481 |
| 3732 | AAUAAUUA G UAGGAGUU | 2062 | AACTCCTA GGCTAGCTACAACGA TAATTATT | 5482 |
| 3738 | UAGUAGGA G UUCAUCUU | 2063 | AAGATGAA GGCTAGCTACAACGA TCCTACTA | 5483 |
| 3781 | GGGGGAGG G UCAGGGAA | 2064 | TTCCCTGA GGCTAGCTACAACGA CCTCCCCC | 5484 |
| 3804 | ACCUUGAC G UUGCAGUG | 2065 | CACTGCAA GGCTAGCTACAACGA GTCAAGGT | 5485 |
| 3810 | ACGUUGCA G UGCAGUUU | 2066 | AAACTGCA GGCTAGCTACAACGA TGCAACGT | 5486 |
| 3815 | GCAGUGCA G UUUCACAG | 2067 | CTGTGAAA GGCTAGCTACAACGA TGCACTGC | 5487 |
| 3827 | CACAGAUC G UUGUUAGA | 2068 | TCTAACAA GGCTAGCTACAACGA GATCTGTG | 5488 |
| 3830 | AGAUCGUU G UUAGAUCU | 2069 | AGATCTAA GGCTAGCTACAACGA AACGATCT | 5489 |
| 3848 | UAUUUUUA G CCAUGCAC | 2070 | GTGCATGG GGCTAGCTACAACGA TAAAAATA | 5490 |
| 3858 | CAUGCACU G UUGUGAGG | 2071 | CCTCACAA GGCTAGCTACAACGA AGTGCATG | 5491 |
| 3861 | GCACUGUU G UGAGGAAA | 2072 | TTTCCTCA GGCTAGCTACAACGA AACAGTGC | 5492 |
| 3878 | AAUUACCU G UCUUGACU | 2073 | AGTCAAGA GGCTAGCTACAACGA AGGTAATT | 5493 |
| 3892 | ACUGCCAU G UGUUCAUC | 2074 | GATGAACA GGCTAGCTACAACGA ATGGCAGT | 5494 |
| 3894 | UGCCAUGU G UUCAUCAU | 2075 | ATGATGAA GGCTAGCTACAACGA ACATGGCA | 5495 |
| 3908 | CAUCUUAA G UAUUGUAA | 2076 | TTACAATA GGCTAGCTACAACGA TTAAGATG | 5496 |
| 3913 | UAAGUAUU G UAAGCUGC | 2077 | GCAGCTTA GGCTAGCTACAACGA AATACTTA | 5497 |
| 3917 | UAUUGUAA G CUGCUAUG | 2078 | CATAGCAG GGCTAGCTACAACGA TTACAATA | 5498 |
| 3925 | GCUGCUAU G UAUGGAUU | 2079 | AATCCATA GGCTAGCTACAACGA ATAGCAGC | 5499 |
| 3940 | UUUAAACC G UAAUCAUA | 2080 | TATGATTA GGCTAGCTACAACGA GGTTTAAA | 5500 |
| 3966 | UAUCUGAG G CACUGGUG | 2081 | CACCAGTG GGCTAGCTACAACGA CTCAGATA | 5501 |
| 3972 | AGGCACUG G UGGAAUAA | 2082 | TTATTCCA GGCTAGCTACAACGA CAGTGCCT | 5502 |
| 3988 | AAAAACCU G UAUAUUUU | 2083 | AAAATATA GGCTAGCTACAACGA AGGTTTTT | 5503 |
| 4002 | UUUACUUU G UUGCAGAU | 2084 | ATCTGCAA GGCTAGCTACAACGA AAAGTAAA | 5504 |
| 4012 | UGCAGAUA G UCUUGCCG | 2085 | CGGCAAGA GGCTAGCTACAACGA TATCTGCA | 5505 |
| 4028 | GCAUCUUG G CAAGUUGC | 2086 | GCAACTTG GGCTAGCTACAACGA CAAGATGC | 5506 |
| 4032 | CUUGGCAA G UUGCAGAG | 2087 | CTCTGCAA GGCTAGCTACAACGA TTGCCAAG | 5507 |
| 4044 | CAGAGAUG G UGGAGCUA | 2088 | TAGCTCCA GGCTAGCTACAACGA CATCTCTG | 5508 |
| 54 | CCUCCCCA A CCCCCACA | 2089 | TGTGGGGG GGCTAGCTACAACGA TGGGGAGG | 5509 |
| 63 | CCCCCACA A CCGCCCGC | 2090 | GCGGGCGG GGCTAGCTACAACGA TGTGGGGG | 5510 |
| 81 | GCUCUGAG A CGCGGCCC | 2091 | GGGCCGCG GGCTAGCTACAACGA CTCAGAGC | 5511 |
| 142 | CAUGGAAG A CCUGGACC | 2092 | GGTCCAGG GGCTAGCTACAACGA CTTCCATG | 5512 |
| 148 | AGACCUGG A CCAGUCUC | 2093 | GAGACTGG GGCTAGCTACAACGA CCAGGTCT | 5513 |
| 175 | GUCCUCGG A CAGCCCAC | 2094 | GTGGGCTG GGCTAGCTACAACGA CCGAGGAC | 5514 |
| 232 | GCCCGAGG A CGAGGAGG | 2095 | CCTCCTCG GGCTAGCTACAACGA CCTCGGGC | 5515 |
| 265 | AGAGGAGG A CGAGGACG | 2096 | CGTCCTCG GGCTAGCTACAACGA CCTCCTCT | 5516 |
| 271 | GGACGAGG A CGAAGACC | 2097 | GGTCTTCG GGCTAGCTACAACGA CCTCGTCC | 5517 |
| 277 | GGACGAAG A CCUGGAGG | 2098 | CCTCCAGG GGCTAGCTACAACGA CTTCGTCC | 5518 |
| 369 | CGCCCCUG A UGGACUUC | 2099 | GAAGTCCA GGCTAGCTACAACGA CAGGGGCG | 5519 |
| 373 | CCUGAUGG A CUUCGGAA | 2100 | TTCCGAAG GGCTAGCTACAACGA CCATCAGG | 5520 |
| 382 | CUUCGGAA A UGACUUCG | 2101 | CGAAGTCA GGCTAGCTACAACGA TTCCGAAG | 5521 |
| 385 | CGGAAAUG A CUUCGUGC | 2102 | GCACGAAG GGCTAGCTACAACGA CATTTCCG | 5522 |
| 410 | CCCCGGGG A CCCCUGCC | 2103 | GGCAGGGG GGCTAGCTACAACGA CCCCGGGG | 5523 |
| 460 | GUCUUGGG A CCCGAGCC | 2104 | GGCTCGGG GGCTAGCTACAACGA CCCAAGAC | 5524 |
| 480 | UGUCGUCG A CCGUGCCC | 2105 | GGGCACGG GGCTAGCTACAACGA CGACGACA | 5525 |
| 541 | CCCUGAGG A CGACGAGC | 2106 | GCTCGTCG GGCTAGCTACAACGA CCTCAGGG | 5526 |
| 544 | UGAGGACG A CGAGCCUC | 2107 | GAGGCTCG GGCTAGCTACAACGA CGTCCTCA | 5527 |
| 612 | CCGUGUGG A CCCCGCCA | 2108 | TGGCGGGG GGCTAGCTACAACGA CCACACGG | 5528 |
| 691 | CUCAGUGG A UGAGACCC | 2109 | GGGTCTCA GGCTAGCTACAACGA CCACTGAG | 5529 |
| 696 | UGGAUGAG A CCCUUUUU | 2110 | AAAAAGGG GGCTAGCTACAACGA CTCATCCA | 5530 |
| 732 | AGCCUGUG A UACGCUCC | 2111 | GGAGCGTA GGCTAGCTACAACGA CACAGGCT | 5531 |
| 751 | UGCAGAAA A UAUGGACU | 2112 | AGTCCATA GGCTAGCTACAACGA TTTCTGCA | 5532 |
| 757 | AAAUAUGG A CUUGAAGG | 2113 | CCTTCAAG GGCTAGCTACAACGA CCATATTT | 5533 |
| 778 | GCCAGGUA A CACUAUUU | 2114 | AAATAGTG GGCTAGCTACAACGA TACCTGGC | 5534 |
| 802 | UCAAGAGG A UUUCCCAU | 2115 | ATGGGAAA GGCTAGCTACAACGA CCTCTTGA | 5535 |
| 825 | UGCUUGAA A CUGCUGCU | 2116 | AGCAGCAG GGCTAGCTACAACGA TTCAAGCA | 5536 |
| 878 | UUCAAAGA A CAUGAAUA | 2117 | TATTCATG GGCTAGCTACAACGA TCTTTGAA | 5537 |
| 884 | GAACAUGA A UACCUUGG | 2118 | CCAAGGTA GGCTAGCTACAACGA TCATGTTC | 5538 |
| 895 | CCUUGGUA A UUUGUCAA | 2119 | TTGACAAA GGCTAGCTACAACGA TACCAAGG | 5539 |
| 903 | AUUUGUCA A CAGUAUUA | 2120 | TAATACTG GGCTAGCTACAACGA TGACAAAT | 5540 |
| 924 | CUGAAGGA A CACUUCAA | 2121 | TTGAAGTG GGCTAGCTACAACGA TCCTTCAG | 5541 |
| 937 | UCAAGAAA A UGUCAGUG | 2122 | CACTGACA GGCTAGCTACAACGA TTTCTTGA | 5542 |
| 978 | AGGCAAAA A CUCUACUC | 2123 | GAGTAGAG GGCTAGCTACAACGA TTTTGCCT | 5543 |
| 991 | ACUCAUAG A UAGAGAUU | 2124 | AATCTCTA GGCTAGCTACAACGA CTATGAGT | 5544 |
| 997 | AGAUAGAG A UUUAACAG | 2125 | CTGTTAAA GGCTAGCTACAACGA CTCTATCT | 5545 |
| 1002 | GAGAUUUA A CAGAGUUU | 2126 | AAACTCTG GGCTAGCTACAACGA TAAATCTC | 5546 |
| 1016 | UUUUCAGA A UUAGAAUA | 2127 | TATTCTAA GGCTAGCTACAACGA TCTGAAAA | 5547 |
| 1022 | GAAUUAGA A UACUCAGA | 2128 | TCTGAGTA GGCTAGCTACAACGA TCTAATTC | 5548 |
| 1032 | ACUCAGAA A UGGGAUCA | 2129 | TGATCCCA GGCTAGCTACAACGA TTCTGAGT | 5549 |
| 1037 | GAAAUGGG A UCAUCGUU | 2130 | AACGATGA GGCTAGCTACAACGA CCCATTTC | 5550 |
| 1067 | AAAGCAGA A UCUGCCGU | 2131 | ACGGCAGA GGCTAGCTACAACGA TCTGCTTT | 5551 |
| 1077 | CUGCCGUA A UAGUAGCA | 2132 | TGCTACTA GGCTAGCTACAACGA TACGGCAG | 5552 |
| 1087 | AGUAGCAA A UCCUAGGG | 2133 | CCCTAGGA GGCTAGCTACAACGA TTGCTACT | 5553 |
| 1101 | GGGAAGAA A UAAUCGUG | 2134 | CACGATTA GGCTAGCTACAACGA TTCTTCCC | 5554 |
| 1104 | AAGAAAUA A UCGUGAAA | 2135 | TTTCACGA GGCTAGCTACAACGA TATTTCTT | 5555 |
| 1114 | CGUGAAAA A UAAAGAUG | 2136 | CATCTTTA GGCTAGCTACAACGA TTTTCACG | 5556 |
| 1120 | AAAUAAAG A UGAAGAAG | 2137 | CTTCTTCA GGCTAGCTACAACGA CTTTATTT | 5557 |
| 1144 | AGUUAGUA A UAACAUCC | 2138 | GGATGTTA GGCTAGCTACAACGA TACTAACT | 5558 |
| 1147 | UAGUAAUA A CAUCCUUC | 2139 | GAAGGATG GGCTAGCTACAACGA TATTACTA | 5559 |
| 1159 | CCUUCAUA A UCAACAAG | 2140 | CTTGTTGA GGCTAGCTACAACGA TATGAAGG | 5560 |
| 1163 | CAUAAUCA A CAAGAGUU | 2141 | AACTCTTG GGCTAGCTACAACGA TGATTATG | 5561 |
| 1190 | CUUACUAA A UUGGUUAA | 2142 | TTAACCAA GGCTAGCTACAACGA TTAGTAAG | 5562 |
| 1204 | UAAAGAGG A UGAAGUUG | 2143 | CAACTTCA GGCTAGCTACAACGA CCTCTTTA | 5563 |
| 1234 | AGCAAAAG A CAGUUUUA | 2144 | TAAAACTG GGCTAGCTACAACGA CTTTTGCT | 5564 |
| 1243 | CAGUUUUA A UGAAAAGA | 2145 | TCTTTTCA GGCTAGCTACAACGA TAAAACTG | 5565 |
| 1283 | AGGGAGGA A UAUGCAGA | 2146 | TCTGCATA GGCTAGCTACAACGA TCCTCCCT | 5566 |
| 1291 | AUAUGCAG A CUUCAAAC | 2147 | GTTTGAAG GGCTAGCTACAACGA CTGCATAT | 5567 |
| 1298 | GACUUCAA A CCAUUUGA | 2148 | TCAAATGG GGCTAGCTACAACGA TTGAAGTC | 5568 |
| 1327 | AGUGAAAG A UAGUAAGG | 2149 | CCTTACTA GGCTAGCTACAACGA CTTTCACT | 5569 |
| 1339 | UAAGGAAG A UAGUGAUA | 2150 | TATCACTA GGCTAGCTACAACGA CTTCCTTA | 5570 |
| 1345 | AGAUAGUG A UAUGUUGG | 2151 | CCAACATA GGCTAGCTACAACGA CACTATCT | 5571 |
| 1368 | GAGGUAAA A UCGAGAGC | 2152 | GCTCTCGA GGCTAGCTACAACGA TTTACCTC | 5572 |
| 1378 | CGAGAGCA A CUUGGAAA | 2153 | TTTCCAAG GGCTAGCTACAACGA TGCTCTCG | 5573 |
| 1396 | UAAAGUGG A UAAAAAAU | 2154 | ATTTTTTA GGCTAGCTACAACGA CCACTTTA | 5574 |
| 1403 | GAUAAAAA A UGUUUUGC | 2155 | GCAAAACA GGCTAGCTACAACGA TTTTTATC | 5575 |
| 1414 | UUUUGCAG A UAGCCUUG | 2156 | CAAGGCTA GGCTAGCTACAACGA CTGCAAAA | 5576 |
| 1428 | UUGAGCAA A CUAAUCAC | 2157 | GTGATTAG GGCTAGCTACAACGA TTGCTCAA | 5577 |
| 1432 | GCAAACUA A UCACGAAA | 2158 | TTTCGTGA GGCTAGCTACAACGA TAGTTTGC | 5578 |
| 1444 | CGAAAAAG A UAGUGAGA | 2159 | TCTCACTA GGCTAGCTACAACGA CTTTTTCG | 5579 |
| 1459 | GAGUAGUA A UGAUGAUA | 2160 | TATCATCA GGCTAGCTACAACGA TACTACTC | 5580 |
| 1462 | UAGUAAUG A UGAUACUU | 2161 | AAGTATCA GGCTAGCTACAACGA CATTACTA | 5581 |
| 1465 | UAAUGAUG A UACUUCUU | 2162 | AAGAAGTA GGCTAGCTACAACGA CATCATTA | 5582 |
| 1501 | UAUAAAGG A UCGUUCAG | 2163 | CTGAACGA GGCTAGCTACAACGA CCTTTATA | 5583 |
| 1537 | UCCCUUUA A CCCAGCAG | 2164 | CTGCTGGG GGCTAGCTACAACGA TAAAGGGA | 5584 |
| 1548 | CAGCAGCA A CUGAGAGC | 2165 | GCTCTCAG GGCTAGCTACAACGA TGCTGCTG | 5585 |
| 1563 | GCAUUGCA A CAAACAUU | 2166 | AATGTTTG GGCTAGCTACAACGA TGCAATGC | 5586 |
| 1567 | UGCAACAA A CAUUUUUC | 2167 | GAAAAATG GGCTAGCTACAACGA TTGTTGCA | 5587 |
| 1588 | GUUAGGAG A UCCUACUU | 2168 | AAGTAGGA GGCTAGCTACAACGA CTCCTAAC | 5588 |
| 1603 | UUCAGAAA A UAAGACCG | 2169 | CGGTCTTA GGCTAGCTACAACGA TTTCTGAA | 5589 |
| 1608 | AAAAUAAG A CCGAUGAA | 2170 | TTCATCGG GGCTAGCTACAACGA CTTATTTT | 5590 |
| 1612 | UAAGACCG A UGAAAAAA | 2171 | TTTTTTCA GGCTAGCTACAACGA CGGTCTTA | 5591 |
| 1623 | AAAAAAAA A UAGAAGAA | 2172 | TTCTTCTA GGCTAGCTACAACGA TTTTTTTT | 5592 |
| 1644 | AGGCCCAA A UAGUAACA | 2173 | TGTTACTA GGCTAGCTACAACGA TTGGGCCT | 5593 |
| 1650 | AAAUAGUA A CAGAGAAG | 2174 | CTTCTCTG GGCTAGCTACAACGA TACTATTT | 5594 |
| 1660 | AGAGAAGA A UACUAGCA | 2175 | TGCTAGTA GGCTAGCTACAACGA TCTTCTCT | 5595 |
| 1674 | GCACCAAA A CAUCAAAC | 2176 | GTTTGATG GGCTAGCTACAACGA TTTGGTGC | 5596 |
| 1681 | AACAUCAA A CCCUUUUC | 2177 | GAAAAGGG GGCTAGCTACAACGA TTGATGTT | 5597 |
| 1705 | AGCACAGG A UUCUGAGA | 2178 | TCTCAGAA GGCTAGCTACAACGA CCTGTGCT | 5598 |
| 1713 | AUUCUGAG A CAGAUUAU | 2179 | ATAATCTG GGCTAGCTACAACGA CTCAGAAT | 5599 |
| 1717 | UGAGACAG A UUAUGUCA | 2180 | TGACATAA GGCTAGCTACAACGA CTGTCTCA | 5600 |
| 1728 | AUGUCACA A CAGAUAAU | 2181 | ATTATCTG GGCTAGCTACAACGA TGTGACAT | 5601 |
| 1732 | CACAACAG A UAAUUUAA | 2182 | TTAAATTA GGCTAGCTACAACGA CTGTTGTG | 5602 |
| 1735 | AACAGAUA A UUUAACAA | 2183 | TTGTTAAA GGCTAGCTACAACGA TATCTGTT | 5603 |
| 1740 | AUAAUUUA A CAAAGGUG | 2184 | CACCTTTG GGCTAGCTACAACGA TAAATTAT | 5604 |
| 1749 | CAAAGGUG A CUGAGGAA | 2185 | TTCCTCAG GGCTAGCTACAACGA CACCTTTG | 5605 |
| 1768 | CGUGGCAA A CAUGCCUG | 2186 | CAGGCATG GGCTAGCTACAACGA TTGCCACG | 5606 |
| 1785 | AAGGCCUG A CUCCAGAU | 2187 | ATCTGGAG GGCTAGCTACAACGA CAGGCCTT | 5607 |
| 1792 | GACUCCAG A UUUAGUAC | 2188 | GTACTAAA GGCTAGCTACAACGA CTGGAGTC | 5608 |
| 1820 | GAAAGUGA A UUGAAUGA | 2189 | TCATTCAA GGCTAGCTACAACGA TCACTTTC | 5609 |
| 1825 | UGAAUUGA A UGAAGUUA | 2190 | TAACTTCA GGCTAGCTACAACGA TCAATTCA | 5610 |
| 1845 | GUACAAAG A UUGCUUAU | 2191 | ATAAGCAA GGCTAGCTACAACGA CTTTGTAC | 5611 |
| 1857 | CUUAUGAA A CAAAAAUG | 2192 | CATTTTTG GGCTAGCTACAACGA TTCATAAG | 5612 |
| 1863 | AAACAAAA A UGGACUUG | 2193 | CAAGTCCA GGCTAGCTACAACGA TTTTGTTT | 5613 |
| 1867 | AAAAAUGG A CUUGGUUC | 2194 | GAACCAAG GGCTAGCTACAACGA CCATTTTT | 5614 |
| 1878 | UGGUUCAA A CAUCAGAA | 2195 | TTCTGATG GGCTAGCTACAACGA TTGAACCA | 5615 |
| 1972 | UUUGCCUG A CAUUGUUA | 2196 | TAACAATG GGCTAGCTACAACGA CAGGCAAA | 5616 |
| 1996 | ACCAUUGA A UUCUGCAG | 2197 | CTGCAGAA GGCTAGCTACAACGA TCAATGGT | 5617 |
| 2028 | CUUCCGUG A UACAGCCC | 2198 | GGGCTGTA GGCTAGCTACAACGA CACGGAAG | 5618 |
| 2068 | UUCAGUUA A UUAUGAAA | 2199 | TTTCATAA GGCTAGCTACAACGA TAACTGAA | 5619 |
| 2084 | AGCAUAAA A CAUGAGCC | 2200 | GGCTCATG GGCTAGCTACAACGA TTTATGCT | 5620 |
| 2098 | GCCUGAAA A CCCCCCAC | 2201 | GTGGGGGG GGCTAGCTACAACGA TTTCAGGC | 5621 |
| 2151 | UAUCAGGA A UAAAGGAA | 2202 | TTCCTTTA GGCTAGCTACAACGA TCCTGATA | 5622 |
| 2163 | AGGAAGAA A UUAAAGAG | 2203 | CTCTTTAA GGCTAGCTACAACGA TTCTTCCT | 5623 |
| 2179 | GCCUGAAA A UAUUAAUG | 2204 | CATTAATA GGCTAGCTACAACGA TTTCAGGC | 5624 |
| 2185 | AAAUAUUA A UGCAGCUC | 2205 | GAGCTGCA GGCTAGCTACAACGA TAATATTT | 5625 |
| 2202 | UUCAAGAA A CAGAAGCU | 2206 | AGCTTCTG GGCTAGCTACAACGA TTCTTGAA | 5626 |
| 2233 | UGCAUGUG A UUUAAUUA | 2207 | TAATTAAA GGCTAGCTACAACGA CACATGCA | 5627 |
| 2238 | GUGAUUUA A UUAAAGAA | 2208 | TTCTTTAA GGCTAGCTACAACGA TAAATCAC | 5628 |
| 2247 | UUAAAGAA A CAAAGCUU | 2209 | AAGCTTTG GGCTAGCTACAACGA TTCTTTAA | 5629 |
| 2264 | UCUGCUGA A CCAGCUCC | 2210 | GGAGCTGG GGCTAGCTACAACGA TCAGCAGA | 5630 |
| 2275 | AGCUCCGG A UUUCUCUG | 2211 | CAGAGAAA GGCTAGCTACAACGA CCGGAGCT | 5631 |
| 2284 | UUUCUCUG A UUAUUCAG | 2212 | CTGAATAA GGCTAGCTACAACGA CAGAGAAA | 5632 |
| 2295 | AUUCAGAA A UGGCAAAA | 2213 | TTTTGCCA GGCTAGCTACAACGA TTCTGAAT | 5633 |
| 2309 | AAAGUUGA A CAGCCAGU | 2214 | ACTGGCTG GGCTAGCTACAACGA TCAACTTT | 5634 |
| 2323 | AGUGCCUG A UCAUUCUG | 2215 | CAGAATGA GGCTAGCTACAACGA CAGGCACT | 5635 |
| 2344 | AGUUGAAG A UUCCUCAC | 2216 | GTGAGGAA GGCTAGCTACAACGA CTTCAACT | 5636 |
| 2356 | CUCACCUG A UUCUGAAC | 2217 | GTTCAGAA GGCTAGCTACAACGA CAGGTGAG | 5637 |
| 2363 | GAUUCUGA A CCAGUUGA | 2218 | TCAACTGG GGCTAGCTACAACGA TCAGAATC | 5638 |
| 2371 | ACCAGUUG A CUUAUUUA | 2219 | TAAATAAG GGCTAGCTACAACGA CAACTGGT | 5639 |
| 2383 | AUUUAGUG A UGAUUCAA | 2220 | TTGAATCA GGCTAGCTACAACGA CACTAAAT | 5640 |
| 2386 | UAGUGAUG A UUCAAUAC | 2221 | GTATTGAA GGCTAGCTACAACGA CATCACTA | 5641 |
| 2391 | AUGAUUCA A UACCUGAC | 2222 | GTCAGGTA GGCTAGCTACAACGA TGAATCAT | 5642 |
| 2398 | AAUACCUG A CGUUCCAC | 2223 | GTGGAACG GGCTAGCTACAACGA CAGGTATT | 5643 |
| 2411 | CCACAAAA A CAAGAUGA | 2224 | TCATCTTG GGCTAGCTACAACGA TTTTGTGG | 5644 |
| 2416 | AAAACAAG A UGAAACUG | 2225 | CAGTTTCA GGCTAGCTACAACGA CTTGTTTT | 5645 |
| 2421 | AAGAUGAA A CUGUGAUG | 2226 | CATCACAG GGCTAGCTACAACGA TTCATCTT | 5646 |
| 2427 | AAACUGUG A UGCUUGUG | 2227 | CACAAGCA GGCTAGCTACAACGA CACAGTTT | 5647 |
| 2454 | UCACUGAG A CUUCAUUU | 2228 | AAATGAAG GGCTAGCTACAACGA CTCAGTGA | 5648 |
| 2469 | UUGAGUCA A UGAUAGAA | 2229 | TTCTATCA GGCTAGCTACAACGA TGACTCAA | 5649 |
| 2472 | AGUCAAUG A UAGAAUAU | 2230 | ATATTCTA GGCTAGCTACAACGA CATTGACT | 5650 |
| 2477 | AUGAUAGA A UAUGAAAA | 2231 | TTTTCATA GGCTAGCTACAACGA TCTATCAT | 5651 |
| 2485 | AUAUGAAA A UAAGGAAA | 2232 | TTTCCTTA GGCTAGCTACAACGA TTTCATAT | 5652 |
| 2495 | AAGGAAAA A CUCAGUGC | 2233 | GCACTGAG GGCTAGCTACAACGA TTTTCCTT | 5653 |
| 2537 | UAUUUGGA A UCUUUUAA | 2234 | TTAAAAGA GGCTAGCTACAACGA TCCAAATA | 5654 |
| 2557 | CAGUUUAG A UAACACAA | 2235 | TTGTGTTA GGCTAGCTACAACGA CTAAACTG | 5655 |
| 2560 | UUUAGAUA A CACAAAAG | 2236 | CTTTTGTG GGCTAGCTACAACGA TATCTAAA | 5656 |
| 2569 | CACAAAAG A UACCCUGU | 2237 | ACAGGGTA GGCTAGCTACAACGA CTTTTGTG | 5657 |
| 2584 | GUUACCUG A UGAAGUUU | 2238 | AAACTTCA GGCTAGCTACAACGA CAGGTAAC | 5658 |
| 2595 | AAGUUUCA A CAUUGAGC | 2239 | GCTCAATG GGCTAGCTACAACGA TGAAACTT | 5659 |
| 2616 | AGGAGAAA A UUCCUUUG | 2240 | CAAAGGAA GGCTAGCTACAACGA TTTCTCCT | 5660 |
| 2628 | CUUUGCAG A UGGAGGAG | 2241 | CTCCTCCA GGCTAGCTACAACGA CTGCAAAG | 5661 |
| 2659 | UUAUUCAA A UGAUGACU | 2242 | AGTCATCA GGCTAGCTACAACGA TTGAATAA | 5662 |
| 2662 | UUCAAAUG A UGACUUAU | 2243 | ATAAGTCA GGCTAGCTACAACGA CATTTGAA | 5663 |
| 2665 | AAAUGAUG A CUUAUUUA | 2244 | TAAATAAG GGCTAGCTACAACGA CATCATTT | 5664 |
| 2691 | AAGCACAG A UAAGAGAA | 2245 | TTCTCTTA GGCTAGCTACAACGA CTGTGCTT | 5665 |
| 2700 | UAAGAGAA A CUGAAACG | 2246 | CGTTTCAG GGCTAGCTACAACGA TTCTCTTA | 5666 |
| 2706 | AAACUGAA A CGUUUUCA | 2247 | TGAAAACG GGCTAGCTACAACGA TTCAGTTT | 5667 |
| 2716 | GUUUUCAG A UUCAUCUC | 2248 | GAGATGAA GGCTAGCTACAACGA CTGAAAAC | 5668 |
| 2727 | CAUCUCCA A UUGAAAUU | 2249 | AATTTCAA GGCTAGCTACAACGA TGGAGATG | 5669 |
| 2733 | CAAUUGAA A UUAUAGAU | 2250 | ATCTATAA GGCTAGCTACAACGA TTCAATTG | 5670 |
| 2740 | AAUUAUAG A UGAGUUCC | 2251 | GGAACTCA GGCTAGCTACAACGA CTATAATT | 5671 |
| 2757 | CUACAUUG A UCAGUUCU | 2252 | AGAACTGA GGCTAGCTACAACGA CAATGTAG | 5672 |
| 2769 | GUUCUAAA A CUGAUUCA | 2253 | TGAATCAG GGCTAGCTACAACGA TTTAGAAC | 5673 |
| 2773 | UAAAACUG A UUCAUUUU | 2254 | AAAATGAA GGCTAGCTACAACGA CAGTTTTA | 5674 |
| 2786 | UUUUCUAA A UUAGCCAG | 2255 | CTGGCTAA GGCTAGCTACAACGA TTAGAAAA | 5675 |
| 2798 | GCCAGGGA A UAUACUGA | 2256 | TCAGTATA GGCTAGCTACAACGA TCCCTGGC | 5676 |
| 2806 | AUAUACUG A CCUAGAAG | 2257 | CTTCTAGG GGCTAGCTACAACGA CAGTATAT | 5677 |
| 2832 | AAAGUGAA A UUGCUAAU | 2258 | ATTAGCAA GGCTAGCTACAACGA TTCACTTT | 5678 |
| 2839 | AAUUGCUA A UGCCCCGG | 2259 | CCGGGGCA GGCTAGCTACAACGA TAGCAATT | 5679 |
| 2848 | UGCCCCGG A UGGAGCUG | 2260 | CAGCTCCA GGCTAGCTACAACGA CCGGGGCA | 5680 |
| 2876 | UGCACAGA A UUGCCCCA | 2261 | TGGGGCAA GGCTAGCTACAACGA TCTGTGCA | 5681 |
| 2887 | GCCCCAUG A CCUUUCUU | 2262 | AAGAAAGG GGCTAGCTACAACGA CATGGGGC | 5682 |
| 2902 | UUUGAAGA A CAUACAAC | 2263 | GTTGTATG GGCTAGCTACAACGA TCTTCAAA | 5683 |
| 2909 | AACAUACA A CCCAAAGU | 2264 | ACTTTGGG GGCTAGCTACAACGA TGTATGTT | 5684 |
| 2928 | AAGAGAAA A UCAGUUUC | 2265 | GAAACTGA GGCTAGCTACAACGA TTTCTCTT | 5685 |
| 2941 | UUUCUCAG A UGACUUUU | 2266 | AAAAGTCA GGCTAGCTACAACGA CTGAGAAA | 5686 |
| 2944 | CUCAGAUG A CUUUUCUA | 2267 | TAGAAAAG GGCTAGCTACAACGA CATCTGAG | 5687 |
| 2956 | UUCUAAAA A UGGGUCUG | 2268 | CAGACCCA GGCTAGCTACAACGA TTTTAGAA | 5688 |
| 2995 | GCCUCCAG A UGUUUCUG | 2269 | CAGAAACA GGCTAGCTACAACGA CTGGAGGC | 5689 |
| 3024 | AAGCAGAG A UAGAGAGC | 2270 | GCTCTCTA GGCTAGCTACAACGA CTCTGCTT | 5690 |
| 3041 | AUAGUUAA A CCCAAAGU | 2271 | ACTTTGGG GGCTAGCTACAACGA TTAACTAT | 5691 |
| 3074 | GAGAAAAA A CUUCCUUC | 2272 | GAAGGAAG GGCTAGCTACAACGA TTTTTCTC | 5692 |
| 3085 | UCCUUCCG A UACAGAAA | 2273 | TTTCTGTA GGCTAGCTACAACGA CGGAAGGA | 5693 |
| 3100 | AAAAGAGG A CAGAUCAC | 2274 | GTGATCTG GGCTAGCTACAACGA CCTCTTTT | 5694 |
| 3104 | GAGGACAG A UCACCAUC | 2275 | GATGGTGA GGCTAGCTACAACGA CTGTCCTC | 5695 |
| 3141 | UGAGUAAA A CUUCAGUU | 2276 | AACTGAAG GGCTAGCTACAACGA TTTACTCA | 5696 |
| 3154 | AGUUGUUG A CCUCCUGU | 2277 | ACAGGAGG GGCTAGCTACAACGA CAACAACT | 5697 |
| 3172 | CUGGAGAG A CAUUAAGA | 2278 | TCTTAATG GGCTAGCTACAACGA CTCTCCAG | 5698 |
| 3183 | UUAAGAAG A CUGGAGUG | 2279 | CACTCCAG GGCTAGCTACAACGA CTTCTTAA | 5699 |
| 3228 | UUUCAUUG A CAGUAUUC | 2280 | GAATACTG GGCTAGCTACAACGA CAATGAAA | 5700 |
| 3252 | UGAGCGUA A CAGCCUAC | 2281 | GTAGGCTG GGCTAGCTACAACGA TACGCTCA | 5701 |
| 3285 | UCUCUGUG A CCAUCAGC | 2282 | GCTGATGG GGCTAGCTACAACGA CACAGAGA | 5702 |
| 3300 | GCUUUAGG A UAUACAAG | 2283 | CTTGTATA GGCTAGCTACAACGA CCTAAAGC | 5703 |
| 3315 | AGGGUGUG A UCCAAGCU | 2284 | AGCTTGGA GGCTAGCTACAACGA CACACCCT | 5704 |
| 3332 | AUCCAGAA A UCAGAUGA | 2285 | TCATCTGA GGCTAGCTACAACGA TTCTGGAT | 5705 |
| 3337 | GAAAUCAG A UGAAGGCC | 2286 | GGCCTTCA GGCTAGCTACAACGA CTGATTTC | 5706 |
| 3368 | UAUCUGGA A UCUGAAGU | 2287 | ACTTCAGA GGCTAGCTACAACGA TCCAGATA | 5707 |
| 3412 | GUACAGUA A UUCUGCUC | 2288 | GAGCAGAA GGCTAGCTACAACGA TACTGTAC | 5708 |
| 3433 | UCAUGUGA A CUGCACGA | 2289 | TCGTGCAG GGCTAGCTACAACGA TCACATGA | 5709 |
| 3441 | ACUGCACG A UAAAGGAA | 2290 | TTCCTTTA GGCTAGCTACAACGA CGTGCAGT | 5710 |
| 3449 | AUAAAGGA A CUCAGGCG | 2291 | CGCCTGAG GGCTAGCTACAACGA TCCTTTAT | 5711 |
| 3472 | CUUAGUUG A UGAUUUAG | 2292 | CTAAATCA GGCTAGCTACAACGA CAACTAAG | 5712 |
| 3475 | AGUUGAUG A UUUAGUUG | 2293 | CAACTAAA GGCTAGCTACAACGA CATCAACT | 5713 |
| 3484 | UUUAGUUG A UUCUCUGA | 2294 | TCAGAGAA GGCTAGCTACAACGA CAACTAAA | 5714 |
| 3507 | CAGUGUUG A UGUGGGUA | 2295 | TACCCACA GGCTAGCTACAACGA CAACACTG | 5715 |
| 3541 | CUUGUUUA A UGGUCUGA | 2296 | TCAGACCA GGCTAGCTACAACGA TAAACAAG | 5716 |
| 3549 | AUGGUCUG A CACUACUG | 2297 | CAGTAGTG GGCTAGCTACAACGA CAGACCAT | 5717 |
| 3558 | CACUACUG A UUUUGGCU | 2298 | AGCCAAAA GGCTAGCTACAACGA CAGTAGTG | 5718 |
| 3602 | AUUUAUGA A CGGCAUCA | 2299 | TGATGCCG GGCTAGCTACAACGA TCATAAAT | 5719 |
| 3618 | AGGCACAG A UAGAUCAU | 2300 | ATGATCTA GGCTAGCTACAACGA CTGTGCCT | 5720 |
| 3622 | ACAGAUAG A UCAUUAUC | 2301 | GATAATGA GGCTAGCTACAACGA CTATCTGT | 5721 |
| 3635 | UAUCUAGG A CUUGCAAA | 2302 | TTTGCAAG GGCTAGCTACAACGA CCTAGATA | 5722 |
| 3643 | ACUUGCAA A UAAGAAUG | 2303 | CATTCTTA GGCTAGCTACAACGA TTGCAAGT | 5723 |
| 3649 | AAAUAAGA A UGUUAAAG | 2304 | CTTTAACA GGCTAGCTACAACGA TCTTATTT | 5724 |
| 3658 | UGUUAAAG A UGCUAUGG | 2305 | CCATAGCA GGCTAGCTACAACGA CTTTAACA | 5725 |
| 3672 | UGGCUAAA A UCCAAGCA | 2306 | TGCTTGGA GGCTAGCTACAACGA TTTAGCCA | 5726 |
| 3684 | AAGCAAAA A UCCCUGGA | 2307 | TCCAGGGA GGCTAGCTACAACGA TTTTGCTT | 5727 |
| 3692 | AUCCCUGG A UUGAAGCG | 2308 | CGCTTCAA GGCTAGCTACAACGA CCAGGGAT | 5728 |
| 3710 | AAAGCUGA A UGAAAACG | 2309 | CGTTTTCA GGCTAGCTACAACGA TCAGCTTT | 5729 |
| 3716 | GAAUGAAA A CGCCCAAA | 2310 | TTTGGGCG GGCTAGCTACAACGA TTTCATTC | 5730 |
| 3725 | CGCCCAAA A UAAUUAGU | 2311 | ACTAATTA GGCTAGCTACAACGA TTTGGGCG | 5731 |
| 3728 | CCAAAAUA A UUAGUAGG | 2312 | CCTACTAA GGCTAGCTACAACGA TATTTTGG | 5732 |
| 3755 | UAAAGGGG A UAUUCAUU | 2313 | AATGAATA GGCTAGCTACAACGA CCCCTTTA | 5733 |
| 3766 | UUCAUUUG A UUAUACGG | 2314 | CCGTATAA GGCTAGCTACAACGA CAAATGAA | 5734 |
| 3792 | AGGGAAGA A CGAACCUU | 2315 | AAGGTTCG GGCTAGCTACAACGA TCTTCCCT | 5735 |
| 3796 | AAGAACGA A CCUUGACG | 2316 | CGTCAAGG GGCTAGCTACAACGA TCGTTCTT | 5736 |
| 3802 | GAACCUUG A CGUUGCAG | 2317 | CTGCAACG GGCTAGCTACAACGA CAAGGTTC | 5737 |
| 3824 | UUUCACAG A UCGUUGUU | 2318 | AACAACGA GGCTAGCTACAACGA CTGTGAAA | 5738 |
| 3835 | GUUGUUAG A UCUUUAUU | 2319 | AATAAAGA GGCTAGCTACAACGA CTAACAAC | 5739 |
| 3871 | GAGGAAAA A UUACCUGU | 2320 | ACAGGTAA GGCTAGCTACAACGA TTTTCCTC | 5740 |
| 3884 | CUGUCUUG A CUGCCAUG | 2321 | CATGGCAG GGCTAGCTACAACGA CAAGACAG | 5741 |
| 3931 | AUGUAUGG A UUUAAACC | 2322 | GGTTTAAA GGCTAGCTACAACGA CCATACAT | 5742 |
| 3937 | GGAUUUAA A CCGUAAUC | 2323 | GATTACGG GGCTAGCTACAACGA TTAAATCC | 5743 |
| 3943 | AAACCGUA A UCAUAUCU | 2324 | AGATATGA GGCTAGCTACAACGA TACGGTTT | 5744 |
| 3977 | CUGGUGGA A UAAAAAAC | 2325 | GTTTTTTA GGCTAGCTACAACGA TCCACCAG | 5745 |
| 3984 | AAUAAAAA A CCUGUAUA | 2326 | TATACAGG GGCTAGCTACAACGA TTTTTATT | 5746 |
| 4009 | UGUUGCAG A UAGUCUUG | 2327 | CAAGACTA GGCTAGCTACAACGA CTGCAACA | 5747 |
| 4041 | UUGCAGAG A UGGUGGAG | 2328 | CTCCACCA GGCTAGCTACAACGA CTCTGCAA | 5748 |

Input Sequence = AB-020693. Cut Site = R/Y
Stem Length = 8. Core Sequence = GGCTAGCTACAACGA
AB020693 (Homo sapiens mRNA for KIAA0886 protein (Nogo-A); 4053 bp)

TABLE VIII

Human NOGO Amberzyme Ribozyme and Substrate Sequence

| | | | | |
|---|---|---|---|---|
| | | | | Rz Seq |
| Pos | Substrate | Seq ID | Ribozyme | ID |

| | | | | |
|---|---|---|---|---|
| 66 | CCACAACC G CCCGCGGC | 1545 | GCCGCGGG GGAGGAAACUCC CU UCAAGGACAUCGUCCGGG GGUUGUGG | 5749 |
| 70 | AACCGCCC G CGGCUCUG | 1546 | CAGAGCCG GGAGGAAACUCC CU UCAAGGACAUCGUCCGGG GGGCGGUU | 5750 |
| 78 | GCGGCUCU G AGACGCGG | 1547 | CCGCGUCU GGAGGAAACUCC CU UCAAGGACAUCGUCCGGG AGAGCCGC | 5751 |
| 83 | UCUGAGAC G CGGCCCCG | 1548 | CGGGGCCG GGAGGAAACUCC CU UCAAGGACAUCGUCCGGG GUCUCAGA | 5752 |
| 110 | CAGCAGCU G CAGCAUCA | 1549 | UGAUGCUG GGAGGAAACUCC CU UCAAGGACAUCGUCCGGG AGCUGCUG | 5753 |
| 191 | CCCCGGCC G CAGCCCGC | 1550 | GCGGGCUG GGAGGAAACUCC CU UCAAGGACAUCGUCCGGG GGCCGGGG | 5754 |
| 198 | CGCAGCCC G CGUUCAAG | 1551 | CUUGAACG GGAGGAAACUCC CU UCAAGGACAUCGUCCGGG GGGCUGCG | 5755 |
| 218 | CAGUUCGU G AGGGAGCC | 1552 | GGCUCCCU GGAGGAAACUCC CU UCAAGGACAUCGUCCGGG ACGAACUG | 5756 |
| 228 | GGGAGCCC G AGGACGAG | 1553 | CUCGUCCU GGAGGAAACUCC CU UCAAGGACAUCGUCCGGG GGGCUCCC | 5757 |
| 234 | CCGAGGAC G AGGAGGAA | 1554 | UUCCUCCU GGAGGAAACUCC CU UCAAGGACAUCGUCCGGG GUCCUCGG | 5758 |
| 267 | AGGAGGAC G AGGACGAA | 1555 | UUCGUCCU GGAGGAAACUCC CU UCAAGGACAUCGUCCGGG GUCCUCCU | 5759 |
| 273 | ACGAGGAC G AAGACCUG | 1556 | CAGGUCUU GGAGGAAACUCC CU UCAAGGACAUCGUCCGGG GUCCUCGU | 5760 |
| 296 | CUGGAGGU G CUGGAGAG | 1557 | CUCUCCAG GGAGGAAACUCC CU UCAAGGACAUCGUCCGGG ACCUCCAG | 5761 |
| 312 | GGAAGCCC G CCGCCGGG | 1558 | CCCGGCGG GGAGGAAACUCC CU UCAAGGACAUCCUCCGGG GGGCUUCC | 5762 |
| 315 | AGCCCGCC G CCGGGCUC | 1559 | CAGCCCGG GGAGGAAACUCC CU UCAAGGACAUCGUCCGGG GGCGGGCU | 5763 |
| 327 | GGCUGUCC G CGGCCCCA | 1560 | UGGGGCCG GGAGGAAACUCC CU UCAAGGACAUCGUCCGGG GGACAGCC | 5764 |
| 338 | GCCCCAGU G CCCACCGC | 1561 | GCGGUGGG GGAGGAAACUCC CU UCAAGGACAUCGUCCGGG ACUGGGGC | 5765 |
| 345 | UGCCCACC G CCCCUGCC | 1562 | GGCAGGGG GGAGGAAACUCC CU UCAAGGACAUCGUCCGGG GGUGGGCA | 5766 |
| 351 | CCGCCCCU G CCGCCGGC | 1563 | GCCGGCGG GGAGGAAACUCC CU UCAAGGACAUCGUCCGGG AGGGGCGG | 5767 |
| 354 | CCCCUGCC G CCGGCGCG | 1564 | CGCGCCGG GGAGGAAACUCC CU UCAAGGACAUCGUCCGGG GGCAGGGG | 5768 |
| 360 | CCGCCGGC G CGCCCCUG | 1565 | CAGGGGCG GGAGGAAACUCC CU UCAAGGACAUCGUCCGGG GCCGGCGG | 5769 |
| 362 | GCCGGCGC G CCCCUGAU | 1566 | AUCAGGGG GGAGGAAACUCC CU UCAAGGACAUCGUCCGGG GCGCCGGC | 5770 |
| 368 | GCGCCCCU G AUGGACUU | 1567 | AAGUCCAU GGAGGAAACUCC CU UCAAGGACAUCGUCCGGG AGGGGCGC | 5771 |
| 384 | UCGGAAAU G ACUUCGUG | 1568 | CACGAAGU GGAGGAAACUCC CU UCAAGGACAUCGUCCGGG AUUUCCGA | 5772 |
| 392 | GACUUCGU G CCGCCGGC | 1569 | GCCGGCGG GGAGGAAACUCC CU UCAAGGACAUCGUCCGGG ACGAAGUC | 5773 |
| 395 | UUCGUGCC G CCGGCGCC | 1570 | GGCGCCGG GGAGGAAACUCC CU UCAAGGACAUCGUCCGGG GGCACGAA | 5774 |
| 401 | CCGCCGGC G CCCCGGGG | 1571 | CCCCGGGG GGAGGAAACUCC CU UCAAGGACAUCGUCCGGG GCCGGCGG | 5775 |
| 416 | GGACCCCU G CCGGCCGC | 1572 | GCGGCCGG GGAGGAAACUCC CU UCAAGGACAUCGUCCGGG AGGGGUCC | 5776 |
| 423 | UGCCGGCC G CUCCCCCC | 1573 | GGGGGGAG GGAGGAAACUCC CU UCAAGGACAUCGUCCGGG GGCCGGCA | 5777 |
| 435 | CCCCCGUC G CCCCGGAG | 1574 | CUCCGGGG GGAGGAAACUCC CU UCAAGGACAUCGUCCGGG GACGGGGG | 5778 |
| 464 | UGGGACCC G ACCCCGGU | 1575 | ACCGGGCU GGAGGAAACUCC CU UCAAGGACAUCGUCCGGG GGGUCCCA | 5779 |
| 479 | GUGUCGUC G ACCGUGCC | 1576 | GGCACGGU GGAGGAAACUCC CU UCAAGGACAUCGUCCGGG GACGACAC | 5780 |
| 485 | UCGACCGU G CCCGCGCC | 1577 | GGCGCGGG GGAGGAAACUCC CU UCAAGGACAUCGUCCGGG ACGGUCGA | 5781 |
| 489 | CCGUGCCC G CGCCAUCC | 1578 | GGAUGGCG GGAGGAAACUCC CU UCAAGGACAUCGUCCGGG GGGCACGG | 5782 |
| 491 | GUGCCCGC G CCAUCCCC | 1579 | GGGGAUGG GGAGGAAACUCC CU UCAAGGACAUCGUCCGGG GCGGGCAC | 5783 |
| 500 | CCAUCCCC G CUGUCUGC | 1580 | GCAGACAG GGAGGAAACUCC CU UCAAGGACAUCGUCCGGG GGGGAUGG | 5784 |
| 507 | CGCUGUCU G CUGCCGCA | 1581 | UGCGGCAG GGAGGAAACUCC CU UCAAGGACAUCGUCCGGG AGACAGCG | 5785 |
| 510 | UGUCUGCU G CCGCAGUC | 1582 | GACUGCGG GGAGGAAACUCC CU UCAAGGACAUCGUCCGGG AGCAGACA | 5786 |
| 513 | CUGCUGCC G CAGUCUCG | 1583 | CGAGACUG GGAGGAAACUCC CU UCAAGGACAUCGUCCGGG GGCAGCAG | 5787 |
| 521 | GCAGUCUC G CCCUCCAA | 1584 | UUGGAGGG GGAGGAAACUCC CU UCAAGGACAUCGUCCGGG GAGACUGC | 5788 |
| 537 | AGCUCCCU G AGGACGAC | 1585 | GUCGUCCU GGAGGAAACUCC CU UCAAGGACAUCGUCCGGG AGGGAGCU | 5789 |
| 543 | CUGAGGAC G ACGAGCCU | 1586 | AGGCUCGU GGAGGAAACUCC CU UCAAGGACAUCGUCCGGG GUCCUCAG | 5790 |
| 546 | AGGACGAC G AGCCUCCG | 1587 | CGGAGGCU GGAGGAAACUCC CU UCAAGGACAUCGUCCGGG GUCGUCCU | 5791 |
| 587 | GCCAGCGU G AGCCCCCA | 1588 | UGGGGGCU GGAGGAAACUCC CU UCAAGGACAUCGUCCGGG ACGCUGGC | 5792 |
| 617 | UGGACCCC G CCAGCCCC | 1589 | GGGGCUGG GGAGGAAACUCC CU UCAAGGACAUCGUCCGGG GGGGUCCA | 5793 |
| 633 | CGGCUCCC G CCGCGCCC | 1590 | GGGCGCGG GGAGGAAACUCC CU UCAAGGACAUCGUCCGGG GGGAGCCG | 5794 |
| 636 | CUCCCGCC G CGCCCCCC | 1591 | GGGGGGCG GGAGGAAACUCC CU UCAAGGACAUCGUCCGGG GGCGGGAG | 5795 |
| 638 | CCCGCCGC G CCCCCCUC | 1592 | GAGGGGGG GGAGGAAACUCC CU UCAAGGACAUCGUCCGGG GCGGCGGG | 5796 |
| 657 | CCCCGGCC G CGCCCAAG | 1593 | CUUGGGCG GGAGGAAACUCC CU UCAAGGACAUCGUCCGGG GGCCGGGG | 5797 |
| 659 | CCGGCCGC G CCCAAGCG | 1594 | CGCUUGGG GGAGGAAACUCC CU UCAAGGACAUCGUCCGGG GCGGCCGG | 5798 |
| 667 | GCCCAAGC G CAGGGGCU | 1595 | AGCCCCUG GGAGGAAACUCC CU UCAAGGACAUCGUCCGGG GCUUGGGC | 5799 |
| 693 | CAGUGGAU G AGACCCUU | 1596 | AAGGGUCU GGAGGAAACUCC CU UCAAGGACAUCGUCCGGG AUCCACUG | 5800 |
| 705 | CCCUUUUU G CUCUUCCU | 1597 | AGGAAGAG GGAGGAAACUCC CU UCAAGGACAUCGUCCGGG AAAAAGGG | 5801 |
| 714 | CUCUUCCU G CUGCAUCU | 1598 | AGAUGCAG GGAGGAAACUCC CU UCAAGGACAUCGUCCGGG AGGAAGAG | 5802 |
| 717 | UUCCUGCU G CAUCUGAC | 1599 | CUCAGAUG GGAGGAAACUCC CU UCAAGGACAUCGUCCGGG AGCAGGAA | 5803 |
| 723 | CUGCAUCU G AGCCUGUG | 1600 | CACAGGCU GGAGGAAACUCC CU UCAAGGACAUCGUCCGGG AGAUGCAG | 5804 |
| 731 | GAGCCUGU G AUACGCUC | 1601 | GAGCGUAU GGAGGAAACUCC CU UCAAGGACAUCGUCCGGG ACAGGCUC | 5805 |
| 736 | UGUGAUAC G CUCCUCUG | 1602 | CAGAGGAG GGAGGAAACUCC CU UCAAGGACAUCGUCCGGG GUAUCACA | 5806 |
| 744 | GCUCCUCU G CAGAAAAU | 1603 | AUUUUCUG GGAGGAAACUCC CU UCAAGGACAUCGUCCGGG AGAGGAGC | 5807 |
| 761 | AUGGACUU G AAGGAGCA | 1604 | UGCUCCUU GGAGGAAACUCC CU UCAAGGACAUCGUCCGGG AAGUCCAU | 5808 |
| 818 | UCUGUCCU G CUUGAAAC | 1605 | GUUUCAAG GGAGGAAACUCC CU UCAAGGACAUCGUCCGGG AGGACAGA | 5809 |
| 822 | UCCUGCUU G AAACUGCU | 1606 | AGCAGUUU GGAGGAAACUCC CU UCAAGGACAUCGUCCGGG AAGCAGGA | 5810 |
| 828 | UUGAAACU G CUGCUUCU | 1607 | AGAAGCAC GGAGGAAACUCC CU UCAAGGACAUCGUCCGGG AGUUUCAA | 5811 |
| 831 | AAACUGCU G CUUCUCUU | 1608 | AAGAGAAG GGAGGAAACUCC CU UCAAGGACAUCGUCCGGG AGCAGUUU | 5812 |
| 864 | UCUCAGCC G CUUCUUUC | 1609 | GAAAGAAG GGAGGAAACUCC CU UCAAGGACAUCGUCCGGG GGCUGAGA | 5813 |
| 882 | AAGAACAU G AAUACCUU | 1610 | AAGGUAUU GGAGGAAACUCC CU UCAAGGACAUCGUCCGGG AUGUUCUU | 5814 |
| 918 | UACCCACU G AAGGAACA | 1611 | UGUUCCUU GGAGGAAACUCC CU UCAAGGACAUCGUCCGGG AGUGGGUA | 5815 |
| 945 | AUGUCAGU G AAGCUUCU | 1612 | AGAAGCUU GGAGGAAACUCC CU UCAAGGACAUCGUCCGGG ACUGACAU | 5816 |
| 1071 | CAGAAUCU G CCGUAAUA | 1613 | UAUUACGG GGAGGAAACUCC CU UCAAGCACAUCGUCCGGG AGAUUCUG | 5817 |
| 1109 | AUAAUCGU G AAAAAUAA | 1614 | UUAUUUUU GGAGGAAACUCC CU UCAAGGACAUCGUCCGGG ACGAUUAU | 5818 |
| 1122 | AUAAAGAU G AAGAAGAG | 1615 | CUCUUCUU GGAGGAAACUCC CU UCAAGGACAUCGUCCGGG AUCUUUAU | 5819 |
| 1206 | AAGAGGAU G AAGUUGUG | 1616 | CACAACUU GGAGGAAACUCC CU UCAAGGACAUCGUCCGGG AUCCUCUU | 5820 |
| 1245 | GUUUUAAU G AAAAGAGA | 1617 | UCUCUUUC GGAGGAAACUCC CU UCAAGGACAUCGUCCGGG AUUAAAAC | 5821 |
| 1257 | AGAGAGUU G CAGUGGAA | 1618 | UUCCACUG GGAGGAAACUCC CU UCAAGGACAUCGUCCGGG AACUCUCU | 5822 |
| 1274 | GCUCCUAU G AGGGAGGA | 1619 | UCCUCCCU GGAGGAAACUCC CU UCAAGGACAUCGUCCGGG AUAGGAGC | 5823 |
| 1287 | AGGAAUAU G CAGACUUC | 1620 | GAAGUCUG GGAGGAAACUCC CU UCAAGGACAUCGUCCGGG AUAUUCCU | 5824 |
| 1305 | AACCAUUU G AGCGAGUA | 1621 | UACUCGCU GGAGGAAACUCC CU UCAAGGACAUCGUCCGGG AAAUGGUU | 5825 |
| 1309 | AUUUGAGC G AGUAUGGG | 1622 | CCCAUACU GGAGGAAACUCC CU UCAAGGACAUCGUCCGGG GCUCAAAU | 5826 |
| 1322 | UGGGAAGU G AAAGAUAG | 1623 | CUAUCUUU GGAGGAAACUCC CU UCAAGGACAUCGUCCGGG ACUUCCCA | 5827 |
| 1344 | AAGAUAGU G AUAUGUUG | 1624 | CAACAUAU GGAGGAAACUCC CU UCAAGGACAUCGUCCGGG ACUAUCUU | 5828 |
| 1356 | UGUUGGCU G CUGGAGGU | 1625 | ACCUCCAG GGAGGAAACUCC CU UCAAGGACAUCGUCCGGG AGCCAACA | 5829 |
| 1371 | GUAAAAUC G AGAGCAAC | 1626 | GUUGCUCU GGAGGAAACUCC CU UCAAGGACAUCGUCCGGG GAUUUUAC | 5830 |
| 1410 | AAUGUUUU G CAGAUAGC | 1627 | GCUAUCUG GGAGGAAACUCC CU UCAAGGACAUCGUCCGGG AAAACAUU | 5831 |
| 1422 | AUAGCCUU G AGCAAACU | 1628 | AGUUUGCU GGAGGAAACUCC CU UCAAGGACAUCGUCCGGG AAGGCUAU | 5832 |
| 1437 | CUAAUCAC G AAAAAGAU | 1629 | AUCUUUUU GGAGGAAACUCC CU UCAAGGACAUCGUCCGGG GUGAUUAG | 5833 |
| 1449 | AAGAUAGU G AGAGUAGU | 1630 | ACUACUCU GGAGGAAACUCC CU UCAAGGACAUCGUCCGGG ACUAUCUU | 5834 |
| 1461 | GUAGUAAU G AUGAUACU | 1631 | AGUAUCAU GGAGGAAACUCC CU UCAAGGACAUCGUCCGGG AUUACUAC | 5835 |
| 1464 | GUAAUGAU G AUACUUCU | 1632 | AGAAGUAU GGAGGAAACUCC CU UCAAGGACAUCGUCCGGG AUCAUUAC | 5836 |
| 1484 | CCCAGUAC G CCAGAAGG | 1633 | CCUUCUGG GGAGGAAACUCC CU UCAAGGACAUCGUCCGGG GUACUGGG | 5837 |
| 1527 | UCACAUGU G CUCCCUUU | 1634 | AAAGGGAG GGAGGAAACUCC CU UCAAGGACAUCGUCCGGG ACAUGUGA | 5838 |
| 1551 | CAGCAACU G AGAUGCUU | 1635 | AAUGCUCU GGAGGAAACUCC CU UCAAGGACAUCGUCCGGG AGUUGCUG | 5839 |
| 1560 | AGAGCACU G CAACAAAC | 1636 | GUUUGUUG GGAGGAAACUCC CU UCAAGGACAUCGUCCGGG AAUGCUCU | 5840 |
| 1611 | AUAAGACC G AUGAAAAA | 1637 | UUUUUCAU GGAGGAAACUCC CU UCAAGGACAUCGUCCGGG GGUCUUAU | 5841 |
| 1614 | AGACCGAU G AAAAAAAA | 1638 | UUUUUUUU GGAGGAAACUCC CU UCAAGGACAUCGUCCGGG AUCGGUCU | 5842 |
| 1710 | AGGAUUCU G AGACAGAU | 1639 | AUCUGUCU GGAGGAAACUCC CU UCAAGGACAUCGUCCGGG AGAAUCCU | 5843 |
| 1748 | ACAAAGGU G ACUGAGGA | 1640 | UCCUCAGU GGAGGAAACUCC CU UCAAGGACAUCGUCCGGG ACCUUUGU | 5844 |
| 1752 | AGGUGACU G AGGAAGUC | 1641 | GACUUCCU GGAGGAAACUCC CU UCAAGGACAUCGUCCGGG AGUCACCU | 5845 |
| 1772 | GCAAACAU G CCUGAAGG | 1642 | CCUUCAGG GGAGGAAACUCC CU UCAAGGACAUCGUCCGGG AUGUUUGC | 5846 |
| 1776 | ACAUGCCU G AAGGCCUG | 1643 | CAGGCCUU GGAGGAAACUCC CU UCAAGGACAUCGUCCGGG AGGCAUGU | 5847 |
| 1784 | GAAGGCCU G ACUCCAGA | 1644 | UCUGGAGU GGAGGAAACUCC CU UCAAGGACAUCGUCCGGG AGGCCUUC | 5848 |
| 1812 | AAGCAUUU G AAAGUGAA | 1645 | UUCACUUU GGAGGAAACUCC CU UCAAGGACAUCGUCCGGG ACAUGCUU | 5849 |
| 1818 | GUGAAAUU G AAUUGAAU | 1646 | AUUCAAUU GGAGGAAACUCC CU UCAAGGACAUCGUCCGGG ACUUUCUU | 5850 |
| 1823 | AGUGAAUU G AAUGAAGU | 1647 | ACUUCAUU GGAGGAAACUCC CU UCAAGGACAUCGUCCGGG AAUUCACU | 5851 |
| 1827 | AAUUGAAU G AAGUUACU | 1648 | AGUAACUU GGAGGAAACUCC CU UCAAGGACAUCGUCCGGG AUUCAAUU | 5852 |
| 1848 | CAAAGAUU G CUUAUGAA | 1649 | UUCAUAAG GGAGGAAACUCC CU UCAAGGACAUCGUCCGGG AAUCUUUG | 5853 |
| 1854 | UUGCUUAU G AAACAAAA | 1650 | UUUUGUUU GGAGGAAACUCC CU UCAAGGACAUCGUCCGGG AUAAGCAA | 5854 |
| 1892 | GAAGUUAU G CAAGAGUC | 1651 | GACUCUUG GGAGGAAACUCC CU UCAAGGACAUCGUCCGGG AUAACUUC | 5855 |
| 1911 | UCUAUCCU G CAGCACAG | 1652 | CUGUGCUG GGAGGAAACUCC CU UCAAGGACAUCGUCCGGG AGGAUAGA | 5856 |
| 1924 | ACAUCUUU G CCCAUCAU | 1653 | AUGAUGGG GGAGGAAACUCC CU UCAAGGACAUCGUCCGGG AAAGCUGU | 5857 |
| 1935 | CAUCAUUU G AAGAGUCA | 1654 | UGACUCUU GGAGGAAACUCC CU UCAAGGACAUCGUCCGGG AAAUGAUG | 5858 |
| 1967 | CCAGUUUU G CCUGACAU | 1655 | AUGUCAGG GGAGGAAACUCC CU UCAAGGACAUCGUCCGGG AAAACUGG | 5859 |
| 1971 | UUUUGCCU G ACAUUGUU | 1656 | AACAAUGU GGAGGAAACUCC CU UCAAGGACAUCGUCCGGG AGGCAAAA | 5860 |
| 1994 | GCACCAUU G AAUUCUGC | 1657 | GCAGAAUU GGAGGAAACUCC CU UCAAGGACAUCGUCCGGG AAUGGUGC | 5861 |
| 2001 | UGAAUUCU G CAGUUCCU | 1658 | AGGAACUG GGAGGAAACUCC CU UCAAGGACAUCGUCCGGG AGAAUUCA | 5862 |
| 2013 | UUCCUAGU G CUGGUGCU | 1659 | AGCACCAG GGAGGAAACUCC CU UCAAGGACAUCGUCCGGG ACUAGGAA | 5863 |
| 2019 | GUGCUGGU G CUUCCGUG | 1660 | CACGGAAG GGAGGAAACUCC CU UCAAGGACAUCGUCCGGG ACCAGCAC | 5864 |
| 2027 | GCUUCCGU G AUACAGCC | 1661 | GGCUGUAU GGAGGAAACUCC CU UCAAGGACAUCGUCCGGG ACGGAAGC | 5865 |
| 2073 | UUAAUUAU G AAAGCAUA | 1662 | UAUGCUUU GGAGGAAACUCC CU UCAAGGACAUCGUCCGGG AUAAUUAA | 5866 |
| 2088 | UAAAACAU G AGCCUGAA | 1663 | UUCAGGCU GGAGGAAACUCC CU UCAAGGACAUCGUCCGGG AUGUUUUA | 5867 |
| 2094 | AUGAGCCU G AAAACCCC | 1664 | GGGGUUUU GGAGGAAACUCC CU UCAAGGACAUCGUCCGGG AGGCUCAU | 5868 |
| 2112 | CACCAUAU G AAGAGGCC | 1665 | GGCCUCUU GGAGGAAACUCC CU UCAAGGACAUCGUCCGGG AUAUGGUG | 5869 |
| 2123 | GAGGCCAU G AGUGUAUC | 1666 | GAUACACU GGAGGAAACUCC CU UCAAGGACAUCGUCCGGG AUGGCCUC | 5870 |
| 2175 | AAGAGCCU G AAAAUAUU | 1667 | AAUAUUUU GGAGGAAACUCC CU UCAAGGACAUCGUCCGGG AGGCUCUU | 5871 |
| 2187 | AUAUUAAU G CAGCUCUU | 1668 | AAGAGCUG GGAGGAAACUCC CU UCAAGGACAUCGUCCGGG AUUAAUAU | 5872 |
| 2226 | UAUCUAUU G CAUGUGAU | 1669 | AUCACAUG GGAGGAAACUCC CU UCAAGGACAUCGUCCGGG AAUAGAUA | 5873 |
| 2232 | UUGCAUGU G AUUUAAUU | 1670 | AAUUAAAU GGAGGAAACUCC CU UCAAGGACAUCGUCCGGG ACAUGCAA | 5874 |
| 2259 | AGCUUUCU G CUGAACCA | 1671 | UGGUUCAG GGAGGAAACUCC CU UCAAGGACAUCGUCCGGG AGAAAGCU | 5875 |
| 2262 | UUUCUGCU G AACCAGCU | 1672 | AGCUGGUU GGAGGAAACUCC CU UCAAGGACAUCGUCCGGG AGCAGAAA | 5876 |
| 2283 | AUUUCUCU G AUUAUUCA | 1673 | UGAAUAAU GGAGGAAACUCC CU UCAAGGACAUCGUCCGGG AGAGAAAU | 5877 |
| 2307 | CAAAAGUU G AACAGCCA | 1674 | UGGCUGUU GGAGGAAACUCC CU UCAAGGACAUCGUCCGGG AACUUUUG | 5878 |
| 2318 | CAGCCAGU G CCUGAUCA | 1675 | UGAUCAGG GGAGGAAACUCC CU UCAAGGACAUCGUCCGGG ACUGGCUG | 5879 |
| 2322 | CAGUGCCU G AUCAUUCU | 1676 | AGAAUGAU GGAGGAAACUCC CU UCAAGGACAUCGUCCGGG AGGCACUG | 5880 |
| 2331 | AUCAUUCU G AGCUAGUU | 1677 | AACUAGCU GGAGGAAACUCC CU UCAAGGACAUCGUCCGGG AGAAUGAU | 5881 |
| 2340 | AGCUAGGG G AAGAUUCC | 1678 | GGAAUCUU GGAGGAAACUCC CU UCAAGGACAUCGUCCGGG AACUAGCU | 5882 |
| 2355 | CCUCACCU G AUUCUGAA | 1679 | UUCAGAAU GGAGGAAACUCC CU UCAAGGACAUCGUCCGGG AGGUGAGG | 5883 |
| 2361 | CUGAUUCU G AACCAGUU | 1680 | AACUGGUU GGAGGAAACUCC CU UCAAGGACAUCGUCCGGG AGAAUCAG | 5884 |
| 2370 | AACCAGUU G ACUUAUUU | 1681 | AAAUAAGU GGAGGAAACUCC CU UCAAGGACAUCGUCCGGG AACUGGUU | 5885 |
| 2382 | UAUUUAGU G AUGAUUCA | 1682 | UGAAUCAU GGAGGAAACUCC CU UCAAGGACAUCGUCCGGG ACUAAAUA | 5886 |
| 2385 | UUAGUGAU G AUUCAAUA | 1683 | UAUUGAAU GGAGGAAACUCC CU UCAAGGACAUCGUCCGGG AUCACUAA | 5887 |
| 2397 | CAAUACCU G ACGUUCCA | 1684 | UGGAACGU GGAGGAAACUCC CU UCAAGGACAUCGUCCGGG AGGUAUUG | 5888 |
| 2418 | AACAAGAU G AAACUGUG | 1685 | CACAGUUU GGAGGAAACUCC CU UCAAGGACAUCGUCCGGG AUCUUGUU | 5889 |
| 2426 | GAAACUGU G AUGCUUGU | 1686 | ACAAGCAU GGAGGAAACUCC CU UCAAGGACAUCGUCCGGG ACAGUUUC | 5890 |
| 2429 | ACUGUGAU G CUUGUCAA | 1687 | UUCACAAG GGAGGAAACUCC CU UCAAGGACAUCGUCCGGG AUCACAGU | 5891 |
| 2435 | AUGCUUGU G AAAGAAAG | 1688 | CUUUCUUU GGAGGAAACUCC CU UCAAGGACAUCGUCCGGG ACAAGCAU | 5892 |
| 2451 | GUCUCACU G AGACUUCA | 1689 | UGAAGUCU GGAGGAAACUCC CU UCAAGGACAUCGUCCGGG AGUGAGAC | 5893 |
| 2463 | CUUCAUUU G AGUCAAUG | 1690 | CAUUGACU GGAGGAAACUCC CU UCAAGGACAUCGUCCGGG AAAUGAAG | 5894 |
| 2471 | CAGUCAAU G AUAGAAUA | 1691 | UAUUCUAU GGAGGAAACUCC CU UCAAGGACAUCGUCCGGG AUUGACUC | 5895 |
| 2481 | UAGAAUAU G AAAAUAAG | 1692 | CUUAUUUU GGAGGAAACUCC CU UCAAGCACAUCGUCCGGG AUAUUCUA | 5896 |
| 2502 | AACUCAGU G CUUUGCCA | 1693 | UGGCAAAG GGAGGAAACUCC CU UCAAGGACAUCGUCCGGG ACUGAGUU | 5897 |
| 2507 | AGUGCUUU G CCACCUGA | 1694 | UCAGGUGG GGAGGAAACUCC CU UCAAGGACAUCGUCCGGG AAAGCACU | 5898 |
| 2514 | UGCCACCU G AGGGAGGA | 1695 | UCCUCCCU GGAGGAAACUCC CU UCAAGGACAUCGUCCGGG AGGUGGCA | 5899 |
| 2583 | UGUUACCU G AUGAAGUU | 1696 | AACUUCAU GGAGGAAACUCC CU UCAAGGACAUCGUCCGGG AGGUAACA | 5900 |
| 2586 | UACCUGAU G AAGUUUCA | 1697 | UGAAACUU GGAGGAAACUCC CU UCAAGGACAUCGUCCGGG AUCAGGUA | 5901 |
| 2600 | UCAACAUU G AGCAAAAA | 1698 | UUUUUGCU GGAGGAAACUCC CU UCAAGGACAUCGUCCGGG AAUGUUGA | 5902 |
| 2624 | AUUCCUUU G CAGAUGGA | 1699 | UCCAUCUG GGAGGAAACUCC CU UCAAGGACAUCGUCCGGG AAAGGAAU | 5903 |
| 2646 | UCAGUACU G CAGUUUAU | 1700 | AUAAACUG GGAGGAAACUCC CU UCAAGGACAUCGUCCGGG AGUACUGA | 5904 |
| 2661 | AUUCAAAU G AUGACUUA | 1701 | UAAGUCAU GGAGGAAACUCC CU UCAAGGACAUCGUCCGGG AUUUGAAU | 5905 |
| 2664 | CAAAUGAU G ACUUAUUU | 1702 | AAAUAAGU GGAGGAAACUCC CU UCAAGGACAUCGUCCGGG AUCAUUUG | 5906 |
| 2703 | GAGAAACU G AAACGUUU | 1703 | AAACGUUU GGAGGAAACUCC CU UCAAGGACAUCGUCCGGG AGUUUCUC | 5907 |
| 2730 | CUCCAAUU G AAAUUAUA | 1704 | UAUAAUUU GGAGGAAACUCC CU UCAAGGACAUCGUCCGGG AAUUGGAG | 5908 |
| 2742 | UUAUAGAU G AGUUCCCU | 1705 | AGGGAACU GGAGGAAACUCC CU UCAAGGACAUCGUCCGGG AUCUAUAA | 5909 |
| 2756 | CCUACAUU G AUCAGUUC | 1706 | GAACUGAU GGAGGAAACUCC CU UCAAGGACAUCGUCCGGG AAUGUAGG | 5910 |
| 2772 | CUAAAACU G AUUCAUUU | 1707 | AAAUGAAU GGAGGAAACUCC CU UCAAGGACAUCGUCCGGG AGUUUUAG | 5911 |
| 2805 | AAUAUACU G ACCUAGAA | 1708 | UUCUAGGU GGAGGAAACUCC CU UCAAGGACAUCGUCCGGG AGUAUAUU | 5912 |
| 2829 | ACAAAAGU G AAAUUGCU | 1709 | AGCAAUUU GGAGGAAACUCC CU UCAAGGACAUCGUCCGGG ACUUUUGU | 5913 |
| 2835 | GUGAAAUU G CUAAUGCC | 1710 | GGCAUUAG GGAGGAAACUCC CU UCAAGGACAUCGUCCGGG AAUUUCAC | 5914 |
| 2841 | UUGCUAAU G CCCCGGAU | 1711 | AUCCGGGG GGAGGAAACUCC CU UCAAGGACAUCGUCCGGG AUUAGCAA | 5915 |
| 2864 | GGGUCAUU G CCUUGCAC | 1712 | GUGCAAGG GGAGGAAACUCC CU UCAAGGACAUCGUCCGGG AAUGACCC | 5916 |
| 2869 | AUUGCCUU G CACAGAAU | 1713 | AUUCUGUG GGAGGAAACUCC CU UCAAGGACAUCGUCCGGG AAGGCAAU | 5917 |
| 2879 | ACAGAAUU G CCCCAUGA | 1714 | UCAUGGGG GGAGGAAACUCC CU UCAAGGACAUCGUCCGGG AAUUCUGU | 5918 |
| 2886 | UGCCCCAU G ACCUUUCU | 1715 | AGAAAGGU GGAGGAAACUCC CU UCAAGGACAUCGUCCGGG AUGGGGCA | 5919 |
| 2897 | CUUUCUUU G AAGAACAU | 1716 | AUGUUCUU GGAGGAAACUCC CU UCAAGGACAUCGUCCGGG AAAGAAAG | 5920 |
| 2919 | CCAAAGUU G AAGAGAAA | 1717 | UUUCUCUU GGAGGAAACUCC CU UCAAGGACAUCGUCCGGG AACUUUGG | 5921 |
| 2943 | UCUCAGAU G ACUUUUCU | 1718 | AGAAAAGU GGAGGAAACUCC CU UCAAGGACAUCGUCCGGG AUCUGAGA | 5922 |
| 2964 | AUGGGUCU G CUACAUCA | 1719 | UGAUGUAG GGAGGAAACUCC CU UCAAGGACAUCGUCCGGG AGACCCAU | 5923 |
| 2978 | UCAAAGGU G CUCUUAUU | 1720 | AAUAAGAG GGAGGAAACUCC CU UCAAGGACAUCGUCCGGG ACCUUUGA | 5924 |
| 2987 | CUCUUAUU G CCUCCAGA | 1721 | UCUGGAGG GGAGGAAACUCC CU UCAAGGACAUCGUCCGGG AAUAAGAG | 5925 |
| 3003 | AUGUUUCU G CUUUGGCC | 1722 | GGCCAAAG GGAGGAAACUCC CU UCAAGGACAUCGUCCGGG AGAAACAU | 5926 |
| 3056 | GUUCUUGU G AAAGAACC | 1723 | GCUUCUUU GGAGGAAACUCC CU UCAAGGACAUCGUCCGGG ACAAGAAC | 5927 |
| 3066 | AAGAAGCU G AGAAAAAA | 1724 | UUUUUUCU GGAGGAAACUCC CU UCAAGGACAUCGUCCGGG AGCUUCUU | 5928 |
| 3084 | UUCCUUCC G AUACAGAA | 1725 | UUCUGUAU GGAGGAAACUCC CU UCAAGGACAUCGUCCGGG GGAAGGAA | 5929 |
| 3114 | CACCAUCU G CUAUAUUU | 1726 | AAAUAUAG GGAGGAAACUCC CU UCAAGGACAUCGUCCGGG AGAUGGUG | 5930 |
| 3134 | GCAGAGCU G AGUAAAAC | 1727 | GUUUUACU GGAGGAAACUCC CU UCAAGGACAUCGUCCGGG AGCUCUGC | 5931 |
| 3153 | CAGUUGUU G ACCUCCUG | 1728 | CAGGAGGU GGAGGAAACUCC CU UCAAGGACAUCGUCCGGG AACAACUG | 5932 |
| 3201 | UCUUUGGU G CCAGCCUA | 1729 | UAGGCUGG GGAGGAAACUCC CU UCAAGGACAUCGUCCGGG ACCAAACA | 5933 |
| 3215 | CUAUUCCU G CUGCUUUC | 1730 | GAAAGCAG GGAGGAAACUCC CU UCAAGGACAUCGUCCGGG AGGAAUAG | 5934 |
| 3218 | UUCCUGCU G CUUUCAUU | 1731 | AAUGAAAG GGAGGAAACUCC CU UCAAGGACAUCGUCCGGG AGCAGGAA | 5935 |
| 3227 | CUUUCAUU G ACAGUAUU | 1732 | AAUACUGU GGAGGAAACUCC CU UCAAGGACAUCGUCCGGG AAUGAAAG | 5936 |
| 3245 | AGCAUUGU G AGCGUAAC | 1733 | GUUACGCU GGAGGAAACUCC CU UCAAGGACAUCGUCCGGG ACAAUGCU | 5937 |
| 3264 | CCUACAUU G CCUUGGCC | 1734 | GGCCAAGG GGAGGAAACUCC CU UCAAGGACAUCGUCCGGG AAUGUAGG | 5938 |
| 3275 | UUGGCCCU G CUCUCUGU | 1735 | ACAGAGAG GGAGGAAACUCC CU UCAAGGACAUCGUCCGGG AGGGCCAA | 5939 |
| 3284 | CUCUCUGU G ACCAUCAG | 1736 | CUGAUGGU GGAGGAAACUCC CU UCAAGGACAUCGUCCGGG ACAGAGAG | 5940 |
| 3314 | AAGGGUGU G AUCCAAGC | 1737 | GCUUGGAU GGAGGAAACUCC CU UCAAGGACAUCGUCCGGG ACACCCUU | 5941 |
| 3339 | AAUCAGAU G AAGGCCAC | 1738 | GUGGCCUU GGAGGAAACUCC CU UCAAGGACAUCGUCCGGG AUCUGAUU | 5942 |
| 3372 | UGGAAUCU G AAGUUGCU | 1739 | AGCAACUU GGAGGAAACUCC CU UCAAGGACAUCGUCCGGG AGAUUCCA | 5943 |
| 3378 | CUGAAGGG G CUAUAUCU | 1740 | AGAUAUAG GGAGGAAACUCC CU UCAAGGACAUCGUCCGGG AACUUCAG | 5944 |
| 3387 | CUAUAUCU G AGGAGUUG | 1741 | CAACUCCU GGAGGAAACUCC CU UCAAGGACAUCGUCCGGG AGAUAUAG | 5945 |
| 3417 | GUAAUUCU G CUCUUGGU | 1742 | ACCAAGAG GGAGGAAACUCC CU UCAAGGACAUCGUCCGGG AGAAUUAC | 5946 |
| 3431 | GGUCAUGU G AACUGCAC | 1743 | GUGCAGUU GGAGGAAACUCC CU UCAAGGACAUCGUCCGGG ACAUGACC | 5947 |
| 3436 | UGUGAACU G GACGAUAA | 1744 | UUAUCGUG GGAGGAAACUCC CU UCAAGGACAUCGUCCGCG AGUUCACA | 5948 |
| 3440 | AACUGCAC G AUAAAGGA | 1745 | UCCUUUAU GGAGGAAACUCC CU UCAAGGACAUCGUCCGGG GUGCAGUU | 5949 |
| 3457 | ACUCAGUC G CCUCUUCU | 1746 | AGAAGAGG GGAGGAAACUCC CU UCAAGGACAUCGUCCGGG GCCAGUGU | 5950 |
| 3471 | UCUUAGUU G AUGAUUUA | 1747 | UAAAUCAU GGAGGAAACUCC CU UCAAGGACAUCGUCCGGG AACUAAGA | 5951 |
| 3474 | UAGUUGAU G AUUUAGUU | 1748 | AACUAAAU GGAGGAAACUCC CU UCAAGGACAUCGUCCGGG AUCAACUA | 5952 |
| 3483 | AUUUAGGG G AUUCUCUG | 1749 | CAGAGAAU GGAGGAAACUCC CU UCAAGGACAUCGUCCGGG AACUAAAU | 5953 |
| 3491 | GAUUCUCU G AAGUUUGC | 1750 | GCAAACUU GGAGGAAACUCC CU UCAAGGACAUCGUCCGGG AGAGAAUC | 5954 |
| 3498 | UGAAGUUU G CAGUGUUG | 1751 | CAACACUG GGAGGAAACUCC CU UCAAGGACAUCGUCCGGG AAACUUCA | 5955 |
| 3506 | GCAGUGUU G AUGUGGGU | 1752 | ACCCACAU GGAGGAAACUCC CU UCAAGGACAUCGUCCGGG AACACUGC | 5956 |
| 3531 | AUGUUGGU G CCUUGUUU | 1753 | AAACAAGG GGAGGAAACUCC CU UCAAGGACAUCGUCCGGG ACCAACAU | 5957 |
| 3548 | AAUGGUCU G ACACUACU | 1754 | AGUAGUGU GGAGGAAACUCC CU UCAAGGACAUCGUCCGGG AGACCAUU | 5958 |
| 3557 | ACACUACU G AUUUUGGC | 1755 | GCCAAAAU GGAGGAAACUCC CU UCAAGGACAUCGUCCGGG AGUAGUGU | 5959 |
| 3600 | UUAUUUAU G AACGGCAU | 1756 | AUGCCGUU GGAGGAAACUCC CU UCAAGGACAUCGUCCGGG AUAAAUAA | 5960 |
| 3639 | UAGGACUU G CAAAUAAG | 1757 | CUUAUUUG GGAGGAAACUCC CU UCAAGGACAUCGUCCGGG AAGUCCUA | 5961 |
| 3660 | UUAAAGAU G CUAUGGCU | 1758 | AGCCAUAG GGAGGAAACUCC CU UCAAGGACAUCGUCCGGG AUCUUUAA | 5962 |
| 3695 | CCUGGAUU G AAGCGCAA | 1759 | UUGCGCUU GGAGGAAACUCC CU UCAAGGACAUCGUCCGGG AAUCCAGG | 5963 |
| 3700 | AUUGAAGC G CAAAGCUG | 1760 | CAGCUUUG GGAGGAAACUCC CU UCAAGGACAUCGUCCGGG GCUUCAAU | 5964 |
| 3708 | GCAAAGCU G AAUGAAAA | 1761 | UUUUCAUU GGAGGAAACUCC CU UCAAGGACAUCGUCCGGG AGCUUUGC | 5965 |
| 3712 | AGCUGAAU G AAAACGCC | 1762 | GGCGUUUU GGAGGAAACUCC CU UCAAGGACAUCGUCCGGG AUUCAGCU | 5966 |
| 3718 | AUGAAAAC G CCCAAAAU | 1763 | AUUUUGGG GGAGGAAACUCC CU UCAAGGACAUCGUCCGGG GUUUUCAU | 5967 |
| 3765 | AUUCAUUU G AUUAUACG | 1764 | CGUAUAAU GGAGGAAACUCC CU UCAAGGACAUCGUCCGGG AAAUGAAU | 5968 |
| 3794 | GGAAGAAC G AACCUUGA | 1765 | UCAAGGUU GGAGGAAACUCC CU UCAAGGACAUCGUCCGGG GUUCUUCC | 5969 |
| 3801 | CGAACCUU G ACGUUGCA | 1766 | UGCAACGU GGAGGAAACUCC CU UCAAGGACAUCGUCCGGG AAGGUUCG | 5970 |
| 3807 | UUGACGUU G CAGUGCAC | 1767 | CUGCACUG GGAGGAAACUCC CU UCAAGGACAUCGUCCGGG AACGUCAA | 5971 |
| 3812 | GUUGCACU G CAGUUUCA | 1768 | UGAAACUG GGAGGAAACUCC CU UCAAGGACAUCGUCCGGG ACUGCAAC | 5972 |
| 3853 | UUAGCCAU G CACUGUUG | 1769 | CAACAGUG GGAGGAAACUCC CU UCAAGGACAUCGUCCGGG AUGGCUAA | 5973 |
| 3863 | ACUGUUGU G AGGAAAAA | 1770 | UUUUUCCU GGAGGAAACUCC CU UCAAGGACAUCGUCCGGG ACAACAGU | 5974 |
| 3883 | CCUGUCUU G ACUGCCAU | 1771 | AUGGCAGU GGAGGAAACUCC CU UCAAGGACAUCGUCCGGG AAGACAGG | 5975 |
| 3887 | UCUUGACU G CCAUGUGU | 1772 | ACACAUGG GGAGGAAACUCC CU UCAAGGACAUCGUCCGGG AGUCAAGA | 5976 |
| 3920 | UGUAAGCU G CUAUGUAU | 1773 | AUACAUAG GGAGGAAACUCC CU UCAAGGACAUCGUCCGGG AGCUUACA | 5977 |
| 3963 | UCCUAUCU G AGGCACUG | 1774 | CAGUGCCU GGAGGAAACUCC CU UCAAGGACAUCGUCCGGG AGAUAGGA | 5978 |
| 4005 | ACUUUGUU G CAGAUAGU | 1775 | ACUAUCUG GGAGGAAACUCC CU UCAAGGACAUCGUCCGGG AACAAAGU | 5979 |
| 4017 | AUAGUCUU G CCGCAUCU | 1776 | AGAUGCGG GGAGGAAACUCC CU UCAAGGACAUCGUCCGGG AAGACUAU | 5980 |
| 4020 | GUCUUGCC G CAUCUUGG | 1777 | CCAAGAUG GGAGGAAACUCC CU UCAAGGACAUCGUCCGGG GGCAAGAC | 5981 |
| 4035 | GGCAAGUU G CAGACAUG | 1778 | CAUCUCUG GGAGGAAACUCC CU UCAAGGACAUCGUCCGGG AACUUGCC | 5982 |
| 12 | CACAGUAG G UCCCUCGG | 1779 | CCGAGGGA GGAGGAAACUCC CU UCAAGGACAUCGUCCGGG CUACUGUG | 5983 |
| 20 | GUCCCUCG G CUCAGUCG | 1780 | CGACUGAG GGAGGAAACUCC CU UCAAGGACAUCGUCCGGG CGAGGGAC | 5984 |
| 25 | UCGGCUCA G UCGGCCCA | 1781 | UGGGCCGA GGAGGAAACUCC CU UCAAGGACAUCGUCCGGG UGAGCCGA | 5985 |
| 29 | CUCAGUCG G CCCAGCCC | 1782 | GGGCUGGG GGAGGAAACUCC CU UCAAGGACAUCGUCCGGG CGACUGUG | 5986 |
| 34 | UCGGCCCA G CCCCUCUC | 1783 | GAGAGGGG GGAGGAAACUCC CU UCAAGGACAUCGUCCGGG UGGGCCGA | 5987 |
| 44 | CCCUCUCA G UCCUCCCC | 1784 | GGGGAGGA GGAGGAAACUCC CU UCAAGGACAUCGUCCGGG UGAGAGGG | 5988 |
| 73 | CGCCCGCG G CUCUGAGA | 1785 | UCUCAGAG GGAGGAAACUCC CU UCAAGGACAUCGUCCGGG CGCGGGCG | 5989 |
| 86 | GAGACGCG G CCCCGGCG | 1786 | CGCCGGGG GGAGGAAACUCC CU UCAAGGACAUCGUCCGGG CGCGUCUC | 5990 |
| 92 | CGGCCCGG G CGGCGGCG | 1787 | CGCCGCCG GGAGGAAACUCC CU UCAAGGACAUCGUCCGGG CGGGGCCG | 5991 |
| 95 | CCCCGGCG G CGGCGGCA | 1788 | UGCCGCCG GGAGGAAACUCC CU UCAAGGACAUCGUCCGGG CGCCGGGG | 5992 |
| 98 | CGGCGGCG G CGGCAGCA | 1789 | UGCUGCCG GGAGGAAACUCC CU UCAAGGACAUCGUCCGGG CGCCGCCG | 5993 |
| 101 | CGGCGGCG G CAGCAGCU | 1790 | AGCUGCUG GGAGGAAACUCC CU UCAAGGACAUCGUCCGGG CGCCGCCG | 5994 |
| 104 | CGGCGGCA G CAGCUGCA | 1791 | UGCAGCUG GGAGGAAACUCC CU UCAAGGACAUCGUCCGGG UGCCGCCG | 5995 |
| 107 | CGGCAGCA G CUGCAGCA | 1792 | UGCUGCAG GGAGGAAACUCC CU UCAAGGACAUCGUCCGGG UGCUGCCG | 5996 |
| 113 | CAGCUGCA G CAUCAUCU | 1793 | AGAUGAUG GGAGGAAACUCC CU UCAAGGACAUCGUCCGGG UGCAGCUG | 5997 |
| 132 | ACCCUCCA G CCAUGGAA | 1794 | UUCCAUGG GGAGGAAACUCC CU UCAAGGACAUCGUCCGGG UGGAGGGU | 5998 |
| 152 | CUGGACCA G UCUCCUCU | 1795 | AGAGGAGA GGAGGAAACUCC CU UCAAGGACAUCGUCCGGG UGGUCCAG | 5999 |
| 162 | CUCCUCUG G UCUCGUCC | 1796 | GGACGAGA GGAGGAAACUCC CU UCAAGGACAUCGUCCGGG CAGAGGAG | 6000 |
| 167 | CUGGUCUC G UCCUCGGA | 1797 | UCCGAGGA GGAGGAAACUCC CU UCAAGGACAUCGUCCGGG GAGACCAG | 6001 |
| 178 | CUCGGACA G CCCACCCC | 1798 | GGGGUGGG GGAGGAAACUCC CU UCAAGGACAUCGUCCGGG UGUCCGAG | 6002 |
| 188 | CCACCCCG G CCGCAGCC | 1799 | GGCUGCGG GGAGGAAACUCC CU UCAAGGACAUCGUCCGGG CGGGGUGG | 6003 |
| 194 | CGGCCGCA G CCCGCGUU | 1800 | AACGCGGG GGAGGAAACUCC CU UCAAGGACAUCGUCCGGG UGCGGCCG | 6004 |
| 200 | CAGCCCGC G UUCAAGUA | 1801 | UACUUGAA GGAGGAAACUCC CU UCAAGGACAUCGUCCGGG GCGGGCUG | 6005 |
| 206 | GCGUUCAA G UACCAGUU | 1802 | AACUGGUA GGAGGAAACUCC CU UCAAGGACAUCGUCCGGG UUGAACGC | 6006 |
| 221 | AAGUACCA G UUCGUGAG | 1803 | CUCACGAA GGAGGAAACUCC CU UCAAGGACAUCGUCCGGG UGGUACUU | 6007 |
| 216 | ACCAGUUC G UGAGGGAG | 1804 | CUCCCUCA GGAGGAAACUCC CU UCAAGGACAUCGUCCGGG GAACUGGU | 6008 |
| 224 | GUGAGGGA G CCCGAGGA | 1805 | UCCUCGGG GGAGGAAACUCC CU UCAAGGACAUCGUCCGGG UCCCUCAC | 6009 |
| 287 | CUGGAGGA G CUGGAGGU | 1806 | ACCUCCAG GGAGGAAACUCC CU UCAAGGACAUCGUCCGGG UCCUCCAG | 6010 |
| 294 | AGCUGGAG G UGCUGGAG | 1807 | CUCCAGCA GGAGGAAACUCC CU UCAAGGACAUCGUCCGGG CUCCAGCU | 6011 |
| 308 | GAGAGGAA G CCCGCCGC | 1808 | GCGGCGGG GGAGGAAACUCC CU UCAAGGACAUCGUCCGGG UUCCUCUC | 6012 |
| 320 | GCCGCCGG G CUGUCCGC | 1809 | GCGGACAG GGAGGAAACUCC CU UCAAGGACAUCGUCCGGG CCGGCGGC | 6013 |
| 323 | GCCGGGCU G UCCGCGGC | 1810 | GCCGCGGA GGAGGAAACUCC CU UCAAGGACAUCGUCCGGG AGCCCGGC | 6014 |
| 330 | UGUCCGCC G CCCCAGUG | 1811 | CACUGGGG GGAGGAAACUCC CU UCAAGGACAUCGUCCGGG CGCGGACA | 6015 |
| 336 | CGGCCCCA G UGCCCACC | 1812 | GGUGGGCA GGAGGAAACUCC CU UCAAGGACAUCGUCCGGG UGGGGCCG | 6016 |
| 358 | UGCCGCCG G CGCGCCCC | 1813 | GGGGCGCG GGAGGAAACUCC CU UCAAGGACAUCGUCCGGG CGGCGGCA | 6017 |
| 390 | AUGACUUC G UGCCGCCG | 1814 | CGGCGGCA GGAGGAAACUCC CU UCAAGGACAUCGUCCGGG GAAGUCAU | 6018 |
| 399 | UGCCGCCG G CGCCCGGG | 1815 | CCGGGGCG GGAGGAAACUCC CU UCAAGGACAUCGUCCGGG CGGCGGCA | 6019 |
| 420 | CCCUGCCG G CCGCUCCC | 1816 | GGGAGCGG GGAGGAAACUCC CU UCAAGGACAUCGUCCGGG CGGCAGGG | 6020 |
| 432 | CUCCCCCC G UCGCCCCG | 1817 | CGGGGCGA GGAGGAAACUCC CU UCAAGGACAUCGUCCGGG GGGGGGAG | 6021 |
| 443 | GCCCCGGA G CGGCAGCC | 1818 | GGCUGCCG GGAGGAAACUCC CU UCAAGGACAUCGUCCGGG UCCGGGGC | 6022 |
| 446 | CCGGAGCG G CAGCCGUC | 1819 | GACGGCUG GGAGGAAACUCC CU UCAAGGACAUCGUCCGGG CGCUCCGG | 6023 |
| 449 | GAGCGGCA G CCGUCUUG | 1820 | CAAGACGG GGAGGAAACUCC CU UCAAGGACAUCGUCCGGG UGCCGCUC | 6024 |
| 452 | CGGCACCC G UCUUGGGA | 1821 | UCCCAAGA GGAGGAAACUCC CU UCAAGGACAUCGUCCGGG GGCUGCCG | 6025 |
| 466 | GGACCCGA G CCCGGUGU | 1822 | ACACCGGG GGAGGAAACUCC CU UCAAGGACAUCGUCCGGG UCGGGUCC | 6026 |
| 471 | CGAGCCCG G UGUCGUCG | 1823 | CGACGACA GGAGGAAACUCC CU UCAAGGACAUCGUCCGGG CGGGCUCG | 6027 |
| 473 | AGCCCGGU G UCGUCGAC | 1824 | GUCGACGA GGAGGAAACUCC CU UCAAGGACAUCGUCCGGG ACCGGGCU | 6028 |
| 476 | CCGGUGUC G UCGACCGU | 1825 | ACGGUCGA GGAGGAAACUCC CU UCAAGGACAUCGUCCGGG GACACCGG | 6029 |
| 483 | CGUCGACC G UGCCCGCG | 1826 | CGCGGGCA GGAGGAAACUCC CU UCAAGGACAUCGUCCGGG GGUCGACG | 6030 |
| 503 | UCCCCGCU G UCUGCUGC | 1827 | GCAGCAGA GGAGGAAACUCC CU UCAAGGACAUCGUCCGGG AGCGGGGA | 6031 |
| 516 | CUGCCGCA G UCUCGCCC | 1828 | GGGCGAGA GGAGGAAACUCC CU UCAAGGACAUCGUCCGGG UGCGGCAG | 6032 |
| 530 | CCCUCCAA G CUCCCUGA | 1829 | UCAGGGAG GGAGGAAACUCC CU UCAAGGACAUCGUCCGGG UUGGGGAG | 6033 |
| 548 | GACGACGA G CCUCCGGC | 1830 | GCCGGAGG GGAGGAAACUCC CU UCAAGGACAUCGUCCGGG UCGUCGUC | 6034 |
| 555 | AGCCUCCC G CCCGCCCU | 1831 | AGGCCGGG GGAGGAAACUCC CU UCAAGGACAUCGUCCGGG CGGAGGCU | 6035 |
| 560 | CCGGCCCG G CCUCCCCC | 1832 | GGGGGAGG GGAGGAAACUCC CU UCAAGGACAUCGUCCGGG CGGGCCGG | 6036 |
| 579 | CUCCCCCG G CCAGCGUG | 1833 | CACGCUGG GGAGGAAACUCC CU UCAAGGACAUCGUCCGGG CGGGGGAG | 6037 |
| 583 | CCCGGCCA G CGUGAGCC | 1834 | GGCUCACG GGAGGAAACUCC CU UCAAGGACAUCGUCCGGG UGGCCGGG | 6038 |
| 585 | CGGCCAGC G UGAGCCCC | 1835 | GGGGCUCA GGAGGAAACUCC CU UCAAGGACAUCGUCCGGG GCUGGCCG | 6039 |
| 589 | CAGCGUGA G CCCCCAGG | 1836 | CCUGGGGG GGAGGAAACUCC CU UCAAGGACAUCGUCCGGG UCACGCUG | 6040 |
| 597 | GCCCCCAG G CAGACCCC | 1837 | GGGCUCUG GGAGGAAACUCC CU UCAAGGACAUCGUCCGGG CUGGGGGC | 6041 |
| 602 | CAGGCACA G CCCGUGUG | 1838 | CACACGGG GGAGGAAACUCC CU UCAAGGACAUCGUCCGGG UCUGCCUG | 6042 |
| 606 | CAGAGCCC G UGUGGACC | 1839 | GGUCCACA GGAGGAAACUCC CU UCAAGGACAUCGUCCGGG GGGCUCUG | 6043 |
| 608 | GAGCCCGU G UGCACCCC | 1840 | GUGGUCCA GGAGGAAACUCC CU UCAAGGACAUCGUCCGGG ACGGGCUC | 6044 |
| 621 | CCCCGCCA G CCCCGGCU | 1841 | AGCCGGGG GGAGGAAACUCC CU UCAAGGACAUCGUCCGGG UGGCGGGG | 6045 |
| 627 | CAGCCCCG G CUCCCGCC | 1842 | GGCGGGAG GGAGGAAACUCC CU UCAAGGACAUCGUCCGGG CGGGGCUG | 6046 |
| 654 | CCACCCCG G CCGCGCCC | 1843 | GGGCGCGG GGAGGAAACUCC CU UCAAGGACAUCGUCCGGG CGGGGUGG | 6047 |
| 665 | GCGCCCAA G CGCAGGGG | 1844 | CCCCUUCG GGAGGAAACUCC CU UCAAGGACAUCGUCCGGG UUGGGCGC | 6048 |
| 673 | GCGCAGGG G CUCCUCGG | 1845 | CCGAGGAG GGAGGAAACUCC CU UCAAGGACAUCGUCCGGG CCCUGCGC | 6049 |
| 682 | CUCCUCGG G CUCAGUGG | 1846 | CCACUGAG GGAGGAAACUCC CU UCAAGGACAUCGUCCGGG CCGAGGAG | 6050 |
| 687 | CGGGCUCA G UGGAUGAG | 1847 | CUCAUCCA GGAGGAAACUCC CU UCAAGGACAUCGUCCGGG UGAGCCCG | 6051 |
| 725 | GCAUCUGA G CCUGUGAU | 1848 | AUCACAGG GGAGGAAACUCC CU UCAAGGACAUCGUCCGGG UCAGAUGC | 6052 |
| 729 | CUGAGCCU G UGAUACGC | 1849 | GCGUAUCA GGAGGAAACUCC CU UCAAGGACAUCGUCCGGG AGGCUCAG | 6053 |
| 767 | UUGAAGGA G CAGCCAGG | 1850 | CCUGGCUG GGAGGAAACUCC CU UCAAGGACAUCGUCCGGG UCCUUCAA | 6054 |
| 770 | AAGGAGCA G CCAGGUAA | 1851 | UUACCUGG GGAGGAAACUCC CU UCAAGGACAUCGUCCGGG UGCUCCUU | 6055 |
| 775 | GCAGCCAG G UAACACUA | 1852 | UAGUGUUA GGAGGAAACUCC CU UCAAGGACAUCGUCCGGG CUGGCUGC | 6056 |
| 789 | CUAUUUCG G CUGGUCAA | 1853 | UUGACCAG GGAGGAAACUCC CU UCAAGGACAUCGUCCGGG CGAAAUAG | 6057 |
| 793 | UUCGGCUG G UCAAGAGG | 1854 | CCUCUUGA GGAGGAAACUCC CU UCAAGGACAUCGUCCGGG CAGCCGAA | 6058 |
| 813 | UCCCAUCU G UCCUGCUU | 1855 | AAGCAGGA GGAGGAAACUCC CU UCAAGGACAUCGUCCGGG AGAUGGGA | 6059 |
| 848 | CCUUCUCU G UCUCCUCU | 1856 | AGAGGAGA GGAGGAAACUCC CU UCAAGGACAUCGUCCGGG AGAGAAGG | 6060 |
| 861 | CUCUCUCA G CCGCUUCU | 1857 | AGAAGCGG GGAGGAAACUCC CU UCAAGGACAUCGUCCGGG UGAGAGAG | 6061 |
| 892 | AUACCUUG G UAAUUUGU | 1858 | ACAAAUUA GGAGGAAACUCC CU UCAAGGACAUCGUCCGGG CAAGGUAU | 6062 |
| 899 | GGUAAUUU G UCAACAGU | 1859 | ACUGUUGA GGAGGAAACUCC CU UCAAGGACAUCGUCCGGG AAAUUACC | 6063 |
| 906 | UGUCAACA G UAUUACCC | 1860 | GGGUAAUA GGAGGAAACUCC CU UCAAGGACAUCGUCCGGG UGUUGACA | 6064 |
| 939 | AAGAAAAU G UCAGUGAA | 1861 | UUCACUGA GGAGGAAACUCC CU UCAAGGACAUCGUCCGGG AUUUUCUU | 6065 |
| 943 | AAAUGUCA G UGAAGCUU | 1862 | AAGCUUCA GGAGGAAACUCC CU UCAAGGACAUCGUCCGGG UGACAUUU | 6066 |
| 948 | UCAGUGAA G CUUCUAAA | 1863 | UUUAGAAG GGAGGAAACUCC CU UCAAGGACAUCGUCCGGG UUCACUGA | 6067 |
| 960 | CUAAAGAG G UCUCAGAG | 1864 | CUCUGAGA GGAGGAAACUCC CU UCAAGGACAUCGUCCGGG CUCUUUAG | 6068 |
| 972 | CAGAGAAG G CAAAAACU | 1865 | AGUUUUUG GGAGGAAACUCC CU UCAAGGACAUCGUCCGGG CUUCUCUG | 6069 |
| 1007 | UUAACAGA G UUUUCAGA | 1866 | UCUGAAAA GGAGGAAACUCC CU UCAAGGACAUCGUCCGGG UCUGUUAA | 6070 |
| 1043 | GGAUCAUC G UUCAGUGU | 1867 | ACACUGAA GGAGGAAACUCC CU UCAAGGACAUCGUCCGGG GUAGAUCC | 6071 |
| 1048 | AUCGUUCA G UGUCUCUC | 1868 | GAGAGACA GGAGGAAACUCC CU UCAAGGACAUCGUCCGGG UGAACGAU | 6072 |
| 1050 | CGUUCAGU G UCUCUCCA | 1869 | UGGAGAGA GGAGGAAACUCC CU UCAAGGACAUCGUCCGGG ACUGAACG | 6073 |
| 1062 | CUCCAAAA G CAGAAUCU | 1870 | AGAUUCUG GGAGGAAACUCC CU UCAAGGACAUCGUCCGGG UUUUGGAG | 6074 |
| 1074 | AAUCUGCC G UAAUAGUA | 1871 | UACUAUUA GGAGGAAACUCC CU UCAAGGACAUCGUCCGGG GGCAGAUU | 6075 |
| 1080 | CCGUAAUA G UAGCAAAU | 1872 | AUUUGCUA GGAGGAAACUCC CU UCAAGGACAUCGUCCGGG UAUUACGG | 6076 |
| 1083 | UAAUAGUA G CAAAUCCU | 1873 | AGGAUUUG GGAGGAAACUCC CU UCAAGGACAUCGUCCGGG UACUAUUA | 6077 |
| 1107 | AAAUAAUC G UGAAAAAU | 1874 | AUUUUUCA GGAGGAAACUCC CU UCAAGGACAUCGUCCGGG GAUUAUUU | 6078 |
| 1133 | GAAGAGAA G UUAGUUAG | 1875 | CUAACUAA GGAGGAAACUCC CU UCAAGGACAUCGUCCGGG UUCUCUUC | 6079 |
| 1137 | AGAAGUUA G UUAGUAAU | 1876 | AUUACUAA GGAGGAAACUCC CU UCAAGGACAUCGUCCGGG UAACUUCU | 6080 |
| 1141 | GUUAGUUA G UAAUAACA | 1877 | UGUUAUUA GGAGGAAACUCC CU UCAAGGACAUCGUCCGGG UAACUAAC | 6081 |
| 1169 | CAACAAGA G UUACCUAC | 1878 | GUAGGUAA GGAGGAAACUCC CU UCAAGGACAUCGUCCGGG UCUUGUUG | 6082 |
| 1179 | UACCUACA G CUCUUACU | 1879 | AGUAAGAG GGAGGAAACUCC CU UCAAGGACAUCGUCCGGG UGUAGGUA | 6083 |
| 1194 | CUAAAUUG G UUAAAGAG | 1880 | CUCUUUAA GGAGGAAACUCC CU UCAAGGACAUCGUCCGGG CAAUUUAG | 6084 |
| 1209 | AGGAUGAA G UUGUGUCU | 1881 | AGACACAA GGAGGAAACUCC CU UCAAGGACAUCGUCCGGG UUCAUCCU | 6085 |
| 1212 | AUGAAGGG G UGUCUUCA | 1882 | UGAAGACA GGAGGAAACUCC CU UCAAGGACAUCGUCCGGG AACUUCAU | 6086 |
| 1214 | GAAGUUGU G UCUUCAGA | 1883 | UCUGAAGA GGAGGAAACUCC CU UCAAGGACAUCGUCCGGG ACAACUUC | 6087 |
| 1227 | CAGAAAAA G CAAAAGAC | 1884 | GUCUUUUG GGAGGAAACUCC CU UCAAGGACAUCGUCCGGG UUUUUCUG | 6088 |
| 1237 | AAAAGACA G UUUUAAUG | 1885 | CAUUAAAA GGAGGAAACUCC CU UCAAGGACAUCGUCCGGG UGUCUUUU | 6089 |
| 1254 | AAAAGAGA G UUGCAGUG | 1886 | CACUGCAA GGAGGAAACUCC CU UCAAGGACAUCGUCCGGG UCUCUUUU | 6090 |
| 1260 | GAGUUGCA G UGGAAGCU | 1887 | AGCUUCCA GGAGGAAACUCC CU UCAAGGACAUCGUCCGGG UGCAACUC | 6091 |
| 1266 | CAGUGGAA G CUCCUAUG | 1888 | CAUAGGAG GGAGGAAACUCC CU UCAAGGACAUCGUCCGGG UUCCACUG | 6092 |
| 1307 | CCAUUUGA G CGAGUAUG | 1889 | CAUACUCG GGAGGAAACUCC CU UCAAGGACAUCGUCCGGG UCAAAUGG | 6093 |
| 1311 | UUGAGCGA G UAUGGGAA | 1890 | UUCCCAUA GGAGGAAACUCC CU UCAAGGACAUCGUCCGGG UCGCUCAA | 6094 |
| 1320 | UAUGGGAA G UGAAAGAU | 1891 | AUCUUUCA GGAGGAAACUCC CU UCAAGGACAUCGUCCGGG UUCCCAUA | 6095 |
| 1330 | GAAAGAUA G UAAGGAAG | 1892 | CUUCCUUA GGAGGAAACUCC CU UCAAGGACAUCGUCCGGG UAUCUUUC | 6096 |
| 1342 | GGAAGAUA G UGAUAUGU | 1893 | ACAUAUCA GGAGGAAACUCC CU UCAAGGACAUCGUCCGGG UAUCUUCC | 6097 |
| 1349 | AGUGAUAU G UUGGCUGC | 1894 | GCAGCCAA GGAGGAAACUCC CU UCAAGGACAUCGUCCGGG AUAUCACU | 6098 |
| 1353 | AUAUGUUG G CUGCUGGA | 1895 | UCCAGCAG GGAGGAAACUCC CU UCAAGGACAUCGUCCGGG CAACAUAU | 6099 |
| 1363 | UGCUCGAG G UAAAAUCG | 1896 | CGAUUUUA GGAGGAAACUCC CU UCAAGGACAUCGUCCGGG CUCCAGCA | 6100 |
| 1375 | AAUCGAGA G CAACUUGG | 1897 | CCAAGUUG GGAGGAAACUCC CU UCAAGGACAUCGUCCGGG UCUCGAUU | 6101 |
| 1387 | CUUGGAAA G UAAAGUGG | 1898 | CCACUUUA GGAGGAAACUCC CU UCAAGGACAUCGUCCGGG UUUCCAAG | 6102 |
| 1392 | AAAGUAAA G UGGAUAAA | 1899 | UUUAUCCA GGAGGAAACUCC CU UCAAGGACAUCGUCCGGG UUUACUUU | 6103 |
| 1405 | UAAAAAAU G UUUUGCAG | 1900 | CUGCAAAA GGAGGAAACUCC CU UCAAGGACAUCGUCCGGG AUUUUUUA | 6104 |
| 1417 | UGCAGAUA G CCUUGAGC | 1901 | GCUCAAGG GGAGGAAACUCC CU UCAAGGACAUCGUCCGGG UAUCUGCA | 6105 |
| 1424 | AGCCUUGA G CAAACUAA | 1902 | UUAGUUUG GGAGGAAACUCC CU UCAAGGACAUCGUCCGGG UCAAGGCU | 6106 |
| 1447 | AAAAGAUA G UGAGAGUA | 1903 | UACUCUCA GGAGGAAACUCC CU UCAAGGACAUCGUCCGGG UAUCUUUU | 6107 |
| 1453 | UAGUGAGA G UAGUAAUG | 1904 | CAUUACUA GGAGGAAACUCC CU UCAAGGACAUCGUCCGGG UCUCACUA | 6108 |
| 1456 | UGAGAGUA G UAAUGAUG | 1905 | CAUCAUUA GGAGGAAACUCC CU UCAAGGACAUCGUCCGGG UACUCUCA | 6109 |
| 1480 | UUUCCCCA G UACGCCAG | 1906 | CUGGCGUA GGAGGAAACUCC CU UCAAGGACAUCGUCCGGG UGGGGAAA | 6110 |
| 1492 | GCCAGAAG G UAUAAAGG | 1907 | CCUUUAUA GGAGGAAACUCC CU UCAAGGACAUCGUCCGGG CUUCUGGC | 6111 |
| 1504 | AAAGGAUC G UUCAGGAG | 1908 | CUCCUGAA GGAGGAAACUCC CU UCAAGGACAUCGUCCGGG GAUCCUUU | 6112 |
| 1512 | GUUCAGGA G CAUAUAUC | 1909 | GAUAUAUG GGAGGAAACUCC CU UCAAGGACAUCGUCCGGG UCCUGAAC | 6113 |
| 1525 | UAUCACAU G UGCUCCCU | 1910 | AGGGAGCA GGAGGAAACUCC CU UCAAGGACAUCGUCCGGG AUGUGAUA | 6114 |
| 1542 | UUAACCCA G CAGCAACU | 1911 | AGUUGCUC GGAGGAAACUCC CU UCAAGGACAUCGUCCGGG UGGGUUAA | 6115 |
| 1545 | ACCCAGCA G CAACUCAG | 1912 | CUCAGUUG GGAGGAAACUCC CU UCAAGGACAUCGUCCGGG UGCUGGGU | 6116 |
| 1555 | AACUGAGA G CAUUGCAA | 1913 | UUGCAAUG GGAGGAAACUCC CU UCAAGGACAUCGUCCGGG UCUCAGUU | 6117 |
| 1580 | UUUCCUUU G UUAGGAGA | 1914 | UCUCCUAA GGAGGAAACUCC CU UCAAGGACAUCGUCCGGG AAAGGAAA | 6118 |
| 1638 | AAAAGAAG G CCCAAAUA | 1915 | UAUUUGGG GGAGGAAACUCC CU UCAAGGACAUCGUCCGGG CUUCUUUU | 6119 |
| 1647 | CCCAAAUA G UAACAGAG | 1916 | CUCUGUUA CCAGGAAACUCC CU UCAAGGACAUCGUCCGGG UAUUUGGG | 6120 |
| 1666 | GAAUACUA G CACCAAAA | 1917 | UUUUGGUG GGAGGAAACUCC CU UCAAGGACAUCGUCCGGG UAGUAUUC | 6121 |
| 1692 | CUUUUCUU G UAGCAGCA | 1918 | UGCUGCUA GGAGGAAACUCC CU UCAAGGACAUCGUCCGGG AAGAAAAG | 6122 |
| 1695 | UUCUUCUA G CAGCACAG | 1919 | CUGUGCUG GGAGGAAACUCC CU UCAAGGACAUCGUCCGGG UACAAGAA | 6123 |
| 1698 | UUGUAGCA G CACAGGAU | 1920 | AUCCUGUG GGAGGAAACUCC CU UCAAGGACAUCGUCCGGG UGCUACAA | 6124 |
| 1722 | CAGAUUAU G UCACAACA | 1921 | UGUUGUGA GGAGGAAACUCC CU UCAAGGACAUCGUCCGGG AUAAUCUG | 6125 |
| 1746 | UAACAAAG G UGACUGAG | 1922 | CUCAGUCA GGAGGAAACUCC CU UCAAGGACAUCGUCCGGG CUUUGUUA | 6126 |
| 1758 | CUGAGGAA G UCGUGGCA | 1923 | UGCCACGA GGAGGAAACUCC CU UCAAGGACAUCGUCCGGG UUCCUCAG | 6127 |
| 1761 | AGGAAGUC G UGGCAAAC | 1924 | GUUUGCCA GGAGGAAACUCC CU UCAAGGACAUCGUCCGGG GACUUCCU | 6128 |
| 1764 | AAGUCGUG G CAAACAUG | 1925 | CAUGUUUG GGAGGAAACUCC CU UCAAGGACAUCGUCCGGG CACGACUU | 6129 |
| 1780 | GCCUGAAG G CCUGACUC | 1926 | GAGUCAGG GGAGGAAACUCC CU UCAAGGACAUCGUCCGGG CUUCAGGC | 6130 |
| 1797 | CAGAUUUA G UACAGGAA | 1927 | UUCCUGUA GGAGGAAACUCC CU UCAAGGACAUCGUCCGGG UAAAUCUG | 6131 |
| 1806 | UACAGGAA G CAUGUGAA | 1928 | UUCACAUG GGAGGAAACUCC CU UCAAGGACAUCGUCCGGG UUCCUGUA | 6132 |
| 1810 | GGAAGCAU G UGAAAGUG | 1929 | CACUUUCA GGAGGAAACUCC CU UCAAGGACAUCGUCCGGG AUGCUUCC | 6133 |
| 1816 | AUGUGAAA G UGAAUUGA | 1930 | UCAAUUCA GGAGGAAACUCC CU UCAAGGACAUCGUCCGGG UUUCACAU | 6134 |
| 1830 | UGAAUGAA G UUACUGGU | 1931 | ACCAGUAA GGAGGAAACUCC CU UCAAGGACAUCGUCCGGG UUCAUUCA | 6135 |
| 1837 | AGUUACUG G UACAAAGA | 1932 | UCUUUGUA GGAGGAAACUCC CU UCAAGGACAUCGUCCGGG CAGUAACU | 6136 |
| 1872 | UGGACUUG G UUCAAACA | 1933 | UGUUUGAA GGAGGAAACUCC CU UCAAGGACAUCGUCCGGG CAAGUCCA | 6137 |
| 1887 | CAUCAGAA G UUAUGCAA | 1934 | UUGCAUAA GGAGGAAACUCC CU UCAAGGACAUCGUCCGCG UUCUGAUG | 6138 |
| 1898 | AUGCAAGA G UCACUCUA | 1935 | UAGAGUGA GGAGGAAACUCC CU UCAAGGACAUCGUCCGGG UCUUGCAU | 6139 |
| 1914 | AUCCUGCA G CACAGCUU | 1936 | AAGCUGUG GGAGGAAACUCC CU UCAAGGACAUCGUCCGGG UGCAGGAU | 6140 |
| 1919 | GCAGCACA G CUUUGCCC | 1937 | GGGCAAAG GGAGGAAACUCC CU UCAAGGACAUCGUCCGGG UGUGCUGC | 6141 |
| 1940 | UUUGAAGA G UCACAACC | 1938 | GCUUCUGA GGAGGAAACUCC CU UCAAGGACACCGUCCGGG UCUUCAAA | 6142 |
| 1947 | AGUCAGAA G CUACUCCU | 1939 | AGGAGUAG GGAGGAAACCCC CU UCAAGGACAUCGUCCGGG UUCUGACU | 6143 |
| 1962 | CUUCACCA G UUCUGCCU | 1940 | AGGCAAAA GGAGGAAACUCC CU UCAAGGACAUCGUCCGGG UGGUGAAG | 6144 |
| 1977 | CUGACAUC G UUAUGGAA | 1941 | UUCCAUAA GGAGGAAACUCC CU UCAAGGACAUCGUCCGGG AAUGUCAG | 6145 |
| 1986 | UUAUGGAA G CACCAUUG | 1942 | CAAUGGUG GGAGGAAACUCC CU UCAAGGACAUCGUCCGGG UUCCAUAA | 6146 |
| 2004 | AUUCUGCA G UUCCUAGU | 1943 | ACUAGGAA GGAGGAAACUCC CU UCAAGGACAUCGUCCGGG UGCAGAAU | 6147 |
| 2011 | AGUUCCUA G UGCUGGUG | 1944 | CACCAGCA GGAGGAAACUCC CC UCAAGGACAUCCUCCGGG UAGGAACU | 6148 |
| 2017 | UAGUGCUG G UGCUUCCG | 1945 | CGGAAGCA GGAGGAAACUCC CU UCAAGGACAUCGUCCGGG CAGCACUA | 6149 |
| 2025 | GUGCUUCC G UGAUACAG | 1946 | CUGUAUCA GGAGGAAACUCC CU UCAAGGACAUCGUCCGGG GGAAGCAC | 6150 |
| 2033 | GUGAUACA G CCCAGCUC | 1947 | GAGCUGGG GGAGGAAACUCC CU UCAAGGACAUCGUCCGGG UGUAUCAC | 6151 |
| 2038 | ACAGCCCA G CUCAUCAC | 1948 | GUGAUGAG GGAGGAAACUCC CU UCAAGGACAUCGUCCGGG UGGGCUGU | 6152 |
| 2055 | CAUUAGAA G CUUCUUCA | 1949 | UGAAGAAG GGAGGAAACUCC CU UCAAGGACAUCGUCCGGG UUCUAAUG | 6153 |
| 2064 | CUCCUCCA G UCAAUUAU | 1950 | AUAAUUAA GGAGGAAACUCC CU UCAAGGACAUCGUCCGGG UGAAGAAG | 6154 |
| 2077 | UUAUGAAA G CAUAAAAC | 1951 | GUUUUAUG GGAGGAAACUCC CU UCAAGGACAUCGUCCGGG UUUCAUAA | 6155 |
| 2090 | AAACAUGA G CCUGAAAA | 1952 | UUUUCAGG GGAGGAAACUCC CU UCAAGGACAUCGUCCGGG UCAUGUUU | 6156 |
| 2118 | AUGAAGAG G CCAUGAGU | 1953 | ACUCAUGG GGAGGAAACUCC CU UCAAGGACAUCGUCCGGG CUCUUCAU | 6157 |
| 2125 | GGCCAUGA G UGUAUCAC | 1954 | GUGAUACA GGAGGAAACUCC CU UCAAGGACAUCGUCCGGG UCAUGGCC | 6158 |
| 2127 | CCAUGAGU G UAUCACUA | 1955 | UAGUGAUA GGAGGAAACUCC CU UCAAGGACAUCGUCCGGG ACUCAUGG | 6159 |
| 2142 | UAAAAAAA G UAUCAGGA | 1956 | UCCUGAUA GGAGGAAACUCC CU UCAAGGACAUCGUCCGGG UUUUUUUA | 6160 |
| 2171 | AUUAAAGA G CCUGAAAA | 1957 | UUUUCAGG GGAGGAAACUCC CU UCAAGGACAUCGUCCGGG UCUUUAAU | 6161 |
| 2190 | UUAAUGCA G CUCUUCAA | 1958 | UUGAAGAG GGAGGAAACUCC CU UCAAGGACAUCGUCCGGG UGCAUUAA | 6162 |
| 2208 | AAACAGAA G CUCCUUAU | 1959 | AUAAGGAG GGAGGAAACUCC CU UCAAGGACAUCGUCCGGG UUCUGUUU | 6163 |
| 2230 | UAUUGCAU G UGAUUUAA | 1960 | UUAAAUCA GGAGGAAACUCC CU UCAAGGACAUCGUCCGGG AUGCAAUA | 6164 |
| 2252 | GAAACAAA G CUUUCUGC | 1961 | GCAGAAAG GGAGGAAACUCC CU UCAAGGACAUCGUCCGGG UUUGUUUC | 6165 |
| 2268 | CUGAACCA G CUCCGGAU | 1962 | AUCCGGAG GGAGGAAACUCC CU UCAAGGACAUCGUCCGGG UGGUUCAG | 6166 |
| 2298 | CAGAAAUG G CAAAAGUU | 1963 | AACUUUUG GGAGGAAACUCC CU UCAAGGACAUCGUCCGGG CAUUUCUG | 6167 |
| 2304 | UGGCAAAA G UUGAACAG | 1964 | CUGUUCAA GGAGGAAACUCC CU UCAAGGACAUCGUCCGGG UUUUGCCA | 6168 |
| 2312 | GUUGAACA G CCAGUGCC | 1965 | GGCACUGG GGAGGAAACUCC CU UCAAGGACAUCGUCCGGG UGUUCAAC | 6169 |
| 2316 | AACAGCCA G AGCCUGAU | 1966 | AUCAGGCA GGAGGAAACUCC CU UCAAGGACAUCGUCCGGG UGGCUGUU | 6170 |
| 2333 | CAUUCUUA G CUAGUUGA | 1967 | UCAACUAG GGAGGAAACUCC CU UCAAGGACAUCGUCCGGG UCAGAAUG | 6171 |
| 2337 | CUGAGCUA G UUGAAGAU | 1968 | AUCUUCAA GGAGGAAACUCC CU UCAAGGACAUCGUCCGGG UAGCUCAG | 6172 |
| 2367 | CUGAACCA G UUGACUUA | 1969 | UAAGUCAA GGAGGAAACUCC CU UCAAGGACAUCGUCCGGG UGGUUCAG | 6173 |
| 2380 | CUUAUUUA G UGAUGAUU | 1970 | AAUCAUCA GGAGGAAACUCC CU UCAAGCACAUCGUCCGGG UAAAUAAG | 6174 |
| 2400 | UACCUGAC G UUCCACAA | 1971 | UUGUGGAA GGAGGAAACUCC CU UCAAGGACAUCGUCCGGG GUCAGGUA | 6175 |
| 2424 | AUGAAACU G UGAUGCUU | 1972 | AAGCAUCA GGAGGAAACUCC CU UCAAGGACAUCGUCCGGG AGUUUCAU | 6176 |
| 2433 | UGAUGCUU G UGAAAGAA | 1973 | UUCUUUCA GGAGGAAACUCC CU UCAAGGACAUCGUCCGGG AAGCAUCA | 6177 |
| 2443 | GAAACAAA G UCUCACUG | 1974 | CAGUGAGA GGAGGAAACUCC CU UCAAGGACAUCGUCCGGG UUUCUUUC | 6178 |
| 2465 | UCAUUUGA G UCAAUGAU | 1975 | AUCAUUGA GGAGGAAACUCC CU UCAAGGACAUCGUCCGGG UCAAAUGA | 6179 |
| 2500 | AAAACUCA G UGCUUUGC | 1976 | GCAAAGCA GGAGGAAACUCC CU UCAAGGACAUCGUCCGGG UGAGUUUU | 6180 |
| 2525 | GGAGGUAA G CCAUAUUU | 1977 | AAAUAUGG GGAGGAAACUCC CU UCAAGGACAUCGUCCGGG UUUCCUCC | 6181 |
| 2546 | UCUUUUAA G CUCAGUUU | 1978 | AAACUGAG GGAGGAAACUCC CU UCAAGGACAUCGUCCGGG UUAAAAGA | 6182 |
| 2551 | UAAGCUCA G UUUAGAUA | 1979 | UAUCUAAA GGAGGAAACUCC CU UCAAGGACAUCGUCCGGG UGAGCUUA | 6183 |
| 2576 | GAUACCCU G UUACCUGA | 1980 | UCAGGUAA GGAGGAAACUCC CU UCAAGGACAUCGUCCGGG AGGGUAUC | 6184 |
| 2589 | CUGAUGAA G UUUCAACA | 1981 | UGUUGAAA GGAGGAAACUCC CU UCAAGGACAUCGUCCGGG UUCAUCAG | 6185 |
| 2602 | AACAUUGA G CAAAAAGG | 1982 | CCUUUUUG GGAGGAAACUCC CU UCAAGGACAUCGUCCGGG UCAAUGUU | 6186 |
| 2636 | AUGGAGGA G CUCAGUAC | 1983 | GUACUGAG GGAGGAAACUCC CU UCAAGGACAUCGUCCGGG UCCUCCAU | 6187 |
| 2641 | GGAGCUCA G UACUGCAG | 1984 | CUGCAGUA GGAGGAAACUCC CU UCAAGGACAUCGUCCGGG UGAGCUCC | 6188 |
| 2649 | GUACUGCA G UUUAUUCA | 1985 | UGAAUAAA GGAGGAAACUCC CU UCAAGGACAUCGUCCGGG UGCAGUAC | 6189 |
| 2685 | CUAAGGAA G CACAGAUA | 1986 | UAUCUGUG GGAGGAAACUCC CU UCAAGGACAUCGUCCGGG UUCCUUAG | 6190 |
| 2708 | ACUGAAAC G UUUUCAGA | 1987 | UCUGAAAA GGAGGAAACUCC CU UCAAGGACAUCGUCCGGG GUUUCAGU | 6191 |
| 2744 | AUAGAUGA G UUCCCUAC | 1988 | GUAGGGAA GGAGGAAACUCC CU UCAAGGACAUCGUCCGGG UCAUCUAU | 6192 |
| 2761 | AUUGAUCA G UUCUAAAA | 1989 | UUUUAGAA GGAGGAAACUCC CU UCAAGGACAUCGUCCGGG UGAUCAAU | 6193 |
| 2790 | CUAAAUUA G CCAGGGAA | 1990 | UUCCCUGG GGAGGAAACUCC CU UCAAGGACAUCGUCCGGG UAAUUUAG | 6194 |
| 2814 | ACCUAGAA G UAUCCCAC | 1991 | GUGGGAUA GGAGGAAACUCC CU UCAAGGACAUCGUCCGGG UUCUAGGU | 6195 |
| 2827 | CCACAAAA G UGAAAUUG | 1992 | CAAUUUCA GGAGGAAACUCC CU UCAAGGACAUCGUCCGGG UUUUGUGG | 6196 |
| 2853 | CGGAUGGA G CUGGGUCA | 1993 | UGACCCAG GGAGGAAACUCC CU UCAAGGACAUCGUCCGGG UCCAUCCG | 6197 |
| 2858 | GGAGCUGG G UCAUUGCC | 1994 | GGCAAUGA GGAGGAAACUCC CU UCAAGGACAUCGUCCGGG CCAGCUCC | 6198 |
| 2916 | AACCCAAA G UUGAAGAG | 1995 | CUCUUCAA GGAGGAAACUCC CU UCAAGGACAUCGUCCGGG UUUGGGUU | 6199 |
| 2932 | GAAAAUCA G UUUCUCAG | 1996 | CUGAGAAA GGAGGAAACUCC CU UCAAGGACAUCGUCCGGG UGAUUUUC | 6200 |
| 2960 | AAAAAUGG G UCUGCUAC | 1997 | GUAGCAGA GGAGGAAACUCC CU UCAAGGACAUCGUCCGGG CCAUUUUU | 6201 |
| 2976 | CAUCAAAG G UGCUCUUA | 1998 | UAAGAGCA GGAGGAAACUCC CU UCAAGGACAUCGUCCGGG CUUUGAUG | 6202 |
| 2997 | CUCCAGAU G UUUCUGCU | 1999 | AGCAGAAA GGAGGAAACUCC CU UCAAGGACAUCGUCCGGG AUCUGGAG | 6203 |
| 3009 | CUGCUUUG G CCACUCAA | 2000 | UUGAGUGG GGAGGAAACUCC CU UCAAGGACAUCGUCCGGG CAAAGCAG | 6204 |
| 3018 | CCACUCAA G CAGAGAUA | 2001 | UAUCUCUG GGAGGAAACUCC CU UCAAGGACAUCGUCCGGG UUGAGUGG | 6205 |
| 3031 | GAUAGAGA G CAUAGUUA | 2002 | UAACUAUG GGAGGAAACUCC CU UCAAGGACAUCGUCCGGG UCUCUAUC | 6206 |
| 3036 | AGAGCAUA G UUAAACCC | 2003 | GGGUUUAA GGAGGAAACUCC CU UCAAGGACAUCGUCCGGG UAUGCUCU | 6207 |
| 3048 | AACCCAAA G UUCUUGUG | 2004 | CACAAGAA GGAGGAAACUCC CU UCAAGGACAUCGUCCGGG UUUGGGUU | 6208 |
| 3054 | AAGUUCUU G UGAAAGAA | 2005 | UUCUUUCA GGAGGAAACUCC CU UCAAGGACAUCGUCCGGG AAGAACUU | 6209 |
| 3063 | UGAAAGAA G CUGAGAAA | 2006 | UUUCUCAG GGAGGAAACUCC CU UCAAGGACAUCGUCCGGG UUCUUUCA | 6210 |
| 3126 | UAUUUUCA G CAGAGCUG | 2007 | CAGCUCUG GGAGGAAACUCC CU UCAAGGACAUCGUCCGGG UGAAAAUA | 6211 |
| 3131 | UCAGCAGA G CUGAGUAA | 2008 | UUACUCAG GGAGGAAACUCC CU UCAAGGACAUCGUCCGGG UCUGCUGA | 6212 |
| 3136 | AGAGCUGA G UAAAACUU | 2009 | AAGUUUUA GGAGGAAACUCC CU UCAAGGACAUCGUCCGGG UCAGCUCU | 6213 |
| 3147 | AAACUUCA G UUGUUGAC | 2010 | GUCAACAA GGAGGAAACUCC CU UCAAGGACAUCGUCCGGG UGAAGUUU | 6214 |
| 3150 | CUUCAGUU G UUGACCUC | 2011 | GAGGUCAA GGAGGAAACUCC CU UCAAGGACAUCGUCCGGG AACUGAAG | 6215 |
| 3161 | GACCUCCU G UACUGGAG | 2012 | CUCCAGUA GGAGGAAACUCC CU UCAAGGACAUCGUCCGGG AGGAGGUC | 6216 |
| 3189 | AGACUGGA G UGGUGUUU | 2013 | AAACACCA GGAGGAAACUCC CU UCAAGGACAUCGUCCGGG UCCAGUCU | 6217 |
| 3192 | CUGGAGUG G UGUUUGGU | 2014 | ACCAAACA GGAGGAAACUCC CU UCAAGGACAUCGUCCGGG CACUCCAG | 6218 |
| 3194 | GGAGUGGU G UUUGGUGC | 2015 | GCACCAAA GGAGGAAACUCC CU UCAAGGACAUCGUCCGGG ACCACUCC | 6219 |
| 3199 | GGUGUUUG G UGCCAGCC | 2016 | GGCUGGCA GGAGGAAACUCC CU UCAAGGACAUCGUCCGGG CAAACACC | 6220 |
| 3205 | UGGUGCCA G CCUAUUCC | 2017 | GGAAUAGG GGAGGAAACUCC CU UCAAGGACAUCGUCCGGG UGGCACCA | 6221 |
| 3231 | CAUUGACA G UAUUCAGC | 2018 | GCUGAAUA GGAGGAAACUCC CU UCAAGGACAUCGUCCGGG UGUCAAUG | 6222 |
| 3238 | AGUAUUCA G CAUUGUGA | 2019 | UCACAAUG GGAGGAAACUCC CU UCAAGGACAUCGUCCGGG UGAAUACU | 6223 |
| 3243 | UCAGCAUU G UGAGCGUA | 2020 | UACGCUCA GGAGGAAACUCC CU UCAAGGACAUCGUCCGGG AAUGCUGA | 6224 |
| 3247 | CAUUGUGA G CGUAACAG | 2021 | CUGUUACG GGAGGAAACUCC CU UCAAGGACAUCGUCCGGG UCACAAUG | 6225 |
| 3249 | UUGUGAGC G UAACAGCC | 2022 | GGCUGUUA GGAGGAAACUCC CU UCAAGGACAUCGUCCGGG GCUCACAA | 6226 |
| 3255 | GCGUAACA G CCUACAUU | 2023 | AAUGUAGG GGAGGAAACUCC CU UCAAGGACAUCGUCCGGG UGUUACGC | 6227 |
| 3270 | UUGCCUUG G CCCUGCUC | 2024 | GAGCAGGG GGAGGAAACUCC CU UCAAGGACAUCGUCCGGG CAAGGCAA | 6228 |
| 3282 | UGCUCUCU G UGACCAUC | 2025 | GAUGGUCA GGAGGAAACUCC CU UCAAGGACAUCGUCCGGG AGAGAGCA | 6229 |
| 3292 | GACCAUCA G CUUUAGGA | 2026 | UCCUAAAG GGAGGAAACUCC CU UCAAGGACAUCGUCCGGG UGAUGGUC | 6230 |
| 3310 | AUACAAGG G UGUGAUCC | 2027 | GGAUCACA GGAGGAAACUCC CU UCAAGGACAUCGUCCGGG CCUUGUAU | 6231 |
| 3312 | ACAAGGGU G UGAUCCAA | 2028 | UUGGAUCA GGAGGAAACUCC CU UCAAGGACAUCGUCCGGG ACCCUUGU | 6232 |
| 3321 | UGAUCCAA G CUAUCCAG | 2029 | CUGGAUAG GGAGGAAACUCC CU UCAAGGACAUCGUCCGGG UUGGAUCA | 6233 |
| 3343 | AGAUGAAG G CCACCCAU | 2030 | AUGGGUGG GGAGGAAACUCC CU UCAAGGACAUCGUCCGGG CUUCAUCU | 6234 |
| 3357 | CAUUCAGG G CAUAUCUG | 2031 | CAGAUAUG GGAGGAAACUCC CU UCAAGGACAUCGUCCGGG CCUGAAUG | 6235 |
| 3375 | AAUCUGAA G UUGCUAUA | 2032 | UAUAGCAA GGAGGAAACUCC CU UCAAGGACAUCGUCCGGG UUCAGAUU | 6236 |
| 3392 | UCUGAGGA G UUGGUUCA | 2033 | UGAACCAA GGAGGAAACUCC CU UCAAGGACAUCGUCCGGG UCCUCAGA | 6237 |
| 3396 | AGGAGUUG G UUCAGAAG | 2034 | CUUCUGAA GGAGGAAACUCC CU UCAAGGACAUCGUCCGGG CAACUCCU | 6238 |
| 3404 | GUUCAGAA G UACAGUAA | 2035 | UUACUGUA GGAGGAAACUCC CU UCAAGGACAUCGUCCGGG UUCUGAAC | 6239 |
| 3409 | GAAGUACA G UAAUUCUG | 2036 | CAGAAUUA GGAGGAAACUCC CU UCAAGGACAUCGUCCGGG UGUACUUC | 6240 |
| 3424 | UGCUCUUG G UCAUGUGA | 2037 | UCACAUGA GGAGGAAACUCC CU UCAAGGACAUCGUCCGGG CAAGAGCA | 6241 |
| 3429 | UUGGUCAU G UGAACUGC | 2038 | GCAGUUCA GGAGGAAACUCC CU UCAAGGACAUCGUCCGGG AUGACCAA | 6242 |
| 3455 | GAACUCAG G CGCCUCUU | 2039 | AAGAGGCG GGAGGAAACUCC CU UCAAGGACAUCGUCCGGG CUGAGUUC | 6243 |
| 3468 | UCUUCUUA G UUGAUGAU | 2040 | AUCAUCAA GGAGGAAACUCC CU UCAAGGACAUCGUCCGGG UAAGAAGA | 6244 |
| 3480 | AUGAUUUA G UUGAUUCU | 2041 | AGAAUCAA GGAGGAAACUCC CU UCAAGGACAUCGUCCGGG UAAAUCAU | 6245 |
| 3494 | UCUCUGAA G UUUGCAGU | 2042 | ACUGCAAA GGAGGAAACUCC CU UCAAGGACAUCGUCCGGG UUCAGAGA | 6246 |
| 3501 | AGUUUGCA G UGUUGAUG | 2043 | CAUCAACA GGAGGAAACUCC CU UCAAGGACAUCGUCCGGG UGCAAACU | 6247 |
| 3503 | UUUGCAGU G UUGAUGUG | 2044 | CACAUCAA GGAGGAAACUCC CU UCAAGGACAUCGUCCGGG ACUGCAAA | 6248 |
| 3509 | GUGUUGAU G UGGGUAUU | 2045 | AAUACCCA GGAGGAAACUCC CU UCAAGGACAUCGUCCGGG AUCAACAC | 6249 |
| 3513 | UGAUGUGG G UAUUUACC | 2046 | GGUAAAUA GGAGGAAACUCC CU UCAAGGACAUCGUCCGGG CCACAUCA | 6250 |
| 3525 | UUACCUAU G UUGGUGCC | 2047 | GGCACCAA GGAGGAAACUCC CU UCAAGGACAUCGUCCGGG AUAGGUAA | 6251 |
| 3529 | CUAUGUUG G UGCCUUGU | 2048 | ACAAGGCA GGAGGAAACUCC CU UCAAGGACAUCGUCCGGG CAACAUAG | 6252 |
| 3536 | GGUGCCUU G UUUAAUGG | 2049 | CCAUUAAA GGAGGAAACUCC CU UCAAGGACAUCGUCCGGG AAGGCACC | 6253 |
| 3544 | GUUUAAUG G UCUGACAC | 2050 | GUGUCAGA GGAGGAAACUCC CU UCAAGGACAUCGUCCGGG CAUUAAAC | 6254 |
| 3564 | UGAUUUUG G CUCUCAUU | 2051 | AAUGAGAG GGAGGAAACUCC CU UCAAGGACAUCGUCCGGG CAAAAUCA | 6255 |
| 3583 | ACUCUUCA G UGUUCCUG | 2052 | CAGGAACA GGAGGAAACUCC CU UCAAGGACAUCGUCCGGG UGAAGAGU | 6256 |
| 3585 | UCUUCAGU G UUCCUGUU | 2053 | AACAGGAA GGAGGAAACUCC CU UCAAGGACAUCGUCCGGG ACUGAAGA | 6257 |
| 3591 | GUGUUCCU G UUAUUUAU | 2054 | AUAAAUAA GGAGGAAACUCC CU UCAAGGACAUCGUCCGGG AGGAACAC | 6258 |
| 3605 | UAUGAACG G CAUCAGGC | 2055 | GCCUGAUG GGAGGAAACUCC CU UCAAGGACAUCGUCCGGG CGUUCAUA | 6259 |
| 3612 | GGCAUCAG G CACAGAUA | 2056 | UAUCUGUG GGAGGAAACUCC CU UCAAGGACAUCGUCCGGG CUGAUGCC | 6260 |
| 3651 | AUAAGAAU G UUAAAGAU | 2057 | AUCUUUAA GGAGGAAACUCC CU UCAAGGACAUCGUCCGGG AUUCUUAU | 6261 |
| 3666 | AUGCUAUG G CUAAAAUC | 2058 | GAUUUUAG GGAGGAAACUCC CU UCAAGGACAUCGUCCGGG CAUAGCAU | 6262 |
| 3678 | AAAUCCAA G CAAAAAUC | 2059 | GAUUUUUG GGAGGAAACUCC CU UCAAGGACAUCGUCCGGG UUGGAUUU | 6263 |
| 3698 | GGAUUGAA G CGCAAAGC | 2060 | GCUUUGCG GGAGGAAACUCC CU UCAAGGACAUCGUCCGGG UUCAAUCC | 6264 |
| 3705 | AGCGCAAA G CUGAAUGA | 2061 | UCAUUCAG GGAGGAAACUCC CU UCAAGGACAUCGUCCGGG UUUGCGCU | 6265 |
| 3732 | AAUAAUUA G UAGGAGUU | 2062 | AACUCCUA GGAGGAAACUCC CU UCAAGGACAUCGUCCGGG UAAUUAUU | 6266 |
| 3738 | UAGUAGGA G UUCAUCUU | 2063 | AAGAUGAA GGAGGAAACUCC CU UCAAGGACAUCGUCCGGG UCCUACUA | 6267 |
| 3781 | GGGGGAGG G UCAGGGAA | 2064 | UUCCCUGA GGAGGAAACUCC CU UCAAGGACAUCGUCCGGG CCUCCCCC | 6268 |
| 3804 | ACCUUGAC G UUGCAGUG | 2065 | CACUGCAA GGAGGAAACUCC CU UCAAGGACAUCGUCCGGG GUCAAGGU | 6269 |
| 3810 | ACGUUGCA G UGCAGUUU | 2066 | AAACUGCA GGAGGAAACUCC CU UCAAGGACAUCGUCCGGG UGCAACGU | 6270 |
| 3815 | GCAGUGCA G UUUCACAG | 2067 | CUGUGAAA GGAGGAAACUCC CU UCAAGGACAUCGUCCGGG UGCACUGC | 6271 |
| 3827 | CACAGAUC G UUGUUAGA | 2068 | UCUAACAA GGAGGAAACUCC CU UCAAGGACAUCGUCCGGG GAUCUGUG | 6272 |
| 3830 | AGAUCGUU G UUAGAUCU | 2069 | AGAUCUAA GGAGGAAACUCC CU UCAAGGACAUCGUCCGGG AACGAUCU | 6273 |
| 3848 | UAUUUUUA G CCAUGCAC | 2070 | GUGCAUGG GGAGGAAACUCC CU UCAAGGACAUCGUCCGGG UAAAAAUA | 6274 |
| 3858 | CAUGCACU G UUGUGAGG | 2071 | CCUCACAA GGAGGAAACUCC CU UCAAGGACAUCGUCCGGG AGUGCAUG | 6275 |
| 3861 | GCACUGUU G UGAGGAAA | 2072 | UUUCCUCA GGAGGAAACUCC CU UCAAGGACAUCGUCCGGG AACAGUGC | 6276 |
| 3878 | AAUUACCU G UCUUGACU | 2073 | AGUCAAGA GGAGGAAACUCC CU UCAAGGACAUCGUCCGGG AGGUAAUU | 6277 |
| 3892 | ACUGCCAU G UGUUCAUC | 2074 | GAUGAACA GGAGGAAACUCC CU UCAAGGACAUCGUCCGGG AUGGCAGU | 6278 |
| 3894 | UGCCAUGU G UUCAUCAU | 2075 | AUGAUGAA GGAGGAAACUCC CU UCAAGGACAUCGUCCGGG ACAUGGCA | 6279 |
| 3908 | CAUCUUAA G UAUUGUAA | 2076 | UUACAAUA GGAGGAAACUCC CU UCAAGGACAUCGUCCGGG UUAAGAUG | 6280 |
| 3913 | UAAGUAUU G UAAGCUGC | 2077 | GCAGCUUA GGAGGAAACUCC CU UCAAGGACAUCGUCCGGG AAUACUUA | 6281 |
| 3917 | UAUUGUAA G CUGCUAUG | 2078 | CAUAGCAG GGAGGAAACUCC CU UCAAGGACAUCGUCCGGG UUACAAUA | 6282 |
| 3925 | GCUGCUAU G UAUGGAUU | 2079 | AAUCCAUA GGAGGAAACUCC CU UCAAGGACAUCGUCCGGG AUAGCAGC | 6283 |
| 3940 | UUUAAACC G UAAUCAUA | 2080 | UAUGAUUA GGAGGAAACUCC CU UCAAGGACAUCGUCCGGG GGUUUAAA | 6284 |
| 3966 | UAUCUGAG G CACUGGUG | 2081 | CACCAGUG GGAGGAAACUCC CU UCAAGGACAUCGUCCGGG CUCAGAUA | 6285 |
| 3972 | AGGCACUG G UGGAAUAA | 2082 | UUAUUCCA GGAGGAAACUCC CU UCAAGGACAUCGUCCGGG CAGUGCCU | 6286 |
| 3988 | AAAAACCU G UAUAUUUU | 2083 | AAAAUAUA GGAGGAAACUCC CU UCAAGGACAUCGUCCGGG AGGUUUUU | 6287 |
| 4002 | UUUACUUU G UUGCAGAU | 2084 | AUCUGCAA GGAGGAAACUCC CU UCAAGGACAUCGUCCGGG AAAGUAAA | 6288 |
| 4012 | UGCAGAUA G UCUUGCCG | 2085 | CGGCAAGA GGAGGAAACUCC CU UCAAGGACAUCGUCCGGG UAUCUGCA | 6289 |
| 4028 | GGAUCUUG G CAAGUUGC | 2086 | GCAACUUG GGAGGAAACUCC CU UCAAGGACAUCGUCCGGG CAAGAUGC | 6290 |
| 4032 | CUUGGCAA G UUGCAGAG | 2087 | CUCUGCAA GGAGGAAACUCC CU UCAAGGACAUCGUCCGGG UUGCCAAG | 6291 |
| 4044 | CAGAGAUG G UGGAGCUA | 2088 | UAGCUCCA GGAGGAAACUCC CU UCAAGGACAUCGUCCGGG CAUCUCUG | 6292 |
| 11 | CCACAGUA G GUCCCUCG | 2329 | CGAGGGAC GGAGGAAACUCC CU UCAAGGACAUCGUCCGGG UACUGUGG | 6293 |
| 19 | GGUCCCUC G GCUCAGUC | 2330 | GACUGAGC GGAGGAAACUCC CU UCAAGGACAUCGUCCGGG GAGGGACC | 6294 |
| 28 | GCUCAGUC G GCCCAGCC | 2331 | GGCUGGGC GGAGGAAACUCC CU UCAAGGACAUCGUCCGGG GACUGAGC | 6295 |
| 72 | CCGCCCGC G GCUCUGAG | 2332 | CUCAGAGC GGAGGAAACUCC CU UCAAGGACAUCGUCCGGG GCGGGCGG | 6296 |
| 80 | GGCUCUGA G ACGCGGCC | 2333 | GGCCGCGU GGAGGAAACUCC CU UCAAGGACAUCGUCCGGG UCAGAGCC | 6297 |
| 85 | UGAGACGC G GCCCCGGC | 2334 | GCCGGGGC GGAGGAAACUCC CU UCAAGGACAUCGUCCGGG GCGUCUCA | 6298 |
| 91 | GCGGCCCC G GCGGCGGC | 2335 | GCCGCCGC GGAGGAAACUCC CU UCAAGGACAUCGUCCGGG GGGGCCGC | 6299 |
| 94 | GCCCCGGC G GCGGCGGC | 2336 | GCCGCCGC CGAGGAAACUCC CU UCAAGGACAUCGUCCGGG GCCGGGGC | 6300 |
| 97 | CCGGCGGC G GCGGCAGC | 2337 | GCUGCCGC GGAGGAAACUCC CU UCAAGGACAUCGUCCGGG GCCGCCGG | 6301 |
| 100 | GCGGCGGC G GGAGCACC | 2338 | GCUGCUGC GGAGGAAACUCC CU UCAAGGACAUCGUCCGCG GCCGCCGC | 6302 |
| 137 | CCAGCCAU G GAAGACCU | 2339 | AGGUCUUC GGAGGAAACUCC CU UCAAGGACAUCGUCCGGG AUGGCUGG | 6303 |
| 138 | CAGCCAUG G AAGACCUG | 2340 | CAGGUCUU GGAGGAAACUCC CU UCAAGGACAUCGUCCGGG CAUGGCUG | 6304 |
| 141 | CCAUGGAA G ACCUGGAC | 2341 | GUCCAGGU GGAGGAAACUCC CU UCAAGGACAUCGUCCGGG UUCCAUGG | 6305 |
| 146 | GAAGACCU G GACCAGUC | 2342 | GACUGGUC GGAGGAAACUCC CU UCAAGGACAUCGUCCGGG AGGUCUUC | 6306 |
| 147 | AAGACCUG G ACCAGUCU | 2343 | AGACUGGU GGAGGAAACUCC CU UCAAGGACAUCGUCCGGG CAGGUCUU | 6307 |
| 161 | UCUCCUCU G GUCUCGUC | 2344 | GACGAGAC GGAGGAAACUCC CU UCAAGGACAUCGUCCGGG AGAGGAGA | 6308 |
| 173 | UCGUCCUC G GACAGCCC | 2345 | GGGCUGUC GGAGGAAACUCC CU UCAAGGACAUCGUCCGGG GAGGACGA | 6309 |
| 174 | CGUCCUCG G ACAGCCCA | 2346 | UGGGCUGU GGAGGAAACUCC CU UCAAGGACAUCGUCCGGG CGAGGACG | 6310 |
| 187 | CCCACCCC G GCCGCAGC | 2347 | GCUGCGGC GGAGGAAACUCC CU UCAAGGACAUCGUCCGGG GGGGUGGG | 6311 |
| 220 | GUUCGUGA G GGAGCCCG | 2348 | CGGGCUCC GGAGGAAACUCC CU UCAAGGACAUCGUCCGGG UCACGAAC | 6312 |
| 221 | UUCGUGAG G GAGCCCGA | 2349 | UCGGGCUC GGAGGAAACUCC CU UCAAGGACAUCGUCCGGG CUCACGAA | 6313 |
| 222 | UCGUGAGG G AGCCCGAG | 2350 | CUCGGGCU GGAGGAAACUCC CU UCAAGGACAUCGUCCGGG CCUCACGA | 6314 |
| 230 | GAGCCCGA G GACGAGGA | 2351 | UCCUCGUC GGAGGAAACUCC CU UCAAGGACAUCGUCCGGG UCGGGCUC | 6315 |
| 231 | AGCCCGAG G ACGAGGAG | 2352 | CUCCUCGU GGAGGAAACUCC CU UCAAGGACAUCGUCCGGG CUCGGGCU | 6316 |
| 236 | GAGGACGA G GAGGAAGA | 2353 | UCUUCCUC GGAGGAAACUCC CU UCAAGGACAUCGUCCGGG UCGUCCUC | 6317 |
| 237 | AGGACGAG G AGGAAGAA | 2354 | UUCUUCCU GGAGGAAACUCC CU UCAAGGACAUCGUCCGGG CUCGUCCU | 6318 |
| 239 | GACGAGGA G GAAGAAGA | 2355 | UCUUCUUC GGAGGAAACUCC CU UCAAGGACAUCGUCCGGG UCCUCGUC | 6319 |
| 240 | ACGAGGAG G AAGAAGAG | 2356 | CUCUUCUU GGAGGAAACUCC CU UCAAGGACAUCGUCCGGG CUCCUCGU | 6320 |
| 243 | AGGAGGAA G AAGAGGAG | 2357 | CUCCUCUU GGAGGAAACUCC CU UCAAGGACAUCGUCCGGG UUCCUCCU | 6321 |
| 246 | AGGAAGAA G AGGAGGAG | 2358 | CUCCUCCU GGAGGAAACUCC CU UCAAGGACAUCGUCCGGG UUCUUCCU | 6322 |
| 248 | GAAGAAGA G GAGGAGGA | 2359 | UCCUCCUC GGAGGAAACUCC CU UCAAGGACAUCGUCCGGG UCUUCUUC | 6323 |
| 249 | AAGAAGAG G AGGAGCAA | 2360 | UUCCUCCU GGAGGAAACUCC CU UCAAGGACAUCGUCCGGG CUCUUCUU | 6324 |
| 251 | GAAGAGGA G GAGGAAGA | 2361 | UCUUCCUC GGAGGAAACUCC CU UCAAGGACAUCGUCCGGG UCCUCUUC | 6325 |
| 252 | AAGAGGAG G AGGAAGAG | 2362 | CUCUUCCU GGAGGAAACUCC CU UCAAGGACAUCGUCCGGG CUCCUCUU | 6326 |
| 254 | GAGGAGGA G GAAGAGGA | 2363 | UCCUCUUC GGAGGAAACUCC CU UCAAGGACAUCGUCCGGG UCCUCCUC | 6327 |
| 255 | AGGAGGAG G AAGAGGAG | 2364 | CUCCUCUU GGAGGAAACUCC CU UCAAGGACAUCGUCCGGG CUCCUCCU | 6328 |
| 258 | AGGAGGAA G AGGAGGAC | 2365 | GUCCUCCU GGAGGAAACUCC CU UCAAGGACAUCGUCCGGG UUCCUCCU | 6329 |
| 260 | GAGGAAGA G GAGGACGA | 2366 | UCGUCCUC GGAGGAAACUCC CU UCAAGGACAUCGUCCGGG UCUUCCUC | 6330 |
| 261 | AGGAACAG G AGGACGAG | 2367 | CUCGUCCU GGAGGAAACUCC CU UCAAGGACAUCGUCCGGG CUCUUCCU | 6331 |
| 263 | GAAGAGGA G GACGAGGA | 2368 | UCCUCGUC GGAGGAAACUCC CU UCAAGGACAUCGUCCGGG UCCUCUUC | 6332 |
| 264 | AAGAGGAG G ACGAGGAC | 2369 | GUCCUCGU GGAGGAAACUCC CU UCAAGGACAUCGUCCGGG CUCCUCUU | 6333 |
| 269 | GAGGACGA G GACGAAGA | 2370 | UCCUCCUC GGAGGAAACUCC CU UCAAGGACAUCGUCCGGG UCGUCCUC | 6334 |
| 270 | AGGACGAG G ACGAAGAC | 2371 | GUCUUCGU GGAGGAAACUCC CU UCAAGGACAUCGUCCGGG CUCGUCCU | 6335 |
| 276 | AGGACGAA G ACCUGGAG | 2372 | CUCCAGGU GGAGGAAACUCC CU UCAAGGACAUCGUCCGGG UUCGUCCU | 6336 |
| 281 | GAAGACCU G GAGGAGCU | 2373 | AGCUCCUC GGAGGAAACUCC CU UCAAGGACAUCGUCCGGG AGGUCUUC | 6337 |
| 282 | AAGACCUG G AGGAGCUG | 2374 | CAGCUCCU GGAGGAAACUCC CU UCAAGGACAUCGUCCGGG CAGGUCUU | 6338 |
| 284 | GACCUGGA G GAGCUGGA | 2375 | UCCAGCUC GGAGGAAACUCC CU UCAAGGACAUCGUCCGGG UCCAGGUC | 6339 |
| 285 | ACCUGGAG G AGCUGGAG | 2376 | CUCCAGCU GGAGGAAACUCC CU UCAAGGACAUCGUCCGGG CUCCAGGU | 6340 |
| 290 | GAGGAGCU G GAGGUGCU | 2377 | AGCACCUC GGAGGAAACUCC CU UCAAGGACAUCGUCCGGG AGCUCCUC | 6341 |
| 291 | AGGACCUC G AGGUGCUG | 2378 | CAGCACCU GGAGGAAACUCC CU UCAAGGACAUCGUCCGGG CAGCUCCU | 6342 |
| 293 | GAGCUGGA G GUGCUGGA | 2379 | UCCAGCAC GGAGGAAACUCC CU UCAAGGACAUCGUCCGGG UCCAGCUC | 6343 |
| 299 | GAGGUGCU G GAGAGGAA | 2380 | UUCCUCUC GGAGGAAACUCC CU UCAAGGACAUCGUCCGGG AGCACCUC | 6344 |
| 300 | AGGUGCUG G AGAGGAAG | 2381 | CUUCCUCU GGAGGAAACUCC CU UCAAGGACAUCGUCCGGG CAGCACCU | 6345 |
| 302 | GUGCUGGA G AGGAAGCC | 2382 | GGCUUCCU GGAGGAAACUCC CU UCAAGGACAUCGUCCGCG UCCAGCAC | 6346 |
| 304 | GCUGGAGA G GAAGCCCG | 2383 | CGGGCUUC GGAGGAAACUCC CU UCAAGGACAUCGUCCGGG UCUCCAGC | 6347 |
| 305 | CUGGAGAG G AAGCCCGC | 2384 | GCGGGCUU GGAGGAAACUCC CU UCAAGGACAUCGUCCGGG CUCUCCAG | 6348 |
| 318 | CCGCCGCC G GGCUGUCC | 2385 | GGACAGCC GGAGGAAACUCC CU UCAAGGACAUCGUCCGGG GGCGGCGG | 6349 |
| 319 | CGCCGCCG G GCUGUCCG | 2386 | CGCACAGC GGAGGAAACUCC CU UCAAGGACAUCGUCCGGG CGGCGGCG | 6350 |
| 329 | CUGUCCGC G GCCCCAGU | 2387 | ACUGGGGC GGAGGAAACUCC CU UCAAGGACAUCGUCCGGG GCGGACAG | 6351 |
| 357 | CUGCCGCC G GCGCGCCC | 2388 | GGGCCCGC GGAGGAAACUCC CU UCAAGGACAUCGUCCGGG GGCGGCAG | 6352 |
| 371 | CCCCUGAU G GACUUCGG | 2389 | CCGAAGUC GGAGGAAACUCC CU UCAAGGACAUCGUCCGGG AUCAGGGG | 6353 |
| 372 | CCCUGAUG G ACUUCGGA | 2390 | UCCGAAGU GGAGGAAACUCC CU UCAAGGACAUCGUCCGGG CAUCAGGG | 6354 |
| 378 | UGGACUUC G GAAAUGAC | 2391 | GUCAUUUC GGAGGAAACUCC CU UCAAGGACAUCGUCCGGG GAAGUCCA | 6355 |
| 379 | GGACUUCG G AAAUGACU | 2392 | AGUCAUUU GGAGGAAACUCC CU UCAAGGACAUCGUCCGGG CGAAGUCC | 6356 |
| 398 | GUGCCGCC G GCGCCCCG | 2393 | CGGGGCGC GGAGGAAACUCC CU UCAAGGACAUCGUCCGGG GGCGGCAC | 6357 |
| 406 | GGCGCCCC G GGGACCCC | 2394 | GGGGUCCC GGAGGAAACUCC CU UCAAGGACAUCGUCCGGG GGGGCGCC | 6358 |
| 407 | GCGCCCCG G GGACCCCU | 2395 | AGGGGUCC GGAGGAAACUCC CU UCAAGGACAUCGUCCGGG CGGGGCGC | 6359 |
| 408 | CGCCCCGG G GACCCCUG | 2396 | CAGGGGUC GGAGGAAACUCC CU UCAAGGACAUCGUCCGGG CCGGGGCG | 6360 |
| 409 | GCCCCGGC G ACCCCUGC | 2397 | GCAGGGGU GGAGGAAACUCC CU UCAAGGACAUCGUCCGGG CCCGGGGC | 6361 |
| 419 | CCCCUGCC G GCCGCUCC | 2398 | CCAGCGGC GGAGGAAACUCC CU UCAAGGACAUCGUCCGGG GGCAGGGG | 6362 |
| 440 | GUCGCCCC G GAGCGGCA | 2399 | UGCCGCUC GGAGGAAACUCC CU UCAAGGACAUCGUCCGGG GGGGCGAC | 6363 |
| 441 | UCGCCCCG G AGCGGCAG | 2400 | CUGCCGCU GGAGGAAACUCC CU UCAAGGACAUCGUCCGGG CGGGGCGA | 6364 |
| 445 | CCCGGAGC G GGAGCCGU | 2401 | ACGGCUGC GGAGCAAACUCC CU UCAAGGACAUCGUCCGGG GCUCCGGG | 6365 |
| 457 | GCCGUCUU G GGACCCGA | 2402 | UCGGGUCC GGAGGAAACUCC CU UCAAGGACAUCGUCCGGG AAGACGGC | 6366 |
| 458 | CCGUCUUG G GACCCGAG | 2403 | CUCGGGUC GGAGGAAACUCC CU UCAAGGACAUCGUCCGGG CAAGACGG | 6367 |
| 459 | CGUCUUGG G ACCCGAGC | 2404 | GCUCGGGU CGAGCAAACUCC CU UCAAGGACAUCGUCCGGG CCAAGACG | 6368 |
| 470 | CCGAGCCC G GUGUCGUC | 2405 | GACGACAC GGAGGAAACUCC CU UCAAGGACAUCGUCCGGG GGGCUCGG | 6369 |
| 539 | CUCCCUGA G GACGACGA | 2406 | UCGUCGUC GGAGGAAACUCC CU UCAAGGACAUCGUCCGGG UCAGGGAG | 6370 |
| 540 | UCCCUGAG G ACGACGAG | 2407 | CUCGUCGU GGAGGAAACUCC CU UCAAGGACAUCGUCCGGG CUCAGGGA | 6371 |
| 554 | GAGCCUCC G GCCCGGCC | 2408 | GGCCGGGC GGAGGAAACUCC CU UCAAGGACAUCGUCCGGG GGAGGCUC | 6372 |
| 559 | UCCGGCCC G GCCUCCCC | 2409 | GGGGAGGC GGAGGAAACUCC CU UCAAGGACAUCGUCCGGG GGGCCGGA | 6373 |
| 578 | CCUCCCCC G GCCAGCGU | 2410 | ACGCUGGC GGAGGAAACUCC CU UCAAGGACAUCGUCCGGG GGGGGAGG | 6374 |
| 596 | AGCCCCCA G GCAGAGCC | 2411 | GGCUCUGC GGAGGAAACUCC CU UCAAGGACAUCGUCCGGG UGGGGGCU | 6375 |
| 600 | CCCAGGCA G AGCGCGUG | 2412 | CACGGGCU GGAGGAAACUCC CU UCAAGGACAUCGUCCGGG UGCCUGGG | 6376 |
| 610 | GCCCGUGU G GACCCCGC | 2413 | GCGGGGUC GGAGGAAACUCC CU UCAAGGACAUCGUCCCGC ACACGGGC | 6377 |
| 611 | CCCGUGUG G ACCCCGCC | 2414 | GGCGGGGU GGAGGAAACUCC CU UCAAGGACAUCGUCCGGG CACACGGG | 6378 |
| 626 | CCAGCCCC G GCUCCCGC | 2415 | GCGGGAGC GGAGGAAACUCC CU UCAAGGACAUCGUCCGGG GGGGCUGG | 6379 |
| 653 | UCCACCCC G GCCGCGCC | 2416 | GGCGCGGC GGAGGAAACUCC CU UCAAGGACAUCGUCCGGG GGGGUGGA | 6380 |
| 670 | CAAGCGCA G GGGCUCCU | 2417 | AGGAGCCC GGAGGAAACUCC CU UCAAGGACAUCGUCCGGG UGCGCUUG | 6381 |
| 671 | AAGCGCAG G GGCUCCUC | 2418 | GAGGAGCC GGAGGAAACUCC CU UCAAGGACAUCGUCCGGG CUGCGCUU | 6382 |
| 672 | AGCGCAGG G GCUCCUCG | 2419 | CGAGGAGC GGAGGAAACUCC CU UCAAGGACAUCGUCCGGG CCUGCGCU | 6383 |
| 680 | GGCUCCUC G GGCUCAGU | 2420 | ACUGAGCC GGAGGAAACUCC CU UCAAGCACAUCGUCCGGG GAGGAGCC | 6384 |
| 681 | GCUCCUCG G GCUCAGUG | 2421 | CACUGAGC GGAGGAAACUCC CU UCAAGGACAUCGUCCGGG CGAGGAGC | 6385 |
| 689 | GGCUCAGU G GAUGAGAC | 2422 | GUCUCAUC GGAGGAAACUCC CU UCAAGGACAUCGUCCGGG ACUGAGCC | 6386 |
| 690 | GCUCAGUG G AUGAGACC | 2423 | GGUCUCAU CGAGGAAACUCC CU UCAAGGACAUCGUCCGGG CACUGAGC | 6387 |
| 695 | GUGGAUGA G ACCCUUUU | 2424 | AAAAGGGU GGAGGAAACUCC CU UCAAGGACAUCGUCCGGG UCAUCCAC | 6388 |
| 747 | CCUCUGCA G AAAAUAUG | 2425 | CAUAUUUU GGAGGAAACUCC CU UCAAGGACAUCGUCCGGG UGCAGAGG | 6389 |
| 755 | GAAAAUAU G GACUUGAA | 2426 | UUCAAGUC GGAGGAAACUCC CU UCAAGGACAUCGUCCGGG AUAUUUUC | 6390 |
| 756 | AAAAUAUG G ACUUGAAG | 2427 | CUUCAAGU GGAGGAAACUCC CU UCAAGGACAUCGUCCGGG CAUAUUUU | 6391 |
| 764 | GACUUGAA G GAGCAGCC | 2428 | GGCUGCUC GGAGGAAACUCC CU UCAAGGACAUCGUCCGGG UUCAAGUC | 6392 |
| 765 | ACUUGAAG G AGCAGCCA | 2429 | UGGCUGCU GGAGGAAACUCC CU UCAAGGACAUCGUCCGGG CUUCAAGU | 6393 |
| 774 | AGCAGCCA G GUAACACU | 2430 | AGUGUUAC GGAGGAAACUCC CU UCAAGGACAUCGUCCGGG UGGCUGCU | 6394 |
| 788 | ACUAUUUC G GCUGGUCA | 2431 | UGACCAGC GGAGGAAACUCC CU UCAAGGACAUCGUCCGGG GAAAUAGU | 6395 |
| 792 | UUUCGGCU G GUCAAGAG | 2432 | CUCUUGAC GGAGGAAACUCC CU UCAAGGACAUCGUCCGGG AGCCGAAA | 6396 |
| 798 | CUGGUCAA G AGGAUUUC | 2433 | GAAAUCCU GGAGGAAACUCC CU UCAAGGACAUCGUCCGGG UUGACCAG | 6397 |
| 800 | GGUCAAGA G GAUUUCCC | 2434 | GGGAAAUC GGAGGAAACUCC CU UCAAGGACAUCGUCCGGG UCUUGACC | 6398 |
| 801 | GUCAAGAG G AUUUCCCA | 2435 | UGGGAAAU GGAGGAAACUCC CU UCAAGGACAUCGUCCGGG CUCUUGAC | 6399 |
| 876 | CUUUCAAA G AACAUGAA | 2436 | UUCAUGUU GGAGGAAACUCC CU UCAAGGACAUCGUCCGGG UUUGAAAG | 6400 |
| 891 | AAUACCUU G GUAAUUUG | 2437 | CAAAUUAC GGAGGAAACUCC CU UCAAGGACAUCGUCCGGG AAGGUAUU | 6401 |
| 921 | CCACUGAA G GAACACUU | 2438 | AAGUGUUC GGAGGAAACUCC CU UCAAGGACAUCGUCCGGG UUCAGUGG | 6402 |
| 922 | CACUGAAG G AACACUUC | 2439 | GAAGUGUU GGAGGAAACUCC CU UCAAGGACAUCGUCCGGG CUUCAGUG | 6403 |
| 933 | CACUUCAA G AAAAUGUC | 2440 | GACAUUUU GGAGGAAACUCC CU UCAAGGACAUCGUCCGGG UUGAAGUG | 6404 |
| 957 | CUUCUAAA G AGGUCUCA | 2441 | UGAGACCU GGAGGAAACUCC CU UCAAGGACAUCGUCCGGG UUUAGAAG | 6405 |
| 959 | UCUAAAGA G GUCUCAGA | 2442 | UCUGAGAC GGAGGAAACUCC CU UCAAGGACAUCGUCCGGG UCUUUAGA | 6406 |
| 966 | AGGUCUCA G AGAAGGCA | 2443 | UGCCUUCU GGAGGAAACUCC CU UCAAGGACAUCGUCCGGG UGAGACCU | 6407 |
| 968 | GUCUCAGA G AAGGCAAA | 2444 | UUUGCCUU GGAGGAAACUCC CU UCAAGGACAUCGUCCGGG UCUGAGAC | 6408 |
| 971 | UCAGAGAA G GCAAAAAC | 2445 | GUUUUUGC GGAGGAAACUCC CU UCAAGGACAUCGUCCGGG UUCUCUGA | 6409 |
| 990 | UACUCAUA G AUAGAGAU | 2446 | AUCUCUAU GGAGGAAACUCC CU UCAAGGACAUCGUCCGGG UAUGAGUA | 6410 |
| 994 | CAUAGAUA G AGAUUUAA | 2447 | UUAAAUCU GGAGGAAACUCC CU UCAAGGACAUCGUCCGGG UAUCUAUG | 6411 |
| 996 | UAGAUAGA G AUUUAACA | 2448 | UGUUAAAU GGAGGAAACUCC CU UCAAGGACAUCGUCCGGG UCUAUCUA | 6412 |
| 1005 | AUUUAACA G AGUUUUCA | 2449 | UGAAAACU GGAGGAAACUCC CU UCAAGGACAUCGUCCGGG UGUUAAAU | 6413 |
| 1014 | AGUUUUCA G AAUUAGAA | 2450 | UUCUAAUU GGAGGAAACUCC CU UCAAGGACAUCGUCCGGG UGAAAACU | 6414 |
| 1020 | CAGAAUUA G AAUACUCA | 2451 | UGAGUAUU GGAGGAAACUCC CU UCAAGGACAUCGUCCGGG UAAUUCUG | 6415 |
| 1029 | AAUACUCA G AAAUGGGA | 2452 | UCCCAUUU GGAGGAAACUCC CU UCAAGGACAUCGUCCGGG UGAGUAUU | 6416 |
| 1034 | UCAGAAAU G GGAUCAUC | 2453 | GAUGAUCC GGAGGAAACUCC CU UCAAGGACAUCGUCCGGG AUUUCUGA | 6417 |
| 1035 | CAGAAAUG G GAUCAUCG | 2454 | CGAUGAUC GGAGGAAACUCC CU UCAAGGACAUCGUCCGGG CAUUUCUG | 6418 |
| 1036 | AGAAAUGG G AUCAUCGU | 2455 | ACGAUGAU GGAGGAAACUCC CU UCAAGGACAUCGUCCGGG CCAUUUCU | 6419 |
| 1065 | CAAAAGCA G AAUCUGCC | 2456 | GGCAGAUU GGAGGAAACUCC CU UCAAGGACAUCGUCCGGG UGCUUUUG | 6420 |
| 1093 | AAAUCCUA G GGAAGAAA | 2457 | UUUCUUCC GGAGGAAACUCC CU UCAAGGACAUCGUCCGGG UAGGAUUU | 6421 |
| 1094 | AAUCCUAG G GAAGAAAU | 2458 | AUUUCUUC GGAGGAAACUCC CU UCAAGGACAUCGUCCGGG CUAGGAUU | 6422 |
| 1095 | AUCCUAGG G AAGAAAUA | 2459 | UAUUUCUU GGAGGAAACUCC CU UCAAGGACAUCGUCCGGG CCUAGGAU | 6423 |
| 1098 | CUAGGGAA G AAAUAAUC | 2460 | GAUUAUUU GGAGGAAACUCC CU UCAAGGACAUCGUCCGGG UUCCCUAG | 6424 |
| 1119 | AAAAUAAA G AUGAACAA | 2461 | UUCUUCAU GGAGGAAACUCC CU UCAAGGACAUCGUCCGGG UUUAUUUU | 6425 |
| 1125 | AAGAUGAA G AAGAGAAG | 2462 | CUUCUCUU GGAGGAAACUCC CU UCAAGGACAUCGUCCGGG UUCAUCUU | 6426 |
| 1128 | AUGAAGAA G AGAAGUUA | 2463 | UAACUUCU GGAGGAAACUCC CU UCAAGGACAUCGUCCGGG UUCUUCAU | 6427 |
| 1130 | GAAGAAGA G AAGUUAGU | 2464 | ACUAACUU GGAGGAAACUCC CU UCAAGGACAUCGUCCGGG UCUUCUUC | 6428 |
| 1167 | AUCAACAA G AGUUACCU | 2465 | AGGUAACU GGAGGAAACUCC CU UCAAGGACAUCGUCCGGG UUGUUGAU | 6429 |
| 1193 | ACUAAAUU G GUUAAAGA | 2466 | UCUUUAAC GGAGGAAACUCC CU UCAAGGACAUCGUCCGGG AAUUUAGU | 6430 |
| 1200 | UGGUUAAA G AGGAUGAA | 2467 | UUCAUCCU GGAGGAAACUCC CU UCAAGGACAUCGUCCGGG UUUAACCA | 6431 |
| 1202 | GUUAAAGA G GAUGAAGU | 2468 | ACUUCAUC GGAGGAAACUCC CU UCAAGGACAUCGUCCGGG UCUUUAAC | 6432 |
| 1203 | UUAAAGAG G AUGAAGUU | 2469 | AACUUCAU GGAGGAAACUCC CU UCAAGGACAUCGUCCGGG CUCUUUAA | 6433 |
| 1221 | UGUCUUCA G AAAAAGCA | 2470 | UGCUUUUU GGAGGAAACUCC CU UCAAGGACAUCGUCCGGG UGAAGACA | 6434 |
| 1233 | AAGCAAAA G ACAGUUUU | 2471 | AAAACUGU GGAGGAAACUCC CU UCAAGGACAUCGUCCGGG UUUUGCUU | 6435 |
| 1250 | AAUGAUAA G AGAGUUGC | 2472 | GCAACUCU GGAGGAAACUCC CU UCAAGGACAUCGUCCGGG UUUUCAUU | 6436 |
| 1252 | UGAAAAGA G AGUUGCAG | 2473 | CUGCAACU GGAGGAAACUCC CU UCAAGGACAUCGUCCGGG UCUUUUCA | 6437 |
| 1262 | GUUGCAGU G GAAGCUCC | 2474 | GGAGCUUC GGAGGAAACUCC CU UCAAGGACAUCGUCCGGG ACUGCAAC | 6438 |
| 1263 | UUGCAGUG G AAGCUCCU | 2475 | AGGAGCUU GGAGGAAACUCC CU UCAAGGACAUCGUCCGGG CACUGCAA | 6439 |
| 1276 | UCCUAUGA G GGAGGAAU | 2476 | AUUCCUCC GGAGGAAACUCC CU UCAAGGACAUCGUCCGGG UCAUAGGA | 6440 |
| 1277 | CCUAUGAG G GAGGAAUA | 2477 | UAUUCCUC GGAGGAAACUCC CU UCAAGGACAUCGUCCGGG CUCAUAGG | 6441 |
| 1278 | CUAUGAGG G AGGAAUAU | 2478 | AUAUUCCU GGAGGAAACUCC CU UCAAGGACAUCGUCCGGG CCUCAUAG | 6442 |
| 1280 | AUGAGGGA G GAAUAUGC | 2479 | GCAUAUUC GGAGGAAACUCC CU UCAAGGACAUCGUCCGGG UCCCUCAU | 6443 |
| 1281 | UGAGGGAG G AAUAUGCA | 2480 | UGCAUAUU GGAGGAAACUCC CU UCAAGGACAUCGUCCGGG CUCCCUCA | 6444 |
| 1290 | AAUAUGCA G ACUUCAAA | 2481 | UUUGAAGU GGAGGAAACUCC CU UCAAGGACAUCGUCCGGG UGCAUAUU | 6445 |
| 1315 | GCGAGUAU G GGAAGUGA | 2482 | UCACUUCC GGAGGAAACUCC CU UCAAGGACAUCGUCCGGG AUACUCGC | 6446 |
| 1316 | CGAGUAUG G GAACUGAA | 2483 | UUCACUUC GGAGGAAACUCC CU UCAAGGACAUCGUCCGGG CAUACUCG | 6447 |
| 1317 | GAGUAUGG G AAGUGAAA | 2484 | UUUCACUU GGAGGAAACUCC CU UCAAGGACAUCGUCCGGG CCAUACUC | 6448 |
| 1326 | AAGUGAAA G AUAGUAAG | 2485 | CUUACUAU GGAGGAAACUCC CU UCAAGGACAUCGUCCGGG UUUCACUU | 6449 |
| 1334 | GAUAGUAA G GAAGAUAG | 2486 | CUAUCUUC GGAGGAAACUCC CU UCAAGGACAUCGUCCGGG UUACUAUC | 6450 |
| 1335 | AUAGUAAG G AAGAUAGU | 2487 | ACUAUCUU GGAGGAAACUCC CU UCAAGGACAUCGUCCGGG CUUACUAU | 6451 |
| 1338 | GUAAGGAA G AUAGUGAU | 2488 | AUCACUAU GGAGGAAACUCC CU UCAAGGACAUCGUCCGGG UUCCUUAC | 6452 |
| 1352 | GAUAUGUU G GCUGCUGG | 2489 | CCAGCAGC GGAGGAAACUCC CU UCAAGGACAUCGUCCGGG AACAUAUC | 6453 |
| 1359 | UGGCUGCU G GAGGUAAA | 2490 | UUUACCUC GGAGGAAACUCC CU UCAAGGACAUCGUCCGGG AGCAGCCA | 6454 |
| 1360 | GGCUGCUG G AGGUAAAA | 2491 | UUUUACCU GGAGGAAACUCC CU UCAAGGACAUCGUCCGGG CAGCAGCC | 6455 |
| 1362 | CUGCUGGA G GUAAAAUC | 2492 | GAUUUUAC GGAGGAAACUCC CU UCAAGGACAUCGUCCGGG UCCAGCAG | 6456 |
| 1373 | AAAAUCGA G AGCAACUU | 2493 | AAGUUGCU GGAGGAAACUCC CU UCAAGGACAUCGUCCGGG UCGAUUUU | 6457 |
| 1382 | AGCAACUU G GAAAGUAA | 2494 | UUACUUUC GGAGGAAACUCC CU UCAAGGACAUCGUCCGGG AAGUUGCU | 6458 |
| 1383 | GCAACUUG G AAAGUAAA | 2495 | UUUACUUU GGAGGAAACUCC CU UCAAGGACAUCGUCCGGG CAAGUUGC | 6459 |
| 1394 | AGUAAAGU G GAUAAAAA | 2496 | UUUUUAUC GGAGGAAACUCC CU UCAAGCACAUCGUCCGGG ACUUUACU | 6460 |
| 1395 | GUAAAGUC G AUAAAAAA | 2497 | UUUUUUAU GGAGGAAACUCC CU UCAAGGACAUCGUCCGGG CACUUUAC | 6461 |
| 1413 | GUUUUGCA G AUAGCCUU | 2498 | AAGGCUAU GGAGGAAACUCC CU UCAAGGACAUCGUCCGGG UGCAAAAC | 6462 |
| 1443 | ACGAAAAA G AUAGUGAG | 2499 | CUCACUAU GGAGGAAACUCC CU UCAAGGACAUCGUCCGGG UUUUUCGU | 6463 |
| 1451 | GAUAGUGA G AGUAGUAA | 2500 | UUACUACU GGAGGAAACUCC CU UCAAGGACAUCGUCCGGG UCACUAUC | 6464 |
| 1488 | GUACGCCA G AAGGUAUA | 2501 | UAUACCUU GGAGGAAACUCC CU UCAAGGACAUCGUCCGGG UGGCGUAC | 6465 |
| 1491 | CGCCAGAA G GUAUAAAG | 2502 | CUUUAUAC GGAGGAAACUCC CU UCAAGGACAUCGUCCGGG UUCUGGCG | 6466 |
| 1499 | GGUAUAAA G GAUCGUUC | 2503 | GAACGAUC GGAGGAAACUCC CU UCAAGGACAUCGUCCGGG UUUAUACC | 6467 |
| 1500 | GUAUAAAG G AUCGUUCA | 2504 | UGAACGAU GGAGGAAACUCC CU UCAAGGACAUCGUCCGGG CUUUAUAC | 6468 |
| 1509 | AUCGUUCA G GAGCAUAU | 2505 | AUAUGCUC GGAGGAAACUCC CU UCAAGGACAUCGUCCGGG UGAACGAU | 6469 |
| 1510 | UCGUUCAG G AGCAUAUA | 2506 | UAUAUGCU GGAGGAAACUCC CU UCAAGGACAUCGUCCCCG CUGAACGA | 6470 |
| 1553 | GCAACUGA G AGCAUUGC | 2507 | GCAAUGCU GGAGGAAACUCC CU UCAAGGACAUCGUCCCCG UCAGUUGC | 6471 |
| 1584 | CUUUGUUA G GAGAUCCU | 2508 | AGGAUCUC GGAGGAAACUCC CU UCAAGGACAUCGUCCCCG UAACAAAG | 6472 |
| 1585 | UUUGUUAG G AGAUCCUA | 2509 | UAGGAUCU GGAGGAAACUCC CU UCAAGGACAUCGUCCGGG CUAACAAA | 6473 |
| 1587 | UGUUAGGA G AUCCUACU | 2510 | AGUAAGAU GGAGGAAACUCC CU UCAAGGACAUCGUCCGGG UCCUAACA | 6474 |
| 1599 | CUACUUCA G AAAAUAAG | 2511 | CUUAUUUU GGAGGAAACUCC CU UCAAGGACAUCGUCCGGG UGAAGUAG | 6475 |
| 1607 | GAAAAUAA G ACCGAUGA | 2512 | UCAUCGGU GGAGGAAACUCC CU UCAAGGACAUCGUCCGGG UUAUUUUC | 6476 |
| 1626 | AAAAAAUA G AAGAAAAG | 2513 | CUUUUCUU GGAGGAAACUCC CU UCAAGGACAUCGUCCGGG UAUUUUUU | 6477 |
| 1629 | AAAUAGAA G AAAAGAAG | 2514 | CUUCUUUU GGAGGAAACUCC CU UCAAGGACAUCGUCCGGG UUCUAUUU | 6478 |
| 1634 | GAAGAAAA G AAGGCCCA | 2515 | UGGGCCUU GGAGGAAACUCC CU UCAAGGACAUCGUCCGGG UUUUCUUC | 6479 |
| 1637 | GAAAAGAA G GCCCAAAU | 2516 | AUUUGGGC GGAGGAAACUCC CU UCAAGGACAUCGUCCGGG UUCUUUUC | 6480 |
| 1653 | UAGUAACA G AGAAGAAU | 2517 | AUUCUUCU GGAGGAAACUCC CU UCAAGGACAUCGUCCGGG UGUUACUA | 6481 |
| 1655 | GUAACACA G AAGAAUAC | 2518 | GUAUUCUU GGAGGAAACUCC CU UCAAGGACAUCGUCCGGG UCUGUUAC | 6482 |
| 1658 | ACAGAGAA G AAUACUAG | 2519 | CUAGUAUU GGAGGAAACUCC CU UCAAGGACAUCGUCCGGG UUCUCUGU | 6483 |
| 1703 | GGAGCACA G GAUUCUGA | 2520 | UCAGAAUC GGAGGAAACUCC CU UCAAGGACAUCGUCCGGG UGUGCUGC | 6484 |
| 1704 | CAGCACAG G AUUCUGAG | 2521 | CUCAGAAU GGAGGAAACUCC CU UCAAGGACAUCGUCCGGG CUGUGCUG | 6485 |
| 1712 | GAUUCUGA G ACAGAUUA | 2522 | UAAUCUGU GGAGGAAACUCC CU UCAAGGACAUCGUCCGGG UCAGAAUC | 6486 |
| 1716 | CUGAGACA G AUUAUGUC | 2523 | GACAUAAU GGAGGAAACUCC CU UCAAGGACAUCGUCCGGG UGUCUCAG | 6487 |
| 1731 | UCACAACA G AUAAUUUA | 2524 | UAAAUUAU GGAGGAAACUCC CU UCAAGGACAUCGUCCGGG UGUUGUGA | 6488 |
| 1745 | UUAACAAA G GUGACUGA | 2525 | UCAGUCAC GGAGGAAACUCC CU UCAAGGACAUCGUCCGGG UUUGUUAA | 6489 |
| 1754 | GUGACUGA G GAAGUCGU | 2526 | ACGACUUC GGAGGAAACUCC CU UCAAGGACAUCGUCCGGG UCAGUCAC | 6490 |
| 1755 | UGACUGAG G AAGUCGUG | 2527 | CACGACUU GGAGGAAACUCC CU UCAAGGACAUCGUCCGGG CUCAGUCA | 6491 |
| 1763 | GAAGUCGU G GCAAACAU | 2528 | AUGUUUGC GGAGGAAACUCC CU UCAAGGACAUCGUCCGGG ACGACUUC | 6492 |
| 1779 | UGCCUGAA G GCCUGACU | 2529 | AGUCACGC GGAGGAAACUCC CU UCAAGGACAUCGUCCGGG UUCAGGCA | 6493 |
| 1791 | UGACUCCA G AUUUAGUA | 2530 | UACUAAAU GGAGGAAACUCC CU UCAAGGACAUCGUCCGGG UGGAGUCA | 6494 |
| 1802 | UUAGUACA G GAAGCAUG | 2531 | CAUGCUUC GGAGGAAACUCC CU UCAAGGACAUCGUCCGGG UGUACUAA | 6495 |
| 1803 | UAGUACAG G AAGCAUGU | 2532 | ACAUGCUU GGAGGAAACUCC CU UCAAGGACAUCGUCCGGG CUGUACUA | 6496 |
| 1836 | AAGUUACU G GUACAAAG | 2533 | CUUUGUAC GGAGGAAACUCC CU UCAAGGACAUCGUCCGGG AGUAACUU | 6497 |
| 1844 | GGUACAAA G AUUGCUUA | 2534 | UAAGCAAU GGAGGAAACUCC CU UCAAGGACAUCGUCCGGG UUUGUACC | 6498 |
| 1865 | ACAAAAAU G GACUUGGU | 2535 | ACCAAGUC GGAGGAAACUCC CU UCAAGGACAUCGUCCGGG AUUUUUGU | 6499 |
| 1866 | CAAAAAUG G ACUUGGUU | 2536 | AACCAAGU GGAGGAAACUCC CU UCAAGGACAUCGUCCGGG CAUUUUUG | 6500 |
| 1871 | AUGGACUU G GUUCAAAG | 2537 | GUUUGAAC GGAGGAAACUCC CU UCAAGGACAUCGUCCGGG AAGUCCAU | 6501 |
| 1884 | AAACAUCA G AAGUUAUC | 2538 | CAUAACUU GGAGGAAACUCC CU UCAAGGACAUCGUCCGGG UGAUGUUU | 6502 |
| 1896 | UUAUGCAA G AGUCACUC | 2539 | GAGUGACU GGAGGAAACUCC CU UCAAGGACAUCGUCCGGG UUGCAUAA | 6503 |
| 1938 | CAUUUGAA G AGUCAGAA | 2540 | UUCUGACU GGAGGAAACUCC CU UCAAGGACAUCGUCCGGG UUCAAAUG | 6504 |
| 1944 | AAGAGUCA G AAGCUACU | 2541 | AGUAGCUU GGAGGAAACUCC CU UCAAGGACAUCGUCCGGG UGACUCUU | 6505 |
| 1982 | AUUGUUAU G GAAGCACC | 2542 | GGUGCUUC GGAGGAAACUCC CU UCAAGGACAUCGUCCGGG AUAACAAU | 6506 |
| 1983 | UUGUUAUG G AAGCACCA | 2543 | UGGUGCUU GGAGGAAACUCC CU UCAAGGACAUCGUCCGGG CAUAACAA | 6507 |
| 2016 | CUAGUGCU G GUGCUUCC | 2544 | GGAAGCAC GGAGGAAACUCC CU UCAAGGACAUCGUCCGGG AGCACUAG | 6508 |
| 2052 | CACCAUUA G AAGCUUCU | 2545 | AGAAGCUU GGAGGAAACUCC CU UCAAGGACAUCGUCCGGG UAAUGGUG | 6509 |
| 2115 | CAUAUGAA G AGGCCAUG | 2546 | CAUGGCCU GGAGGAAACUCC CU UCAAGGACAUCGUCCGGG UUCAUAUG | 6510 |
| 2117 | UAUGAAGA G GCCAUGAG | 2547 | CUCAUGGC GGAGGAAACUCC CU UCAAGGACAUCGUCCGGG UCUUCAUA | 6511 |
| 2148 | AAGUAUCA G GAAUAAAG | 2548 | CUUUAUUC GGAGGAAACUCC CU UCAAGGACAUCGUCCGGG UGAUACUU | 6512 |
| 2149 | AGUAUCAG G AAUAAAGG | 2549 | CCUUUAUU GGAGGAAACUCC CU UCAAGGACAUCGUCCGGG CUGAUACU | 6513 |
| 2156 | GGAAUAAA G GAAGAAAU | 2550 | AUUUCUUC GGAGGAAACUCC CU UCAAGGACAUCGUCCGGG UUUAUUCC | 6514 |
| 2157 | GAAUAAAG G AAGAAAUU | 2551 | AAUUUCUU GGAGGAAACUCC CU UCAAGGACAUCGUCCGGG CUUUAUUC | 6515 |
| 2160 | UAAAGGAA G AAAUUAAA | 2552 | UUUAAUUU GGAGGAAACUCC CU UCAAGGACAUCGUCCGGG UUCCUUUA | 6516 |
| 2169 | AAAUUAAA G AGCCUGAA | 2553 | UUCAGGCU GGAGGAAACUCC CU UCAAGGACAUCGUCCGGG UUUAAUUU | 6517 |
| 2199 | CUCUUCAA G AAACAGAA | 2554 | UUCUGUUU CGAGGAAACUCC CU UCAAGCACAUCGUCCGGG UUGAAGAG | 6518 |
| 2205 | AAGAAACA G AAGCUCCU | 2555 | AGGAGCUU GGAGGAAACUCC CU UCAAGGACAUCGUCCGGG UGUUUCUU | 6519 |
| 2244 | UAAUUAAA G AAACAAAG | 2556 | CUUUGUUU GGAGGAAACUCC CU UCAAGGACAUCGUCCGGG UUUAAUUA | 6520 |
| 2273 | CCAGCUCC G GAUUUCUC | 2557 | GAGAAAUC GGAGGAAACUCC CU UCAAGGACAUCGUCCGGG GGAGCUGG | 6521 |
| 2274 | CAGCUCCG G AUUUCUCU | 2558 | AGAGAAAU GGAGGAAACUCC CU UCAAGGACAUCGUCCGGG CGGAGCUG | 6522 |
| 2292 | AUUAUUCA G AAAUGGCA | 2559 | UGCCAUUU GGAGGAAACUCC CU UCAAGGACAUCGUCCGGG UGAAUAAU | 6523 |
| 2297 | UCAGAAAU G GGAAAAGU | 2560 | ACUUUUGC GGAGGAAACUCC CU UCAAGGACAUCGUCCGGG AUUUCUGA | 6524 |
| 2343 | UAGUUGAA G AUUCCUCA | 2561 | UGAGGAAU GGAGGAAACUCC CU UCAAGGACAUCGUCCGGG UUCAACUA | 6525 |
| 2415 | AAAAACAA G AUGAAACU | 2562 | AGUUUCAU GGAGGAAACUCC CU UCAAGGACAUCGUCCGGG UUGUUUUU | 6526 |
| 2439 | UUGUGAAA G AAAGUCUC | 2563 | GAGACUUU GGAGGAAACUCC CU UCAAGGACAUCGUCCGGG UUUCACAA | 6527 |
| 2453 | CUCACUGA G ACUUCAUU | 2564 | AAUGAAGU CGAGGAAACUCC CU UCAAGGACAUCGUCCGGG UCAGUGAG | 6528 |
| 2475 | CAAUGAUA G AAUAUGAA | 2565 | UUCAUAUU GGAGGAAACUCC CU UCAAGGACAUCGUCCGGG UAUCAUUG | 6529 |
| 2489 | GAAAAUAA G GAAAAACU | 2566 | AGUUUUUC GGAGGAAACUCC CU UCAAGGACAUCGUCCGGG UUAUUUUC | 6530 |
| 2490 | AAAAUAAG G AAAAACUC | 2567 | GAGUUUUU GGAGGAAACUCC CU UCAAGGACAUCGUCCGGG CUUAUUUU | 6531 |
| 2516 | CCACCUGA G GGAGGAAA | 2568 | UUUCCUCC GGAGGAAACUCC CU UCAAGGACAUCGUCCGGG UCAGGUGG | 6532 |
| 2517 | CACCUGAG G GAGGAAAG | 2569 | CUUUCCUC GGAGGAAACUCC CU UCAAGGACAUCGUCCGGG CUCAGGUG | 6533 |
| 2518 | ACCUGAGG G AGGAAAGC | 2570 | GCUUUCCU GGAGGAAACUCC CU UCAAGGACAUCGUCCGGG CCUCAGGU | 6534 |
| 2520 | CUGAGGGA G GAAAGCCA | 2571 | UGGCUUUC GGAGGAAACUCC CU UCAAGGACAUCGUCCGGG UCCCUCAG | 6535 |
| 2521 | UGAGGGAG G AAAGCCAU | 2572 | AUGGCUUU GGAGGAAACUCC CU UCAAGGACAUCGUCCGGG CUCCCUCA | 6536 |
| 2534 | CCAUAUUU G GAAUCUUU | 2573 | AAAGAUUC GGAGGAAACUCC CU UCAAGGACAUCGUCCGGG AAAUAUGG | 6537 |
| 2535 | CAUAUUUC G AAUCUUUU | 2574 | AAAAGAUU GGAGGAAACUCC CU UCAAGGACAUCGUCCGGG CAAAUAUG | 6538 |
| 2556 | UCAGUUUA G AUAACACA | 2575 | UGUGUUAU GGAGGAAACUCC CU UCAAGGACAUCGUCCCCG UAAACUGA | 6539 |
| 2568 | ACACAAAA G AUACCCUG | 2576 | CAGGGUAU GGAGGAAACUCC CU UCAAGGACAUCGUCCGGG UUUUGUGU | 6540 |
| 2609 | AGCAAAAA G GAGAAAAU | 2577 | AUUUUCUC GGAGGAAACUCC CU UCAAGGACAUCGUCCGGG UUUUUGCU | 6541 |
| 2610 | GCAAAAAG G AGAAAAUU | 2578 | AAUUUUCU GGAGGAAACUCC CU UCAAGGACAUCGUCCGGG CUUUUUGC | 6542 |
| 2612 | AAAAAGGA G AAAAUUCC | 2579 | GGAAUUUU GGAGGAAACUCC CU UCAAGGACAUCGUCCGGG UCCUUUUU | 6543 |
| 2627 | CCUUUGCA G AUGGAGGA | 2580 | UCCUCCAU GGAGGAAACUCC CU UCAAGGACAUCGUCCGGG UGCAAAGG | 6544 |
| 2630 | UUGCAGAU G GAGGAGCU | 2581 | AGCUCCUC GGAGGAAACUCC CU UCAAGGACAUCGUCCGGG AUCUGCAA | 6545 |
| 2631 | UGCAGAUG G AGGAGCUC | 2582 | GAGCUCCU GGAGGAAACUCC CU UCAAGGACAUCGUCCGGG CAUCUGCA | 6546 |
| 2633 | CAGAUGGA G GAGCUCAG | 2583 | CUGAGCUC GGAGGAAACUCC CU UCAAGGACAUCGUCCGGG UCCAUCUG | 6547 |
| 2634 | AGAUGGAG G AGCUCAGU | 2584 | ACUGAGCU GGAGGAAACUCC CU UCAAGGACAUCGUCCGGG CUCCAUCU | 6548 |
| 2681 | AUUUCUAA G GAAGCACA | 2585 | UGUGCUUC GGAGGAAACUCC CU UCAAGGACAUCGUCCGGG UUAGAAAU | 6549 |
| 2682 | UUUCUAAG G AAGCACAG | 2586 | CUGUGCUU GGAGGAAACUCC CU UCAAGGACAUCGUCCGGG CUUAGAAA | 6550 |
| 2690 | GAAGCACA G AUAAGAGA | 2587 | UCUCUUAU GGAGGAAACUCC CU UCAAGGACAUCGUCCGGG UGUGCUUC | 6551 |
| 2695 | ACAGAUAA G AGAAACUG | 2588 | CAGUUUCU GGAGGAAACUCC CU UCAAGGACAUCGUCCGGG UUAUCUGU | 6552 |
| 2697 | AGAUAAGA G AAACUGAA | 2589 | UUCAGUUU GGAGGAAACUCC CU UCAAGGACAUCGUCCGGG UCUUAUCU | 6553 |
| 2715 | CGUUUUCA G AUUCAUCU | 2590 | AGAUGAAU GGAGGAAACUCC CU UCAAGGACAUCGUCCGGG UGAAAACG | 6554 |
| 2739 | AAAUUAUA G AUGAGUUC | 2591 | GAACUCAU GGAGGAAACUCC CU UCAAGGACAUCGUCCGGG UAUAAUUU | 6555 |
| 2794 | AUUAGCCA G GGAAUAUA | 2592 | UAUAUUCC GGAGGAAACUCC CU UCAAGGACAUCGUCCGGG UGGCUAAU | 6556 |
| 2795 | UUAGCCAG G GAAUAUAC | 2593 | GUAUAUUC CCAGGAAACUCC CU UCAAGGACAUCGUCCGGG CUGGCUAA | 6557 |
| 2796 | UAGCCAGG G AAUAUACU | 2594 | AGUAUAUU GGAGGAAACUCC CU UCAAGGACAUCGUCCGGG CCUGGCUA | 6558 |
| 2811 | CUCACCUA G AAGUAUCC | 2595 | GGAUACUU GGAGGAAACUCC CU UCAAGGACAUCGUCCGGG UAGGUCAG | 6559 |
| 2846 | AAUGCCCC G GAUGGAGC | 2596 | GCUCCAUC GGAGGAAACUCC CU UCAAGGACAUCGUCCGGG GGGGCAUU | 6560 |
| 2847 | AUGCCCCG G AUGGAGCU | 2597 | AGCUCCAU GGAGGAAACUCC CU UCAAGGACAUCGUCCGGG CGGGGCAU | 6561 |
| 2850 | CCCCGGAU G GAGCUGGG | 2598 | CCCAGCUC GGAGGAAACUCC CU UCAAGGACAUCGUCCGGG AUCCGGGG | 6562 |
| 2851 | CCCGGAUC G AGCUGGGU | 2599 | ACCCAGCU GGAGGAAACUCC CU UCAAGGACAUCGUCCGGG CAUCCGGG | 6563 |
| 2856 | AUGGAGCU G GGUCAUUG | 2600 | CAAUGACC CGAGGAAACUCC CU UCAAGGACAUCGUCCGGG AGCUCCAU | 6564 |
| 2857 | UGGAGCUG G GUCAUUGC | 2601 | GCAAUGAC GGAGGAAACUCC CU UCAAGGACAUCGUCCGGG CAGCUCCA | 6565 |
| 2874 | CUUGCACA G AAUUGCCC | 2602 | GGGCAAUU GGAGGAAACUCC CU UCAAGGACAUCGUCCGGG UGUGCAAG | 6566 |
| 2900 | UCUUUGAA G AACAUACA | 2603 | UGUAUGUU GGAGGAAACUCC CU UCAAGGACAUCGUCCGGG UUCAAAGA | 6567 |
| 2922 | AAGUUCAA G AGAAAAUC | 2604 | GAUUUUCU GGAGGAAACUCC CU UCAAGGACAUCGUCCGGG UUCAACUU | 6568 |
| 2924 | GUUGAAGA G AAAAUCAG | 2605 | CUGAUUUU GGAGGAAACUCC CU UCAAGGACAUCGUCCGGG UCUUCAAC | 6569 |
| 2940 | GUUUCUCA G AUGACUUU | 2606 | AAAGUCAU GGAGGAAACUCC CU UCAAGGACAUCGUCCGGG UGAGAAAC | 6570 |
| 2958 | CUAAAAAU G GGUCUGCU | 2607 | AGCAGACC GGAGGAAACUCC CU UCAAGGACAUCGUCCGGG AUUUUUAG | 6571 |
| 2959 | UAAAAAUG G GUCUGCUA | 2608 | UAGCAGAC GGAGGAAACUCC CU UCAAGGACAUCGUCCGGG CAUUUUUA | 6572 |
| 2975 | ACAUCAAA G GUGCUCUU | 2609 | AAGAGCAC GGAGGAAACUCC CU UCAAGGACAUCGUCCGGG UUUGAUGU | 6573 |
| 2994 | UGCCUCCA G AUGUUUCU | 2610 | AGAAACAU CGAGGAAACUCC CU UCAAGGACAUCGUCCGGG UGGAGGCA | 6574 |
| 3008 | UCUGCUUU G GCCACUCA | 2611 | UGAGUGGC GGAGGAAACUCC CU UCAAGGACAUCGUCCCCG UAAGCAGA | 6575 |
| 3021 | CUCAAGCA G ACAUAGAG | 2612 | CUCUAUCU GGAGGAAACUCC CU UCAAGGACAUCGUCCGGG AGCUUGAG | 6576 |
| 3023 | CAAGCAGA G AUAGAGAG | 2613 | CUCUCUAU GGAGGAAACUCC CU UCAAGGACAUCGUCCGGG UCUGCUUG | 6577 |
| 3027 | CAGAGAUA G AGAGCAUA | 2614 | UAUGCUCU GGAGGAAACUCC CU UCAAGGACAUCGUCCGGG UAUCUCUG | 6578 |
| 3029 | GAGAUAGA G AGCAUAGU | 2615 | ACUAUGCU GGAGGAAACUCC CU UCAAGGACAUCGUCCGGG UCUAUCUC | 6579 |
| 3060 | UUGUGAAA G AAGCUGAG | 2616 | CUCAGCUU GGAGGAAACUCC CU UCAAGGACAUCGUCCGGG UUUCACAA | 6580 |
| 3068 | GAAGCUGA G AAAAAACU | 2617 | AGUUUUUU GGAGGAAACUCC CU UCAAGGACAUCGUCCGGG UCAGCUUC | 6581 |
| 3090 | CCGAUACA G AAAAAGAG | 2618 | CUCUUUUU GGAGGAAACUCC CU UCAAGGACAUCGUCCGGG UGUAUCGG | 6582 |
| 3096 | CAGAAAAA G AGGACAGA | 2619 | UCUGUCCU GGAGGAAACUCC CU UCAAGGACAUCGUCCGGG UUUUUCUG | 6583 |
| 3098 | GAAAAAGA G GACAGAUC | 2620 | GAUCUGUC GGAGGAAACUCC CU UCAAGGACAUCGUCCGGG UCUUUUUC | 6584 |
| 3099 | AAAAAGAG G ACAGAUCA | 2621 | UGAUCUGU GGAGGAAACUCC CU UCAAGGACAUCGUCCGGG CUCUUUUU | 6585 |
| 3103 | AGAGGACA G AUCACCAU | 2622 | AUGGUGAU GGAGGAAACUCC CU UCAAGGACAUCGUCCGGG UGUCCUCU | 6586 |
| 3129 | UUUCAGCA G AGCUGAGU | 2623 | ACUCAGCU GGAGGAAACUCC CU UCAAGGACAUCGUCCGGG UGCUGAAA | 6587 |
| 3166 | CCUGUACU G GAGAGACA | 2624 | UGUCUCUC GGAGGAAACUCC CU UCAAGGACAUCGUCCGGG AGUACAGG | 6588 |
| 3167 | CUGUACUG G AGAGACAU | 2625 | AUGUCUCU GGAGGAAACUCC CU UCAAGGACAUCGUCCGGG CAGUACAG | 6589 |
| 3169 | GUACUGGA G AGACAUUA | 2626 | UAAUGUCU GGAGGAAACUCC CU UCAAGGACAUCGUCCGGG UCCAGUAC | 6590 |
| 3171 | ACUGGAGA G ACAUUAAG | 2627 | CUUAAUGU GGAGGAAACUCC CU UCAAGGACAUCGUCCGGG UCUCCAGU | 6591 |
| 3179 | GACAUUAA G AAGACUGG | 2628 | CCAGUCUU GGAGGAAACUCC CU UCAAGGACAUCGUCCGGG UUAAUGUC | 6592 |
| 3182 | AUUAAGAA G ACUGGAGU | 2629 | ACUCCAGU GGAGGAAACUCC CU UCAAGGACAUCGUCCGGG UUCUUAAU | 6593 |
| 3186 | AGAAGACU G GAGUGGUG | 2630 | CACCACUC GGAGGAAACUCC CU UCAAGGACAUCGUCCGGG AGUCUUCU | 6594 |
| 3187 | GAAGACUG G AGUGGUGU | 2631 | ACACCACU GGAGGAAACUCC CU UCAAGGACAUCGUCCGGG CAGUCUUC | 6595 |
| 3191 | ACUGGAGU G GUGUUUGG | 2632 | CCAAACAC GGAGGAAACUCC CU UCAAGGACAUCGUCCGGG ACUCCAGU | 6596 |
| 3198 | UGGUGUUU G GUGCCAGC | 2633 | GCUGGCAC GGAGGPAACUCC CU UCAAGGACAUCGUCCGGG AAACACCA | 6597 |
| 3269 | AUUGCCUU G GCCCUGCU | 2634 | AGCAGGGC GGAGGAAACUCC CU UCAAGGACAUCGUCCGGG AAGGCAAU | 6598 |
| 3298 | CAGCUUUA G GAUAUACA | 2635 | UGUAUAUC GGAGGAAACUCC CU UCAAGGACAUCGUCCGGG UAAAGCUG | 6599 |
| 3299 | AGCUUUAG G AUAUACAA | 2636 | UUGUAUAU GGAGGAAACUCC CU UCAAGGACAUCGUCCGGG CUAAAGCU | 6600 |
| 3308 | AUAUACAA G GGUGUGAU | 2637 | AUCACACC GGAGGAAACUCC CU UCAAGGACAUCGUCCGGG UUGUAUAU | 6601 |
| 3309 | UAUACAAG G GUGUGAUC | 2638 | GAUCACAC GGAGGAAACUCC CU UCAAGGACAUCGUCCGGG CUUGUAUA | 6602 |
| 3329 | GCUAUCCA G AAAUCAGA | 2639 | UCUGAUUU GGAGGAAACUCC CU UCAAGGACAUCGUCCGGG UGGAUAGC | 6603 |
| 3336 | AGAAAUCA G AUGAAGGC | 2640 | GCCUUCAU GGAGGAAACUCC CU UCAAGGACAUCGUCCGGG UGAUUUCU | 6604 |
| 3342 | CAGAUGAA G GCCACCCA | 2641 | UGGGUGGC GGAGGAAACUCC CU UCAAGGACAUCGUCCGGG UUCAUCUG | 6605 |
| 3355 | CCCAUUCA G GGCAUAUC | 2642 | GAUAUGCC GGAGGAAACUCC CU UCAAGGACAUCGUCCGGG UGAAUGGG | 6606 |
| 3356 | CCAUUCAG G GCAUAUCU | 2643 | AGAUAUGC GGAGGAAACUCC CU UCAAGGACAUCGUCCGGG CUGAAUGG | 6607 |
| 3365 | GGAUAUCU G GAAUCUGA | 2644 | UCAGAUUC GGAGGAAACUCC CU UCAAGGACAUCGUCCGGG AGAUAUGC | 6608 |
| 3366 | CAUAUCUG G AAUCUGAA | 2645 | UUCAGAUU GGAGGAAACUCC CU UCAAGGACAUCGUCCGGG CAGAUAUG | 6609 |
| 3389 | AUAUCUGA G GAGUUGGU | 2646 | ACCAACUC GGAGGAAACUCC CU UCAAGGACAUCGUCCGGG UCAGAUAU | 6610 |
| 3390 | UAUCUGAG G AGUUGGUU | 2647 | AACCAACU GGAGGAAACUCC CU UCAAGGACAUCGUCCGGG CUCAGAUA | 6611 |
| 3395 | GAGGAGUU G GUUCAGAA | 2648 | UUCUGAAC GGAGGAAACUCC CU UCAAGGACAUCGUCCGGG AACUCCUC | 6612 |
| 3401 | UUGGUUCA G AAGUACAG | 2649 | CUGUACUU GGAGGAAACUCC CU UCAAGGACAUCGUCCGGG UGAACCAA | 6613 |
| 3423 | CUGCUCUU G GUCAUGUG | 2650 | CACAUGAC GGAGGAAACUCC CU UCAAGGACAUCGUCCGGG AAGAGCAG | 6614 |
| 3446 | ACGAUAAA G GAACUCAG | 2651 | CUGAGUUC GGAGGAAACUCC CU UCAAGGACAUCGUCCGGG UUUAUCGU | 6615 |
| 3447 | CGAUAAAG G AACUCAGG | 2652 | CCUGAGUU GGAGGAAACUCC CU UCAAGGACAUCGUCCGGG CUUUAUCG | 6616 |
| 3454 | GGAACUCA G GCGCCUCU | 2653 | AGAGGCGC GGAGGAAACUCC CU UCAAGGACAUCGUCCGGG UGAGUUCC | 6617 |
| 3511 | GUUGAUGU G GGUAUUUA | 2654 | UAAAUACC GGAGGAAACUCC CU UCAAGGACAUCGUCCGGG ACAUCAAC | 6618 |
| 3512 | UUGAUGUG G GUAUUUAC | 2655 | GUAAAUAC GGAGGAAACUCC CU UCAAGGACAUCGUCCGGG CACAUCAA | 6619 |
| 3528 | CCUAUGUU G GUGCCUUG | 2656 | CAAGGCAC GGAGGAAACUCC CU UCAAGGACAUCGUCCGGG AACAUAGG | 6620 |
| 3543 | UGUUUAAU G GUCUGACA | 2657 | UGUCAGAC GGAGGAAACUCC CU UCAAGGACAUCGUCCGGG AUUAAACA | 6621 |
| 3563 | CUGAUUUU G GCUCACAU | 2658 | AUGAGAGC GGAGGAAACUCC CU UCAAGGACAUCGUCCGGG AAAAUCAG | 6622 |
| 3604 | UUAUGAAC G GCAUCAGG | 2659 | CCUGAUGC GGAGGAAACUCC CU UCAAGGACAUCGUCCGGG GUUCAUAA | 6623 |
| 3611 | CGGCAUCA G GCACAGAU | 2660 | AUCUGUGC GGAGGAAACUCC CU UCAAGGACAUCGUCCGGG UGAUGCCG | 6624 |
| 3617 | CAGGCACA G AUAGAUCA | 2661 | UGAUCUAU GGAGGAAACUCC CU UCAAGGACAUCGUCCGGG UGUGCCUG | 6625 |
| 3621 | CACAGAUA G AUCAUUAU | 2662 | AUAAUGAU GGAGGAAACUCC CU UCAAGGACAUCGUCCGGG UAUCUGUG | 6626 |
| 3633 | AUUAUCUA G GACUUGCA | 2663 | UGCAAGUC GGAGGAAACUCC CU UCAAGGACAUCGUCCGGG UAGAUAAU | 6627 |
| 3634 | UUAUCUAG G ACUUGCAA | 2664 | UUGCAAGU GGAGGAAACUCC CU UCAAGGACAUCGUCCGGG CUAGAUAA | 6628 |
| 3647 | GGAAAUAA G AAUGUUAA | 2665 | UUAACAUU GGAGGAAACUCC CU UCAAGGACAUCGUCCGGG UUAUUUGC | 6629 |
| 3657 | AUGUUAAA G AUGCUAUG | 2666 | CAUAGCAU GGAGGAAACUCC CU UCAAGGACAUCGUCCGGG UUUAACAU | 6630 |
| 3665 | GAUGCUAU G GCUAAAAU | 2667 | AUUUUACC GGAGGAAACUCC CU UCAAGGACAUCGUCCGGG AUAGCAUC | 6631 |
| 3690 | AAAUCCCU G GAUUGAAG | 2668 | CUUCAAUC GGAGGAAACUCC CU UCAAGGACAUCGUCCGGG AGGGAUUU | 6632 |
| 3691 | AAUCCCUG G AUUGAAGC | 2669 | GCUUCAAU GGAGGAAACUCC CU UCAAGGACAUCGUCCGGG CAGGGAUU | 6633 |
| 3735 | AAUUAGUA G GAGUUCAU | 2670 | AUGAACUC GGAGGAAACUCC CU UCAAGGACAUCGUCCGGG UACUAAUU | 6634 |
| 3736 | AUUAGUAG G AGUUCAUC | 2671 | GAUGAACU GGAGGAAACUCC CU UCAAGGACAUCGUCCGGG CUACUAAU | 6635 |
| 3751 | UCUUUAAA G GGGAUAUU | 2672 | AAUAUCCC GGAGGAAACUCC CU UCAAGGACAUCGUCCGGG UUUAAAGA | 6636 |
| 3752 | CUUUAAAG G GGAUAUUC | 2673 | GAAUAUCC GGAGGAAACUCC CU UCAAGGACAUCGUCCGGG CUUUAAAG | 6637 |
| 3753 | UUUAAAGG G GAUAUUCA | 2674 | UGAAUAUC GGAGGAAACUCC CU UCAAGGACAUCGUCCGGG CCUUUAAA | 6638 |
| 3754 | UUAAAGGG G AUAUUCAU | 2675 | AUGAAUAU GGAGGAAACUCC CU UCAAGGACAUCGUCCGGG CCCUUUAA | 6639 |
| 3773 | GAUUAUAC G GGGGAGGG | 2676 | CCCUCCCC GGAGGAAACUCC CU UCAAGGACAUCGUCCGGG GUAUAAUC | 6640 |
| 3774 | AUUAUACG G GGGAGGGU | 2677 | ACCCUCCC GGAGGAAACUCC CU UCAAGGACAUCGUCCGGG CGUAUAAU | 6641 |
| 3775 | UUAUACGG G GGAGGGUC | 2678 | GACCCUCC GGAGGAAACUCC CU UCAAGGACAUCGUCCGGG CCGUAUAA | 6642 |
| 3776 | UAUACGGG G GAGGGUCA | 2679 | UGACCCUC GGAGGAAACUCC CU UCAAGGACAUCGUCCGGG CCCGUAUA | 6643 |
| 3777 | AUACGGCG G ACGGUCAG | 2680 | CUGACCCU GGAGGAAACUCC CU UCAAGGACAUCGUCCGGG CCCCGUAU | 6644 |
| 3779 | ACGGGGGA G GGUCAGGG | 2681 | CCCUGACC GGAGGAAACUCC CU UCAAGGACAUCGUCCGGG UCCCCCGU | 6645 |
| 3780 | CGGGGGAG G GUCAGGGA | 2682 | UCCCUGAC GGAGGAAACUCC CU UCAAGGACAUCGUCCGGG CUCCCCCG | 6646 |
| 3785 | GAGGGUCA G GGAAGAAC | 2683 | GUUCUUCC GGAGGAAACUCC CU UCAAGGACAUCGUCCGGG UGACCCUC | 6647 |
| 3786 | AGGGUCAG G GAAGAACG | 2684 | CGUUCUUC GGAGGAAACUCC CU UCAAGGACAUCGUCCGGG CUGACCCU | 6648 |
| 3787 | GGGUCAGG G AAGAACGA | 2685 | UCGUUCUU GGAGGAAACUCC CU UCAAGGACAUCGUCCGGG CCUGACCC | 6649 |
| 3790 | UCAGGGAA G AACGAACC | 2686 | GGUUCGGG GGAGGAAACUCC CU UCAAGGACAUCGUCCGGG UUCCCUGA | 6650 |
| 3823 | GUUUCACA G AUCGUUGU | 2687 | ACAACGAU GGAGGAAACUCC CU UCAAGGACAUCGUCCGGG UGUGAAAC | 6651 |
| 3834 | CGUUGUUA G AUCUUUAU | 2688 | AUAAAGAU GGAGGAAACUCC CU UCAAGGACAUCGUCCGGG UAACAACG | 6652 |
| 3865 | UGUUGUGA G GAAAAAUU | 2689 | AAUUUUUC GGAGGAAACUCC CU UCAAGGACAUCGUCCGGG UCACAACA | 6653 |
| 3866 | GUUGUGAG G AAAAAUUA | 2690 | UAAUUUUU GGAGGAAACUCC CU UCAAGGACAUCGUCCGGG CUCACAAC | 6654 |
| 3929 | CUAUGUAU G GAUUUAAA | 2691 | UUUAAAUC GGAGGAAACUCC CU UCAAGGACAUCGUCCGGG AUACAUAG | 6655 |
| 3930 | UAUGUAUG G AUUUAAAC | 2692 | GUUUAAAU GGAGGAAACUCC CU UCAAGGACAUCGUCCGGG CAUACAUA | 6656 |

What we claim is:

1. A nucleic acid molecule which down regulates expression of a neurite growth inhibitor gene.

2. A nucleic acid molecule of claim 1, wherein said neurite growth inhibitor gene is a NOGO gene.

3. The nucleic acid of claim 1, wherein said nucleic acid molecule is adapted for use to treat conditions selected from the group consisting of CNS injury and cerebrovascular accident.

4. The nucleic acid molecule of claim 1 or claim 2, wherein said nucleic acid molecule is an enzymatic nucleic acid molecule having at least one binding arm.

5. The nucleic acid molecule of claim 4, wherein said enzymatic nucleic acid molecule has an endonuclease activity to cleave RNA encoded by a NOGO gene.

6. The nucleic acid of claim 4, wherein one or more binding arms of the enzymatic nucleic acid molecule comprises a sequence complementary to a sequence selected from the group consisting of SEQ ID NOs. 1-2701.

7. An enzymatic nucleic acid molecule comprising a sequence selected from the group consisting of SEQ ID NOs. 2702-6665.

8. The nucleic acid molecule of claim 1, wherein said nucleic acid molecule is an antisense nucleic acid molecule.

9. An antisense nucleic acid molecule comprising a sequence complementary to a sequence selected from the group consisting of SEQ ID NOs. 1-2701.

10. The enzymatic nucleic acid molecule of claim 4, wherein said enzymatic nucleic acid molecule is in a hammerhead (HH) motif.

11. The enzymatic nucleic acid molecule of claim 4, wherein said enzymatic nucleic acid molecule is in a hairpin, hepatitis Delta virus, group I intron, VS nucleic acid, amberzyme, zinzyme or RNAse P nucleic acid motif.

12. The enzymatic nucleic acid molecule of claim 11, wherein said zinzyme motif comprises a sequence selected from the group consisting of SEQ ID NOs. 4480-4895.

13. The enzymatic nucleic acid molecule of claim 11, wherein said amberzyme motif comprises a sequence selected from the group consisting of SEQ ID NOs. 5749-6665.

14. The enzymatic nucleic acid molecule of claim 4, wherein said enzymatic nucleic acid molecule is in a NCH motif.

15. The enzymatic nucleic acid molecule of claim 4, wherein said enzymatic nucleic acid molecule is in a G-cleaver motif.

16. The enzymatic nucleic acid molecule of claim 4, wherein said enzymatic nucleic acid molecule is a DNAzyme.

17. The nucleic acid molecule of claim 2, wherein said nucleic acid molecule comprises between 12 and 100 bases complementary to the RNA of NOGO gene.

18. The nucleic acid molecule of claim 2, wherein said nucleic acid molecule comprises between 14 and 24 bases complementary to the RNA of NOGO gene.

19. The nucleic acid molecule of claim 1, wherein said nucleic acid molecule is chemically synthesized.

20. The nucleic acid molecule of claim 1, wherein said nucleic acid molecule comprises at least one 2′-sugar modification.

21. The nucleic acid molecule of claim 1, wherein said nucleic acid molecule comprises at least one nucleic acid base modification.

22. The nucleic acid molecule of claim 1, wherein said nucleic acid molecule comprises at least one phosphate backbone modification.

23. A mammalian cell including the nucleic acid molecule of claim 1, wherein said mammalian cell is not a living human.

24. The mammalian cell of claim 23, wherein said mammalian cell is a human cell.

25. A method of reducing NOGO activity in a cell, comprising the step of contacting said cell with the nucleic acid molecule of claim 2, under conditions suitable for said inhibition.

26. A method of treatment of a patient having a condition associated with the level of NOGO, comprising contacting cells of said patient with the nucleic acid molecule of claim 2, under conditions suitable for said treatment.

27. The method of claim 26 further comprising the use of one or more drug therapies under conditions suitable for said treatment.

28. A method of cleaving RNA of NOGO gene contacting the nucleic acid molecule of claim 2 with said RNA under conditions suitable for the cleavage of said RNA.

29. The method of claim 28, wherein said cleavage is carried out in the presence of a divalent cation.

30. The method of claim 29, wherein said divalent cation is Mg²⁺.

31. The nucleic acid molecule of claim 1, wherein said nucleic acid comprises a cap structure, wherein the cap structure is at the 5′-end or 3′-end or both the 5′-end and the 3′-end.

32. The enzymatic nucleic acid molecule of claim 10, wherein said hammerhead motif comprises a sequence selected from the group consisting of SEQ ID NOs. 2702-3431.

33. The enzymatic nucleic acid molecule of claim 14, wherein said NCH motif comprises a sequence selected from the group consisting of SEQ ID NOs. 3432-4245.

34. The enzymatic nucleic acid molecule of claim 15, wherein said G-cleaver motif comprises a sequence selected from the group consisting of SEQ ID NOs. 4246-4479.

35. The enzymatic nucleic acid molecule of claim 16, wherein said DNAzyme comprise a sequence selected from the group consisting of SEQ ID NOs. 4896-5748.

36. The method of claim 25, wherein said nucleic acid molecule is in a hammerhead motif.

37. The method of claim 25, wherein said nucleic acid molecule is a DNAzyme.

38. An expression vector comprising a nucleic acid sequence encoding at least one nucleic acid molecule of claim 1 in a manner which allows expression of the nucleic acid molecule.

39. A mammalian cell including an expression vector of claim 38, wherein said mammalian cell is not a living human.

40. The mammalian cell of claim 39, wherein said mammalian cell is a human cell.

41. The expression vector of claim 38, wherein said nucleic acid molecule is in a hammerhead motif.

42. The expression vector of claim 38, wherein said expression vector further comprises a sequence for an antisense nucleic acid molecule complementary to the RNA of NOGO gene.

43. The expression vector of claim 38, wherein said expression vector comprises a nucleic acid sequence encoding two or more of said nucleic acid molecules, which may be the same or different.

44. The expression vector of claim 43, wherein said expression vector comprises a sequence encoding antisense nucleic acid molecule complementary to the RNA of NOGO gene.

45. A method for treatment of conditions selected from the group consisting of CNS injury and cerebrovascular accident comprising the step of administering to a patient the nucleic acid molecule of claim 1 under conditions suitable for said treatment.

46. The method of claim 45, wherein said treatment of CNS injury is treatment of spinal cord injury.

47. A method for treatment of conditions selected from the group consisting of CNS injury and cerebrovascular accident comprising the step of administering to a patient the antisense nucleic acid molecule of claim 9 under conditions suitable for said treatment.

48. The method of claim 45, wherein said nucleic acid molecule is in a hammerhead motif.

49. The method of claim 45, wherein said method further comprises administering to said patient one or more other therapies.

50. The nucleic acid molecule of claim 1, wherein said nucleic acid molecule comprises at least five ribose residues, at least ten 2′-O-methyl modifications, and a 3′-end modification.

51. The nucleic acid molecule of claim 50, wherein said nucleic acid molecule further comprises phosphorothioate linkages on at least three of the 5′ terminal nucleotides.

52. The nucleic acid molecule of claim 50, wherein said 3′-end modification is 3′-3′ inverted abasic moiety.

53. The enzymatic nucleic acid molecule of claim 16, wherein said DNAzyme comprises at least ten 2′-O-methyl modifications and a 3′-end modification.

54. The enzymatic nucleic acid molecule of claim 53, wherein said DNAzyme further comprises phosphorothioate linkages on at least three of the 5′ terminal nucleotides.

55. The enzymatic nucleic acid molecule of claim 53, wherein said 3′-end modification is 3′-3′ inverted abasic moiety.
